(19) United States
(12) Patent Application Publication
(10) Pub. No.: US 2024/0395358 A1
(43) Pub. Date: Nov. 28, 2024

(54) METHODS AND COMPOSITIONS FOR ANALYZING NUCLEIC ACID

(71) Applicant: Claret Bioscience, LLC, Santa Cruz, CA (US)

(72) Inventor: Charles VASKE, Santa Cruz, CA (US)

(21) Appl. No.: 18/029,550

(22) PCT Filed: Oct. 6, 2021
(86) PCT No.: PCT/US2021/053791

§ 371 Date: Mar. 30, 2023

Related U.S. Application Data
(60) Provisional application No. 63/089,191, filed on Oct. 8, 2020.

(51) Int. Cl. G16B 20/20; G16B 30/00

(52) U.S. Cl. CPC G16B 20/20; G16B 30/00

(57) ABSTRACT

The technology relates in part to methods and compositions for analyzing nucleic acid. In some aspects, the technology relates to methods and compositions for generating one or more genotypes for a sample. In some aspects, the technology relates to methods and compositions for generating one or more genotypes for a mixed sample.

### CROSS-REFERENCE TO RELATED APPLICATIONS

This application is a 35 U.S.C. 371 national phase application of International Patent Cooperation Treaty (PCT) Application No. PCT/US2021/053791, filed on Oct. 6, 2021, entitled METHODS AND COMPOSITIONS FOR ANALYZING NUCLEIC ACID, naming Charles Vaske as inventor, and designated by attorney docket no. CBS-2006PCT, which is based on, claims the benefit of, and claims priority to U.S. Provisional Patent Application No. 63/089,191, filed Oct. 8, 2020. The entire content of the foregoing patent applications is incorporated herein by reference for all purposes, including all text, tables and drawings.

### FIELD

The technology relates in part to methods and compositions for analyzing nucleic acid. In some aspects, the technology relates to methods and compositions for generating one or more genotypes for a sample. In some aspects, the technology relates to methods and compositions for generating one or more genotypes for a mixed sample.

### BACKGROUND

Genetic information of living organisms (e.g., animals, plants and microorganisms) and other forms of replicating genetic information (e.g., viruses) is encoded in nucleic acid (i.e., deoxyribonucleic acid (DNA) or ribonucleic acid (RNA)). Genetic information is a succession of nucleotides or modified nucleotides representing the primary structure of chemical or hypothetical nucleic acids. A genotype is a part of the genetic information of a living organism, which may determine one or more of its characteristics (phenotypes). A genotype may refer to particular gene of interest, a particular mutation or marker, and/or an allele or a combination of alleles.

Existing technology, such as high throughput nucleic acid sequencing and genotype arrays, can generate genotype data for large numbers of markers. However, certain types of samples contain nucleic acid from more than one subject or source (a mixed sample). In some instances, one of the subjects can be genotyped completely from a different (unmixed) sample. For a second subject, an additional (unmixed) sample often cannot be obtained. Accordingly, it is difficult to accurately genotype the second subject from a mixture. Provided herein are methods for accurately obtaining genotypes for an unknown subject from sequencing and/or microarray data generated from a mixed sample. Using a method described herein, genotype files may be generated that are suitable for further analysis (e.g., genetic genealogy analysis).

### SUMMARY

Provided in certain aspects are methods for generating a genotype for a target genomic locus for an unknown subject, comprising a) for a first test sample comprising a mixture of nucleic acid from the unknown subject and a known subject, obtaining sequence reads aligned to a reference genome; b) from the sequence reads, quantifying a linked reference allele and quantifying a linked alternative allele, thereby generating allele quantifications for a linked genomic locus; c) estimating the fraction of nucleic acid from the unknown subject in the mixture; d) for the unknown subject, generating a genotype likelihood for the linked genomic locus given the fraction estimated in (c) and according to the allele quantifications in (b); e) for the unknown subject, generating a set of genotype likelihoods for a target reference allele and a target alternative allele at the target genomic locus based on the linked genomic locus genotype likelihood generated in (d); and f) for the unknown subject, generating a genotype at the target genomic locus based on the set of genotype likelihoods generated in (e).

Also provided in certain aspects are methods for generating a genotype for a target genomic locus for an unknown subject, comprising a) for a first test sample comprising a mixture of nucleic acid from the unknown subject and a known subject, obtaining sequence reads aligned to a reference genome; b) for a haplotype group comprising a target genomic locus and a plurality of linked genomic loci, quantifying a linked reference allele and quantifying a linked alternative allele for each linked genomic locus in the group according to the sequence reads generated in (a), thereby generating allele quantifications for each linked genomic locus in the haplotype group; c) estimating the fraction of nucleic acid from the unknown subject in the mixture; d) for the unknown subject, generating genotype likelihoods for the plurality of linked genomic loci in the haplotype group given the fraction estimated in (c) and according to the allele quantifications in (b); e) for the unknown subject, generating a haplotype pair likelihood set for the haplotype group according to the genotype likelihoods generated in (d); and f) for the unknown subject, generating a genotype at the target genomic locus based on the haplotype pair likelihood set generated in (e).

Also provided in certain aspects are methods for generating a genotype for a target genomic locus for an unknown subject, comprising a) for a test sample comprising a mixture of nucleic acid from two or more unknown subjects, obtaining sequence reads aligned to a reference genome; b) from the sequence reads, quantifying a linked reference allele and quantifying a linked alternative allele, thereby generating allele quantifications for a linked genomic locus; c) estimating the fraction of nucleic acid from a first unknown subject in the mixture; d) for the first unknown subject and a second unknown subject, generating a combined genotype likelihood for the linked genomic locus given the fraction estimated in (c) and according to the allele quantifications in (b); e) for the first unknown subject, generating a set of genotype likelihoods for a target reference allele and a target alternative allele at the target genomic locus based on the combined genotype likelihood for the linked genomic locus generated in (d); and f) for the first unknown subject, generating a genotype at the target genomic locus based on the set of genotype likelihoods generated in (e).

Also provided in certain aspects are methods for generating a genotype for a target genomic locus for an unknown subject, comprising a) for a first test sample comprising a mixture of nucleic acid from two or more unknown subjects, obtaining sequence reads aligned to a reference genome; b) for a haplotype group comprising a target genomic locus and a plurality of linked genomic loci, quantifying a linked reference allele and quantifying a linked alternative allele for each linked genomic locus in the group according to the sequence reads generated in (a), thereby generating allele quantifications for each linked genomic locus in the haplotype group; c) estimating the fraction of nucleic acid from a first unknown subject in the mixture; d) for the first unknown subject and a second unknown subject, generating combined genotype likelihoods for the plurality of linked genomic loci in the haplotype group given the fraction estimated in (c) and according to the allele quantifications in (b); e) generating a haplotype pair likelihood set for the haplotype group according to the combined genotype likelihoods generated in (d); and f) for the first unknown subject, generating a genotype at the target genomic locus based on the haplotype pair likelihood set generated in (e).

Also provided in certain aspects are methods for generating a genotype for a target genomic locus for an unknown subject, comprising a) for a plurality of test samples each comprising a mixture of nucleic acid from two or more unknown subjects, obtaining sequence reads aligned to a reference genome; b) from the sequence reads, and for each test sample, quantifying a linked reference allele and quantifying a linked alternative allele, thereby generating allele quantifications for a linked genomic locus; c) for each test sample, estimating the fraction of nucleic acid from a first unknown subject in the mixture; d) for each test sample, and for the first unknown subject and at least one further unknown subject, generating a combined genotype likelihood for the linked genomic locus given the fraction estimated in (c) and according to the allele quantifications in (b); e) for the first unknown subject, generating a set of genotype likelihoods for a target reference allele and a target alternative allele at the target genomic locus based on the combined genotype likelihood for the linked genomic locus generated in (d); and f) for the first unknown subject, generating a genotype at the target genomic locus based on the set of genotype likelihoods generated in (e).

Also provided in certain aspects are methods for generating a genotype for a target genomic locus for an unknown subject, comprising a) for a plurality of test samples each comprising a mixture of nucleic acid from two or more unknown subjects, obtaining sequence reads aligned to a reference genome; b) for each test sample, and for a haplotype group comprising a target genomic locus and a plurality of linked genomic loci, quantifying a linked reference allele and quantifying a linked alternative allele for each linked genomic locus in the group according to the sequence reads generated in (a), thereby generating allele quantifications for each linked genomic locus in the haplotype group; c) for each test sample, estimating the fraction of nucleic acid from a first unknown subject in the mixture; d) for each test sample, and for the first unknown subject and at least one further unknown subject, generating combined genotype likelihoods for the plurality of linked genomic loci in the haplotype group given the fraction estimated in (c) and according to the allele quantifications in (b); e) generating a haplotype pair likelihood set for the haplotype group according to the combined genotype likelihoods generated in (d); and f) for the first unknown subject, generating a genotype at the target genomic locus based on the haplotype pair likelihood set generated in (e).

Certain implementations are described further in the following description, examples and claims, and in the drawings.

### BRIEF DESCRIPTION OF THE DRAWINGS

The drawings illustrate certain implementations of the technology and are not limiting. For clarity and ease of illustration, the drawings are not made to scale and, in some instances, various aspects may be shown exaggerated or enlarged to facilitate an understanding of particular implementations.

FIG. 1 illustrates a genomic region showing a target site (T) and several nearby linked sites (L1, L2, L3, L4, L5, L6) within a fixed window of size w.

FIG. 2 illustrates a genomic region as in FIG. 1 with sequence reads from a forensic sample carrying allelic information at the target site and/or linked sites. Alleles are denoted 0=reference and 1=alternative alleles.

FIG. 3 shows haplotypes at any genomic region exist within the context of a phylogenetic tree, although the topology of the tree may not be known.

FIG. 4 shows alternate allele frequencies for bi-allelic markers for target sites from direct to consumer arrays.

FIG. 5 shows a general workflow for certain library preparation methods described herein.

FIG. 6 shows how unknown fraction (i.e., DNA fraction of an unknown sample in a mixture) influences expected variant allele fraction (VAF) according to both a known sample's genotype and the unknown sample's genotype. β, unknown fraction; A, sample; X, unknown sample; 0/0, homozygous reference genotype; 0/1, heterozygous reference/alternative; 1/1, homozygous alternative

FIG. 7 shows example estimations of unknown fraction (p) from SNPs with A=0/1 or A=1/1. Rare (PAF<1%) SNVs that are heterozygous in A provide an estimate of p=0.117 (top panel, FIG. 7), and rare SNVs at homozygous non-reference sites in A provide an estimate of p=0.108 (bottom panel, FIG. 7). A, known sample; B, mixed sample; VAF, variant allele fraction

FIG. 8 shows the distribution of read depths for 380 heterozygous sites in the genome with a read depth of exactly 30 reads. In the top panel, the histogram of fraction of alternative reads roughly follows a binomial distribution. The bottom panel shows a quantile-quantile plot of the alternative read depth versus a binomial distribution with its single parameter set to one half. The concordance of the empirical and theoretical distributions is used to justify the use of the binomial distribution in the model.

FIG. 9 shows the theoretical distribution of alternate read counts for various mixture amounts (beta=0.1, 0.2, and 0.3), versus the read counts to a base-calling error rate of 2%, when the known sample is homozygous reference and the unknown sample is heterozygous. As both depth and mixture percentage increase, it is increasingly possible to distinguish heterozygous sites in the unknown sample from normal base calling error.

FIG. 10 shows the theoretical distribution of alternate read counts for various mixture amounts (beta=0.1, 0.2, and 0.3), versus the best-case distribution from a sequencer with no-error (beta=0), when the known sample is heterozygous and the unknown sample is homozygous. Though homozygous sites become more distinguishable as depth and mixture fraction increase, as in FIG. 9, it takes far more sequencing depth or a much higher mixture fraction for the distributions to become as distinguishable as they are for the homozygous sites in FIG. 9.

FIG. 11 shows an example workflow for certain genotype deconvolution methods described herein. In certain workflow variations, only one of INPUT 2a or INPUT 2b is needed. In certain workflow variations, at certain sequencing depths for INPUT 1 and/or certain mixture percentages, neither of INPUT 2a or INPUT 2b is required.

FIG. 12 shows an example of mixture deconvolution using genotyping microarrays.

FIG. 13 shows an example of a of a system for analyzing nucleic acid in accordance with some embodiments of the disclosed subject matter.

FIG. 14 shows an example of hardware that can be used to implement a computing device, and a server, shown in FIG. 13 in accordance with some embodiments of the disclosed subject matter.

FIG. 15 shows an example of a process for determining relative quality of oligonucleotide preparations in accordance with some embodiments of the disclosed subject matter.

### DETAILED DESCRIPTION

Provided herein are methods and compositions useful for analyzing nucleic acid. Also provided herein are methods and compositions for generating one or more genotypes for a sample. In certain aspects, a method herein includes generating one or more genotypes for a mixed sample (i.e., a sample comprising nucleic acid from two or more subjects or sources). In certain aspects, a method herein includes generating a genotype for a target genomic locus based on quantifications of a reference allele and an alternative allele for a linked genomic locus. In certain aspects, a method herein includes generating a genotype for a target genomic locus based on haplotype quantifications.

### Genotyping

Provided herein are methods for generating a genotype for a target genomic locus. Also provided herein are methods for generating a plurality of genotypes for a plurality of genomic loci. In certain implementations of the methods herein, the identity of an individual and/or the identities of one or more relatives of an individual may be determined based on the plurality of genotypes generated for a plurality of genomic loci and in connection with a genealogy analysis. Certain features described below may be applicable to generating a genotype for a target genomic locus according to independent quantifications of alleles at loci that are in linkage disequilibrium with a target locus. Certain features described below may be applicable to generating a genotype for a target genomic locus using a haplotype analysis described herein. Certain features described below may be applicable to generating a genotype for a known subject or an unknown subject, and generating a genotype from a pure (unmixed) sample or from a sample comprising a mixture of nucleic acid from an unknown subject and a known subject. Certain features described below may be applicable to generating genotypes for more than one unknown subject, and generating genotypes from one or more samples comprising a mixture of nucleic acid from more than one unknown subject. Certain features described below may be applicable to generating genotypes based on sequencing data and/or microarray data.

A genotype generally refers to the genetic makeup of an organism. In particular, a genotype may refer to the alleles (e.g., variant forms of a gene and/or variant forms of a polymorphic site), that are carried by an organism. A polymorphic site may include, for example, a single nucleotide polymorphism (SNP), an insertion polymorphism, or a deletion polymorphism. Insertion and deletion polymorphisms are sometimes referred to as indels. Polymorphic sites are found throughout the genome, in coding regions and non-coding regions, and may be referred to herein as markers, target sites, and/or linked sites. Humans are diploid organisms and typically have two alleles at each genetic position, or genomic locus, with one allele inherited from each parent. When two alleles are present, the gene, trait, genomic site or polymorphism in connection with the alleles may be referred to a bi-allelic. Each pair of alleles represents the genotype of a specific gene or polymorphic site. A particular genotype is considered homozygous if it features two identical alleles and heterozygous if the two alleles differ. An allele that is more prevalent in a population (relative to the other allele at a genomic locus) may be referred to as a major allele or a reference allele. An allele that is less prevalent in a population (relative to the other allele at a genomic locus) may be referred to as a minor allele, non-reference allele, an alternate allele, or an alternative allele. The process of determining a genotype is referred to as genotyping.

A method herein may comprise generating a genotype for a target genomic locus for a test sample. A target genomic locus is the locus in a genome for which a genotype is determined. A target genomic locus may be a polymorphic site in a genome. In some embodiments, a target genomic locus is a location of a single nucleotide polymorphism (SNP). In some embodiments, a target genomic locus is a location of a bi-allelic single nucleotide polymorphism (SNP). A target genomic locus may be selected based on its inclusion in one or more genealogy databases.

A genotype for a target genomic locus may be generated according to a pair of alleles. A pair of alleles may include a reference allele (i.e., major allele) and an alternative allele (i.e., minor allele, non-reference allele). A reference allele, when determined at a target genomic locus, may be referred to as a target reference allele. An alternative allele, when determined at a target genomic locus, may be referred to as a target alternative allele. Possible genotypes for a target genomic locus generally include homozygous for the target reference allele (i.e., two copies of the target reference allele), heterozygous for the target reference allele and the target alternative allele (i.e., one copy of the target reference allele and one copy of the target alternative allele), and homozygous for the target alternative allele (i.e., two copies of the target alternative allele).

In some embodiments, a genotype generated for a target genomic locus is an unphased genotype. An unphased genotype refers to a genotype lacking a designation as to which one of the pair of chromosomes (i.e., maternally inherited and paternally inherited) holds each allele. In some embodiments, a genotype generated for a target genomic locus is for a single nucleotide polymorphism (SNP). In some embodiments, a genotype generated for a target genomic locus is for a bi-allelic single nucleotide polymorphism (SNP). In some embodiments, a genotype generated for a target genomic locus is an unphased genotype for a single nucleotide polymorphism (SNP). In some embodiments, a genotype generated for a target genomic locus is an unphased genotype for a bi-allelic single nucleotide polymorphism (SNP).

In some embodiments, a method herein comprises generating a plurality of genotypes at a plurality of target genomic loci for a test sample. A plurality of genomic loci generally refers to two or more genomic loci. In some embodiments, a plurality of genomic loci comprises about 1,000 or more genomic loci. In some embodiments, a plurality of genomic loci comprises about 10,000 or more genomic loci. In some embodiments, a plurality of genomic loci comprises about 100,000 or more genomic loci. In some embodiments, a plurality of genomic loci comprises about 200,000 or more genomic loci. In some embodiments, a plurality of genomic loci comprises about 300,000 or more genomic loci. In some embodiments, a plurality of genomic loci comprises about 400,000 or more genomic loci. In some embodiments, a plurality of genomic loci comprises about 500,000 or more genomic loci. In some embodiments, a plurality of genomic loci comprises about 600,000 or more genomic loci. In some embodiments, a plurality of genomic loci comprises about 700,000 or more genomic loci. In some embodiments, a plurality of genomic loci comprises about 800,000 or more genomic loci. In some embodiments, a plurality of genomic loci comprises about 900,000 or more genomic loci. In some embodiments, a plurality of genomic loci comprises about 1,000,000 or more genomic loci.

In some embodiments, a method herein comprises filtering one or more target genomic loci. Filtering one or more target genomic loci refers to removing one or more target genomic loci from a genotyping analysis herein. In some embodiments, one or more target genomic loci are filtered by removing genomic loci that are within a certain proximity of an insertion polymorphism or a deletion polymorphism. In some embodiments, one or more target genomic loci are filtered by removing genomic loci that are within 1 to 10 bases of an insertion polymorphism or a deletion polymorphism. For example, a target genomic locus may be removed from a genotyping analysis herein if the target genomic locus is within 1 base, 2, bases, 3 bases, 4 bases, 5 bases, 6 bases, 7 bases, 8 bases, 9 bases, or 10 bases of an insertion polymorphism or a deletion polymorphism. In some embodiments, a target genomic locus is removed from a genotyping analysis herein if the target genomic locus is within 4 bases of an insertion polymorphism or a deletion polymorphism. A genomic target site filtered according to criteria described above may be referred to as a blacklisted site.

In some embodiments, alleles are analyzed at a linked genomic locus. A linked genomic locus generally refers to a genomic locus located within a certain proximity of a target genomic locus. In some embodiments, a linked genomic locus refers to a genomic locus located within about 1 kilobase (kb) to about 20 kb (upstream or downstream) of a target genomic locus. For example, a linked genomic locus may be within about 1 kb, about 2 kb, about 3 kb, about 4 kb, about 5 kb, about 6 kb, about 7 kb, about 8 kb, about 9 kb, about 10 kb, about 11 kb, about 12 kb, about 13 kb, about 14 kb, about 15 kb, about 16 kb, about 17 kb, about 18 kb, about 19 kb, or about 20 kb of a target genomic locus. In some embodiments, a linked genomic locus is a genomic locus located within about 10 kb upstream or within 10 kb downstream of a target genomic locus. A linked genomic locus may be a polymorphic site in a genome. In some embodiments, a linked genomic locus is a location of a single nucleotide polymorphism (SNP). In some embodiments, a linked genomic locus is a location of a bi-allelic single nucleotide polymorphism (SNP). A linked genomic locus may be selected based on its inclusion in one or more databases. For example, linked genomic loci may be selected according to one or more human genome sequencing projects (e.g., the 1000 Genomes project). In some embodiments, genotypes and genotype likelihoods are not determined for linked genomic loci. In such instances, genotypes and genotype likelihoods are generated for one or more target genomic loci without generating genotypes or genotype likelihoods for one or more linked genomic loci. In some embodiments, genotypes and genotype likelihoods are determined for linked genomic loci. In such instances, genotypes and genotype likelihoods are generated for one or more target genomic loci, based, in part, on genotypes or genotype likelihoods generated for one or more linked genomic loci.

In some embodiments, alleles are analyzed at a plurality of linked genomic loci. In some embodiments, alleles are analyzed at a plurality of linked genomic loci for each target genomic locus. A plurality of linked genomic loci may comprise about 10 linked genomic loci to about 1000 linked genomic loci (e.g., for each target genomic locus). For example, a plurality of linked genomic loci may comprise about 10 linked genomic loci, about 20 linked genomic loci, about 30 linked genomic loci, about 40 linked genomic loci, about 50 linked genomic loci, about 60 linked genomic loci, about 70 linked genomic loci, about 80 linked genomic loci, about 90 linked genomic loci, about 100 linked genomic loci, about 200 linked genomic loci, about 300 linked genomic loci, about 400 linked genomic loci, about 500 linked genomic loci, about 600 linked genomic loci, about 700 linked genomic loci, about 800 linked genomic loci, about 900 linked genomic loci, or about 1000 linked genomic loci. A plurality of linked genomic loci may comprise about 5 linked genomic loci to about 50 linked genomic loci (e.g., for each target genomic locus). For example, a plurality of linked genomic loci may comprise about 5 linked genomic loci, about 10 linked genomic loci, about 15 linked genomic loci, about 20 linked genomic loci, about 25 linked genomic loci, about 30 linked genomic loci, about 35 linked genomic loci, about 40 linked genomic loci, about 45 linked genomic loci, or about 50 linked genomic loci.

In some embodiments, allele quantifications are generated. In some embodiments, allele quantifications are generated for alleles at a linked genomic locus. For example, a method herein may comprise quantifying a linked reference allele (major allele at a linked genomic locus) and quantifying a linked alternative allele (minor allele at a linked genomic locus). In some embodiments, allele quantifications are generated for alleles at a target genomic locus. For example, a method herein may comprise quantifying a target reference allele and quantifying a target alternative allele. Each allele quantification may be generated according to the amount of sequence reads, or an adjusted amount of sequence reads, carrying a particular allele at a genomic locus. For example, an allele quantification may be generated according to the amount of sequence reads carrying a reference allele at a linked genomic locus. In some embodiments, an allele quantification is generated according to the amount of sequence reads carrying an alternative allele at a linked genomic locus. In some embodiments, an allele quantification is generated according to the amount of sequence reads carrying a reference allele at a target genomic locus. In some embodiments, an allele quantification is generated according to the amount of sequence reads carrying an alternative allele at a target genomic locus. The amount of sequence reads for an allele quantification may be adjusted, for example, according to a measure of sequencing error. In some embodiments, a measure of sequencing error is a fixed error rate (e.g., a fixed error rate associated with a particular sequencing platform and/or sequencing library preparation method). In some embodiments, a measure of sequencing error is an error associated with a sequencing run, a test sample, or group of test samples. In some embodiments, a measure of sequencing error is an error associated with a particular genomic locus or region.

In some embodiments, a method herein comprises quantifying a plurality of linked reference alleles and quantifying a plurality of linked alternative alleles, thereby generating a plurality of allele quantifications for a plurality of linked genomic loci. In some embodiments, a plurality of linked reference alleles and a plurality of linked alternative alleles are quantified at a plurality of linked genomic loci for each target genomic locus. Accordingly, generating a genotype call at a target genomic locus may be based on allele quantifications at a plurality of linked genomic loci. Furthermore, generating a plurality of genotypes at a plurality of linked genomic loci may be based on allele quantifications at multiple pluralities of linked genomic loci (i.e., each genotype call is based on allele quantifications at its own set of linked genomic loci).

An allele quantification can be determined by a suitable method, operation or mathematical process. An allele quantification sometimes is the direct sum of all sequence reads carrying a particular allele (e.g., reference allele, alternative allele) at a genomic locus (e.g., a liked genomic locus, a target genomic locus). An allele quantification may be expressed as a ratio (e.g., a ratio of a quantification for a particular allele to a quantification for a different allele or all alleles).

In some embodiments, an allele quantification is derived from raw sequence reads and/or filtered sequence reads. In certain embodiments, an allele quantification is an average, mean or sum of sequence reads carrying a particular allele (e.g., reference allele, alternative allele) at a genomic locus (e.g., a liked genomic locus, a target genomic locus). In some embodiments, an allele quantification is associated with an uncertainty value. An allele quantification sometimes is adjusted. An allele quantification may be adjusted according to sequence reads that have been weighted, removed, filtered, normalized, adjusted, averaged, derived as a mean, derived as a median, added, or combination thereof.

In some embodiments, a method herein comprises filtering one or more sequence reads. Filtering one or more sequence reads refers to removing one or more sequence reads from a genotyping analysis herein. In some embodiments, one or more sequence reads are filtered by removing sequence reads that align to a genomic position within a certain proximity of an insertion polymorphism or a deletion polymorphism. In some embodiments, one or more sequence reads are filtered by removing sequence reads that align to a genomic position within 1 to 10 bases of an insertion polymorphism or a deletion polymorphism. For example, a sequence read may be removed from a genotyping analysis herein if the sequence read aligns to a genomic position within 1 base, 2, bases, 3 bases, 4 bases, 5 bases, 6 bases, 7 bases, 8 bases, 9 bases, or 10 bases of an insertion polymorphism or a deletion polymorphism. In some embodiments, a sequence read is removed from a genotyping analysis herein if the sequence read aligns to a genomic position within 4 bases of an insertion polymorphism or a deletion polymorphism.

In some embodiments, a method herein comprises filtering sequence reads according to mapping/alignment parameters and/or quality score. For example, sequence reads that do not map well and/or do not have a suitable alignment score may be removed from an analysis herein. Reads that may be filtered out include, for example, discordant reads, ambiguous reads, off-target reads, reads having one or more undetermined base calls, and reads having a low quality sequences and/or base quality scores. Low quality sequences may be identified according to base quality scores for one or more nucleotide positions in a sequence. A base quality score, or quality score, is a prediction of the probability of an error in base calling. Quality scores may be generated according to one or more sets of quality predictor values, and can depend on certain characteristics of the sequencing platform used for generating sequence reads. Generally, a high quality score indicates a base call is more reliable and less likely is an incorrect base call. In some embodiments, individual bases within sequence reads are filtered. For example, individual bases that do not have a suitable base quality score may be removed from an analysis herein.

In some embodiments, a method herein comprises filtering one or more allele quantifications. Filtering one or more allele quantifications refers to removing one or more allele quantifications from a genotyping analysis herein. In some embodiments, one or more allele quantifications are filtered by removing allele quantifications derived from sequence reads that align to a genomic position within a certain proximity of an insertion polymorphism or a deletion polymorphism. In some embodiments, one or more allele quantifications are filtered by removing allele quantifications derived from sequence reads that align to a genomic position within 1 to 10 bases of an insertion polymorphism or a deletion polymorphism. For example, an allele quantification may be removed from a genotyping analysis herein if the allele quantification is derived from sequence reads aligned to a genomic position within 1 base, 2, bases, 3 bases, 4 bases, 5 bases, 6 bases, 7 bases, 8 bases, 9 bases, or 10 bases of an insertion polymorphism or a deletion polymorphism. In some embodiments, an allele quantification is removed from a genotyping analysis herein if the allele quantification is derived from sequence reads aligned to a genomic position within 4 bases of an insertion polymorphism or a deletion polymorphism.

In some embodiments, a method herein comprises determining one or more genotype likelihoods for a target genomic locus. In some embodiments, a method herein comprises determining one or more genotype likelihoods for a target reference allele and a target alternative allele at a target genomic locus. In some embodiments, a method herein comprises determining a set of genotype likelihoods for a target reference allele and a target alternative allele at a target genomic locus. A set of genotype likelihoods may comprise one or more likelihoods for genotypes chosen from homozygous for the target reference allele, heterozygous for the target reference allele and the target alternative allele, and homozygous for the target alternative allele. In some embodiments, a set of genotype likelihoods comprises likelihoods for a homozygous for the target reference allele genotype, a heterozygous for the target reference allele and the target alternative allele genotype, and a homozygous for the target alternative allele genotype.

In some embodiments, a method herein comprises generating a genotype likelihood for a target reference allele and a target alternative allele at a target genomic locus according to one or more probabilities of a genotype at the target genomic locus. In some embodiments, a method herein comprises generating a set of genotype likelihoods for a target reference allele and a target alternative allele at a target genomic locus according to probabilities for each genotype (i.e., homozygous reference, heterozygous reference and alternative, and homozygous alternative) at the target genomic locus. A probability of a genotype at a target genomic locus may be based, in part, on one or more of allele frequency, haplotype frequency, genotype frequency, allele quantifications, and prior probabilities.

In some embodiments, a method herein comprises generating a genotype likelihood for a target reference allele and a target alternative allele at a target genomic locus according to one or more probabilities of observing certain data obtained for a test sample (e.g., allele quantifications obtained for a test sample) given a particular genotype at a target genomic locus. The phrase “given a particular genotype at a target genomic locus” refers to an assumption of a particular genotype at a target genomic locus. The phrase “given a particular genotype at a target genomic locus” may be used interchangeably with “for a particular assumed genotype at a target genomic locus.” For example, a method herein may comprise using observed allele quantifications at one or more linked genomic loci to query 1) how likely such observed allele quantifications would be if the genotype at the target genomic locus was homozygous reference; 2) how likely such observed allele quantifications would be if the genotype at the target genomic locus was heterozygous; and/or 3) how likely such observed allele quantifications would be if the genotype at the target genomic locus was homozygous alternative.

In some embodiments, a probability of a genotype at a target genomic locus is based, in part, on data obtained for a test sample (e.g., allele quantifications obtained for a test sample). For example, a probability of a genotype at a target genomic locus may be based, in part, on allele quantifications for a linked reference allele and a linked alternative allele. In some embodiments, a probability of a genotype at a target genomic locus may be further based, in part, on allele quantifications for a target reference allele and a target alternative allele.

In some embodiments, a probability of a genotype at a target genomic locus is generated according to a probability of observing a particular allele (e.g., reference or alternative) at a linked genomic locus, given a particular allele (e.g., reference or alternative) or genotype at a target genomic locus. The phrase “given a particular allele (e.g., reference or alternative) or genotype at a target genomic locus” refers to an assumption of a particular allele or genotype at a target genomic locus. The phrase “given a particular allele (e.g., reference or alternative) or genotype at a target genomic locus” may be used interchangeably with “for a particular assumed allele (e.g., reference or alternative) or assumed genotype at a target genomic locus.” In some embodiments, a probability of a genotype at a target genomic locus is generated according to a probability of observing a linked reference allele at a linked genomic locus, given a target reference allele at a target genomic locus. In some embodiments, a probability of a genotype at a target genomic locus is generated according to a probability of observing a linked reference allele at a linked genomic locus, given a target alternative allele at a target genomic locus. In some embodiments, a probability of a genotype at a target genomic locus is generated according to a probability of observing a linked reference allele at a linked genomic locus, given a target reference allele at a target genomic locus, and a probability of observing a linked reference allele at a linked genomic locus, given a target alternative allele at a target genomic locus. In some embodiments, the probability of a genotype that is homozygous for a target reference allele is generated according to a probability of observing a linked reference allele at a linked genomic locus, given a target reference allele at a target genomic locus. In some embodiments, the probability of a genotype that is heterozygous for a target reference allele and a target alternative allele is generated according to a probability of observing a linked reference allele at a linked genomic locus, given a target reference allele at a target genomic locus, and a probability of observing a linked reference allele at a linked genomic locus, given a target alternative allele at a target genomic locus. In some embodiments, the probability of a genotype that is homozygous for a target alternative allele is generated according to a probability of observing the linked reference allele at the linked genomic locus, given a target alternative allele at the target genomic locus.

In some embodiments, a probability of a genotype at a target genomic locus is generated according to a probability of observing a linked reference allele at a linked genomic locus, given a target reference genotype at a target genomic locus. In some embodiments, a probability of a genotype at a target genomic locus is generated according to a probability of observing a linked reference allele at the linked genomic locus, given a target alternative genotype at a target genomic locus. In some embodiments, a probability of a genotype at a target genomic locus is generated according to a probability of observing a linked reference allele at a linked genomic locus, given a target reference genotype at a target genomic locus, and a probability of observing a linked reference allele at a linked genomic locus, given a target alternative genotype at a target genomic locus. In some embodiments, the probability of a genotype that is homozygous for a target reference allele is generated according to a probability of observing a linked reference allele at a linked genomic locus, given a target reference genotype (e.g., homozygous reference) at a target genomic locus. In some embodiments, the probability of a genotype that is heterozygous for a target reference allele and a target alternative allele is generated according to a probability of observing a linked reference allele at a linked genomic locus, given a target reference genotype at a target genomic locus, and a probability of observing a linked reference allele at a linked genomic locus, given a target alternative genotype at a target genomic locus. In some embodiments, the probability of a genotype that is heterozygous for a target reference allele and a target alternative allele is generated according to a probability of observing a linked reference allele at a linked genomic locus, given a heterozygous genotype at a target genomic locus. In some embodiments, the probability of a genotype that is homozygous for a target alternative allele is generated according to a probability of observing the linked reference allele at the linked genomic locus, given a target alternative genotype (e.g., homozygous alternative) at the target genomic locus.

In some embodiments, a probability of observing a linked reference allele at a linked genomic locus, given a particular target allele or genotype at a target genomic locus, is based, in part, on a measure of linkage disequilibrium for a linked reference allele and a target reference allele. Linkage disequilibrium refers to a non-random association of alleles at two or more loci (e.g., in a population). In some embodiments, a measure of disequilibrium is based on a haplotype frequency (e.g., a haplotype frequency in a population for a linked reference allele and a particular target allele (e.g., reference or alternative)).

In some embodiments, a probability of observing a linked reference allele at a linked genomic locus, given a particular target allele or genotype at a target genomic locus, is combined with an allele quantification (e.g., an allele quantification of linked reference alleles, an allele quantification of linked alternative alleles). A probability may be combined with an allele quantification by applying a mathematical manipulation. A mathematical manipulation may include, for example, multiplication, division, addition, subtraction, integration, symbolic computation, algebraic computation, algorithm, trigonometric or geometric function, transformation, and a combination thereof. Examples of a probability of observing a linked reference allele at a linked genomic locus, given a particular target allele or genotype at a target genomic locus, combined with an allele quantification are provided in equations (1) and (2) herein.

In some embodiments, a probability of observing a linked reference allele at a linked genomic locus, given a particular target allele or genotype at a target genomic locus, may be adjusted. In some embodiments, a probability is adjusted according to a measure of sequencing error. In some embodiments, a measure of sequencing error is a fixed error rate (e.g., a fixed error rate associated with a particular sequencing platform and/or sequencing library preparation method). In some embodiments, a measure of sequencing error is an error associated with a sequencing run, a test sample, or group of test samples. In some embodiments, a measure of sequencing error is an error associated with a particular genomic locus or region.

In some embodiments, a probability of a genotype at a target genomic locus is based, in part, on one or more prior probabilities. Prior probabilities may be based on certain frequencies in a population (e.g., allele frequencies, genotype frequencies, haplotype frequencies). In some embodiments, a probability of a genotype at a target genomic locus is based on prior probabilities of the target reference allele and the target alternative allele. Prior probabilities may be based, in part, on haplotype frequencies (e.g., haplotype frequencies in a population). For example, prior probabilities may be based on haplotype frequencies for one or more or all of (i) a target reference allele and a linked reference allele, (ii) a target reference allele and a linked alternative allele, (iii) a target alternative allele and a linked reference allele, and (iv) a target alternative allele and a linked alternative allele.

In some embodiments, a likelihood (L) for a homozygous target reference allele genotype (T00) is generated according to a process derived from equation (1):

$$L ( T 00 ) = P ( D \vert T 00 ) \times P ( T 00 ) = {\prod }_{{L}_{i}} ( \begin{matrix}{L}_{i} 0 + {L}_{i} 1 \\ {L}_{i} 0\end{matrix} ) \times {PL}_{i} {0}^{{L}_{i} 0} \times {( 1 - P {L}_{i} 0 )}^{{L}_{i} 1} \times {( \frac{T 0 {L}_{i} 0 + T 0 {L}_{i} 1}{T 0 {L}_{i} 0 + T 0 {L}_{i} 1 + T 1 {L}_{i} 0 + T 1 {L}_{i} 1} )}^{2} \qquad ( 1 )$$

where
L(T00) is a likelihood of genotype 00 at target genomic locus T,
Lᵢ0 is an allele quantification of linked reference alleles observed at linked genomic locus Lᵢ (where ᵢ refers to the linked genomic locus being analyzed),
Lᵢ1 is an allele quantification of linked alternative alleles observed at linked genomic locus Lᵢ,
PLᵢ0 is a probability of observing a linked reference allele at linked genomic locus Lᵢ, given allele T0, and T0ᵢL0, T0Lᵢ1, T1Lᵢ0 and T1Lᵢ1 are haplotype frequencies for (i) a target reference allele and a linked reference allele, (ii) a target reference allele and a linked alternative allele, (iii) a target alternative allele and a linked reference allele, and (iv) a target alternative allele and a linked alternative allele.

In some embodiments, a likelihood (L) for a homozygous target alternative allele genotype (T11) is generated according to a process derived from equation (2):

$$L ( T 11 ) = P ( D \vert T 11 ) \times P ( T 11 ) = {\prod }_{{L}_{i}} ( \begin{matrix}{L}_{i} 0 + {L}_{i} 1 \\ {L}_{i} 0\end{matrix} ) \times P \text{} {{L}_{i}}^{{L}_{i} 0} \times {( 1 - P {L}_{i} 0 )}^{{L}_{i} 1} \times {( \frac{T 1 {L}_{i} 0 + T 1 {L}_{i} 1}{T 0 {L}_{i} 0 + T 0 {L}_{i} 1 + T 1 {L}_{i} 0 + T 1 {L}_{i} 1} )}^{2} \qquad ( 2 )$$

where:
L(T11) is a likelihood of genotype 11 at target genomic locus T
Lᵢ0 is an allele quantification of linked reference alleles observed at linked genomic locus Lᵢ
Lᵢ1 is an allele quantification of linked alternative alleles observed at linked genomic locus Lᵢ
PLᵢ0 is a probability of observing a linked reference allele at linked genomic locus Lᵢ, given allele T1, and
T0ᵢL0, T0Lᵢ1, T1Lᵢ0 and T1Lᵢ1 are haplotype frequencies for (i) a target reference allele and a linked reference allele, (ii) a target reference allele and a linked alternative allele, (iii) a target alternative allele and a linked reference allele, and (iv) a target alternative allele and a linked alternative allele.

In some embodiments, a likelihood (L) for a heterozygous target reference allele and target alternative allele genotype (T01) is generated according to a process derived from equation (1) and equation (2). For example, a likelihood (L) for a heterozygous target reference allele and target alternative allele genotype (T01) is generated according to a process derived from equation (3):

$$L ( T 01 ) = \text{} P ( D \vert T 01 ) \times P ( T 01 ) = \prod {L}_{i} ( \begin{matrix}{L}_{i} 0 + {L}_{i} 1 \\ {L}_{i} 0\end{matrix} ) \times {PL}_{i} \text{} {0}^{{L}_{i} 0} \times {( 1 - P {L}_{i} 0 )}^{{L}_{i} 0} \times ( 2 \times ( \frac{T 0 {L}_{i} 0 + T 0 {L}_{i} 1}{T 0 {L}_{i} 0 + T 0 {L}_{i} 1 + T 1 {L}_{i} 0 + T 1 {L}_{i} 1} ) \times ( \frac{T 1 {L}_{i} 0 + T 1 {L}_{i} 1}{T 0 {L}_{i} 0 + T 0 {L}_{i} 1 + T 1 {L}_{i} 0 + T 1 {L}_{i} 1} ) ) \qquad ( 3 )$$

where:

$$P {L}_{i} 0 = ( 0.5 \times \frac{T 0 {L}_{i} 0}{T 0 {L}_{i} 0 + T 0 {L}_{i} 1} ) + ( 0.5 \times \frac{T 1 {L}_{i} 0}{T 1 {L}_{i} 0 + T 1 {L}_{i} 1} )$$

In some embodiments, a plurality of genotype likelihood sets for a target genomic locus is generated. In some embodiments, a plurality of genotype likelihood sets for a target genomic locus is generated according to a plurality of allele quantifications for a plurality of linked genomic loci. For example, for a target genomic locus having 10 linked genomic loci, 10 genotype likelihood sets may be generated. In another example, for a target genomic locus having 100 linked genomic loci, 100 genotype likelihood sets may be generated. In some embodiments, a genotype at a target genomic locus is generated based on a plurality of genotype likelihood sets.

A genotype at a target genomic locus may be generated based on a set of genotype likelihoods. As described above each set of genotype likelihoods is generated according to allele quantifications and probabilities for a linked genomic locus, and each set may contain a likelihood for each genotype possibility at the target genomic locus: homozygous reference, heterozygous reference/alternative, and homozygous alternative. In instances where three genotype likelihoods are generated for a target genomic locus, based on a first linked genomic site, the most likely genotype is selected. The likelihood of the most likely genotype may be compared to the likelihood of the second most likely genotype to generate a likelihood ratio. Genotype calls may be filtered according to this ratio, calling only genotypes with a high likelihood ratio and/or a ratio above a particular threshold or cutoff value. For example, genotypes calls in which the most likely call is at least 10 times, at least 100 times, at least 1000 times, or at least 10,000 times more likely than the second most likely call may be reported. Thus, a high likelihood ratio and/or a ratio above a particular threshold or cutoff value generally refers to a ratio value of about 10 or more, 20 or more, 30 or more, 40 or more, 50 or more, 60 or more, 70 or more, 80 or more, 90 or more, 100 or more, 200 or more, 300 or more, 400 or more, 500 or more, 600 or more, 700 or more, 800 or more, 900 or more, 1000 or more, 2000 or more, 3000 or more, 4000 or more, 5000 or more, 6000 or more, 7000 or more, 8000 or more, 9000 or more, or 10,000 or more. In certain instances, when a likelihood ratio is below a threshold or cutoff, a partial genotype may be generated by calling one of the alleles at the target genomic locus.

In some embodiments, each genotype for a target genomic locus in a plurality of genotypes for a plurality of genomic loci is generated independently from the other genotypes in the plurality of genotypes. Accordingly, a genotype generated at a first locus has no bearing on a genotype generated at a second locus, even if the first locus and the second locus are within a certain proximity to each other (e.g., are considered linked target loci). Thus, if a genotype generated at a first locus is an erroneous genotype, the genotype determined at the second locus is not any more likely to be erroneous. Methods herein typically generate genotypes at target genomic loci without generating a haplotype for two or more target genomic loci. A haplotype generally refers to a group of alleles that are inherited together from one parent. In some contexts, a haplotype refers to a collection of specific alleles in a cluster of tightly linked genes on a chromosome that are likely to be inherited together. In some contexts, a set of linked single nucleotide polymorphism (SNP) alleles that are associated statistically. Certain existing genotyping approaches use a few alleles of a specific haplotype sequence to identify other polymorphic sites that are nearby on the chromosome. For example, certain existing genotyping approaches generate genotypes at all sites (all target sites and all linked sites) in a panel, and attempt to learn which two haplotypes are present. Using this approach, when a haplotype is called incorrectly, correlated errors are made, outputting alleles of the wrong haplotype. In contrast, the genotyping approach described herein considers every target genomic locus (i.e., every target genomic locus along with its linked genomic locus) independently. Thus, errors are independent and uncorrelated. Generally, genotype calls are made at target genomic loci (and not linked genomic loci). Linked genomic loci generally are used independently for target sites. In certain instances, a linked genomic locus could be a linked genomic locus for two or more target genomic loci. In such instance, the linked genomic loci is used independently for each target site.

Because each sequence read is independent data from other sequence reads, genotype likelihoods calculated from data at each linked site generally are treated as independent observations. In some embodiments, a composite genotype likelihood is generated according to a plurality of linked genomic loci likelihoods for each target genomic locus genotype possibility. In some embodiments, a composite genotype likelihood is generated by multiplying all linked genomic loci likelihoods for each target genomic locus genotype possibility. In some embodiments, results may be filtered according to a level of linkage disequilibrium and/or observed coverage. In some embodiments, a genotype likelihood ratio is generated according to a comparison of composite likelihoods of each target genomic locus genotype (homozygous reference, heterozygous reference/alternative, and homozygous alternative). This ratio may be used to filter results in the context of one or more considerations of probability, non-limiting examples of which include, sensitivity, specificity, standard deviation, median absolute deviation (MAD), measure of certainty, measure of confidence, measure of certainty or confidence that a value obtained for a genotype likelihood ratio is inside or outside a particular range of values, measure of uncertainty, measure of uncertainty that a value obtained for a genotype likelihood ratio is inside or outside a particular range of values, coefficient of variation (CV), confidence level, confidence interval (e.g., about 95% confidence interval), standard score (e.g., z-score), chi value, phi value, result of a t-test, p-value, ploidy value, fitted minority species fraction, area ratio, median level, the like or combination thereof. For example, a genotype likelihood ratio may be used to filter results at any desired confidence level. In some embodiments, suitable genotype likelihood ratios for genetic genealogy range from about 10 to 10,000.

Using a genotyping method described herein, genotype calls may be made for a majority of target genomic loci. In some embodiments, genotype calls are made for at least about 75% of the target genomic loci. For example, genotype calls may be made for at least 80%, at least 85%, at least 90%, at least 91%, at least 92%, at least 93%, at least 94%, at least 95%, at least 96%, at least 97%, at least 98%, at least 99%, or 100% of target genomic loci. In some embodiments, genotype calls are made for about 92% of the target genomic loci.

In some embodiments, a method herein comprises identifying a subject based on a plurality of genotypes generated for a test sample. In some embodiments, genotypes generated according to a method provided herein are entered into a file format suitable for downstream analysis (e.g., uploaded to a genetic genealogy service). In some embodiments, a subject from which the sample was derived is identified according to a downstream analysis (e.g., analysis performed by a genetic genealogy service or analysis performed using a database connected to a genetic genealogy service). In some embodiments, one or more relatives of a subject from which the sample was derived is/are identified according to a downstream analysis (e.g., analysis performed by a genetic genealogy service or analysis performed using a database connected to a genetic genealogy service). Generally, accurate genotype calls at a large number of target genomic loci are required for a positive identification of a subject or a relative of a subject. For example, using certain genealogy platforms, at least about 100,000, 200,000, 300,000, 400,000, 500,000, 600,000, 700,000, 800,000, 900,000 or 1,000,000 accurate genotype calls are required. In some embodiments, at least 500,000 accurate genotype calls are required for a positive identification of a subject or a relative of a subject. In some embodiments, at least 600,000 accurate genotype calls are required for a positive identification of a subject or a relative of a subject.

### Genotyping Using a Haplotype Analysis

Provided herein are methods for generating a genotype for a target genomic locus according to a haplotype analysis. A haplotype generally refers to a group of alleles that are inherited together (i.e., on the same chromosome or chromosome section) from a single parent. In some embodiments, a method herein comprises analyzing a haplotype group. A haplotype group herein generally refers a section of a genome comprising a target genomic locus and a plurality of linked genomic loci. A haplotype group may be referred to herein as a haplotype set, a haplotype panel, or a haplotype description. A haplotype group may be described as a matrix where the rows are unique haplotypes and the columns are the genomic loci in the haplotypes, as described in Example 2.

The linked genomic loci that make up a haplotype group may be selected according to one or more criteria described herein. For example, a haplotype group may comprise linked genomic loci in linkage disequilibrium with a target genomic locus. A haplotype group may comprise linked genomic loci generally present in nucleic acid recovered from a particular type of test sample (e.g., hair DNA, damaged or degraded DNA). A haplotype group may comprise linked genomic loci having suitable mapping characteristics (e.g., loci that avoid repetitive regions, loci that avoid insertion-deletion polymorphisms, and loci that avoid other genome features that may disrupt accurate mapping). Genomic loci in a haplotype group may be adequately spaced from one another (e.g., spaced such that a single sequencing read generally does not comprise multiple loci, thus avoiding over counting data from single reads). In some embodiments, each locus in the plurality of linked genomic loci in the haplotype group is at least about 1 base away to at least about 250 bases away from other loci in the haplotype group. For example, each locus in the plurality of linked genomic loci in the haplotype group may be at least about 10 bases away from other loci in the haplotype group, at least about 20 bases away from other loci in the haplotype group, at least about 30 bases away from other loci in the haplotype group, at least about 40 bases away from other loci in the haplotype group, at least about 50 bases away from other loci in the haplotype group, at least about 60 bases away from other loci in the haplotype group, at least about 70 bases away from other loci in the haplotype group, at least about 80 bases away from other loci in the haplotype group, at least about 90 bases away from other loci in the haplotype group, at least about 100 bases away from other loci in the haplotype group, at least about 150 bases away from other loci in the haplotype group, at least about 200 bases away from other loci in the haplotype group, or at least about 250 bases away from other loci in the haplotype group. In some embodiments, each locus in the plurality of linked genomic loci in the haplotype group is at least about 70 bases away from other loci in the haplotype group.

In some embodiments, allele quantifications are generated for a haplotype group. Generating allele quantifications is described above. In some embodiments, a method herein comprises quantifying linked alleles in a haplotype group. In some embodiments, a method herein comprises quantifying a linked reference allele and quantifying a linked alternative allele for each linked genomic locus in a haplotype group. In some embodiments, a method herein comprises quantifying a target allele in a haplotype group. In some embodiments, a method herein comprises quantifying a target reference allele and quantifying a target alternative allele for a target genomic locus in a haplotype group. In some embodiments, allele quantifications are generated for a plurality of haplotype groups. In some embodiments, a method herein comprises quantifying linked alleles in a plurality of haplotype groups. In some embodiments, a method herein comprises quantifying a linked reference allele and quantifying a linked alternative allele for each linked genomic locus in each haplotype group, thereby generating allele quantifications for each linked genomic locus for each group in the plurality of haplotype groups.

In some embodiments, a likelihood for one or more haplotype pairs is generated. A haplotype pair refers to, for a diploid organism, two haplotypes (two haplotype species) from the same haplotype group, where the first haplotype is on one chromosome (inherited from one parent) and the second haplotype is on the homologous chromosome (inherited from the other parent). A haplotype pair may comprise any two possible haplotype species for a haplotype group. A haplotype pair may comprise two identical haplotype species (e.g., AA from the haplotypes in Table 2 in Example 2) or may comprise two different haplotype species (e.g., AB from the haplotypes in Table 2 in Example 2). A likelihood for a haplotype pair generally refers to a measure of how likely a test sample (e.g., from a test subject) has particular haplotype pair given the observed data (e.g., allele quantifications). In some embodiments, a likelihood for each possible haplotype pair is generated. A haplotype pair likelihood may be based, in part, on one or more of allele frequency, haplotype frequency, genotype frequency, allele quantifications, and prior probabilities.

In some embodiments, a haplotype pair likelihood set is generated for a haplotype group. In some embodiments, a haplotype pair likelihood set is generated according to allele quantifications for each linked genomic locus for a haplotype group. A haplotype pair likelihood set generally refers to a collection of likelihoods generated for a plurality of haplotype pair possibilities (e.g., where the set comprises a separate likelihood for each haplotype pair possibility in Table 2: AA, AB, BB, BC, CC, etc.). In some embodiments, a plurality of haplotype pair likelihood sets are generated for a plurality of haplotype groups. In some embodiments, a plurality of haplotype pair likelihood sets are generated according to allele quantifications for each linked genomic locus for each group in a plurality of haplotype groups.

A haplotype pair likelihood set may be generated according to any suitable statistical process or model. In some embodiments, a haplotype pair likelihood set is generated according to an evidential probability. In some embodiments, a haplotype pair likelihood set is generated according to a Bayesian probability. A Bayesian probability generally refers to an interpretation of the concept of probability, where probability is interpreted as reasonable expectation. A Bayesian interpretation of probability may be considered an extension of propositional logic that enables reasoning with hypotheses, with propositions whose truth or falsity is unknown, where a probability is assigned to a hypothesis. To evaluate the probability of a hypothesis, a prior probability is specified, which may be updated to a posterior probability in view of relevant data.

In some embodiments, a method herein comprises generating a haplotype pair likelihood set for a haplotype group according to i) allele quantifications and ii) a probability of each haplotype pair. In some embodiments, a haplotype pair likelihood set for a haplotype group is generated given the observed data (e.g., allele quantifications). In some embodiments, a haplotype pair likelihood set for a haplotype group is generated, given the observed data (e.g., allele quantifications), according to i) allele quantifications and ii) a probability of each haplotype pair. In some embodiments, a haplotype pair likelihood set for a haplotype group is generated, given the observed data (e.g., allele quantifications), according to i) a probability of the observed data (e.g., allele quantifications) given each haplotype pair, and ii) a probability of each haplotype pair. In some embodiments, a haplotype pair likelihood set for a haplotype group is generated, given the allele quantifications, according to i) a probability of the allele quantifications given each haplotype pair, and ii) a probability of each haplotype pair. In some embodiments, the probability in (i) is determined according to which genotype is most likely observed at each genomic locus across a haplotype group, given a particular haplotype pair. In some embodiments, a method herein comprises calculating the probability of the allele quantifications at each at each genomic locus and generating a product across all genomic loci in the haplotype group. In some embodiments, the probability in (i) is adjusted according to a measure of sequencing error, as described herein.

In some embodiments, a probability of each haplotype pair is determined, in part, according to haplotype frequencies (e.g., haplotype frequencies described herein, haplotype frequencies in a population, haplotype frequencies in a database). In some embodiments, a probability of each haplotype pair is determined, in part, according to haplotype frequencies for each haplotype species (e.g., for haplotype pair AB, the frequency of haplotype species A in a population and the frequency of haplotype species B in a population). In some embodiments, a probability of each haplotype pair is determined, in part, according to haplotype frequencies for (i) a target reference allele and a linked reference allele, (ii) a target reference allele and a linked alternative allele, (iii) a target alternative allele and a linked reference allele, and (iv) a target alternative allele and a linked alternative allele. In some embodiments, a probability of each haplotype pair is determined, in part, according to haplotype frequencies for (i) a first linked reference allele and a second linked reference allele, (ii) a first linked reference allele and a second linked alternative allele, (iii) a first linked alternative allele and a second linked reference allele, and (iv) a first linked alternative allele and a second linked alternative allele.

In some embodiments, a haplotype pair likelihood set for a haplotype group is generated according to a probability that the test sample has a particular haplotype pair, i and j, given the allele quantifications in (b), D, where the probability, P(Hᵢ,Hⱼ|D), is derived from equation A:

$$P ( {H}_{i} , {H}_{j} \vert D ) = \frac{P ( D \vert {H}_{i} , {H}_{j} ) \times P ( {H}_{i} , {H}_{j} )}{P ( D )} \qquad ( A )$$

where P(D|Hᵢ,Hⱼ) is the probability of the allele quantifications, given the allele quantifications derive from haplotype pair Hᵢ, Hⱼ; P(Hᵢ, Hⱼ) is the probability of each haplotype pair derived from haplotype frequencies; and P(D) is the probability of the data (allele quantifications). Generally it is not necessary to explicitly calculate P(D) because this term is cancelled out when the ratios of P(Hᵢ, Hⱼ|D) are taken later. In some embodiments, P(D|Hᵢ,Hⱼ) is determined according to which genotype is most likely observed at each genomic locus, s, across the haplotype group, given haplotype pair Hᵢ, Hⱼ is present.

In some embodiments, a method herein comprises calculating the probability of the allele quantifications, D, at each at each genomic locus, s, and generating a product across all genomic loci in the haplotype group according to equation B:

$$P ( D \vert {H}_{i} , {H}_{j} ) = {\prod }_{s = 1}^{n} P ( {D}_{s} \vert {H}_{i s} , {H}_{j s} ) . \qquad ( B )$$

A method herein may comprise generating a genotype at a target genomic locus. A method herein may comprise generating a genotype at a target genomic locus based on a haplotype pair likelihood set. A genotype at a target genomic locus may be chosen from homozygous for a target reference allele, heterozygous for a target reference allele and a target alternative allele, and homozygous for a target alternative allele. In some embodiments, a method herein comprises identifying the most probable haplotype pair from the haplotype pair likelihood set. A genotype at a target genomic locus may be generated according to the most probable haplotype pair. For example, a most probable haplotype pair may comprise a specific allele at a target locus in a first haplotype species in the pair and specific allele at a target locus in a second haplotype species in the pair. Thus, the genotype at a target genomic locus is both target alleles ([reference, reference]; [reference, alternative]; or [alternative, alternative]) in the selected haplotype pair. In some embodiments, a method herein comprises aggregating haplotype pair likelihoods across all haplotype pairs for the haplotype group, thereby generating aggregate likelihoods. For example, each haplotype pair corresponds to one of three possible target genomic locus genotypes (homozygous reference, heterozygous, and homozygous alternative). The probability of the data (e.g., allele quantifications) given a haplotype pair, calculated as described above, may be added to an aggregate probability of the corresponding target genomic locus genotype. A genotype at a target genomic locus may be generated according to the highest aggregate likelihood.

In some embodiments, a plurality of genotypes at a plurality of target genomic loci are generated. In some embodiments, a plurality of genotypes at a plurality of target genomic loci are generated based on a plurality of haplotype pair likelihood sets. In some embodiments, a method herein comprises identifying a subject based on a plurality of genotypes generated for a test sample, as described herein.

### Genotyping One or More Subjects/Sources in a Mixed Sample

Provided herein are methods for generating a genotype for a target genomic locus (e.g., a target genomic locus described herein) for one or more sources or subjects in a mixed sample. Provided herein are methods for generating a genotype for a target genomic locus (e.g., a target genomic locus described herein) for an unknown source or subject. Methods herein may comprise generating a genotype for a target genomic locus for an unknown source or subject from a mixed sample (e.g., a sample comprising nucleic acid from a known source and an unknown source; a sample comprising nucleic acid from a known subject and an unknown subject). Also provided herein are methods for generating a plurality of genotypes for a plurality of genomic loci for an unknown source or subject. Methods herein may comprise generating a plurality of genotypes for a plurality of genomic loci for an unknown source or subject from a mixed sample. In certain implementations of the methods herein, the identity of an unknown subject and/or the identities of one or more relatives of an unknown subject may be determined based on the plurality of genotypes generated for a plurality of genomic loci and in connection with a genealogy analysis. Genotyping data obtained from mixed sample may be referred as convoluted, and genotyping data obtained from a mixed sample using a genotyping method described herein may be referred to as deconvoluted. Accordingly, a genotyping method for a mixed sample described herein may be referred to as a genotype deconvolution method.

In some embodiments, a method herein comprises obtaining sequence reads aligned to a reference genome for a test sample. Methods for obtaining sequence reads and methods for aligning sequence reads to a reference genome are described herein. Sequence reads may be obtained by directly sequencing nucleic acid in a test sample to obtain the sequence information, or may be obtained by receiving sequence information obtained by another (e.g., another party that generated the sequence reads by a sequencing process). In some embodiments, a method herein comprises obtaining sequence reads aligned to a reference genome for a test sample comprising a mixture of nucleic acid (e.g., nucleic acid from more than one subject or source). In some embodiments, a method herein comprises obtaining sequence reads aligned to a reference genome for a test sample comprising unmixed or pure nucleic acid (e.g., nucleic acid from a single subject or source).

In some embodiments, a test sample comprising a mixture of nucleic acid comprises nucleic acid from more than one source or subject. More than one subject generally refers to more than one individual (e.g., more than one animal, more than one human). More than one source generally refers to more than one individual or more than one source within a single individual (e.g., nucleic acid obtained from a tumor source and nucleic acid obtained from a host source from the same individual). The terms source and subject may be used interchangeably herein in the context of a mixed sample. In some embodiments, a test sample comprising a mixture of nucleic acid comprises nucleic acid from two sources or subjects. In some embodiments, a test sample comprising a mixture of nucleic acid comprises nucleic acid from two human subjects. In some embodiments, a test sample comprising a mixture of nucleic acid comprises nucleic acid from a known subject and an unknown subject. In some embodiments, a test sample comprising a mixture of nucleic acid comprises nucleic acid from two unknown subjects. In some embodiments, a test sample comprising a mixture of nucleic acid comprises nucleic acid from two or more subjects. In some embodiments, a test sample comprising a mixture of nucleic acid comprises nucleic acid from two or more subjects, where one or more of the subjects is known and one or more of the subjects in unknown. In some embodiments, a test sample comprising unmixed or pure nucleic acid comprises nucleic acid from one subject. In some embodiments, a test sample comprising unmixed or pure nucleic acid consists of nucleic acid from one subject. In some embodiments, a test sample comprising unmixed or pure nucleic acid comprises nucleic acid from a known subject and no nucleic acid from an unknown subject. In some embodiments, a test sample comprising unmixed or pure nucleic acid consists of nucleic acid from a known subject. A test sample comprising a mixture of nucleic acid may be referred to herein as a first test sample and a test sample comprising unmixed or pure nucleic acid may be referred to as a second test sample. In some embodiments, a first test sample is a forensic sample. In some embodiments, a first test sample is a non-forensic sample. In some embodiments, a second test sample is a forensic sample. In some embodiments, a second test sample is a non-forensic sample.

A known subject generally is an individual whose identity is known and/or documented. Often, a nucleic acid sample (e.g., an unmixed or pure nucleic acid sample) can readily be obtained from a known subject. A known subject sometimes may be referred to herein as a known sample. An unknown subject generally is an individual whose identity is not known or documented. Often, a nucleic acid sample (e.g., an unmixed or pure nucleic acid sample) cannot readily be obtained from an unknown subject. An unknown subject sometimes may be referred to herein as an unknown sample. A non-limiting example of a known subject is a crime victim and a non-limiting example of an unknown subject is a perpetrator.

A test sample may comprise a mixture of nucleic acid comprising nucleic acid from more than one source or subject. Non-limiting examples of sources/subjects for a mixed sample include victim and perpetrator (e.g., from a forensic/crime sample), cancer and non-cancer tissue (e.g., from a biopsy), rare/minority circulating cells and majority cells, rare/minority circulating cell-free nucleic acid and majority circulating cell-free nucleic acid, a transplanted organ and a host, a pregnant female and a fetus, animal samples from mixed sources (urine, stool, collected hair), mixed food/beverage sources, and the like.

In some embodiments, a method herein comprises generating allele quantifications. In some embodiments, allele quantifications are generated from sequence reads obtained from a mixed sample. In some embodiments, a method herein comprises generating allele quantifications for a linked genomic locus. Allele quantifications for a linked genomic locus may be generated from sequence reads obtained from a mixed sample. Allele quantifications for a linked genomic locus may comprise quantifying a linked reference allele (major allele at a linked genomic locus) and quantifying a linked alternative allele (minor allele at a linked genomic locus). In some embodiments, a method herein comprises quantifying a plurality of linked reference alleles and quantifying a plurality of linked alternative alleles, thereby generating a plurality of allele quantifications for a plurality of linked genomic loci. In some embodiments, a method herein comprises generating allele quantifications for a target genomic locus. Allele quantifications for a target genomic locus may be generated from sequence reads obtained from a mixed sample. Allele quantifications for a target genomic locus may comprise quantifying a target reference allele (major allele at a target genomic locus) and quantifying a target alternative allele (minor allele at a target genomic locus). Allele quantifications may be generated according to the amount of sequence reads, or an adjusted amount of sequence reads, carrying a particular allele at a genomic locus, as described in further detail herein. In some embodiments, allele quantifications are obtained at a locus where the known subject has an alternative allele by counting the sequence reads from the mixture sample that support each allele. In some embodiments, allele quantifications are obtained at a plurality of loci where the known subject has an alternative allele by counting the sequence reads from the mixture sample that support each allele.

In some embodiments, a method herein comprises obtaining one or more genotypes for a known subject. In some embodiments, a method herein comprises obtaining a genotype for a linked genomic locus for a known subject. In some embodiments, a method herein comprises obtaining a plurality of genotypes for a plurality of linked genomic loci for a known subject. In some embodiments, a method herein comprises obtaining a genotype for a target genomic locus for a known subject. In some embodiments, a method herein comprises obtaining a plurality of genotypes for a plurality of target genomic loci for a known subject. Genotypes for a known subject may be obtained according to a method described herein. In some embodiments, one or more genotypes for a known subject may be obtained from sequence reads generated from a sample from the known subject. A sample from a known subject may be an unmixed or pure sample, comprising nucleic acid from the known subject and comprising no nucleic acid from an unknown subject. A sample from a known subject may be an unmixed or pure sample, consisting of nucleic acid from the known subject.

In some embodiments, a method herein comprises estimating a fraction of nucleic acid in a nucleic acid mixture. A fraction of nucleic acid in a nucleic acid mixture generally refers to the proportion of nucleic acid in the mixture derived from each subject or source. In some embodiments, a method herein comprises estimating a fraction of nucleic acid from a known subject and/or an unknown subject in a nucleic acid mixture. In some embodiments, a method herein comprises estimating a fraction of nucleic acid from an unknown subject in a nucleic acid mixture. A fraction of nucleic acid in a nucleic acid mixture may be referred to herein as a mixture fraction, a mixture proportion, mixture percentage, unknown fraction, and the like. A fraction of nucleic acid in a nucleic acid mixture may be represented by β and/or θ (e.g., in certain equations and models provided herein).

In some embodiments, estimating a fraction of nucleic acid from an unknown subject in a mixture is based on a deviation from an expected variant allele frequency (VAF) for a known subject. For example, SNPs with very low population allele frequencies (PAF) (e.g., below 5%, below 4%, below 3%, below 2%, below 1%), often have a genotype that is homozygous reference for an unknown subject. In instances where the known subject contains one or two alternative alleles at a low PAF SNP (i.e., heterozygous or homozygous for the alternative allele), such an SNP site can be used to estimate the fraction of nucleic acid from the unknown subject. For example, the fraction of nucleic acid from the unknown subject may be estimated according to a deviation or shift from the expected VAF value (e.g., a shift from a VAF of 0.5 for a heterozygous genotype or a shift from a VAF of 1.0 for a homozygous genotype). A quantification of the deviation or shift represents the amount of nucleic acid from the unknown subject in the mixed sample. Such fraction estimates may be used in a probability model to genotype the unknown subject, as described herein.

In some embodiments, a fraction of nucleic acid in a nucleic acid mixture is learned. In some embodiments, a fraction of nucleic acid in a nucleic acid mixture is learned according to a probabilistic model (e.g., as described in Examples 3 and 4). Probabilistic models generally incorporate random variables and probability distributions into a model of an event. A probabilistic model can factor elements of randomness and may generate a probability distribution as a solution. Random variables from a distribution (e.g., normal distribution, binomial distribution, Bernoulli distribution) may form the foundation for probabilistic modelling.

In some embodiments, an initial fraction of nucleic acid is estimated or guessed. In some embodiments, a plurality of initial fractions of nucleic acid are estimated or guessed. An initial estimate or guess may be based on a deviation from an expected variant allele frequency (VAF) as described above, and/or may be based on a sampling of fraction values between 0% to 100% (or 0.0 and 1.0). In some embodiments, estimating a fraction of nucleic acid (e.g., from an unknown subject) in a mixture is based on incremental estimates between 0% to 100%. Incremental estimates may be any suitable increment value. For example, incremental estimates may be 1% incremental estimates, 2% incremental estimates, 3% incremental estimates, 4% incremental estimates, 5% incremental estimates, 6% incremental estimates, 7% incremental estimates, 8% incremental estimates, 9% incremental estimates, or 10% incremental estimates.

In some embodiments, a method herein comprises generating a genotype likelihood for an unknown subject. In some embodiments, a method herein comprises generating a plurality of genotype likelihoods for an unknown subject. In some embodiments, a method herein comprises generating a total genotype likelihood based on a plurality of genotype likelihoods. In some embodiments, a method herein comprises generating a genotype likelihood for a linked genomic locus for an unknown subject. In some embodiments, a method herein comprises generating a genotype likelihood for a linked genomic locus according to an allele quantification for the linked genomic locus for an unknown subject. In some embodiments, a method herein comprises generating a plurality of genotype likelihoods for a plurality of linked genomic loci for an unknown subject. Generating a plurality of genotype likelihoods for a plurality of linked genomic loci may refer to genotype likelihoods generated for a group of genetic loci linked to a target genomic locus. In some embodiments, generating a plurality of genotype likelihoods for a plurality of linked genomic loci may refer to genotype likelihoods generated for groups of genetic loci where each group is linked to a different target genomic locus. In some embodiments, generating a plurality of genotype likelihoods for a plurality of linked genomic loci may refer to genotype likelihoods generated for linked loci across the genome. In some embodiments, a plurality of genotype likelihoods for a plurality of linked genomic loci is generated according to a plurality of allele quantifications for the plurality of linked genomic loci. In some embodiments, a method herein comprises generating a total genotype likelihood based on the plurality genotype likelihoods for linked genomic loci for an unknown subject.

In some embodiments, a method herein comprises generating a genotype likelihood for a target genomic locus. In some embodiments, a method herein comprises generating a genotype likelihood for a target genomic locus for an unknown subject. In some embodiments, a method herein comprises generating a genotype likelihood for a target genomic locus according to an allele quantification for the target genomic locus. In some embodiments, only a target genomic locus is used as a linked genomic locus or a plurality of linked genomic loci. In some embodiments, a method herein comprises generating a plurality of genotype likelihoods for a plurality of target genomic loci. In some embodiments, a plurality of genotype likelihoods for a plurality of target genomic loci is generated according to a plurality of allele quantifications for the plurality of target genomic loci. A plurality of genotype likelihoods for a plurality of target genomic loci may refer to genotype likelihoods generated two or more target genomic loci. In some embodiments, a plurality of genotype likelihoods for a plurality of target genomic loci may refer to genotype likelihoods generated for target loci across the genome. In some embodiments, a method herein comprises generating a total genotype likelihood based on the plurality genotype likelihoods for target genomic loci for an unknown subject.

In some embodiments, a method herein comprises generating a plurality of genotype likelihoods for a plurality of genomic loci. A plurality of genomic loci may include linked genomic loci, target genomic loci, or linked genomic loci and target genomic loci. In some embodiments, a plurality of genotype likelihoods for a plurality of linked and target genomic loci is generated according to a plurality of allele quantifications for the plurality of linked and target genomic loci. In some embodiments, a method herein comprises generating a total genotype likelihood based on the plurality genotype likelihoods for linked and target genomic loci. A plurality of genotype likelihoods for a plurality of linked and target genomic loci may refer to genotype likelihoods generated one or more linked genomic loci and one or more target genomic loci. In some embodiments, a plurality of genotype likelihoods for a plurality of linked and target genomic loci may refer to genotype likelihoods generated for linked and target loci across the genome. In some embodiments, a method herein comprises generating a total genotype likelihood based on the plurality genotype likelihoods for linked and target genomic loci for an unknown subject.

In some embodiments, a genotype likelihood for a linked genomic locus for an unknown subject is generated for a locus where a known subject has one or more particular alleles (e.g., reference alleles, alternative alleles) at the locus. In some embodiments, a genotype likelihood for a linked genomic locus for an unknown subject is generated for a locus where a known subject has one or more alternative alleles at the locus. In some embodiments, a genotype likelihood for a linked genomic locus for an unknown subject is generated for a locus where a known subject has one or two alternative alleles at the locus. In some embodiments, the known subject is homozygous for a linked alternative allele at the locus. In some embodiments, the known subject is heterozygous for a linked alternative allele and a linked reference allele at the locus.

In some embodiments, a method herein comprises generating a genotype likelihood for a known subject. In some embodiments, a method herein comprises generating a plurality of genotype likelihoods for a known subject. In some embodiments, a method herein comprises generating a total genotype likelihood based on a plurality of genotype likelihoods. In some embodiments, a method herein comprises generating a genotype likelihood for a linked genomic locus for a known subject. In some embodiments, a method herein comprises generating a genotype likelihood for a linked genomic locus according to an allele quantification for the linked genomic locus for a known subject. In some embodiments, a method herein comprises generating a plurality of genotype likelihoods for a plurality of linked genomic loci for a known subject. In some embodiments, a plurality of genotype likelihoods for a plurality of linked genomic loci is generated according to a plurality of allele quantifications for the plurality of linked genomic loci. In some embodiments, a method herein comprises generating a total genotype likelihood based on the plurality genotype likelihoods for linked genomic loci for a known subject.

A genotype likelihood for a linked genomic locus for an unknown subject may be generated given an estimated fraction of nucleic acid in a nucleic acid mixture. A genotype likelihood for a linked genomic locus for an unknown subject may be generated according to allele quantifications as described above (i.e., allele quantifications generated from sequence reads obtained from a mixed sample). In some embodiments, a genotype likelihood for a linked genomic locus for an unknown subject may be generated given an estimated fraction of nucleic acid in a nucleic acid mixture and according to allele quantifications for the mixture. In some embodiments, a genotype likelihood for a linked genomic locus for an unknown subject may be generated according to an expectation-maximization (EM) algorithm. In some embodiments, a fraction of nucleic acid in a nucleic acid mixture may be estimated according to an expectation-maximization (EM) algorithm. In some embodiments, a genotype likelihood for a linked genomic locus for an unknown subject may be generated according to an expectation-maximization (EM) algorithm and a fraction of nucleic acid in a nucleic acid mixture may be estimated according to an expectation-maximization (EM) algorithm. An expectation-maximization (EM) algorithm is one approach for determining maximum likelihood estimates for model parameters when data is incomplete, has missing data points, or has unobserved (hidden) latent variables. An expectation-maximization (EM) algorithm is an iterative process for approximating a maximum likelihood function. An EM algorithm can find model parameters even if data is missing. This algorithm typically works by choosing random values for the missing data points, and using those guesses to estimate a second set of data. The new values are used to create a better guess for the first set, and the process continues until the algorithm converges on a fixed point. For example, allele quantifications may be used to compute the numerical likelihood of an unknown subject's genotype, Lᵢ, at that locus i. This quantity may be represented by P(D|Lᵢ, θ), where D is the data, and θ is the mixture percentage. Using the likelihoods of the unknown subject's genotype, Lᵢ, and the expectation maximization (EM) equations (provided in Example 4), a new estimate of the fraction of nucleic acid in a nucleic acid mixture, θ, may be calculated. In some instances (e.g., if this this is the first time calculating a genotype likelihood and generating a new fraction estimate, or if the total likelihood has changed by more than a pre-set threshold), a genotype likelihood may be recalculated using the new estimate of the fraction of nucleic acid in the mixture. A genotype likelihood may be recalculated any suitable number of times. In some embodiments, a genotype likelihood may be recalculated until the total likelihood value changes by a percentage that is less than a threshold value. Accordingly, in some embodiments, a method herein comprises iteratively generating a new estimate of the fraction of nucleic acid from an unknown subject in a mixture and calculating a different genotype likelihood according to the new fraction estimate. In some embodiments, a method herein comprises iteratively generating a new estimate of the fraction of nucleic acid from an unknown subject in a mixture and calculating a different genotype likelihood according to the new fraction estimate until a certain measure is reached. In some embodiments, a method herein comprises iteratively generating a new estimate of the fraction of nucleic acid from an unknown subject in a mixture and calculating a different genotype likelihood according to the new fraction estimate until the total likelihood value changes by a percentage that is less than a threshold value. A threshold value may be any suitable threshold value. In some embodiments, a threshold value is at least about 0.001%. For example, a threshold value may be about 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, or 0.1%. In some embodiments, the threshold value is 0.01%. Once a fraction estimation process has completed, the genotype likelihoods for the unknown subject, P(D|Lᵢ, θ), may be used for a genetic imputation process described herein.

In some embodiments, a method herein comprises generating a set of genotype likelihoods. In some embodiments, a method herein comprises generating a set of genotype likelihoods for an unknown subject. In some embodiments, a method herein comprises generating a set of genotype likelihoods for a known subject. In some embodiments, a method herein comprises generating a set of genotype likelihoods for a target genomic locus. In some embodiments, a method herein comprises generating a set of genotype likelihoods for a target reference allele and a target alternative allele at a target genomic locus. In some embodiments, a method herein comprises generating a set of genotype likelihoods for a target reference allele and a target alternative allele at a target genomic locus for an unknown subject. In some embodiments, a method herein comprises generating a set of genotype likelihoods for a target reference allele and a target alternative allele at a target genomic locus for a known subject. In some embodiments, a set of genotype likelihoods comprises one or more likelihoods for genotypes chosen from homozygous for a target reference allele, heterozygous for a target reference allele and a target alternative allele, and homozygous for a target alternative allele. In some embodiments, a plurality of genotype likelihood sets for a target genomic locus is generated. In some embodiments, a plurality of genotype likelihood sets for a target genomic locus is generated according to a plurality of genotype likelihoods for a linked genomic loci. In some embodiments, a composite genotype likelihood is generated for each genotype from a plurality of genotype likelihood sets. Generating a set of genotype likelihoods, a plurality of genotype likelihood sets, and a composite genotype likelihood are described in further detail herein.

Generating a set of genotype likelihoods for a target reference allele and a target alternative allele at a target genomic locus may be based on a linked genomic locus genotype likelihood (e.g., a linked genomic locus genotype likelihood described above). Generating a set of genotype likelihoods for a target reference allele and a target alternative allele at a target genomic locus may be based on a probability of a genotype at the target genomic locus based on prior probabilities of the target reference allele and the target alternative allele. In some embodiments, generating a set of genotype likelihoods for a target reference allele and a target alternative allele at a target genomic locus is based on 1) a linked genomic locus genotype likelihood, and 2) a probability of a genotype at the target genomic locus based on prior probabilities of the target reference allele and the target alternative allele.

In some embodiments, a probability of a genotype at a target genomic locus is based, in part, on haplotype frequencies. For example, a probability of a genotype at a target genomic locus may be based, in part, on haplotype frequencies for (i) a target reference allele and a linked reference allele, (ii) a target reference allele and a linked alternative allele, (iii) a target alternative allele and a linked reference allele, and (iv) a target alternative allele and a linked alternative allele. A probability of a genotype at a target genomic locus, prior probabilities, and haplotype frequencies, are discussed in further detail herein.

In some embodiments a genotype likelihood for a target genomic locus is generated according to the following, where the likelihood (P(D/T,θ)) of the data (D) at a mixture percentage (θ) for a genotype per target site (T) for an unknown subject is generated according to a process represented by equation (1′):

$$P ( D \vert T , θ ) = {\prod }_{i} {\sum }_{{L}_{i} ∈ 0 , 1 , 2} P ( {D}_{i} \vert {L}_{i} , {Y}_{i} , θ ) P ( {L}_{i} \vert T ) \qquad ( {1}^{′} )$$

where
D is sequence read data from the mixture,
θ is the mixture percentage,
i is a linked locus to T,
Dᵢ is sequence read data from the mixture at locus i,
Lᵢ is a genotype of an unknown subject at locus i, and
Yᵢ is a genotype of a known subject at locus i.

Certain components of equation (1′) may be defined by equation (2′):

$$P ( {D}_{i} \vert {L}_{i} , {Y}_{i} , θ ) ∼ Binomial ( \vert {D}_{i} \vert , \frac{{L}_{i} θ + {Y}_{i} ( 1 - θ )}{2} ) \qquad ( {2}^{′} )$$

where Binomial is a standard binomial distribution (e.g., Binomial(n, p) with n trials, and frequency parameter p).

Certain components of equation (1′) may be defined by equation (3′):

$$P ( {L}_{i} \vert T ) = \frac{haplofreq ( {L}_{i} , T )}{haplofreq ( T )} \qquad ( {3}^{′} )$$

where
haplofreq(Lᵢ, T) is a population frequency of co-occurrence of genotypes Lᵢ and T in the same haplotype (e.g., from a population database); generally, haplofreq(x,y) is a population frequency of co-occurrence of genotypes x and y in the same haplotype (e.g., from a population database), and
haplofreq(T) is a population frequency of genotype T, from a population database.

In some embodiments haplofreq(Lᵢ, T) is determined according to one or more haplotype frequencies. In some embodiments haplofreq(Lᵢ, T) is determined according to one or more haplotype frequencies for a linked allele and a target allele. In some embodiments haplofreq(Lᵢ, T) is determined according to one or more haplotype frequencies for a linked reference allele or a linked alternative allele, and a target reference allele or a target alternative allele. In some embodiments haplofreq(Lᵢ, T) is determined according to one or more haplotype frequencies chosen from Lᵢ0T0, Lᵢ1T0, Lᵢ0T1 and Lᵢ1T1, where Lᵢ0T0 is a haplotype frequency for a linked reference allele and a target reference allele, Lᵢ1T0 is a haplotype frequency for a linked alternative allele and a target reference allele, Lᵢ0T1 is a haplotype frequency for a linked reference allele and a target alternative allele, and Lᵢ1T1 is a haplotype frequency for a linked alternative allele and a target alternative allele. Haplotype frequencies may be expressed herein with the linked allele listed first and the target allele listed second (e.g. Lᵢ0T0) or the target allele listed first and the linked allele listed second (e.g., T0Lᵢ0), and both formats may be used interchangeably herein.

In some embodiments, P(Lᵢ|T) in equation (1′) is determined according to one or more haplotype frequencies. In some embodiments P(Lᵢ|T) in equation (1′) is determined according to one or more haplotype frequencies for a linked allele and a target allele. In some embodiments P(Lᵢ|T) in equation (1′) is determined according to one or more haplotype frequencies for a linked reference allele or a linked alternative allele, and a target reference allele or a target alternative allele. In some embodiments P(Lᵢ|T) is determined according to one or more haplotype frequencies chosen from Lᵢ0T0, Lᵢ1T0, Lᵢ0T1 and Lᵢ1T1, where Lᵢ0T0 is a haplotype frequency for a linked reference allele and a target reference allele, Lᵢ1T0 is a haplotype frequency for a linked alternative allele and a target reference allele, Lᵢ0T1 is a haplotype frequency for a linked reference allele and a target alternative allele, and Lᵢ1T1 is a haplotype frequency for a linked alternative allele and a target alternative allele. P(Lᵢ|T) may represent or comprise four possibilities: P(Lᵢ=0|T=0), P(Lᵢ=1|T=0), P(Lᵢ=0|T=1), and P(Lᵢ=1|T=1). In some embodiments, P(Lᵢ|T) represents or comprises four possibilities: P(Lᵢ=0|T=0), P(Lᵢ=1|T=0), P(Lᵢ=0|T=1), and P(Lᵢ=1|T=1), where P(Lᵢ=0|T=0)=Lᵢ0T0/(Lᵢ0T0+Lᵢ1T0), P(Lᵢ=1|T=0)=Lᵢ1T0/(Lᵢ0T0+Lᵢ1T0), P(Lᵢ=0|T=1)=Lᵢ0T1/(Lᵢ0T1+Lᵢ1T1), and P(Lᵢ=1|T=1)=Lᵢ1T1/(Lᵢ0T1+Lᵢ1T1).

In some embodiments, a method herein comprises generating a genotype at a target genomic locus. In some embodiments, a method herein comprises generating a genotype at a target genomic locus for an unknown subject. In some embodiments, a method herein comprises generating a genotype at a target genomic locus for a known subject. A genotype at a target genomic locus may be generated according to a set of genotype likelihoods (e.g., a set of genotype likelihoods described above). In some embodiments, a genotype at a target genomic locus is generated according to a plurality of genotype likelihood sets. In some embodiments, a genotype at a target genomic locus is generated according to a genotype likelihood composite. In some embodiments, a method herein comprises generating a plurality of genotypes at a plurality of target genomic loci. A plurality of genotypes may be generated at a plurality of target genomic loci for an unknown subject. A plurality of genotypes may be generated at a plurality of target genomic loci for a known subject. In some embodiments, each genotype in a plurality of genotypes is generated independently from the other genotypes in the plurality of genotypes. In some embodiments, a method herein comprises identifying an unknown subject based on a plurality of genotypes generated for the unknown subject. Generating a genotype at a target genomic locus, generating a genotype according to a genotype likelihood composite, generating a plurality of genotypes at a plurality of target genomic loci, and independent genotypes are described in further detail herein.

Genotyping one or more subjects/sources in a mixed sample may have a variety of applications. For example, a method described herein may be useful for identifying an unknown perpetrator based on genotyping a mixed sample containing victim nucleic acid and perpetrator nucleic acid. A method described herein may be useful for deconvoluting data from cancer nucleic acid and non-cancer nucleic acid (e.g., from a biopsy). A method described herein may be useful for deconvoluting rare targets of interest from majority cells/samples (e.g., rare circulating cells or cfDNA). A method described herein may be useful for deconvoluting data from cfDNA shed from a transplanted/grafted organ (increased shedding may be a sign of poor organ health/potential rejection) from the bulk of host cfDNA. A method described herein may be useful for non-invasive prenatal testing (NIPT) applications. For example, a described herein may be useful for paternity testing and/or identification of genetic diseases/rhesus (Rh) factors. In particular, a method herein may be useful for deconvoluting data from fetal nucleic acid from maternal nucleic acid to allow greater resolution for non-invasive prenatal testing (NIPT) of chromosomal-level abnormalities and/or other genetic diseases, to allow genotyping of traits (e.g., fetal Rhesus +/− status to assess for Rh disease), and/or ancestry-related analysis such as paternity testing. A method described herein may be useful for wildlife population monitoring. Animal nucleic acid samples may be collected from mixed sources (e.g., animal urine, stool, collected hair) and the data deconvoluted to assess and track number, identity, and/or health of animals (e.g., endangered animals in the wild). A method described herein may be useful for food safety monitoring, wine/beer/liquor quality control, and/or fishing counterfeiting. For example, in a mixed food setting, a method herein may be used to deconvolute data from nucleic acid to determine the various contributors/ingredients.

In some embodiments, a method herein comprises generating a genotype for a target genomic locus (e.g., a target genomic locus described herein) for an unknown source or unknown subject according to a haplotype analysis described herein and described in Example 5. In some embodiments, a method herein comprises generating a genotype for a target genomic locus (e.g., a target genomic locus described herein) for more than one unknown source or unknown subject according to a process described in Example 6. In some embodiments, a method herein comprises generating a genotype for a target genomic locus (e.g., a target genomic locus described herein) for a plurality of unknown sources or unknown subjects for a plurality of mixed samples according to a process described in Example 7. In some embodiments, a method herein comprises generating a genotype for a target genomic locus (e.g., a target genomic locus described herein) for an unknown source or unknown subject based on microarray data according to a process described in Example 8.

### Samples

Provided herein are methods and compositions for processing and/or analyzing nucleic acid. Nucleic acid or a nucleic acid mixture utilized in methods and compositions described herein may be isolated from a sample obtained from a subject (e.g., a test subject). A subject can be any living or non-living organism, including but not limited to a human, a non-human animal, a plant, a bacterium, a fungus, a protist or a pathogen. Any human or non-human animal can be selected, and may include, for example, mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. A subject may be a male or female (e.g., woman, a pregnant woman). A subject may be any age (e.g., an embryo, a fetus, an infant, a child, an adult). A subject may be a cancer patient, a patient suspected of having cancer, a patient in remission, a patient with a family history of cancer, and/or a subject obtaining a cancer screen. A subject may be a patient having an infection or infectious disease or infected with a pathogen (e.g., bacteria, virus, fungus, protozoa, and the like), a patient suspected of having an infection or infectious disease or being infected with a pathogen, a patient recovering from an infection, infectious disease, or pathogenic infection, a patient with a history of infections, infectious disease, pathogenic infections, and/or a subject obtaining an infectious disease or pathogen screen. A subject may be a transplant recipient. A subject may be a patient undergoing a microbiome analysis. In some embodiments, a test subject is a female. In some embodiments, a test subject is a human female. In some embodiments, a test subject is a male. In some embodiments, a test subject is a human male.

A nucleic acid sample may be isolated or obtained from any type of suitable biological specimen or sample (e.g., a test sample). A nucleic acid sample may be isolated or obtained from a single cell, a plurality of cells (e.g., cultured cells), cell culture media, conditioned media, a tissue, an organ, or an organism (e.g., bacteria, yeast, or the like). In some embodiments, a nucleic acid sample is isolated or obtained from a cell(s), tissue, organ, and/or the like of an animal (e.g., an animal subject). In some embodiments, a nucleic acid sample is isolated or obtained from a source such as bacteria, yeast, insects (e.g., Drosophila), mammals, amphibians (e.g., frogs (e.g., Xenopus)), viruses, plants, or any other mammalian or non-mammalian nucleic acid sample source.

A nucleic acid sample may be isolated or obtained from an extant organism or animal. In some instances, a nucleic acid sample may be isolated or obtained from an extinct (or “ancient”) organism or animal (e.g., an extinct mammal; an extinct mammal from the genus Homo). In some instances, a nucleic acid sample may be obtained as part of a diagnostic analysis.

In some instances, a nucleic acid sample may be obtained as part of a forensics analysis. In some embodiments, a genotyping method and/or a genealogy analysis described herein is applied to a forensic sample or specimen (e.g., a sample or specimen associated with a crime; unidentified remains associated with a crime). A forensic sample or specimen may include any biological substance that contains nucleic acid. For example, a forensic sample or specimen may include blood, semen, hair, skin, sweat, saliva, decomposed tissue, bone, fingernail scrapings, licked stamps/envelopes, sluff, touch DNA, razor residue, and the like. In some embodiments, a forensic sample or specimen comprises hair or hair fragments. In some embodiments, a forensic sample or specimen comprises bone or bone fragments.

In some embodiments, a genotyping method and/or a genealogy analysis described herein is applied to a non-forensic sample or specimen (e.g., a sample or specimen that is not associated with a crime; unidentified remains not associated with a crime; historical objects containing biological material of deceased (e.g., for genealogy purposes)). A non-forensic sample or specimen may include any biological substance that contains nucleic acid. For example, a non-forensic sample or specimen may include blood, semen, hair, skin, sweat, saliva, decomposed tissue, bone, fingernail scrapings, licked stamps/envelopes, sluff, touch DNA, razor residue, and the like. In some embodiments, a non-forensic sample or specimen comprises hair or hair fragments. In some embodiments, a non-forensic sample or specimen comprises bone or bone fragments.

A sample or test sample may be any specimen that is isolated or obtained from a subject or part thereof (e.g., a human subject, a pregnant female, a cancer patient, a patient having an infection or infectious disease, a transplant recipient, a fetus, a tumor, an infected organ or tissue, a transplanted organ or tissue, a microbiome). A sample sometimes is from a pregnant female subject bearing a fetus at any stage of gestation (e.g., first, second or third trimester for a human subject), and sometimes is from a post-natal subject. A sample sometimes is from a pregnant subject bearing a fetus that is euploid for all chromosomes, and sometimes is from a pregnant subject bearing a fetus having a chromosome aneuploidy (e.g., one, three (i.e., trisomy (e.g., T21, T18, T13)), or four copies of a chromosome) or other genetic variation. Non-limiting examples of specimens include fluid or tissue from a subject, including, without limitation, blood or a blood product (e.g., serum, plasma, or the like), umbilical cord blood, chorionic villi, amniotic fluid, cerebrospinal fluid, spinal fluid, lavage fluid (e.g., bronchoalveolar, gastric, peritoneal, ductal, ear, arthroscopic), biopsy sample (e.g., from pre-implantation embryo; cancer biopsy), celocentesis sample, cells (blood cells, placental cells, embryo or fetal cells, fetal nucleated cells or fetal cellular remnants, normal cells, abnormal cells (e.g., cancer cells)) or parts thereof (e.g., mitochondrial, nucleus, extracts, or the like), washings of female reproductive tract, urine, feces, sputum, saliva, nasal mucous, prostate fluid, lavage, semen, lymphatic fluid, bile, tears, sweat, breast milk, breast fluid, the like or combinations thereof. In some embodiments, a biological sample is a cervical swab from a subject. A fluid or tissue sample from which nucleic acid is extracted may be acellular (e.g., cell-free). In some embodiments, a fluid or tissue sample may contain cellular elements or cellular remnants. In some embodiments, fetal cells or cancer cells may be included in the sample.

A sample can be a liquid sample. A liquid sample can comprise extracellular nucleic acid (e.g., circulating cell-free DNA). Examples of liquid samples include, but are not limited to, blood or a blood product (e.g., serum, plasma, or the like), urine, cerebral spinal fluid, saliva, sputum, biopsy sample (e.g., liquid biopsy for the detection of cancer), a liquid sample described above, the like or combinations thereof. In certain embodiments, a sample is a liquid biopsy, which generally refers to an assessment of a liquid sample from a subject for the presence, absence, progression or remission of a disease (e.g., cancer). A liquid biopsy can be used in conjunction with, or as an alternative to, a sold biopsy (e.g., tumor biopsy). In certain instances, extracellular nucleic acid is analyzed in a liquid biopsy.

In some embodiments, a biological sample may be blood, plasma or serum. The term “blood” encompasses whole blood, blood product or any fraction of blood, such as serum, plasma, buffy coat, or the like as conventionally defined. Blood or fractions thereof often comprise nucleosomes. Nucleosomes comprise nucleic acids and are sometimes cell-free or intracellular. Blood also comprises buffy coats. Buffy coats are sometimes isolated by utilizing a ficoll gradient. Buffy coats can comprise white blood cells (e.g., leukocytes, T-cells, B-cells, platelets, and the like). Blood plasma refers to the fraction of whole blood resulting from centrifugation of blood treated with anticoagulants. Blood serum refers to the watery portion of fluid remaining after a blood sample has coagulated. Fluid or tissue samples often are collected in accordance with standard protocols hospitals or clinics generally follow. For blood, an appropriate amount of peripheral blood (e.g., between 3 to 40 milliliters, between 5 to 50 milliliters) often is collected and can be stored according to standard procedures prior to or after preparation.

An analysis of nucleic acid found in a subject's blood may be performed using, e.g., whole blood, serum, or plasma. An analysis of fetal DNA found in maternal blood, for example, may be performed using, e.g., whole blood, serum, or plasma. An analysis of tumor or cancer DNA found in a patient's blood, for example, may be performed using, e.g., whole blood, serum, or plasma. An analysis of pathogen DNA found in a patient's blood, for example, may be performed using, e.g., whole blood, serum, or plasma. An analysis of transplant DNA found in a transplant recipient's blood, for example, may be performed using, e.g., whole blood, serum, or plasma. Methods for preparing serum or plasma from blood obtained from a subject (e.g., a maternal subject; patient; cancer patient) are known. For example, a subject's blood (e.g., a pregnant woman's blood; patient's blood; cancer patient's blood) can be placed in a tube containing EDTA or a specialized commercial product such as Cell-Free DNA BCT (Streck, Omaha, NE) or Vacutainer SST (Becton Dickinson, Franklin Lakes, N.J.) to prevent blood clotting, and plasma can then be obtained from whole blood through centrifugation. Serum may be obtained with or without centrifugation-following blood clotting. If centrifugation is used then it is typically, though not exclusively, conducted at an appropriate speed, e.g., 1,500-3,000 times g. Plasma or serum may be subjected to additional centrifugation steps before being transferred to a fresh tube for nucleic acid extraction. In addition to the acellular portion of the whole blood, nucleic acid may also be recovered from the cellular fraction, enriched in the buffy coat portion, which can be obtained following centrifugation of a whole blood sample from the subject and removal of the plasma.

A sample may be a tumor nucleic acid sample (i.e., a nucleic acid sample isolated from a tumor). The term “tumor” generally refers to neoplastic cell growth and proliferation, whether malignant or benign, and may include pre-cancerous and cancerous cells and tissues. The terms “cancer” and “cancerous” generally refer to the physiological condition in mammals that is typically characterized by unregulated cell growth/proliferation. Examples of cancer include, but are not limited to, carcinoma, lymphoma, blastoma, sarcoma, leukemia, squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma, various types of head and neck cancer, and the like.

A sample may be heterogeneous. For example, a sample may include more than one cell type and/or one or more nucleic acid species. In some instances, a sample may include (i) fetal cells and maternal cells, (ii) cancer cells and non-cancer cells, and/or (iii) pathogenic cells and host cells. In some instances, a sample may include (i) cancer and non-cancer nucleic acid, (ii) pathogen and host nucleic acid, (iii) fetal derived and maternal derived nucleic acid, and/or more generally, (iv) mutated and wild-type nucleic acid. In some instances, a sample may include a minority nucleic acid species and a majority nucleic acid species, as described in further detail below. In some instances, a sample may include cells and/or nucleic acid from a single subject or may include cells and/or nucleic acid from multiple subjects. In some embodiments, a sample may include a mixture of nucleic acid from multiple subjects. In some embodiments, a sample may include a mixture of nucleic acid from two subjects. For example, a sample may include a mixture of nucleic acid from a victim and a perpetrator.

In some embodiments, a sample comprises double-stranded nucleic acid fragments. In some embodiments, a sample comprises single-stranded nucleic acid fragments. In some embodiments, a sample comprises double-stranded nucleic acid fragments and single-stranded nucleic acid fragments.

### Nucleic Acid

Provided herein are methods and compositions for processing and/or analyzing nucleic acid. The terms nucleic acid(s), nucleic acid molecule(s), nucleic acid fragment(s), target nucleic acid(s), nucleic acid template(s), template nucleic acid(s), nucleic acid target(s), target nucleic acid(s), polynucleotide(s), polynucleotide fragment(s), target polynucleotide(s), polynucleotide target(s), and the like may be used interchangeably throughout the disclosure. The terms refer to nucleic acids of any composition from, such as DNA (e.g., complementary DNA (cDNA; synthesized from any RNA or DNA of interest), genomic DNA (gDNA), genomic DNA fragments, mitochondrial DNA (mtDNA), recombinant DNA (e.g., plasmid DNA), and the like), RNA (e.g., message RNA (mRNA), short inhibitory RNA (siRNA), ribosomal RNA (rRNA), transfer RNA (tRNA), microRNA, transacting small interfering RNA (ta-siRNA), natural small interfering RNA (nat-siRNA), small nucleolar RNA (snoRNA), small nuclear RNA (snRNA), long non-coding RNA (lncRNA), non-coding RNA (ncRNA), transfer-messenger RNA (tmRNA), precursor messenger RNA (pre-mRNA), small Cajal body-specific RNA (scaRNA), piwi-interacting RNA (piRNA), endoribonuclease-prepared siRNA (esiRNA), small temporal RNA (stRNA), signal recognition RNA, telomere RNA, RNA highly expressed by a fetus or placenta, and the like), and/or DNA or RNA analogs (e.g., containing base analogs, sugar analogs and/or a non-native backbone and the like), RNA/DNA hybrids and polyamide nucleic acids (PNAs), all of which can be in single-stranded form or double-stranded form, and unless otherwise limited, can encompass known analogs of natural nucleotides that can function in a similar manner as naturally occurring nucleotides. A nucleic acid may be, or may be from, a plasmid, phage, virus, bacterium, autonomously replicating sequence (ARS), mitochondria, centromere, artificial chromosome, chromosome, or other nucleic acid able to replicate or be replicated in vitro or in a host cell, a cell, a cell nucleus or cytoplasm of a cell in certain embodiments. A template nucleic acid in some embodiments can be from a single chromosome (e.g., a nucleic acid sample may be from one chromosome of a sample obtained from a diploid organism). Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues. The term nucleic acid is used interchangeably with locus, gene, cDNA, and mRNA encoded by a gene. The term also may include, as equivalents, derivatives, variants and analogs of RNA or DNA synthesized from nucleotide analogs, single-stranded (“sense” or “antisense,” “plus” strand or “minus” strand, “forward” reading frame or “reverse” reading frame), and double-stranded polynucleotides. The term “gene” refers to a section of DNA involved in producing a polypeptide chain; and generally includes regions preceding and following the coding region (leader and trailer) involved in the transcription/translation of the gene product and the regulation of the transcription/translation, as well as intervening sequences (introns) between individual coding regions (exons). A nucleotide or base generally refers to the purine and pyrimidine molecular units of nucleic acid (e.g., adenine (A), thymine (T), guanine (G), and cytosine (C)). For RNA, the base thymine is replaced with uracil. Nucleic acid length or size may be expressed as a number of bases.

Target nucleic acids may be any nucleic acids of interest. Nucleic acids may be polymers of any length composed of deoxyribonucleotides (i.e., DNA bases), ribonucleotides (i.e., RNA bases), or combinations thereof, e.g., 10 bases or longer, 20 bases or longer, 50 bases or longer, 100 bases or longer, 200 bases or longer, 300 bases or longer, 400 bases or longer, 500 bases or longer, 1000 bases or longer, 2000 bases or longer, 3000 bases or longer, 4000 bases or longer, 5000 bases or longer. In certain aspects, nucleic acids are polymers composed of deoxyribonucleotides (i.e., DNA bases), ribonucleotides (i.e., RNA bases), or combinations thereof, e.g., 10 bases or less, 20 bases or less, 50 bases or less, 100 bases or less, 200 bases or less, 300 bases or less, 400 bases or less, 500 bases or less, 1000 bases or less, 2000 bases or less, 3000 bases or less, 4000 bases or less, or 5000 bases or less.

Nucleic acid may be single-stranded or double-stranded, or may be a mixture of single-stranded and double-stranded. Single stranded DNA (ssDNA), for example, can be generated by denaturing double stranded DNA by heating or by treatment with alkali, for example. Accordingly, in some embodiments, ssDNA is derived from double-stranded DNA (dsDNA). In some embodiments, a method herein comprises prior to combining a nucleic acid composition comprising dsDNA with sequencing adapters, denaturing the dsDNA, thereby generating ssDNA.

In certain embodiments, nucleic acid is in a D-loop structure, formed by strand invasion of a duplex DNA molecule by an oligonucleotide or a DNA-like molecule such as peptide nucleic acid (PNA). D loop formation can be facilitated by addition of E. coli RecA protein and/or by alteration of salt concentration, for example, using methods known in the art.

Nucleic acid (e.g., nucleic acid targets, single-stranded nucleic acid (ssNA), polynucleotides, oligonucleotides, overhangs, hybridization regions) may be described herein as being complementary to another nucleic acid, having a complementarity region, being capable of hybridizing to another nucleic acid, or having a hybridization region. The terms “complementary” or “complementarity” or “hybridization” generally refer to a nucleotide sequence that base-pairs by non-covalent bonds to a region of a nucleic acid. In the canonical Watson-Crick base pairing, adenine (A) forms a base pair with thymine (T), and guanine (G) pairs with cytosine (C) in DNA. In RNA, thymine (T) is replaced by uracil (U). As such, A is complementary to T and G is complementary to C. In RNA, A is complementary to U and vice versa. In a DNA-RNA duplex, A (in a DNA strand) is complementary to U (in an RNA strand). In some embodiments, one or more thymine (T) bases are replaced by uracil (U) in a sequencing adapter, and is/are complementary to adenine (A). Typically, “complementary” or “complementarity” or “capable of hybridizing” refer to a nucleotide sequence that is at least partially complementary. These terms may also encompass duplexes that are fully complementary such that every nucleotide in one strand is complementary or hybridizes to every nucleotide in the other strand in corresponding positions.

In certain instances, a nucleotide sequence may be partially complementary to a target, in which not all nucleotides are complementary to every nucleotide in the target nucleic acid in all the corresponding positions. For example, a hybridization region may be perfectly (i.e., 100%) complementary to a target region, or a hybridization region may share some degree of complementarity which is less than perfect (e.g., 70%, 75%, 85%, 90%, 95%, 99%). In another example, a hybridization region may be perfectly (i.e., 100%) complementary to an oligonucleotide, or a hybridization region may share some degree of complementarity which is less than perfect (e.g., 70%, 75%, 85%, 90%, 95%, 99%).

The percent identity of two nucleotide sequences can be determined by aligning the sequences for optimal comparison purposes (e.g., gaps can be introduced in the sequence of a first sequence for optimal alignment). The nucleotides at corresponding positions are then compared, and the percent identity between the two sequences is a function of the number of identical positions shared by the sequences (i.e., % identity=# of identical positions/total # of positions×100). When a position in one sequence is occupied by the same nucleotide as the corresponding position in the other sequence, then the molecules are identical at that position.

In some embodiments, nucleic acids in a mixture of nucleic acids are analyzed. A mixture of nucleic acids can comprise two or more nucleic acid species having the same or different nucleotide sequences, different lengths, different origins (e.g., genomic origins, fetal vs. maternal origins, cell or tissue origins, cancer vs. non-cancer origin, tumor vs. non-tumor origin, host vs. pathogen, host vs. transplant, host vs. microbiome, sample origins, subject origins, and the like), different overhang lengths, different overhang types (e.g., 5′ overhangs, 3′ overhangs, no overhangs), or combinations thereof. In some embodiments, a mixture of nucleic acids comprises single-stranded nucleic acid and double-stranded nucleic acid. In some embodiment, a mixture of nucleic acids comprises DNA and RNA. In some embodiment, a mixture of nucleic acids comprises ribosomal RNA (rRNA) and messenger RNA (mRNA). Nucleic acid provided for processes described herein may contain nucleic acid from one sample or from two or more samples (e.g., from 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, or 20 or more samples).

In some embodiments, target nucleic acids comprise damaged or degraded nucleic acid. Damaged or degraded nucleic acid may be referred to as low-quality nucleic acid or highly damaged/degraded nucleic acid. Damaged or degraded nucleic acid may be highly fragmented, and may include damage such as base analogs and abasic sites subject to miscoding lesions and/or intermolecular crosslinking. For example, sequencing errors resulting from deamination of cytosine residues may be present in certain sequences obtained from damaged or degraded DNA (e.g., miscoding of C to T and G to A). In some embodiments, target nucleic acids are derived from nicked double-stranded nucleic acid fragments. Nicked double-stranded nucleic acid fragments may be denatured (e.g., heat denatured) to generate ssNA fragments.

Nucleic acid may be derived from one or more sources (e.g., biological sample, blood, cells, serum, plasma, buffy coat, urine, lymphatic fluid, skin, hair, bone, soil, and the like) by methods known in the art. In some embodiments, nucleic acid may be derived from a forensic sample or specimen. In some embodiments, a genotyping method and/or a genealogy analysis described herein is applied to nucleic acid derived from a forensic sample or specimen. In some embodiments, nucleic acid derived from a forensic sample or specimen comprises damaged, degraded, and/or fragmented nucleic acid. In some embodiments, nucleic acid is derived from a forensic sample or specimen comprises comprising no living cells. Nucleic acid derived from a forensic sample or specimen may include nucleic acid derived from blood, semen, hair, skin, sweat, saliva, decomposed tissue, bone, fingernail scrapings, licked stamps/envelopes, sluff, touch DNA, razor residue, and the like. In some embodiments, nucleic acid derived from a forensic sample or specimen comprises nucleic acid derived from hair or hair fragments. In some embodiments, nucleic acid derived from a forensic sample or specimen comprises nucleic acid derived from hair or hair fragments, where the hair or hair fragments comprise no roots or living cells. In some embodiments, nucleic acid derived from a forensic sample or specimen comprises nucleic acid derived from bone or bone fragments. In some embodiments, nucleic acid derived from a forensic sample or specimen comprises nucleic acid derived from bone or bone fragments, where the bone or bone fragments comprise no living cells.

Any suitable method can be used for isolating, extracting and/or purifying DNA from a biological sample (e.g., from blood or a blood product), non-limiting examples of which include methods of DNA preparation (e.g., described by Sambrook and Russell, Molecular Cloning: A Laboratory Manual 3d ed., 2001), various commercially available reagents or kits, such as DNeasy®, RNeasy®, QIAprep®, QIAquick®, and QIAamp® (e.g., QIAamp® Circulating Nucleic Acid Kit, QiaAmp© DNA Mini Kit or QiaAmp® DNA Blood Mini Kit) nucleic acid isolation/purification kits by Qiagen, Inc. (Germantown, Md); GenomicPrep™ Blood DNA Isolation Kit (Promega, Madison, Wis.); GFX™ Genomic Blood DNA Purification Kit (Amersham, Piscataway, N.J.); DNAzol®, ChargeSwitch®, Purelink®, GeneCatcher® nucleic acid isolation/purification kits by Life Technologies, Inc. (Carlsbad, CA); NucleoMag®, NucleoSpin®, and NucleoBond® nucleic acid isolation/purification kits by Clontech Laboratories, Inc. (Mountain View, CA); the like or combinations thereof. In certain aspects, the nucleic acid is isolated from a fixed biological sample, e.g., formalin-fixed, paraffin-embedded (FFPE) tissue. Genomic DNA from FFPE tissue may be isolated using commercially available kits—such as the AllPrep® DNA/RNA FFPE kit by Qiagen, Inc. (Germantown, Md), the RecoverAll® Total Nucleic Acid Isolation kit for FFPE by Life Technologies, Inc. (Carlsbad, CA), and the NucleoSpin® FFPE kits by Clontech Laboratories, Inc. (Mountain View, CA).

In some embodiments, nucleic acid is extracted from cells using a cell lysis procedure. Cell lysis procedures and reagents are known in the art and may generally be performed by chemical (e.g., detergent, hypotonic solutions, enzymatic procedures, and the like, or combination thereof), physical (e.g., French press, sonication, and the like), or electrolytic lysis methods. Any suitable lysis procedure can be utilized. For example, chemical methods generally employ lysing agents to disrupt cells and extract the nucleic acids from the cells, followed by treatment with chaotropic salts. Physical methods such as freeze/thaw followed by grinding, the use of cell presses and the like also are useful. In some instances, a high salt and/or an alkaline lysis procedure may be utilized. In some instances, a lysis procedure may include a lysis step with EDTA/Proteinase K, a binding buffer step with high amount of salts (e.g., guanidinium chloride (GuHCl), sodium acetate) and isopropanol, and binding DNA in this solution to silica-based column. In some instances, a lysis protocol includes certain procedures described in Dabney et al., Proceedings of the National Academy of Sciences 110, no. 39 (2013): 15758-15763.

Nucleic acids can include extracellular nucleic acid in certain embodiments. The term “extracellular nucleic acid” as used herein can refer to nucleic acid isolated from a source having substantially no cells and also is referred to as “cell-free” nucleic acid (cell-free DNA, cell-free RNA, or both), “circulating cell-free nucleic acid” (e.g., CCF fragments, ccf DNA) and/or “cell-free circulating nucleic acid.” Extracellular nucleic acid can be present in and obtained from blood (e.g., from the blood of a human subject). Extracellular nucleic acid often includes no detectable cells and may contain cellular elements or cellular remnants. Non-limiting examples of acellular sources for extracellular nucleic acid are blood, blood plasma, blood serum and urine. In certain aspects, cell-free nucleic acid is obtained from a body fluid sample chosen from whole blood, blood plasma, blood serum, amniotic fluid, saliva, urine, pleural effusion, bronchial lavage, bronchial aspirates, breast milk, colostrum, tears, seminal fluid, peritoneal fluid, pleural effusion, and stool. As used herein, the term “obtain cell-free circulating sample nucleic acid” includes obtaining a sample directly (e.g., collecting a sample, e.g., a test sample) or obtaining a sample from another who has collected a sample. Extracellular nucleic acid may be a product of cellular secretion and/or nucleic acid release (e.g., DNA release). Extracellular nucleic acid may be a product of any form of cell death, for example. In some instances, extracellular nucleic acid is a product of any form of type I or type II cell death, including mitotic, oncotic, toxic, ischemic, and the like and combinations thereof. Without being limited by theory, extracellular nucleic acid may be a product of cell apoptosis and cell breakdown, which provides basis for extracellular nucleic acid often having a series of lengths across a spectrum (e.g., a “ladder”). In some instances, extracellular nucleic acid is a product of cell necrosis, necropoptosis, oncosis, entosis, pyrotosis, and the like and combinations thereof. In some embodiments, sample nucleic acid from a test subject is circulating cell-free nucleic acid. In some embodiments, circulating cell free nucleic acid is from blood plasma or blood serum from a test subject. In some aspects, cell-free nucleic acid is degraded. In some embodiments, cell-free nucleic acid comprises cell-free fetal nucleic acid (e.g., cell-free fetal DNA). In certain aspects, cell-free nucleic acid comprises circulating cancer nucleic acid (e.g., cancer DNA). In certain aspects, cell-free nucleic acid comprises circulating tumor nucleic acid (e.g., tumor DNA). In some embodiments, cell-free nucleic acid comprises infectious agent nucleic acid (e.g., pathogen DNA). In some embodiments, cell-free nucleic acid comprises nucleic acid (e.g., DNA) from a transplant. In some embodiments, cell-free nucleic acid comprises nucleic acid (e.g., DNA) from a microbiome (e.g., microbiome of gut, microbiome of blood, microbiome of mouth, microbiome of spinal fluid, microbiome of feces).

Cell-free DNA (cfDNA) may originate from degraded sources and often provides limiting amounts of DNA when extracted. Certain methods for generating nucleic acid libraries (e.g., methods for generating sequencing libraries described in International Patent Application Publication No. WO 2019/140201, U.S. Provisional Patent Application No. 62/830,211, and U.S. Provisional Patent Application No. 62/861,594, each of which is incorporated by reference herein) are able to capture a larger amount of short DNA fragments from cfDNA. cfDNA from cancer samples, for example, tends to have a higher population of short fragments. In certain instances, short fragments in cfDNA may be enriched for fragments originating from transcription factors rather than nucleosomes.

Extracellular nucleic acid can include different nucleic acid species, and therefore is referred to herein as “heterogeneous” in certain embodiments. For example, blood serum or plasma from a person having a tumor or cancer can include nucleic acid from tumor cells or cancer cells (e.g., neoplasia) and nucleic acid from non-tumor cells or non-cancer cells. In another example, blood serum or plasma from a pregnant female can include maternal nucleic acid and fetal nucleic acid. In another example, blood serum or plasma from a patient having an infection or infectious disease can include host nucleic acid and infectious agent or pathogen nucleic acid. In another example, a sample from a subject having received a transplant can include host nucleic acid and nucleic acid from the donor organ or tissue. In some instances, cancer nucleic acid, tumor nucleic acid, fetal nucleic acid, pathogen nucleic acid, or transplant nucleic acid sometimes is about 5% to about 50% of the overall nucleic acid (e.g., about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49% of the total nucleic acid is cancer, tumor, fetal, pathogen, transplant, or microbiome nucleic acid). In another example, heterogeneous nucleic acid may include nucleic acid from two or more subjects (e.g., a sample from a crime scene).

Nucleic acid may be provided for conducting methods described herein with or without processing of the sample(s) containing the nucleic acid. In some embodiments, nucleic acid is provided for conducting methods described herein after processing of the sample(s) containing the nucleic acid. For example, a nucleic acid can be extracted, isolated, purified, partially purified or amplified from the sample(s). The term “isolated” as used herein refers to nucleic acid removed from its original environment (e.g., the natural environment if it is naturally occurring, or a host cell if expressed exogenously), and thus is altered by human intervention (e.g., “by the hand of man”) from its original environment. The term “isolated nucleic acid” as used herein can refer to a nucleic acid removed from a subject (e.g., a human subject). An isolated nucleic acid can be provided with fewer non-nucleic acid components (e.g., protein, lipid) than the amount of components present in a source sample. A composition comprising isolated nucleic acid can be about 50% to greater than 99% free of non-nucleic acid components. A composition comprising isolated nucleic acid can be about 90%, 91%, 92%, 93%, 94%, 95%, 96%, 97%, 98%, 99% or greater than 99% free of non-nucleic acid components. The term “purified” as used herein can refer to a nucleic acid provided that contains fewer non-nucleic acid components (e.g., protein, lipid, carbohydrate) than the amount of non-nucleic acid components present prior to subjecting the nucleic acid to a purification procedure. A composition comprising purified nucleic acid may be about 80%, 81%, 82%, 83%, 84%, 85%, 86%, 87%, 88%, 89%, 90%, 91%, 92%, 93%, 94%, 95%, 96%, 97%, 98%, 99% or greater than 99% free of other non-nucleic acid components. The term “purified” as used herein can refer to a nucleic acid provided that contains fewer nucleic acid species than in the sample source from which the nucleic acid is derived. A composition comprising purified nucleic acid may be about 90%, 91%, 92%, 93%, 94%, 95%, 96%, 97%, 98%, 99% or greater than 99% free of other nucleic acid species. For example, fetal nucleic acid can be purified from a mixture comprising maternal and fetal nucleic acid. In certain examples, small fragments of nucleic acid (e.g., 30 to 500 bp fragments) can be purified, or partially purified, from a mixture comprising nucleic acid fragments of different lengths. In certain examples, nucleosomes comprising smaller fragments of nucleic acid can be purified from a mixture of larger nucleosome complexes comprising larger fragments of nucleic acid. In certain examples, larger nucleosome complexes comprising larger fragments of nucleic acid can be purified from nucleosomes comprising smaller fragments of nucleic acid. In certain examples, small fragments of fetal nucleic acid (e.g., 30 to 500 bp fragments) can be purified, or partially purified, from a mixture comprising both fetal and maternal nucleic acid fragments. In certain examples, nucleosomes comprising smaller fragments of fetal nucleic acid can be purified from a mixture of larger nucleosome complexes comprising larger fragments of maternal nucleic acid. In certain examples, cancer cell nucleic acid can be purified from a mixture comprising cancer cell and non-cancer cell nucleic acid. In certain examples, nucleosomes comprising small fragments of cancer cell nucleic acid can be purified from a mixture of larger nucleosome complexes comprising larger fragments of non-cancer nucleic acid. In some embodiments, nucleic acid is provided for conducting methods described herein without prior processing of the sample(s) containing the nucleic acid. For example, nucleic acid may be analyzed directly from a sample without prior extraction, purification, partial purification, and/or amplification.

Nucleic acids may be amplified under amplification conditions. The term “amplified” or “amplification” or “amplification conditions” as used herein refers to subjecting a target nucleic acid in a sample or a nucleic acid product generated by a method herein to a process that linearly or exponentially generates amplicon nucleic acids having the same or substantially the same nucleotide sequence as the target nucleic acid, or part thereof. In certain embodiments, the term “amplified” or “amplification” or “amplification conditions” refers to a method that comprises a polymerase chain reaction (PCR). In certain instances, an amplified product can contain one or more nucleotides more than the amplified nucleotide region of a nucleic acid template sequence (e.g., a primer can contain “extra” nucleotides such as a transcriptional initiation sequence, in addition to nucleotides complementary to a nucleic acid template gene molecule, resulting in an amplified product containing “extra” nucleotides or nucleotides not corresponding to the amplified nucleotide region of the nucleic acid template gene molecule).

Nucleic acid also may be exposed to a process that modifies certain nucleotides in the nucleic acid before providing nucleic acid for a method described herein. A process that selectively modifies nucleic acid based upon the methylation state of nucleotides therein can be applied to nucleic acid, for example. In addition, conditions such as high temperature, ultraviolet radiation, x-radiation, can induce changes in the sequence of a nucleic acid molecule. Nucleic acid may be provided in any suitable form useful for conducting a sequence analysis.

In some embodiments, target nucleic acids (e.g., ssNAs, dsNAs, or a combination thereof) are not modified prior to combining with sequencing adapters. In some embodiments, target nucleic acids are not modified in length prior to combining with sequencing adapters. In this context, “not modified” means that target nucleic acids are isolated from a sample and then combined with sequencing adapters, without modifying the length or the composition of the target nucleic acids. For example, target nucleic acids may not be shortened (e.g., they are not contacted with a restriction enzyme or nuclease or physical condition that reduces length (e.g., shearing condition, cleavage condition)) and may not be increased in length by one or more nucleotides (e.g., ends are not filled in at overhangs; no nucleotides are added to the ends). Adding a phosphate or chemically reactive group to one or both ends of a target nucleic acid generally is not considered modifying the nucleic acid or modifying the length of the nucleic acid. Denaturing a double-stranded nucleic acid (dsNA) fragment to generate an ssNA fragment generally is not considered modifying the nucleic acid or modifying the length of the nucleic acid.

In some embodiments, one or both native ends of target nucleic acids (e.g., ssNAs, dsNAs, or a combination thereof) are present when the target nucleic acid is combined with sequencing adapters. Native ends generally refer to unmodified ends of a nucleic acid fragment. In some embodiments, native ends of target nucleic acids are not modified in length prior to combining with sequencing adapters. In this context, “not modified” means that target nucleic acids are isolated from a sample and then combined with sequencing adapters, or components thereof, without modifying the length of the native ends of target nucleic acids. For example, target nucleic acids are not shortened (e.g., they are not contacted with a restriction enzyme or nuclease or physical condition that reduces length (e.g., shearing condition, cleavage condition) to generate non-native ends) and are not increased in length by one or more nucleotides (e.g., native ends are not filled in at overhangs; no nucleotides are added to the native ends). Adding a phosphate or chemically reactive group to one or both native ends of a target nucleic acid generally is not considered modifying the length of the nucleic acid.

In some embodiments, target nucleic acids (e.g., ssNAs, dsNAs, or a combination thereof) are not contacting with a cleavage agent (e.g., endonuclease, exonuclease, restriction enzyme) and/or a polymerase prior to combining with sequencing adapters. In some embodiments, target nucleic acids are not subjected to mechanical shearing (e.g., ultrasonication (e.g., Adaptive Focused Acoustics™ (AFA) process by Covaris)) prior to combining with sequencing adapters. In some embodiments, target nucleic acids are not contacting with an exonuclease (e.g., DNAse) prior to combining with sequencing adapters. In some embodiments, target nucleic acids are not amplified prior to combining with sequencing adapters. In some embodiments, target nucleic acids are not attached to a solid support prior to combining with sequencing adapters. In some embodiments, target nucleic acids are not conjugated to another molecule prior to combining with sequencing adapters. In some embodiments, target nucleic acids are not cloned into a vector prior to combining with sequencing adapters. In some embodiments, target nucleic acids may be subjected to dephosphorylation prior to combining with sequencing adapters. In some embodiments, target nucleic acids may be subjected to phosphorylation prior to combining with sequencing adapters.

In some embodiments, combining target nucleic acids (e.g., ssNAs, dsNAs, or a combination thereof) with sequencing adapters, comprises isolating the target nucleic acids, and combining the isolated target nucleic acids with sequencing adapters. In some embodiments, combining target nucleic acids with sequencing adapters comprises isolating the target nucleic acids, phosphorylating the isolated target nucleic acids, and combining the phosphorylated target nucleic acids with sequencing adapters. In some embodiments, combining target nucleic acids with sequencing adapters comprises isolating the target nucleic acids, dephosphorylating the sequencing adapters and combining the isolated target nucleic acids with the dephosphorylated sequencing adapters. In some embodiments, combining target nucleic acids with sequencing adapters comprises isolating the target nucleic acids, dephosphorylating the isolated target nucleic acids, phosphorylating the dephosphorylated target nucleic acids, and combining the phosphorylated target nucleic acids with sequencing adapters. In some embodiments, combining target nucleic acids with sequencing adapters comprises isolating the target nucleic acids, dephosphorylating the isolated target nucleic acids, phosphorylating the dephosphorylated target nucleic acids, dephosphorylating the sequencing adapters, and combining the phosphorylated target nucleic acids with the dephosphorylated sequencing adapters.

In some embodiments, combining target nucleic acids (e.g., ssNAs, dsNAs, or a combination thereof) with sequencing adapters consists of isolating the target nucleic acids, and combining the isolated target nucleic acids with sequencing adapters. In some embodiments, combining target nucleic acids with sequencing adapters consists of isolating the target nucleic acids, phosphorylating the isolated target nucleic acids, and combining the phosphorylated target nucleic acids with sequencing adapters. In some embodiments, combining target nucleic acids with sequencing adapters consists of isolating the target nucleic acids, dephosphorylating the sequencing adapters, and combining the isolated target nucleic acids with the dephosphorylated sequencing adapters. In some embodiments, combining target nucleic acids with sequencing adapters consists of isolating the target nucleic acids, dephosphorylating the isolated target nucleic acids, phosphorylating the dephosphorylated target nucleic acids, and combining the phosphorylated target nucleic acids with sequencing adapters. In some embodiments, combining target nucleic acids with sequencing adapters consists of isolating the target nucleic acids, dephosphorylating the isolated target nucleic acids, phosphorylating the dephosphorylated target nucleic acids, dephosphorylating the sequencing adapters, and combining the phosphorylated target nucleic acids with the dephosphorylated sequencing adapters.

### Enriching Nucleic Acids

In some embodiments, nucleic acid (e.g., extracellular nucleic acid; sample nucleic acid; target nucleic acid (e.g., ssNA, dsNA, or a combination thereof)) is enriched or relatively enriched for a subpopulation or species of nucleic acid. Nucleic acid subpopulations can include, for example, fetal nucleic acid, maternal nucleic acid, cancer nucleic acid, tumor nucleic acid, patient nucleic acid, host nucleic acid, pathogen nucleic acid, transplant nucleic acid, microbiome nucleic acid, nucleic acid comprising fragments of a particular length or range of lengths, or nucleic acid from a particular genome region (e.g., single chromosome, set of chromosomes, and/or certain chromosome regions). Such enriched samples can be used in conjunction with a method provided herein. Thus, in certain embodiments, methods of the technology comprise an additional step of enriching for a subpopulation of nucleic acid in a sample. In certain embodiments, nucleic acid from normal tissue (e.g., non-cancer cells, host cells) is selectively removed (partially, substantially, almost completely or completely) from the sample. In certain embodiments, maternal nucleic acid is selectively removed (partially, substantially, almost completely or completely) from the sample. In certain embodiments, enriching for a particular low copy number species nucleic acid (e.g., cancer, tumor, fetal, pathogen, transplant, microbiome nucleic acid) may improve quantitative sensitivity. Methods for enriching a sample for a particular species of nucleic acid are described, for example, in U.S. Pat. No. 6,927,028, International Patent Application Publication No. WO2007/140417, International Patent Application Publication No. WO2007/147063, International Patent Application Publication No. WO2009/032779, International Patent Application Publication No. WO2009/032781, International Patent Application Publication No. WO2010/033639, International Patent Application Publication No. WO2011/034631, International Patent Application Publication No. WO2006/056480, and International Patent Application Publication No. WO2011/143659, the entire content of each is incorporated herein by reference, including all text, tables, equations and drawings.

In some embodiments, nucleic acid is enriched for certain target fragment species and/or reference fragment species. In certain embodiments, nucleic acid is enriched for a specific nucleic acid fragment length or range of fragment lengths using one or more length-based separation methods described below. In certain embodiments, nucleic acid is enriched for fragments from a select genomic region (e.g., chromosome) using one or more sequence-based separation methods described herein and/or known in the art.

Non-limiting examples of methods for enriching for a nucleic acid subpopulation in a sample include methods that exploit epigenetic differences between nucleic acid species (e.g., methylation-based fetal nucleic acid enrichment methods described in U.S. Patent Application Publication No. 2010/0105049, which is incorporated by reference herein); restriction endonuclease enhanced polymorphic sequence approaches (e.g., such as a method described in U.S. Patent Application Publication No. 2009/0317818, which is incorporated by reference herein); selective enzymatic degradation approaches; massively parallel signature sequencing (MPSS) approaches; amplification (e.g., PCR)-based approaches (e.g., loci-specific amplification methods, multiplex SNP allele PCR approaches; universal amplification methods); pull-down approaches (e.g., biotinylated ultramer pull-down methods); extension and ligation-based methods (e.g., molecular inversion probe (MIP) extension and ligation); and combinations thereof.

In some embodiments, nucleic acid is enriched for fragments from a select genomic region (e.g., chromosome) using one or more sequence-based separation methods described herein. Sequence-based separation generally is based on nucleotide sequences present in the fragments of interest (e.g., target and/or reference fragments) and substantially not present in other fragments of the sample or present in an insubstantial amount of the other fragments (e.g., 5% or less). In some embodiments, sequence-based separation can generate separated target fragments and/or separated reference fragments. Separated target fragments and/or separated reference fragments often are isolated away from the remaining fragments in the nucleic acid sample. In certain embodiments, the separated target fragments and the separated reference fragments also are isolated away from each other (e.g., isolated in separate assay compartments). In certain embodiments, the separated target fragments and the separated reference fragments are isolated together (e.g., isolated in the same assay compartment). In some embodiments, unbound fragments can be differentially removed or degraded or digested.

In some embodiments, a selective nucleic acid capture process is used to separate target and/or reference fragments away from a nucleic acid sample. Commercially available nucleic acid capture systems include, for example, NIMBLEGEN sequence capture system (Roche NIMBLEGEN, Madison, WI); ILLUMINA BEADARRAY platform (Illumina, San Diego, CA); Affymetrix GENECHIP platform (Affymetrix, Santa Clara, CA); Agilent SURESELECT Target Enrichment System (Agilent Technologies, Santa Clara, CA); and related platforms. Such methods typically involve hybridization of a capture oligonucleotide to a part or all of the nucleotide sequence of a target or reference fragment and can include use of a solid phase (e.g., solid phase array) and/or a solution based platform. Capture oligonucleotides (sometimes referred to as “bait”) can be selected or designed such that they preferentially hybridize to nucleic acid fragments from selected genomic regions or loci, or a particular sequence in a nucleic acid target. In certain embodiments, a hybridization-based method (e.g., using oligonucleotide arrays) can be used to enrich for fragments containing certain nucleic acid sequences. Thus, in some embodiments, a nucleic acid sample is optionally enriched by capturing a subset of fragments using capture oligonucleotides complementary to, for example, selected sequences in sample nucleic acid. In certain instances, captured fragments are amplified. For example, captured fragments containing adapters may be amplified using primers complementary to the adapter sequences to form collections of amplified fragments, indexed according to adapter sequence. In some embodiments, nucleic acid is enriched for fragments from a select genomic region (e.g., chromosome, a gene) by amplification of one or more regions of interest using oligonucleotides (e.g., PCR primers) complementary to sequences in fragments containing the region(s) of interest, or part(s) thereof.

In some embodiments, nucleic acid is enriched for a particular nucleic acid fragment length, range of lengths, or lengths under or over a particular threshold or cutoff using one or more length-based separation methods. Nucleic acid fragment length typically refers to the number of nucleotides in the fragment. Nucleic acid fragment length also is sometimes referred to as nucleic acid fragment size. In some embodiments, a length-based separation method is performed without measuring lengths of individual fragments. In some embodiments, a length based separation method is performed in conjunction with a method for determining length of individual fragments. In some embodiments, length-based separation refers to a size fractionation procedure where all or part of the fractionated pool can be isolated (e.g., retained) and/or analyzed. Size fractionation procedures are known in the art (e.g., separation on an array, separation by a molecular sieve, separation by gel electrophoresis, separation by column chromatography (e.g., size-exclusion columns), and microfluidics-based approaches). In certain instances, length-based separation approaches can include selective sequence tagging approaches, fragment circularization, chemical treatment (e.g., formaldehyde, polyethylene glycol (PEG) precipitation), mass spectrometry and/or size-specific nucleic acid amplification, for example.

In some embodiments, a method herein includes enriching an RNA species in a mixture of RNA species. For example, a method herein may comprise enriching messenger RNA (mRNA) present in a mixture of mRNA and ribosomal RNA (rRNA). Any suitable mRNA enrichment method may be used, which includes rRNA depletion and/or mRNA enrichment methods such as rRNA depletion with magnetic beads (e.g., Ribo-zero™, Ribominus™, and MICROBExpress™, which use rRNA depletion probes in combination with magnetic beads to deplete rRNAs from a sample, thus enriching mRNAs), oligo(dT)-based poly(A) enrichment (e.g., BioMag® Oligo (dT)20), nuclease-based rRNA depletion (e.g., digestion of rRNA with Terminator™ 5′-Phosphate Dependent Exonuclease), and combinations thereof.

### Length-Based Separation

In some embodiments, a method herein comprises separating target nucleic acids (e.g., ssNAs, dsNAs, or a combination thereof) according to fragment length. For example, target nucleic acids may be enriched for a particular nucleic acid fragment length, range of lengths, or lengths under or over a particular threshold or cutoff using one or more length-based separation methods. Nucleic acid fragment length typically refers to the number of nucleotides in the fragment. Nucleic acid fragment length also may be referred to as nucleic acid fragment size. In some embodiments, a length-based separation method is performed without measuring lengths of individual fragments. In some embodiments, a length based separation method is performed in conjunction with a method for determining length of individual fragments. In some embodiments, length-based separation refers to a size fractionation procedure where all or part of the fractionated pool can be isolated (e.g., retained) and/or analyzed. Size fractionation procedures are known in the art (e.g., separation on an array, separation by a molecular sieve, separation by gel electrophoresis, separation by column chromatography (e.g., size-exclusion columns), and microfluidics-based approaches). In some embodiments, length-based separation approaches can include fragment circularization, chemical treatment (e.g., formaldehyde, polyethylene glycol (PEG)), mass spectrometry and/or size-specific nucleic acid amplification, for example. In some embodiments, length based-separation is performed using Solid Phase Reversible Immobilization (SPRI) beads.

In some embodiments, nucleic acid fragments of a certain length, range of lengths, or lengths under or over a particular threshold or cutoff are separated from the sample. In some embodiments, fragments having a length under a particular threshold or cutoff (e.g., 500 bp, 400 bp, 300 bp, 200 bp, 150 bp, 100 bp) are referred to as “short” fragments and fragments having a length over a particular threshold or cutoff (e.g., 500 bp, 600 bp, 700 bp, 800 bp, 900 bp, 1000 bp) are referred to as “long” fragments, large fragments, and/or high molecular weight (HMW) fragments. In some embodiments, fragments of a certain length, range of lengths, or lengths under or over a particular threshold or cutoff are retained for analysis while fragments of a different length or range of lengths, or lengths over or under the threshold or cutoff are not retained for analysis. In some embodiments, fragments that are less than about 500 bp are retained. In some embodiments, fragments that are less than about 400 bp are retained. In some embodiments, fragments that are less than about 300 bp are retained. In some embodiments, fragments that are less than about 200 bp are retained. In some embodiments, fragments that are less than about 150 bp are retained. For example, fragments that are less than about 190 bp, 180 bp, 170 bp, 160 bp, 150 bp, 140 bp, 130 bp, 120 bp, 110 bp or 100 bp are retained. In some embodiments, fragments that are about 100 bp to about 200 bp are retained. For example, fragments that are about 190 bp, 180 bp, 170 bp, 160 bp, 150 bp, 140 bp, 130 bp, 120 bp or 110 bp are retained. In some embodiments, fragments that are in the range of about 100 bp to about 200 bp are retained. For example, fragments that are in the range of about 110 bp to about 190 bp, 130 bp to about 180 bp, 140 bp to about 170 bp, 140 bp to about 150 bp, 150 bp to about 160 bp, or 145 bp to about 155 bp are retained.

In some embodiments, target nucleic acids (e.g., ssNAs, dsNAs, or a combination thereof) having fragment lengths of less than about 1000 bp are combined with a plurality or pool of sequencing adapters. In some embodiments, target nucleic acids having fragment lengths of less than about 500 bp are combined with a plurality or pool of sequencing adapters. In some embodiments, target nucleic acids having fragment lengths of less than about 400 bp are combined with a plurality or pool of sequencing adapters. In some embodiments, target nucleic acids having fragment lengths of less than about 300 bp are combined with a plurality or pool of sequencing adapters. In some embodiments, target nucleic acids having fragment lengths of less than about 200 bp are combined with a plurality or pool of sequencing adapters. In some embodiments, target nucleic acids having fragment lengths of less than about 100 bp are combined with a plurality or pool of sequencing adapters.

In some embodiments, target nucleic acids (e.g., ssNAs, dsNAs, or a combination thereof) having fragment lengths of about 100 bp or more are combined with a plurality or pool of sequencing adapters. In some embodiments, target nucleic acids having fragment lengths of about 200 bp or more are combined with a plurality or pool of sequencing adapters. In some embodiments, target nucleic acids having fragment lengths of about 300 bp or more are combined with a plurality or pool of sequencing adapters. In some embodiments, target nucleic acids having fragment lengths of about 400 bp or more are combined with a plurality or pool of sequencing adapters. In some embodiments, target nucleic acids having fragment lengths of about 500 bp or more are combined with a plurality or pool of sequencing adapters. In some embodiments, target nucleic acids having fragment lengths of about 1000 bp or more are combined with a plurality or pool of sequencing adapters.

In some embodiments, target nucleic acids (e.g., ssNAs, dsNAs, or a combination thereof) having any fragment length or any combination of fragment lengths are combined with a plurality or pool of sequencing adapters. For example, target nucleic acids having fragment lengths of less than 500 bp and fragments lengths of 500 bp or more may be combined with a plurality or pool of sequencing adapters.

Certain length-based separation methods that can be used with methods described herein employ a selective sequence tagging approach, for example. In such methods, a fragment size species (e.g., short fragments) nucleic acids are selectively tagged in a sample that includes long and short nucleic acids. Such methods typically involve performing a nucleic acid amplification reaction using a set of nested primers which include inner primers and outer primers. In some embodiments, one or both of the inner can be tagged to thereby introduce a tag onto the target amplification product. The outer primers generally do not anneal to the short fragments that carry the (inner) target sequence. The inner primers can anneal to the short fragments and generate an amplification product that carries a tag and the target sequence. Typically, tagging of the long fragments is inhibited through a combination of mechanisms which include, for example, blocked extension of the inner primers by the prior annealing and extension of the outer primers. Enrichment for tagged fragments can be accomplished by any of a variety of methods, including for example, exonuclease digestion of single-stranded nucleic acid and amplification of the tagged fragments using amplification primers specific for at least one tag.

Another length-based separation method that can be used with methods described herein involves subjecting a nucleic acid sample to polyethylene glycol (PEG) precipitation. Examples of methods include those described in International Patent Application Publication Nos. WO2007/140417 and WO2010/115016. This method in general entails contacting a nucleic acid sample with PEG in the presence of one or more monovalent salts under conditions sufficient to substantially precipitate large nucleic acids without substantially precipitating small (e.g., less than 300 nucleotides) nucleic acids.

Another length-based enrichment method that can be used with methods described herein involves circularization by ligation, for example, using circligase. Short nucleic acid fragments typically can be circularized with higher efficiency than long fragments. Non-circularized sequences can be separated from circularized sequences, and the enriched short fragments can be used for further analysis.

### Nucleic Acid Library

Methods herein may include preparing a nucleic acid library and/or modifying nucleic acids for a nucleic acid library. In some embodiments, ends of nucleic acid fragments are modified such that the fragments, or amplified products thereof, may be incorporated into a nucleic acid library. Generally, a nucleic acid library refers to a plurality of polynucleotide molecules (e.g., a sample of nucleic acids) that are prepared, assembled and/or modified for a specific process, non-limiting examples of which include immobilization on a solid phase (e.g., a solid support, a flow cell, a bead), enrichment, amplification, cloning, detection and/or for nucleic acid sequencing. In certain embodiments, a nucleic acid library is prepared prior to or during a sequencing process. A nucleic acid library (e.g., sequencing library) can be prepared by a suitable method as known in the art. A nucleic acid library can be prepared by a targeted or a non-targeted preparation process.

In some embodiments, a library of nucleic acids is modified to comprise a chemical moiety (e.g., a functional group) configured for immobilization of nucleic acids to a solid support. In some embodiments a library of nucleic acids is modified to comprise a biomolecule (e.g., a functional group) and/or member of a binding pair configured for immobilization of the library to a solid support, non-limiting examples of which include thyroxin-binding globulin, steroid-binding proteins, antibodies, antigens, haptens, enzymes, lectins, nucleic acids, repressors, protein A, protein G, avidin, streptavidin, biotin, complement component C1q, nucleic acid-binding proteins, receptors, carbohydrates, oligonucleotides, polynucleotides, complementary nucleic acid sequences, the like and combinations thereof. Some examples of specific binding pairs include, without limitation: an avidin moiety and a biotin moiety; an antigenic epitope and an antibody or immunologically reactive fragment thereof; an antibody and a hapten; a digoxigenin moiety and an anti-digoxigenin antibody; a fluorescein moiety and an anti-fluorescein antibody; an operator and a repressor; a nuclease and a nucleotide; a lectin and a polysaccharide; a steroid and a steroid-binding protein; an active compound and an active compound receptor; a hormone and a hormone receptor; an enzyme and a substrate; an immunoglobulin and protein A; an oligonucleotide or polynucleotide and its corresponding complement; the like or combinations thereof.

In some embodiments, a library of nucleic acids is modified to comprise one or more polynucleotides of known composition, non-limiting examples of which include an identifier (e.g., a tag, an indexing tag), a capture sequence, a label, an adapter, a restriction enzyme site, a promoter, an enhancer, an origin of replication, a stem loop, a complimentary sequence (e.g., a primer binding site, an annealing site), a suitable integration site (e.g., a transposon, a viral integration site), a modified nucleotide, a unique molecular identifier (UMI) described herein, a palindromic sequence described herein, the like or combinations thereof. Polynucleotides of known sequence can be added at a suitable position, for example on the 5′ end, 3′ end or within a nucleic acid sequence. Polynucleotides of known sequence can be the same or different sequences. In some embodiments, a polynucleotide of known sequence is configured to hybridize to one or more oligonucleotides immobilized on a surface (e.g., a surface in flow cell). For example, a nucleic acid molecule comprising a 5′ known sequence may hybridize to a first plurality of oligonucleotides while the 3′ known sequence may hybridize to a second plurality of oligonucleotides. In some embodiments, a library of nucleic acid can comprise chromosome-specific tags, capture sequences, labels and/or adapters (e.g., oligonucleotide adapters described herein). In some embodiments, a library of nucleic acids comprises one or more detectable labels. In some embodiments one or more detectable labels may be incorporated into a nucleic acid library at a 5′ end, at a 3′ end, and/or at any nucleotide position within a nucleic acid in the library. In some embodiments, a library of nucleic acids comprises hybridized oligonucleotides. In certain embodiments hybridized oligonucleotides are labeled probes. In some embodiments, a library of nucleic acids comprises hybridized oligonucleotide probes prior to immobilization on a solid phase.

In some embodiments, a polynucleotide of known sequence comprises a universal sequence. A universal sequence is a specific nucleotide sequence that is integrated into two or more nucleic acid molecules or two or more subsets of nucleic acid molecules where the universal sequence is the same for all molecules or subsets of molecules that it is integrated into. A universal sequence is often designed to hybridize to and/or amplify a plurality of different sequences using a single universal primer that is complementary to a universal sequence. In some embodiments two (e.g., a pair) or more universal sequences and/or universal primers are used. A universal primer often comprises a universal sequence. In some embodiments adapters (e.g., universal adapters) comprise universal sequences. In some embodiments one or more universal sequences are used to capture, identify and/or detect multiple species or subsets of nucleic acids.

In certain embodiments of preparing a nucleic acid library, (e.g., in certain sequencing by synthesis procedures), nucleic acids are size selected and/or fragmented into lengths of several hundred base pairs, or less (e.g., in preparation for library generation). In some embodiments, library preparation is performed without fragmentation (e.g., when using cell-free DNA).

In certain embodiments, a ligation-based library preparation method is used (e.g., ILLUMINA TRUSEQ, Illumina, San Diego CA). Ligation-based library preparation methods often make use of an adapter (e.g., a methylated adapter) design which can incorporate an index sequence (e.g., a sample index sequence to identify sample origin for a nucleic acid sequence) at the initial ligation step and often can be used to prepare samples for single-read sequencing, paired-end sequencing and multiplexed sequencing. For example, nucleic acids (e.g., fragmented nucleic acids or cell-free DNA) may be end repaired by a fill-in reaction, an exonuclease reaction or a combination thereof. In some embodiments, the resulting blunt-end repaired nucleic acid can then be extended by a single nucleotide, which is complementary to a single nucleotide overhang on the 3′ end of an adapter/primer. Any nucleotide can be used for the extension/overhang nucleotides. In some embodiments, end repair is omitted and sequencing adapters are ligated directly to the native ends of nucleic acids (e.g., double-stranded nucleic acids, single-stranded nucleic acids, fragmented nucleic acids, and/or cell-free nucleic acids).

In some embodiments, nucleic acid library preparation comprises ligating a sequencing adapter, or component thereof, to a nucleic acid (e.g., to a sample nucleic acid, to a sample nucleic acid fragment, to a template nucleic acid, to a target nucleic acid). Examples of adapters useful for generating a nucleic acid library (e.g., a sequencing library) are described in International Patent Application Publication No. WO 2018/013837, International Patent Application Publication No. WO 2019/140201, International Patent Application Publication No. WO2019/236726, and International Patent Application Publication No. WO/2020/206143, each of which is incorporated by reference herein.

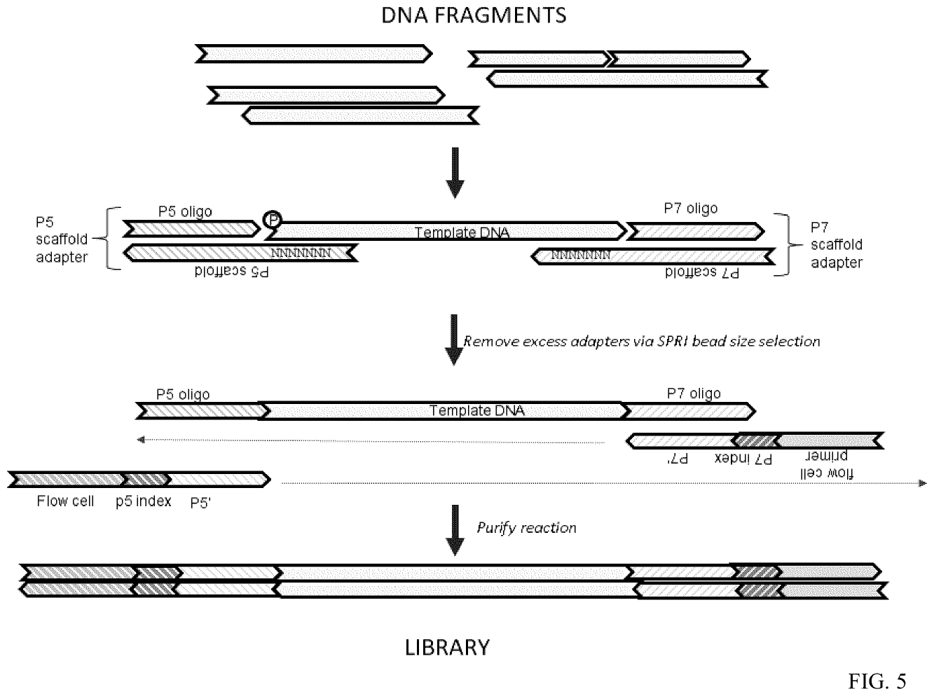

In some embodiments, a nucleic acid library preparation comprises ligating a scaffold adapter, or component thereof, to a nucleic acid (e.g., to a sample nucleic acid, to a sample nucleic acid fragment, to a template nucleic acid, to a target nucleic acid). In some embodiments, a nucleic acid library preparation comprises ligating a scaffold adapter, or component thereof, to a single-stranded nucleic acid (ssNA). Scaffold adapters generally include a scaffold polynucleotide and an oligonucleotide. Accordingly, a “component” of a scaffold adapter may refer to a scaffold polynucleotide and/or an oligonucleotide, or a subcomponent or region thereof. An example of a scaffold adapter is provided in FIG. 5. The oligonucleotide and/or the scaffold polynucleotide can be composed of pyrimidine (C, T, U) and/or purine (A, G) nucleotides. Additional components or subcomponents may include one or more of an index polynucleotide, a unique molecular identifier (UMI), primer binding site (e.g., P5 primer binding site, P7 primer binding site), flow cell binding region, and the like, and complements thereto.

A scaffold polynucleotide is a single-stranded component of a scaffold adapter. A polynucleotide herein generally refers to a single-stranded multimer of nucleotide from 5 to 500 nucleotides, e.g., 5 to 100 nucleotides. Polynucleotides may be synthetic or may be made enzymatically, and, in some embodiments, are about 5 to 50 nucleotides in length. Polynucleotides may contain ribonucleotide monomers (i.e., may be polyribonucleotides or “RNA polynucleotides”), deoxyribonucleotide monomers (i.e., may be polydeoxyribonucleotides or “DNA polynucleotides”), or a combination thereof. Polynucleotides may be 10 to 20, 20 to 30, 30 to 40, 40 to 50, 50 to 60, 60 to 70, 70 to 80, 80 to 100, 100 to 150 or 150 to 200, or up to 500 nucleotides in length, for example. The terms polynucleotide and oligonucleotide may be used interchangeably.

A scaffold polynucleotide may include an ssNA hybridization region (also referred to as scaffold, scaffold region, single-stranded scaffold, single-stranded scaffold region) and an oligonucleotide hybridization region. An ssNA hybridization region and an oligonucleotide hybridization region may be referred to as subcomponents of a scaffold polynucleotide. An ssNA hybridization region typically comprises a polynucleotide that hybridizes, or is capable of hybridizing, to an ssNA terminal region. An oligonucleotide hybridization region typically comprises a polynucleotide that hybridizes, or is capable of hybridizing, to all or a portion of the oligonucleotide component of the scaffold adapter.

An ssNA hybridization region of a scaffold polynucleotide may comprise a polynucleotide that is complementary, or substantially complementary, to an ssNA terminal region. In some embodiments, an ssNA hybridization region comprises a random sequence. In some embodiments, an ssNA hybridization region comprises a sequence complementary to an ssNA terminal region sequence of interest (e.g., targeted sequence). In certain embodiments, an ssNA hybridization region comprises one or more nucleotides that are all capable of non-specific base pairing to bases in the ssNA. Nucleotides capable of non-specific base pairing may be referred to as universal bases. A universal base is a base capable of indiscriminately base pairing with each of the four standard nucleotide bases: A, C, G and T. Universal bases that may be incorporated into the ssNA hybridization region include, but are not limited to, inosine, deoxyinosine, 2′-deoxyinosine (dI, dInosine), nitroindole, 5-nitroindole, and 3-nitropyrrole. In certain embodiments, an ssNA hybridization region comprises one or more degenerate/wobble bases which can replace two or three (but not all) of the four typical bases (e.g., non-natural base P and K).

An ssNA hybridization region of a scaffold polynucleotide may have any suitable length and sequence. In some embodiments, the length of the ssNA hybridization region is 10 nucleotides or less. In certain aspects, the ssNA hybridization region is from 4 to 100 nucleotides in length, e.g., about 5, 10, 15, 20, 25, 30, 35, 40, 45, 50, 55, 60, 65, 70, 75, 80, 85, 90, 95, or 100 nucleotides in length. In certain aspects, the ssNA hybridization region is from 4 to 20 nucleotides in length, e.g., from 5 to 15, 5 to 10, 5 to 9, 5 to 8, or 5 to 7 (e.g., 6 or 7) nucleotides in length. In some embodiments, the ssNA hybridization region is 7 nucleotides in length. In some embodiments, the ssNA hybridization region comprises or consists of a random nucleotide sequence, such that when a plurality of heterogeneous scaffold polynucleotides having various random ssNA hybridization regions are employed, the collection is capable of acting as scaffold polynucleotides for a heterogeneous population of ssNAs irrespective of the sequences of the terminal regions of the ssNAs. Each scaffold polynucleotide having a unique ssNA hybridization region sequence may be referred to as a scaffold polynucleotide species and a collection of multiple scaffold polynucleotide species may be referred to as a plurality of scaffold polynucleotide species (e.g., for a scaffold polynucleotide designed to have 7 random bases in the ssNA hybridization region, a plurality of scaffold polynucleotide species would include 4⁷ unique ssNA hybridization region sequences). Accordingly, each scaffold adapter having a unique scaffold polynucleotide (i.e., comprising a unique ssNA hybridization region sequence) may be referred to as a scaffold adapter species and a collection of multiple scaffold adapter species may be referred to as a plurality of scaffold adapter species. A species of scaffold polynucleotide generally contains a feature that is unique with respect to other scaffold polynucleotide species. For example, a scaffold polynucleotide species may contain a unique sequence feature. A unique sequence feature may include a unique sequence length, a unique nucleotide sequence (e.g., a unique random sequence, a unique targeted sequence), or a combination of a unique sequence length and nucleotide sequence.

An oligonucleotide is a further single-stranded component of a scaffold adapter. An oligonucleotide herein generally refers to a single-stranded multimer of nucleotides from 5 to 500 nucleotides, e.g., 5 to 100 nucleotides. Oligonucleotides may be synthetic or may be made enzymatically, and, in some embodiments, are 5 to 50 nucleotides in length. Oligonucleotides may contain ribonucleotide monomers (i.e., may be oligoribonucleotides or “RNA oligonucleotides”), deoxyribonucleotide monomers (i.e., may be oligodeoxyribonucleotides or “DNA oligonucleotides”), or a combination thereof. Oligonucleotides may be 10 to 20, 20 to 30, 30 to 40, 40 to 50, 50 to 60, 60 to 70, 70 to 80, 80 to 100, 100 to 150 or 150 to 200, or up to 500 nucleotides in length, for example. The terms oligonucleotide and polynucleotide may be used interchangeably.

An oligonucleotide component of a scaffold adapter generally comprises a nucleic acid sequence that is complementary or substantially complementary to the oligonucleotide hybridization region of the scaffold polynucleotide. An oligonucleotide component of a scaffold adapter may include one or more subcomponents useful for one or more downstream applications such as, for example, PCR amplification of the ssNA fragment or derivative thereof, sequencing of the ssNA or derivative thereof, and the like. In some embodiments, a subcomponent of an oligonucleotide is a sequencing adapter. Sequencing adapter generally refers to one or more nucleic acid domains that include at least a portion of a nucleotide sequence (or complement thereof) utilized by a sequencing platform of interest, such as a sequencing platform provided by Illumina® (e.g., the HiSeq™, MiSeq™ and/or Genome Analyzer™ sequencing systems); Oxford Nanopore™ Technologies (e.g., the MinION™ sequencing system), Ion Torrent™ (e.g., the Ion PGM™ and/or Ion Proton™ sequencing systems); Pacific Biosciences (e.g., a Sequel or PACBIO RS II sequencing system); Life Technologies™ (e.g., a SOLiD™ sequencing system); Roche (e.g., the 454 GS FLX+ and/or GS Junior sequencing systems); or any sequencing platform of interest.

In some embodiments, an oligonucleotide component of a scaffold adapter is, or comprises, a nucleic acid domain selected from: a domain (e.g., a “capture site” or “capture sequence”) that specifically binds to a surface-attached sequencing platform oligonucleotide (e.g., a P5 or P7 oligonucleotide attached to the surface of a flow cell in an Illumina® sequencing system); a sequencing primer binding domain (e.g., a domain to which the Read 1 or Read 2 primers of the Illumina® platform may bind); a unique identifier or index (e.g., a barcode or other domain that uniquely identifies the sample source of the ssNA being sequenced to enable sample multiplexing by marking every molecule from a given sample with a specific barcode or “tag”); a barcode sequencing primer binding domain (a domain to which a primer used for sequencing a barcode binds); a molecular identification domain or unique molecular identifier (UMI) (e.g., a molecular index tag, such as a randomized tag of 4, 6, or other number of nucleotides) for uniquely marking molecules of interest, e.g., to determine expression levels based on the number of instances a unique tag is sequenced; a complement of any such domains; or any combination thereof. In some embodiments, a barcode domain (e.g., sample index tag) and a molecular identification domain (e.g., a molecular index tag; UMI) may be included in the same nucleic acid.

When an oligonucleotide component of a scaffold adapter includes one or a portion of a sequencing adapter, one or more additional sequencing adapters and/or a remaining portion of the sequencing adapter may be added using a variety of approaches. For example, additional and/or remaining portions of sequencing adapters may be added by any one of ligation, reverse transcription, PCR amplification, and the like. In the case of PCR, an amplification primer pair may be employed that includes a first amplification primer that includes a 3′ hybridization region (e.g., for hybridizing to an adapter region of the oligonucleotide) and a 5′ region including an additional and/or remaining portion of a sequencing adapter, and a second amplification primer that includes a 3′ hybridization region (e.g., for hybridizing to an adapter region of a second oligonucleotide added to the opposite end of an ssNA molecule) and optionally a 5′ region including an additional and/or remaining portion of a sequencing adapter.

The scaffold polynucleotide may be hybridized to the oligonucleotide, forming a duplex in the scaffold adapter. Accordingly, a scaffold adapter may be referred to as a scaffold duplex, a duplex adapter, a duplex oligonucleotide, or a duplex polynucleotide. Each scaffold duplex having a unique scaffold polynucleotide (i.e., comprising a unique ssNA hybridization region sequence) may be referred to as a scaffold duplex species and a collection of multiple scaffold duplex species may be referred to as a plurality of scaffold duplex species. In some embodiments, the scaffold polynucleotide and the oligonucleotide are on separate DNA strands. In some embodiments, the scaffold polynucleotide and the oligonucleotide are on a single DNA strand (e.g., a single DNA strand capable of forming a hairpin structure).

A method herein may comprise combining one or more scaffold adapters, or components thereof, with a composition comprising single-stranded nucleic acid (ssNA) to form one or more complexes. The scaffold polynucleotide is designed for simultaneous hybridization to an ssNA fragment and an oligonucleotide component such that, upon complex formation, an end of the oligonucleotide component is adjacent to an end of the terminal region of the ssNA fragment. Typically, upon complex formation, a 5′ end of the oligonucleotide component is adjacent to a 3′ end of the terminal region of the ssNA, or a 5′ end of the oligonucleotide component is adjacent to a 3′ end of the terminal region of the ssNA. Upon complex formation in instances where a scaffold adapter is attached to both ends of an ssNA fragment, a 5′ end of one oligonucleotide component is adjacent to a 3′ end of one terminal region of the ssNA, and a 5′ end of a second oligonucleotide component is adjacent to a 3′ end of a second terminal region of the ssNA.

In some embodiments, a method includes forming complexes by combining an ssNA composition, an oligonucleotide, and a plurality of heterogeneous scaffold polynucleotides having various random ssNA hybridization regions capable of acting as scaffolds for a heterogeneous population of ssNA having terminal regions of undetermined sequence.

In some embodiments, an ssNA hybridization region includes a known sequence designed to hybridize to an ssNA terminal region of known sequence. In some embodiments, two or more heterogeneous scaffold polynucleotides having different ssNA hybridization regions of known sequence are designed to hybridize to respective ssNA terminal regions of known sequence. Embodiments in which the ssNA hybridization regions have a known sequence may be useful, for example, for producing a nucleic acid library from a subset of ssNAs having terminal regions of known sequence. Accordingly, in certain embodiments, a method herein comprises forming complexes by combining an ssNA composition, an oligonucleotide, and one or more heterogeneous scaffold polynucleotides having one or more different ssNA hybridization regions of known sequence capable of acting as scaffolds for one or more ssNAs having one or more terminal regions of known sequence.

An ssNA fragment, an oligonucleotide, and scaffold polynucleotide may be combined in various ways. In some configurations, the combining includes combining 1) a complex comprising the scaffold polynucleotide hybridized to the oligonucleotide component via the oligonucleotide hybridization region, and 2) the ssNA fragment. In another configuration, the combining includes combining 1) a complex comprising the scaffold polynucleotide hybridized to the ssNA fragment via the ssNA hybridization region, and 2) the oligonucleotide component. In another configuration, the combining includes combining 1) the ssNA fragment, 2) the oligonucleotide, and 3) the scaffold polynucleotide, where none of the three components are pre-complexed with, or hybridized to, another component prior to the combining.

The combining may be carried out under hybridization conditions such that complexes form including a scaffold polynucleotide hybridized to a terminal region of an ssNA fragment via the ssNA hybridization region, and the scaffold polynucleotide hybridized to an oligonucleotide component via the oligonucleotide hybridization region. Whether specific hybridization occurs may be determined by factors such as the degree of complementarity between the hybridizing regions of the scaffold polynucleotide, the terminal region of the ssNA fragment, and the oligonucleotide component, as well as the length thereof, salt concentration, and the temperature at which the hybridization occurs, which may be informed by the melting temperatures (Tm) of the relevant regions.

Complexes may be formed such that an end of an oligonucleotide component is adjacent to an end of a terminal region of an ssNA fragment. Adjacent to refers the terminal nucleotide at the end of the oligonucleotide and the terminal nucleotide end of the terminal region of the ssNA fragment are sufficiently proximal to each other that the terminal nucleotides may be covalently linked, for example, by chemical ligation, enzymatic ligation, or the like. In some embodiments, the ends are adjacent to each other by virtue of the terminal nucleotide at the end of the oligonucleotide and the terminal nucleotide end of the terminal region of the ssNA being hybridized to adjacent nucleotides of the scaffold polynucleotide. The scaffold polynucleotide may be designed to ensure that an end of the oligonucleotide is adjacent to an end of the terminal region of the ssNA fragment.

Nucleic acid fragments (e.g., ssNA fragments) may be combined with scaffold adapters, or components thereof, thereby generating combined products. In some embodiments, a method herein comprises contacting ssNA with single-stranded nucleic acid binding protein (SSB) to produce SSB-bound ssNA prior to or during combining with scaffold adapters, or components thereof. SSB generally binds in a cooperative manner to ssNA and typically does not bind well to double-stranded nucleic acid (dsNA). Upon binding ssDNA, SSB destabilizes helical duplexes. SSBs may be prokaryotic SSB (e.g., bacterial or archaeal SSB) or eukaryotic SSB. Examples of SSBs may include E. coli SSB, E. coli RecA, Extreme Thermostable Single-Stranded DNA Binding Protein (ET SSB), Thermus thermophilus (Tth) RecA, T4 Gene 32 Protein, replication protein A (RPA—a eukaryotic SSB), and the like. ET SSB, Tth RecA, E. coli RecA, T4 Gene 32 Protein, as well buffers and detailed protocols for preparing SSB-bound ssNA using such SSBs are commercially available (e.g., New England Biolabs, Inc. (Ipswich, MA)).

Combining ssNA fragments with scaffold adapters, or components thereof, may comprise hybridization and/or ligation (e.g., ligation of hybridization products). A combined product may include an ssNA fragment connected to (e.g., hybridized to and/or ligated to) a scaffold adapter, or component thereof, at one or both ends of the ssNA fragment. A combined product may include an ssNA fragment hybridized to a scaffold adapter, or component thereof, at one or both ends of the ssNA fragment, which may be referred to as a hybridization product. A combined product may include an ssNA fragment ligated to a scaffold adapter, or component thereof, at one or both ends of the ssNA fragment, which may be referred to as a ligation product. In some embodiments, products from a cleavage step (i.e., cleaved products) may be combined with scaffold adapters, or components thereof, thereby generating combined products. Certain methods herein comprise generating sets of combined products (e.g., a first set of combined products and a second set of combined products). In some embodiments, a first set of combined products includes ssNAs connected to (e.g., hybridized to and/or ligated to) scaffold adapters, or components thereof, from a first set of scaffold adapters, or components thereof. In some embodiments, a second set of combined products includes the first set of combined products connected to (e.g., hybridized to and/or ligated to) scaffold adapters, or components thereof, from a second set of scaffold adapters, or components thereof.

ssNAs may be combined with scaffold adapters, or components thereof, under hybridization conditions, thereby generating hybridization products. In some embodiments, the scaffold adapters are provided as pre-hybridized products and the hybridization step includes hybridizing the scaffold adapters to the ssNA. In some embodiments, the scaffold adapter components (i.e., oligonucleotides and scaffold polynucleotides) are provided as individual components and the hybridization step includes hybridizing the scaffold adapter components 1) to each other and 2) to the ssNA. In some embodiments, the scaffold adapter components (i.e., oligonucleotides and scaffold polynucleotides) are provided sequentially as individual components and the hybridization steps includes 1) hybridizing the scaffold polynucleotides to the ssNA, and then 2) hybridizing the oligonucleotides to the oligonucleotide hybridization region of the scaffold polynucleotides. The conditions during the combining step are those conditions in which scaffold adapters, or components thereof (e.g., single-stranded scaffold regions), specifically hybridize to ssNAs having a terminal region or terminal regions that are complementary in sequence with respect to the single-stranded scaffold regions. The conditions during the combining step also may include those conditions in which components of the scaffold adapters (e.g., oligonucleotides and oligonucleotide hybridization regions within the scaffold polynucleotides), specifically hybridize, or remain hybridized, to each other.

Specific hybridization may be affected or influenced by factors such as the degree of complementarity between the single-stranded scaffold regions and the ssNA terminal region(s), or between the oligonucleotides and oligonucleotide hybridization regions, the length thereof, and the temperature at which the hybridization occurs, which may be informed by melting temperatures (Tm) of the single-stranded scaffold regions. Melting temperature generally refers to the temperature at which half of the single-stranded scaffold regions/ssNA terminal regions remain hybridized and half of the single-stranded scaffold regions/ssNA terminal regions dissociate into single strands. The Tm of a duplex may be experimentally determined or predicted using the following formula Tm=81.5+16.6(log₁₀[Na+])+0.41 (fraction G+C)−(60/N), where N is the chain length and [Na+] is less than 1 M. Additional models that depend on various parameters also may be used to predict Tm of relevant regions depending on various hybridization conditions. Approaches for achieving specific nucleic acid hybridization are described, e.g., Tijssen, Laboratory Techniques in Biochemistry and Molecular Biology-Hybridization with Nucleic Acid Probes, part I, chapter 2, “Overview of principles of hybridization and the strategy of nucleic acid probe assays,” Elsevier (1993).

In some embodiments, a method herein comprises exposing hybridization products to conditions under which an end of an ssNA is joined to an end of a scaffold adapter to which it is hybridized. In particular, a method herein may comprise exposing hybridization products to conditions under which an end of an ssNA is joined to an end of an oligonucleotide component of a scaffold adapter to which it is hybridized. Joining may be achieved by any suitable approach that permits covalent attachment of ssNA to the scaffold adapter and/or oligonucleotide component of a scaffold adapter to which it is hybridized. When one end of an ssNA is joined to an end of a scaffold adapter and/or oligonucleotide component of a scaffold adapter to which it is hybridized, typically one of two attachment events is conducted: 1) the 3′ end of the ssNA to the 5′ end of the oligonucleotide component of the scaffold adapter, or 2) the 5′ end of the ssNA to the 3′ end of the oligonucleotide component of the scaffold adapter. When both ends of an ssNA are each joined to an end of a scaffold adapter and/or oligonucleotide component of a scaffold adapter to which it is hybridized, typically two attachment events are conducted: 1) the 3′ end of the ssNA to the 5′ end of the oligonucleotide component of a first scaffold adapter, and 2) the 5′ end of the ssNA to the 3′ end of the oligonucleotide component of a second scaffold adapter.

In some embodiments, a method herein comprises contacting hybridization products with an agent comprising a ligase activity under conditions in which an end of an ssNA is covalently linked to an end of a scaffold adapter and/or oligonucleotide component of a scaffold adapter to which the target nucleic acid (ssNA) is hybridized. Ligase activity may include, for example, blunt-end ligase activity, nick-sealing ligase activity, sticky end ligase activity, circularization ligase activity, cohesive end ligase activity, DNA ligase activity, RNA ligase activity, single-stranded ligase activity, and double-stranded ligase activity. Ligase activity may include ligating a 5′ phosphorylated end of one polynucleotide to a 3′ OH end of another polynucleotide (5′P to 3′OH). Ligase activity may include ligating a 3′ phosphorylated end of one polynucleotide to a 5′ OH end of another polynucleotide (3′P to 5′OH). Ligase activity may include ligating a 5′ end of an ssNA to a 3′ end of a scaffold adapter and/or oligonucleotide component of a scaffold adapter hybridized thereto in a ligation reaction. Ligase activity may include ligating a 3′ end of an ssNA to a 5′ end of a scaffold adapter and/or oligonucleotide component of a scaffold adapter hybridized thereto in a ligation reaction. Suitable reagents (e.g., ligases) and kits for performing ligation reactions are known and available. For example, Instant Sticky-end Ligase Master Mix available from New England Biolabs (Ipswich, MA) may be used. Ligases that may be used include, for example, T4 DNA ligase (e.g., at low or high concentration), T7 DNA Ligase, E. coli DNA Ligase, Electro Ligase®, RNA ligases, T4 RNA ligase 2, SplintR® Ligase, RtcB ligase, and the like and combinations thereof. When needed, a phosphate group may be added at the 5′ end of the oligonucleotide component or ssNA fragment using a suitable kinase, for example, such as T4 polynucleotide kinase (PNK). Such kinases and guidance for using such kinases to phosphorylate 5′ ends are available, for example, from New England BioLabs, Inc. (Ipswich, MA).

In some embodiments, a method comprises covalently linking the adjacent ends of an oligonucleotide component and an ssNA terminal region, thereby generating covalently linked hybridization products. In some embodiments, the covalently linking comprises contacting the hybridization products (e.g., ssNA fragments hybridized to at least one scaffold adapter herein) with an agent comprising a ligase activity under conditions in which the end of an ssNA terminal region is covalently linked to an end of the oligonucleotide component. In some embodiments, a method comprises covalently linking the adjacent ends of a first oligonucleotide component and a first ssNA terminal region, and covalently linking the adjacent ends of a second oligonucleotide component and a second ssNA terminal region, thereby generating covalently linked hybridization products. In some embodiments, the covalently linking comprises contacting hybridization products (e.g., ssNA fragments each hybridized two scaffold adapters herein) with an agent comprising a ligase activity under conditions in which an end of a first ssNA terminal region is covalently linked to an end of a first oligonucleotide component and an end of a second ssNA terminal region is covalently linked to an end of a second oligonucleotide component. In some embodiments, the agent comprising a ligase activity is a T4 DNA ligase.

In some embodiments, hybridization products are contacted with a first agent comprising a first ligase activity and a second agent comprising a second ligase activity different than the first ligase activity. For example, the first ligase activity and the second ligase activity independently may be chosen from blunt-end ligase activity, nick-sealing ligase activity, sticky end ligase activity, circularization ligase activity, and cohesive end ligase activity, double-stranded ligase activity, single-stranded ligase activity, 5′P to 3′OH ligase activity, and 3′P to 5′OH ligase activity.

Covalently linking the adjacent ends of an oligonucleotide and an ssNA fragment produces a covalently linked product, which may be referred to a ligation product. A covalently linked product that includes an ssNA fragment covalently linked to an oligonucleotide component, which remain hybridized to a scaffold polynucleotide, may be referred to as a covalently linked hybridization product. A covalently linked hybridization product may be denatured (e.g., heat-denatured) to separate the ssNA fragment covalently linked to an oligonucleotide component from the scaffold polynucleotide. A covalently linked product that includes an ssNA fragment covalently linked to an oligonucleotide component, which is no longer hybridized to a scaffold polynucleotide (e.g., after denaturing), may be referred to as a single-stranded ligation product. In some cases, portions of a scaffold polynucleotide can be cleaved and/or degraded, for example by using uracil-DNA glycosylase and an endonuclease at one or more uracil bases in the scaffold polynucleotide.

A covalently linked hybridization product and/or single-stranded ligation product may be purified prior to use as input in a downstream application of interest (e.g., amplification; sequencing). For example, covalently linked hybridization products and/or single-stranded ligation products may be purified from certain components present during the combining, hybridization, and/or covalently linking (ligation) steps (e.g., by solid phase reversible immobilization (SPRI), column purification, and/or the like).

Sequencing adapters may comprise sequences complementary to flow-cell anchors, and sometimes are utilized to immobilize a nucleic acid library to a solid support, such as the inside surface of a flow cell, for example. In some embodiments, a sequencing adapter comprises an identifier, one or more sequencing primer hybridization sites (e.g., sequences complementary to universal sequencing primers, single end sequencing primers, paired end sequencing primers, multiplexed sequencing primers, and the like), or combinations thereof (e.g., adapter/sequencing, adapter/identifier, adapter/identifier/sequencing). In some embodiments, a sequencing adapter comprises one or more of primer annealing polynucleotide, also referred to herein as priming sequence or primer binding domain, (e.g., for annealing to flow cell attached oligonucleotides and/or to free amplification primers), an index polynucleotide (e.g., sample index sequence for tracking nucleic acid from different samples; also referred to as a sample ID), a barcode polynucleotide (e.g., single molecule barcode (SMB) for tracking individual molecules of sample nucleic acid that are amplified prior to sequencing; also referred to as a molecular barcode or a unique molecular identifier (UMI)). In some embodiments, a primer annealing component (or priming sequence or primer binding domain) of a sequencing adapter comprises one or more universal sequences (e.g., sequences complementary to one or more universal amplification primers). In some embodiments, an index polynucleotide (e.g., sample index; sample ID) is a component of a sequencing adapter. In some embodiments, an index polynucleotide (e.g., sample index; sample ID) is a component of a universal amplification primer sequence.

In some embodiments, sequencing adapters when used in combination with amplification primers (e.g., universal amplification primers) are designed generate library constructs comprising one or more of: universal sequences, molecular barcodes, sample ID sequences, spacer sequences, and a sample nucleic acid sequence. In some embodiments, sequencing adapters when used in combination with universal amplification primers are designed to generate library constructs comprising an ordered combination of one or more of: universal sequences, molecular barcodes, sample ID sequences, spacer sequences, and a sample nucleic acid sequence. For example, a library construct may comprise a first universal sequence, followed by a second universal sequence, followed by first molecular barcode, followed by a spacer sequence, followed by a template sequence (e.g., sample nucleic acid sequence), followed by a spacer sequence, followed by a second molecular barcode, followed by a third universal sequence, followed by a sample ID, followed by a fourth universal sequence. In some embodiments, sequencing adapters when used in combination with amplification primers (e.g., universal amplification primers) are designed generate library constructs for each strand of a template molecule (e.g., sample nucleic acid molecule). In some embodiments, sequencing adapters are duplex adapters.

An identifier can be a suitable detectable label incorporated into or attached to a nucleic acid (e.g., a polynucleotide) that allows detection and/or identification of nucleic acids that comprise the identifier. In some embodiments, an identifier is incorporated into or attached to a nucleic acid during a sequencing method (e.g., by a polymerase). In some embodiments, an identifier is incorporated into or attached to a nucleic acid prior to a sequencing method (e.g., by an extension reaction, by an amplification reaction, by a ligation reaction). Non-limiting examples of identifiers include nucleic acid tags, nucleic acid indexes or barcodes, a radiolabel (e.g., an isotope), metallic label, a fluorescent label, a chemiluminescent label, a phosphorescent label, a fluorophore quencher, a dye, a protein (e.g., an enzyme, an antibody or part thereof, a linker, a member of a binding pair), the like or combinations thereof. In some embodiments, an identifier (e.g., a nucleic acid index or barcode) is a unique, known and/or identifiable sequence of nucleotides or nucleotide analogues. In some embodiments, identifiers are six or more contiguous nucleotides. A multitude of fluorophores are available with a variety of different excitation and emission spectra. Any suitable type and/or number of fluorophores can be used as an identifier. In some embodiments 1 or more, 2 or more, 3 or more, 4 or more, 5 or more, 6 or more, 7 or more, 8 or more, 9 or more, 10 or more, 20 or more, 30 or more or 50 or more different identifiers are utilized in a method described herein (e.g., a nucleic acid detection and/or sequencing method). In some embodiments, one or two types of identifiers (e.g., fluorescent labels) are linked to each nucleic acid in a library. Detection and/or quantification of an identifier can be performed by a suitable method, apparatus or machine, non-limiting examples of which include flow cytometry, quantitative polymerase chain reaction (qPCR), gel electrophoresis, a luminometer, a fluorometer, a spectrophotometer, a suitable gene-chip or microarray analysis, Western blot, mass spectrometry, chromatography, cytofluorimetric analysis, fluorescence microscopy, a suitable fluorescence or digital imaging method, confocal laser scanning microscopy, laser scanning cytometry, affinity chromatography, manual batch mode separation, electric field suspension, a suitable nucleic acid sequencing method and/or nucleic acid sequencing apparatus, the like and combinations thereof.

In some embodiments, an identifier, a sequencing-specific index/barcode, and a sequencer-specific flow-cell binding primer sites are incorporated into a nucleic acid library by single-primer extension (e.g., by a strand displacing polymerase).

In some embodiments, a nucleic acid library or parts thereof are amplified (e.g., amplified by a PCR-based method) under amplification conditions. In some embodiments, a sequencing method comprises amplification of a nucleic acid library. A nucleic acid library can be amplified prior to or after immobilization on a solid support (e.g., a solid support in a flow cell). Nucleic acid amplification includes the process of amplifying or increasing the numbers of a nucleic acid template and/or of a complement thereof that are present (e.g., in a nucleic acid library), by producing one or more copies of the template and/or its complement. Amplification can be carried out by a suitable method. A nucleic acid library can be amplified by a thermocycling method or by an isothermal amplification method. In some embodiments, a rolling circle amplification method is used. In some embodiments, amplification takes place on a solid support (e.g., within a flow cell) where a nucleic acid library or portion thereof is immobilized. In certain sequencing methods, a nucleic acid library is added to a flow cell and immobilized by hybridization to anchors under suitable conditions. This type of nucleic acid amplification is often referred to as solid phase amplification. In some embodiments of solid phase amplification, all or a portion of the amplified products are synthesized by an extension initiating from an immobilized primer. Solid phase amplification reactions are analogous to standard solution phase amplifications except that at least one of the amplification oligonucleotides (e.g., primers) is immobilized on a solid support. In some embodiments, modified nucleic acid (e.g., nucleic acid modified by addition of adapters) is amplified.

In some embodiments, solid phase amplification comprises a nucleic acid amplification reaction comprising only one species of oligonucleotide primer immobilized to a surface. In certain embodiments, solid phase amplification comprises a plurality of different immobilized oligonucleotide primer species. In some embodiments, solid phase amplification may comprise a nucleic acid amplification reaction comprising one species of oligonucleotide primer immobilized on a solid surface and a second different oligonucleotide primer species in solution. Multiple different species of immobilized or solution-based primers can be used. Non-limiting examples of solid phase nucleic acid amplification reactions include interfacial amplification, bridge amplification, emulsion PCR, WILDFIRE amplification (e.g., U.S. Patent Application Publication No. 2013/0012399), the like or combinations thereof.

### Nucleic Acid Sequencing

In some embodiments, nucleic acid (e.g., nucleic acid fragments, target nucleic acid, sample nucleic acid, cell-free nucleic acid, double-stranded nucleic acid, double-stranded DNA, single-stranded nucleic acid, single-stranded DNA, single-stranded RNA) is sequenced. In some embodiments, nucleic acids hybridized to sequencing adapters (“hybridization products”) are sequenced by a sequencing process. In some embodiments, nucleic acids ligated to sequencing adapters (“ligation products”) are sequenced by a sequencing process. In some embodiments, hybridization products and/or ligation products are amplified by an amplification process, and the amplification products are sequenced by a sequencing process. In some embodiments, hybridization products and/or ligation products are not amplified by an amplification process, and the hybridization products and/or ligation products are sequenced without prior amplification by a sequencing process. In some embodiments, the sequencing process generates sequence reads (or sequencing reads). In some embodiments, a method herein comprises determining the sequence of a nucleic acid molecule based on the sequence reads.

For certain sequencing platforms (e.g., paired-end sequencing), generating sequence reads may include generating forward sequence reads and generating reverse sequence reads. For example, sequencing using certain paired-end sequencing platforms sequence each nucleic acid fragment from both directions, generally resulting in two reads per nucleic acid fragment, with the first read in a forward orientation (forward read) and the second read in reverse-complement orientation (reverse read). For certain platforms, a forward read is generated off a particular primer within a sequencing adapter (e.g., ILLUMINA adapter, P5 primer), and a reverse read is generated off a different primer within a sequencing adapter (e.g., ILLUMINA adapter, P7 primer).

Nucleic acid may be sequenced using any suitable sequencing platform including a Sanger sequencing platform, a high throughput or massively parallel sequencing (next generation sequencing (NGS)) platform, or the like, such as, for example, a sequencing platform provided by Illumina® (e.g., HiSeq™, MiSeq™ and/or Genome Analyzer™ sequencing systems); Oxford Nanopore™ Technologies (e.g., MINION sequencing system), Ion Torrent™ (e.g., Ion PGM™ and/or Ion Proton™ sequencing systems); Pacific Biosciences (e.g., PACBIO RS II sequencing system); Life Technologies™ (e.g., SOLID sequencing system); Roche (e.g., 454 GS FLX+ and/or GS Junior sequencing systems); or any other suitable sequencing platform. In some embodiments, the sequencing process is a highly multiplexed sequencing process. In certain instances, a full or substantially full sequence is obtained and sometimes a partial sequence is obtained. Nucleic acid sequencing generally produces a collection of sequence reads. As used herein, “reads” (e.g., “a read,” “a sequence read”) are short sequences of nucleotides produced by any sequencing process described herein or known in the art. Reads can be generated from one end of nucleic acid fragments (single-end reads), and sometimes are generated from both ends of nucleic acid fragments (e.g., paired-end reads, double-end reads). In some embodiments, a sequencing process generates short sequencing reads or “short reads.” In some embodiments, the nominal, average, mean or absolute length of short reads sometimes is about 10 continuous nucleotides to about 250 or more contiguous nucleotides. In some embodiments, the nominal, average, mean or absolute length of short reads sometimes is about 50 continuous nucleotides to about 150 or more contiguous nucleotides.

The length of a sequence read is often associated with the particular sequencing technology utilized. High-throughput methods, for example, provide sequence reads that can vary in size from tens to hundreds of base pairs (bp). Nanopore sequencing, for example, can provide sequence reads that can vary in size from tens to hundreds to thousands of base pairs. In some embodiments, sequence reads are of a mean, median, average or absolute length of about 15 bp to about 900 bp long. In certain embodiments sequence reads are of a mean, median, average or absolute length of about 1000 bp or more. In some embodiments sequence reads are of a mean, median, average or absolute length of about 1500, 2000, 2500, 3000, 3500, 4000, 4500, or 5000 bp or more. In some embodiments, sequence reads are of a mean, median, average or absolute length of about 100 bp to about 200 bp.

In some embodiments, the nominal, average, mean or absolute length of single-end reads sometimes is about 10 continuous nucleotides to about 250 or more contiguous nucleotides, about 15 contiguous nucleotides to about 200 or more contiguous nucleotides, about 15 contiguous nucleotides to about 150 or more contiguous nucleotides, about 15 contiguous nucleotides to about 125 or more contiguous nucleotides, about 15 contiguous nucleotides to about 100 or more contiguous nucleotides, about 15 contiguous nucleotides to about 75 or more contiguous nucleotides, about 15 contiguous nucleotides to about 60 or more contiguous nucleotides, 15 contiguous nucleotides to about 50 or more contiguous nucleotides, about 15 contiguous nucleotides to about 40 or more contiguous nucleotides, and sometimes about 15 contiguous nucleotides or about 36 or more contiguous nucleotides. In certain embodiments the nominal, average, mean or absolute length of single-end reads is about 20 to about 30 bases, or about 24 to about 28 bases in length. In certain embodiments the nominal, average, mean or absolute length of single-end reads is about 1, 2, 3, 4, 5, 6, 7, 8, 9, 10, 11, 12, 13, 14, 15, 16, 17, 18, 19, 21, 22, 23, 24, 25, 26, 27, 28 or about 29 bases or more in length. In certain embodiments the nominal, average, mean or absolute length of single-end reads is about 20 to about 200 bases, about 100 to about 200 bases, or about 140 to about 160 bases in length. In certain embodiments the nominal, average, mean or absolute length of single-end reads is about 30, 40, 50, 60, 70, 80, 90, 100, 110, 120, 130, 140, 150, 160, 170, 180, 190, or about 200 bases or more in length. In certain embodiments, the nominal, average, mean or absolute length of paired-end reads sometimes is about 10 contiguous nucleotides to about 25 contiguous nucleotides or more (e.g., about 10, 11, 12, 13, 14, 15, 16, 17, 18, 19, 20, 21, 22, 23, 24 or 25 nucleotides in length or more), about 15 contiguous nucleotides to about 20 contiguous nucleotides or more, and sometimes is about 17 contiguous nucleotides or about 18 contiguous nucleotides. In certain embodiments, the nominal, average, mean or absolute length of paired-end reads sometimes is about 25 contiguous nucleotides to about 400 contiguous nucleotides or more (e.g., about 25, 30, 40, 50, 60, 70, 80, 90, 100, 110, 120, 130, 140,150, 160, 170, 180, 190, 200, 210, 220, 230, 240, 250, 260, 270, 280, 290, 300, 310, 320, 330, 340, 350, 360, 370, 380, 390, or 400 nucleotides in length or more), about 50 contiguous nucleotides to about 350 contiguous nucleotides or more, about 100 contiguous nucleotides to about 325 contiguous nucleotides, about 150 contiguous nucleotides to about 325 contiguous nucleotides, about 200 contiguous nucleotides to about 325 contiguous nucleotides, about 275 contiguous nucleotides to about 310 contiguous nucleotides, about 100 contiguous nucleotides to about 200 contiguous nucleotides, about 100 contiguous nucleotides to about 175 contiguous nucleotides, about 125 contiguous nucleotides to about 175 contiguous nucleotides, and sometimes is about 140 contiguous nucleotides to about 160 contiguous nucleotides. In certain embodiments, the nominal, average, mean, or absolute length of paired-end reads is about 150 contiguous nucleotides, and sometimes is 150 contiguous nucleotides.

Reads generally are representations of nucleotide sequences in a physical nucleic acid. For example, in a read containing an ATGC depiction of a sequence, “A” represents an adenine nucleotide, “T” represents a thymine nucleotide, “G” represents a guanine nucleotide and “C” represents a cytosine nucleotide, in a physical nucleic acid. Sequence reads obtained from a sample from a subject can be reads from a mixture of a minority nucleic acid and a majority nucleic acid. For example, sequence reads obtained from the blood of a cancer patient can be reads from a mixture of cancer nucleic acid and non-cancer nucleic acid. In another example, sequence reads obtained from the blood of a pregnant female can be reads from a mixture of fetal nucleic acid and maternal nucleic acid. In another example, sequence reads obtained from the blood of a patient having an infection or infectious disease can be reads from a mixture of host nucleic acid and pathogen nucleic acid. In another example, sequence reads obtained from the blood of a transplant recipient can be reads from a mixture of host nucleic acid and transplant nucleic acid. In another example, sequence reads obtained from a sample can be reads from a mixture of nucleic acid from microorganisms collectively comprising a microbiome (e.g., microbiome of gut, microbiome of blood, microbiome of mouth, microbiome of spinal fluid, microbiome of feces) in a subject. In another example, sequence reads obtained from a sample can be reads from a mixture of nucleic acid from microorganisms collectively comprising a microbiome (e.g., microbiome of gut, microbiome of blood, microbiome of mouth, microbiome of spinal fluid, microbiome of feces), and nucleic acid from the host subject. A mixture of relatively short reads can be transformed by processes described herein into a representation of genomic nucleic acid present in the subject, and/or a representation of genomic nucleic acid present in a tumor, a fetus, a pathogen, a transplant, or a microbiome.

In certain embodiments, “obtaining” nucleic acid sequence reads of a sample from a subject and/or “obtaining” nucleic acid sequence reads of a biological specimen from one or more reference persons can involve directly sequencing nucleic acid to obtain the sequence information. In some embodiments, “obtaining” can involve receiving sequence information obtained directly from a nucleic acid by another.

In some embodiments, some or all nucleic acids in a sample are enriched and/or amplified (e.g., non-specifically, e.g., by a PCR based method) prior to or during sequencing. In certain embodiments, specific nucleic acid species or subsets in a sample are enriched and/or amplified prior to or during sequencing. In some embodiments, a species or subset of a pre-selected pool of nucleic acids is sequenced randomly. In some embodiments, nucleic acids in a sample are not enriched and/or amplified prior to or during sequencing.

In some embodiments, a representative fraction of a genome is sequenced and is sometimes referred to as “coverage” or “fold coverage.” For example, a 1-fold coverage indicates that roughly 100% of the nucleotide sequences of the genome are represented by reads. In some instances, fold coverage is referred to as (and is directly proportional to) “sequencing depth.” In some embodiments, “fold coverage” is a relative term referring to a prior sequencing run as a reference. For example, a second sequencing run may have 2-fold less coverage than a first sequencing run. In some embodiments, a genome is sequenced with redundancy, where a given region of the genome can be covered by two or more reads or overlapping reads (e.g., a “fold coverage” greater than 1, e.g., a 2-fold coverage). In some embodiments, a genome (e.g., a whole genome) is sequenced with about 0.01-fold to about 100-fold coverage, about 0.1-fold to 20-fold coverage, or about 0.1-fold to about 1-fold coverage (e.g., about 0.015-, 0.02-, 0.03-, 0.04-, 0.05-, 0.06-, 0.07-, 0.08-, 0.09-, 0.1-, 0.2-, 0.3-, 0.4-, 0.5-, 0.6-, 0.7-, 0.8-, 0.9-, 1-, 2-, 3-, 4-, 5-, 6-, 7-, 8-, 9-, 10-, 15-, 20-, 30-, 40-, 50-, 60-, 70-, 80-, 90-fold or greater coverage). In some embodiments, a genome (e.g., a whole genome) is sequenced with about 1-fold to about 200-fold coverage, or about 50-fold to 100-fold coverage (e.g., about 1-, 2-, 3-, 4-, 5-, 6-, 7-, 8-, 9-, 10-, 20-, 30-, 40-, 50-, 60-, 70-, 80-, 90-, 100-, 150-, 200-fold or greater coverage). In some embodiments, a genome (e.g., a whole genome) is sequenced with at least about 1-fold coverage. In some embodiments, a genome (e.g., a whole genome) is sequenced with at least about 2-fold coverage. In some embodiments, a genome (e.g., a whole genome) is sequenced with about 10-fold coverage. In some embodiments, a genome (e.g., a whole genome) is sequenced with about 50-fold coverage. In some embodiments, a genome (e.g., a whole genome) is sequenced with about 100-fold coverage.

In some embodiments, a test sample is sequenced using low coverage sequencing. Low coverage sequencing may be referred to as shallow depth sequencing. Low coverage sequencing may refer to sequencing at about 10-fold coverage or less. In some embodiments, a test sample is sequenced at about 10-fold coverage or less. In some embodiments, a test sample is sequenced at about 9-fold coverage or less. In some embodiments, a test sample is sequenced at about 8-fold coverage or less. In some embodiments, a test sample is sequenced at about 7-fold coverage or less. In some embodiments, a test sample is sequenced at about 6-fold coverage or less. In some embodiments, a test sample is sequenced at about 5-fold coverage or less. In some embodiments, a test sample is sequenced at about 4-fold coverage or less. In some embodiments, a test sample is sequenced at about 3-fold coverage or less. In some embodiments, a test sample is sequenced at about 2-fold coverage or less. In some embodiments, a test sample is sequenced at about 1-fold coverage or less. In some embodiments, a test sample is sequenced at a fold coverage between about 0.5-fold to about 2-fold. In some embodiments, a test sample is sequenced at about 2-fold coverage. In some embodiments, a test sample is sequenced at about 1-fold coverage. In some embodiments, a test sample is sequenced at about 0.9-fold coverage or less. In some embodiments, a test sample is sequenced at about 0.8-fold coverage or less. In some embodiments, a test sample is sequenced at about 0.7-fold coverage or less. In some embodiments, a test sample is sequenced at about 0.6-fold coverage or less. In some embodiments, a test sample is sequenced at about 0.5-fold coverage or less.

In some embodiments, specific parts of a genome (e.g., genomic parts from targeted methods) are sequenced and fold coverage values generally refer to the fraction of the specific genomic parts sequenced (i.e., fold coverage values do not refer to the whole genome). In some instances, specific genomic parts are sequenced at 1000-fold coverage or more. For example, specific genomic parts may be sequenced at 2000-fold, 5,000-fold, 10,000-fold, 20,000-fold, 30,000-fold, 40,000-fold or 50,000-fold coverage. In some embodiments, sequencing is at about 1,000-fold to about 100,000-fold coverage. In some embodiments, sequencing is at about 10,000-fold to about 70,000-fold coverage. In some embodiments, sequencing is at about 20,000-fold to about 60,000-fold coverage. In some embodiments, sequencing is at about 30,000-fold to about 50,000-fold coverage.

In some embodiments, one nucleic acid sample from one individual is sequenced. In certain embodiments, nucleic acids from each of two or more samples are sequenced, where samples are from one individual or from different individuals. In certain embodiments, nucleic acid samples from two or more biological samples are pooled, where each biological sample is from one individual or two or more individuals, and the pool is sequenced. In the latter embodiments, a nucleic acid sample from each biological sample often is identified by one or more unique identifiers.

In some embodiments, a sequencing method utilizes identifiers that allow multiplexing of sequence reactions in a sequencing process. The greater the number of unique identifiers, the greater the number of samples and/or chromosomes for detection, for example, that can be multiplexed in a sequencing process. A sequencing process can be performed using any suitable number of unique identifiers (e.g., 4, 8, 12, 24, 48, 96, or more).

A sequencing process sometimes makes use of a solid phase, and sometimes the solid phase comprises a flow cell on which nucleic acid from a library can be attached and reagents can be flowed and contacted with the attached nucleic acid. A flow cell sometimes includes flow cell lanes, and use of identifiers can facilitate analyzing a number of samples in each lane. A flow cell often is a solid support that can be configured to retain and/or allow the orderly passage of reagent solutions over bound analytes. Flow cells frequently are planar in shape, optically transparent, generally in the millimeter or sub-millimeter scale, and often have channels or lanes in which the analyte/reagent interaction occurs. In some embodiments, the number of samples analyzed in a given flow cell lane is dependent on the number of unique identifiers utilized during library preparation and/or probe design. Multiplexing using 12 identifiers, for example, allows simultaneous analysis of 96 samples (e.g., equal to the number of wells in a 96 well microwell plate) in an 8-lane flow cell. Similarly, multiplexing using 48 identifiers, for example, allows simultaneous analysis of 384 samples (e.g., equal to the number of wells in a 384 well microwell plate) in an 8-lane flow cell. Non-limiting examples of commercially available multiplex sequencing kits include Illumina's multiplexing sample preparation oligonucleotide kit and multiplexing sequencing primers and PhiX control kit (e.g., Illumina's catalog numbers PE-400-1001 and PE-400-1002, respectively).

Any suitable method of sequencing nucleic acids can be used, non-limiting examples of which include Maxim & Gilbert, chain-termination methods, sequencing by synthesis, sequencing by ligation, sequencing by mass spectrometry, microscopy-based techniques, the like or combinations thereof. In some embodiments, a first-generation technology, such as, for example, Sanger sequencing methods including automated Sanger sequencing methods, including microfluidic Sanger sequencing, can be used in a method provided herein. In some embodiments, sequencing technologies that include the use of nucleic acid imaging technologies (e.g., transmission electron microscopy (TEM) and atomic force microscopy (AFM)), can be used.

In some embodiments, a shotgun sequencing method is used. Shotgun sequencing generally refers to sequencing random nucleic acid strands. For example, DNA may be broken up randomly into numerous small fragments or DNA may be present as small fragments in a sample (e.g., cell-free DNA, degraded DNA). The DNA fragments are sequenced to obtain sequence reads. Multiple overlapping reads for the target DNA are obtained, and the overlapping reads are used to assemble the reads into a continuous sequence (typically performed using a computer program).

In some embodiments, a high-throughput sequencing method is used. High-throughput sequencing methods generally involve clonally amplified DNA templates or single DNA molecules that are sequenced in a massively parallel fashion, sometimes within a flow cell. Next generation (e.g., 2nd and 3rd generation) sequencing techniques capable of sequencing DNA in a massively parallel fashion can be used for methods described herein and are collectively referred to herein as “massively parallel sequencing” (MPS). In some embodiments, MPS sequencing methods utilize a targeted approach, where specific chromosomes, genes or regions of interest are sequenced. In certain embodiments, a non-targeted approach is used where most or all nucleic acids in a sample are sequenced, amplified and/or captured randomly.

In certain embodiments, sequence reads are generated using a whole genome sequencing approach. In certain embodiments, sequence reads are generated using a genome-wide sequencing approach. In certain embodiments, sequence reads are generated using a massively parallel sequencing approach. In certain embodiments, sequence reads are generated by a non-targeted sequencing approach. In certain embodiments, sequence reads are generated using a genome-wide, massively parallel sequencing approach. In certain embodiments, sequence reads are generated using a non-targeted, genome-wide sequencing approach. In certain embodiments, sequence reads are generated using a non-targeted, massively parallel sequencing approach. In certain embodiments, sequence reads are generated using a non-targeted, genome-wide, massively parallel sequencing approach.

Whole genome, genome-wide, massively parallel, and/or non-targeted sequencing approaches generate massive amounts of data. The human genome is approximately 3 billion base pairs in size. An example sequencing process performed on a test sample at 1-fold coverage would generate at least 3 million 1 kb reads. Sequencing processes that produce smaller reads and/or are performed at greater than 1-fold coverage would generate more than 3 million reads. Accordingly, such sequence data typically is processed (e.g., aligned, analyzed for alleles at target and linked loci, quantified, assessed for genotypes) using a computer, as the sheer volume of such data makes it impractical or impossible for a human to perform such a task without the use of a computer and/or software. In some embodiments, a method herein comprises generating, obtaining, and/or processing at least 100,000 sequence reads. In some embodiments, a method herein comprises generating, obtaining, and/or processing at least 500,000 sequence reads. In some embodiments, a method herein comprises generating, obtaining, and/or processing at least 1,000,000 sequence reads. In some embodiments, a method herein comprises generating, obtaining, and/or processing at least 2,000,000 sequence reads. In some embodiments, a method herein comprises generating, obtaining, and/or processing at least 3,000,000 sequence reads.

In some embodiments a targeted enrichment, amplification and/or sequencing approach is used. A targeted approach often isolates, selects and/or enriches a subset of nucleic acids in a sample for further processing by use of sequence-specific oligonucleotides. In some embodiments, a library of sequence-specific oligonucleotides are utilized to target (e.g., hybridize to) one or more sets of nucleic acids in a sample. Sequence-specific oligonucleotides and/or primers are often selective for particular sequences (e.g., unique nucleic acid sequences) present in one or more chromosomes, genes, exons, introns, and/or regulatory regions of interest. Any suitable method or combination of methods can be used for enrichment, amplification and/or sequencing of one or more subsets of targeted nucleic acids. In some embodiments targeted sequences are isolated and/or enriched by capture to a solid phase (e.g., a flow cell, a bead) using one or more sequence-specific anchors. In some embodiments targeted sequences are enriched and/or amplified by a polymerase-based method (e.g., a PCR-based method, by any suitable polymerase-based extension) using sequence-specific primers and/or primer sets. Sequence specific anchors often can be used as sequence-specific primers.

MPS sequencing sometimes makes use of sequencing by synthesis and certain imaging processes. A nucleic acid sequencing technology that may be used in a method described herein is sequencing-by-synthesis and reversible terminator-based sequencing (e.g., Illumina's Genome Analyzer; Genome Analyzer II; HISEQ 2000; HISEQ 2500 (Illumina, San Diego CA)). With this technology, millions of nucleic acid (e.g., DNA) fragments can be sequenced in parallel. In one example of this type of sequencing technology, a flow cell is used which contains an optically transparent slide with 8 individual lanes on the surfaces of which are bound oligonucleotide anchors (e.g., adapter primers).

Sequencing by synthesis generally is performed by iteratively adding (e.g., by covalent addition) a nucleotide to a primer or preexisting nucleic acid strand in a template directed manner. Each iterative addition of a nucleotide is detected and the process is repeated multiple times until a sequence of a nucleic acid strand is obtained. The length of a sequence obtained depends, in part, on the number of addition and detection steps that are performed. In some embodiments of sequencing by synthesis, one, two, three or more nucleotides of the same type (e.g., A, G, C or T) are added and detected in a round of nucleotide addition. Nucleotides can be added by any suitable method (e.g., enzymatically or chemically). For example, in some embodiments a polymerase or a ligase adds a nucleotide to a primer or to a preexisting nucleic acid strand in a template directed manner. In some embodiments of sequencing by synthesis, different types of nucleotides, nucleotide analogues and/or identifiers are used. In some embodiments, reversible terminators and/or removable (e.g., cleavable) identifiers are used. In some embodiments, fluorescent labeled nucleotides and/or nucleotide analogues are used. In certain embodiments sequencing by synthesis comprises a cleavage (e.g., cleavage and removal of an identifier) and/or a washing step. In some embodiments the addition of one or more nucleotides is detected by a suitable method described herein or known in the art, non-limiting examples of which include any suitable imaging apparatus, a suitable camera, a digital camera, a CCD (Charge Couple Device) based imaging apparatus (e.g., a CCD camera), a CMOS (Complementary Metal Oxide Silicon) based imaging apparatus (e.g., a CMOS camera), a photo diode (e.g., a photomultiplier tube), electron microscopy, a field-effect transistor (e.g., a DNA field-effect transistor), an ISFET ion sensor (e.g., a CHEMFET sensor), the like or combinations thereof.

Any suitable MPS method, system or technology platform for conducting methods described herein can be used to obtain nucleic acid sequence reads. Non-limiting examples of MPS platforms include ILLUMINA/SOLEX/HISEQ (e.g., Illumina's Genome Analyzer; Genome Analyzer II; HISEQ 2000; HISEQ), SOLID, Roche/454, PACBIO and/or SMRT, Helicos True Single Molecule Sequencing, Ion Torrent and Ion semiconductor-based sequencing (e.g., as developed by Life Technologies), WILDFIRE, 5500, 5500xl W and/or 5500xl W Genetic Analyzer based technologies (e.g., as developed and sold by Life Technologies, U.S. Patent Application Publication No. 2013/0012399); Polony sequencing, Pyrosequencing, Massively Parallel Signature Sequencing (MPSS), RNA polymerase (RNAP) sequencing, LASERGEN systems and methods, Nanopore-based platforms, chemical-sensitive field effect transistor (CHEMFET) array, electron microscopy-based sequencing (e.g., as developed by ZS Genetics, Halcyon Molecular), nanoball sequencing, the like or combinations thereof. Other sequencing methods that may be used to conduct methods herein include digital PCR, sequencing by hybridization, nanopore sequencing, chromosome-specific sequencing (e.g., using DANSR (digital analysis of selected regions) technology.

In some embodiments, nucleic acid is sequenced and the sequencing product (e.g., a collection of sequence reads, sequence read data) is processed prior to, or in conjunction with, an analysis of the sequenced nucleic acid. For example, sequence reads and/or sequence read data may be processed according to one or more of the following: aligning, mapping, filtering, quantifying, generating genotype likelihoods, generating genotypes, performing a genealogy analysis, and the like, and combinations thereof. Certain processing steps may be performed in any order and certain processing steps may be repeated.

### Aligning/Mapping Reads

Sequence reads can be aligned/mapped, and the number of reads carrying a particular allele or alleles are referred to as counts or quantifications (e.g., allele counts or allele quantifications). Any suitable aligning/mapping method (e.g., process, algorithm, program, software, module, the like or combination thereof) can be used. Certain aspects of aligning/mapping processes are described hereafter.

Mapping nucleotide sequence reads (i.e., sequence information from a fragment whose physical genomic position is unknown) can be performed in a number of ways, and often comprises alignment of the obtained sequence reads with a matching sequence in a reference genome. In such alignments, sequence reads generally are aligned to a reference sequence and those that align are designated as being “mapped,” as “a mapped sequence read” or as “a mapped read.”

The terms “aligned,” “alignment,” or “aligning” generally refer to two or more nucleic acid sequences that can be identified as a match (e.g., 100% identity) or partial match. Alignments are generally performed by a computer (e.g., a software, program, module, or algorithm), non-limiting examples of which include the Efficient Local Alignment of Nucleotide Data (ELAND) computer program distributed as part of the ILLUMINA Genomics Analysis pipeline. Alignment of a sequence read can be a 100% sequence match. In some cases, an alignment is less than a 100% sequence match (i.e., non-perfect match, partial match, partial alignment). In some embodiments an alignment is about a 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76% or 75% match. In some embodiments, an alignment comprises a mismatch. In some embodiments, an alignment comprises 1, 2, 3, 4 or 5 mismatches. Two or more sequences can be aligned using either strand (e.g., sense or antisense strand). In certain embodiments a nucleic acid sequence is aligned with the reverse complement of another nucleic acid sequence.

Various computational methods can be used to map each sequence read to a reference genome or portion thereof. Non-limiting examples of computer algorithms that can be used to align sequences include, without limitation, BLAST, BLITZ, FASTA, BOWTIE 1, BOWTIE 2, ELAND, MAQ, PROBEMATCH, SOAP, BWA (e.g., BWA-MEM aligner), or SEQMAP, or variations thereof or combinations thereof. In some embodiments, sequence reads are aligned with sequences in a reference genome. In some embodiments, sequence reads are found and/or aligned with sequences in nucleic acid databases known in the art including, for example, GENBANK, dbEST, dbSTS, EMBL (European Molecular Biology Laboratory) and DDBJ (DNA Databank of Japan). BLAST or similar tools can be used to search identified sequences against a sequence database.

In some embodiments, a read may uniquely or non-uniquely map to a reference genome. A read is considered as “uniquely mapped” if it aligns with a single sequence in the reference genome. A read is considered as “non-uniquely mapped” if it aligns with two or more sequences in the reference genome. In some embodiments, non-uniquely mapped reads are eliminated from further analysis (e.g. quantification). A certain, small degree of mismatch (0-1, 1 or more) may be allowed to account for single nucleotide polymorphisms that may exist between the reference genome and the reads from individual samples being mapped, in certain embodiments. In some embodiments, no degree of mismatch is allowed for a read mapped to a reference sequence.

As used herein, the term “reference genome” can refer to any particular known, sequenced or characterized genome, whether partial or complete, of any organism or virus which may be used to reference identified sequences from a subject. For example, a reference genome used for human subjects as well as many other organisms can be found at the National Center for Biotechnology Information at World Wide Web URL ncbi.nlm.nih.gov. A “genome” refers to the complete genetic information of an organism or virus, expressed in nucleic acid sequences. As used herein, a reference sequence or reference genome often is an assembled or partially assembled genomic sequence from an individual or multiple individuals. In some embodiments, a reference genome is an assembled or partially assembled genomic sequence from one or more human individuals. In some embodiments, a reference genome comprises sequences assigned to chromosomes.

In certain embodiments, mappability is assessed for a genomic region (e.g., portion, genomic portion). Mappability is the ability to unambiguously align a nucleotide sequence read to a reference genome, or portion thereof, typically up to a specified number of mismatches, including, for example, 0, 1, 2 or more mismatches. For a given genomic region, the expected mappability can be estimated using a sliding-window approach of a preset read length and averaging the resulting read-level mappability values. Genomic regions comprising stretches of unique nucleotide sequence sometimes have a high mappability value.

For paired-end sequencing, reads may be mapped to a reference genome by use of a suitable mapping and/or alignment program or algorithm, non-limiting examples of which include BWA (Li H. and Durbin R. (2009) Bioinformatics 25, 1754-60), NOVOALIGN [Novocraft (2010)], Bowtie (Langmead B, et al., (2009) Genome Biol. 10:R25), SOAP2 (Li R, et al., (2009) Bioinformatics 25, 1966-67), BFAST (Homer N, et al., (2009) PLoS ONE 4, e7767), GASSST (Rizk, G. and Lavenier, D. (2010) Bioinformatics 26, 2534-2540), and MPSCAN (Rivals E., et al. (2009) Lecture Notes in Computer Science 5724, 246-260), and the like. Reads can be trimmed and/or merged by use of a suitable trimming and/or merging program or algorithm, non-limiting examples of which include Cutadapt, trimmomatic, SeqPrep, and usearch. Some paired-end reads, such as those from nucleic acid templates that are shorter than the sequencing read length, can have portions sequenced by both the forward read and the reverse read; in such cases, the forward and reverse reads can be merged into a single read using the overlap between the forward and reverse reads. Reads that do not overlap or that do not overlap sufficiently can remain unmerged and be mapped as paired reads. Paired-end reads may be mapped and/or aligned using a suitable short read alignment program or algorithm. Non-limiting examples of short read alignment programs include BarraCUDA, BFAST, BLASTN, BLAT, Bowtie, BWA, CASHX, CUDA-EC, CUSHAW, CUSHAW2, drFAST, ELAND, ERNE, GNUMAP, GEM, GensearchNGS, GMAP, Geneious Assembler, iSAAC, LAST, MAQ, mrFAST, mrsFAST, MOSAIK, MPscan, Novoalign, NovoalignCS, Novocraft, NextGENe, Omixon, PALMapper, Partek, PASS, PerM, QPalma, RazerS, REAL, cREAL, RMAP, rNA, RTG, Segemehl, SeqMap, Shrec, SHRiMP, SLIDER, SOAP, SOAP2, SOAP3, SOCS, SSAHA, SSAHA2, Stampy, SToRM, Subread, Subjunc, Taipan, UGENE, VelociMapper, TimeLogic, XpressAlign, ZOOM, the like or combinations thereof. Paired-end reads are often mapped to opposing ends of the same polynucleotide fragment, according to a reference genome. In some embodiments, read mates are mapped independently. In some embodiments, information from both sequence reads (i.e., from each end) is factored in the mapping process. A reference genome is often used to determine and/or infer the sequence of nucleic acids located between paired-end read mates. The term “discordant read pairs” as used herein refers to a paired-end read comprising a pair of read mates, where one or both read mates fail to unambiguously map to the same region of a reference genome defined, in part, by a segment of contiguous nucleotides. In some embodiments discordant read pairs are paired-end read mates that map to unexpected locations of a reference genome. Non-limiting examples of unexpected locations of a reference genome include (i) two different chromosomes, (ii) locations separated by more than a predetermined fragment size (e.g., more than 300 bp, more than 500 bp, more than 1000 bp, more than 5000 bp, or more than 10,000 bp), (iii) an orientation inconsistent with a reference sequence (e.g., opposite orientations), the like or a combination thereof. In some embodiments discordant read mates are identified according to a length (e.g., an average length, a predetermined fragment size) or expected length of template polynucleotide fragments in a sample. For example, read mates that map to a location that is separated by more than the average length or expected length of polynucleotide fragments in a sample are sometimes identified as discordant read pairs. Read pairs that map in opposite orientation are sometimes determined by taking the reverse complement of one of the reads and comparing the alignment of both reads using the same strand of a reference sequence. Discordant read pairs can be identified by any suitable method and/or algorithm known in the art or described herein (e.g., SVDetect, Lumpy, BreakDancer, BreakDancerMax, CREST, DELLY, the like or combinations thereof).

### Classifications and Uses Thereof

Methods described herein can provide an outcome indicative of one or more characteristics of a sample or source described above. Methods described herein sometimes provide an outcome indicative of one or more genotypes for a test sample (e.g., providing an outcome determinative of one or more genotypes). Methods described herein sometimes provide an outcome indicative of an identification of a subject for a test sample (e.g., providing an outcome determinative of the identity of a subject). An outcome often is part of a classification process, and a classification (e.g., classification of one or more characteristics of a sample or source; and/or presence or absence of a genotype, phenotype, genetic variation, medical condition, and/or subject identification for a test sample) sometimes is based on and/or includes an outcome. An outcome and/or classification sometimes is based on and/or includes a result of data processing for a test sample that facilitates determining one or more characteristics of a sample or source and/or presence or absence of a genotype, phenotype, genetic variation, genetic alteration, medical condition, and/or subject identification in a classification process (e.g., a statistic value). An outcome and/or classification sometimes includes or is based on a score determinative of, or a call of, one or more characteristics of a sample or source and/or presence or absence of a genotype, phenotype, genetic variation, genetic alteration, medical condition, and/or subject identification. In certain embodiments, an outcome and/or classification includes a conclusion that predicts and/or determines one or more characteristics of a sample or source and/or presence or absence of a genotype, phenotype, genetic variation, genetic alteration, medical condition, and/or subject identification in a classification process.

Any suitable expression of an outcome and/or classification can be provided. An outcome and/or classification sometimes is based on and/or includes one or more numerical values generated using a processing method described herein in the context of one or more considerations of probability. Non-limiting examples of values that can be utilized include a sensitivity, specificity, standard deviation, median absolute deviation (MAD), measure of certainty, measure of confidence, measure of certainty or confidence that a value obtained for a test sample is inside or outside a particular range of values, measure of uncertainty, measure of uncertainty that a value obtained for a test sample is inside or outside a particular range of values, coefficient of variation (CV), confidence level, confidence interval (e.g., about 95% confidence interval), standard score (e.g., z-score), chi value, phi value, result of a t-test, p-value, ploidy value, area ratio, median level, the like or combination thereof. In some embodiments, an outcome and/or classification comprises a genotype likelihood, a set of genotype likelihoods, a genotype likelihood ratio, and/or a set of genotype likelihood ratios. In certain embodiments, multiple values are analyzed together. A consideration of probability can facilitate determining one or more characteristics of a sample or source.

In certain embodiments, an outcome and/or classification is based on and/or includes a conclusion that predicts and/or determines a risk or probability of the presence or absence of a genotype, phenotype, genetic variation, medical condition, and/or subject identification for a test sample. A conclusion sometimes is based on a value determined from a data analysis method described herein (e.g., a statistics value indicative of probability, certainty and/or uncertainty (e.g., standard deviation, median absolute deviation (MAD), measure of certainty, measure of confidence, measure of certainty or confidence that a value obtained for a test sample is inside or outside a particular range of values, measure of uncertainty, measure of uncertainty that a value obtained for a test sample is inside or outside a particular range of values, coefficient of variation (CV), confidence level, confidence interval (e.g., about 95% confidence interval), standard score (e.g., z-score), chi value, phi value, result of a t-test, p-value, sensitivity, specificity, the like or combination thereof). An outcome and/or classification sometimes is expressed in a laboratory test report for particular test sample as a probability (e.g., odds ratio, p-value), likelihood, or risk factor, associated with the presence or absence of a genotype, phenotype, genetic variation, medical condition, and/or subject identification. An outcome and/or classification for a test sample sometimes is provided as “positive” or “negative” with respect to a particular genotype, phenotype, genetic variation, medical condition, and/or subject identification. For example, an outcome and/or classification sometimes is designated as “positive” in a laboratory test report for a particular test sample where presence of a genotype, phenotype, genetic variation, medical condition, and/or subject identification is determined, and sometimes an outcome and/or classification is designated as “negative” in a laboratory test report for a particular test sample where absence of a genotype, phenotype, genetic variation, medical condition, and/or subject identification is determined. An outcome and/or classification sometimes is determined and sometimes includes an assumption used in data processing.

There typically are four types of classifications generated in a classification process: true positive, false positive, true negative and false negative. The term “true positive” as used herein refers to presence of a genotype, phenotype, genetic variation, medical condition, and/or subject identification correctly determined for a test sample. The term “false positive” as used herein refers to presence of a genotype, phenotype, genetic variation, medical condition, and/or subject identification incorrectly determined for a test sample. The term “true negative” as used herein refers to absence of a genotype, phenotype, genetic variation, medical condition, and/or subject identification correctly determined for a test sample. The term “false negative” as used herein refers to absence of a genotype, phenotype, genetic variation, medical condition, and/or subject identification incorrectly determined for a test sample. Two measures of performance for a classification process can be calculated based on the ratios of these occurrences: (i) a sensitivity value, which generally is the fraction of predicted positives that are correctly identified as being positives; and (ii) a specificity value, which generally is the fraction of predicted negatives correctly identified as being negative.

In certain embodiments, a laboratory test report generated for a classification process includes a measure of test performance (e.g., sensitivity and/or specificity) and/or a measure of confidence (e.g., a confidence level, confidence interval). A measure of test performance and/or confidence sometimes is obtained from a clinical validation study performed prior to performing a laboratory test for a test sample. In certain embodiments, one or more of sensitivity, specificity and/or confidence are expressed as a percentage. In some embodiments, a percentage expressed independently for each of sensitivity, specificity or confidence level, is greater than about 90% (e.g., about 90, 91, 92, 93, 94, 95, 96, 97, 98 or 99%, or greater than 99% (e.g., about 99.5%, or greater, about 99.9% or greater, about 99.95% or greater, about 99.99% or greater)). A confidence interval expressed for a particular confidence level (e.g., a confidence level of about 90% to about 99.9% (e.g., about 95%)) can be expressed as a range of values, and sometimes is expressed as a range or sensitivities and/or specificities for a particular confidence level. Coefficient of variation (CV) in some embodiments is expressed as a percentage, and sometimes the percentage is about 10% or less (e.g., about 10, 9, 8, 7, 6, 5, 4, 3, 2 or 1%, or less than 1% (e.g., about 0.5% or less, about 0.1% or less, about 0.05% or less, about 0.01% or less)). A probability (e.g., that a particular outcome and/or classification is not due to chance) in certain embodiments is expressed as a standard score (e.g., z-score), a p-value, or result of a t-test. In some embodiments, a measured variance, confidence level, confidence interval, sensitivity, specificity and the like (e.g., referred to collectively as confidence parameters) for an outcome and/or classification can be generated using one or more data processing manipulations described herein.

An outcome and/or classification for a test sample often is ordered by, and often is provided to, a health care professional, law enforcement professional, or other qualified individual who transmits an outcome and/or classification to a subject from whom the test sample is obtained. In certain embodiments, an outcome and/or classification is provided using a suitable visual medium (e.g., a peripheral or component of a machine, e.g., a printer or display). A classification and/or outcome often is provided to a healthcare professional, law enforcement professional, or qualified individual in the form of a report. A report typically comprises a display of an outcome and/or classification (e.g., a value, one or more characteristics of a sample or source, or an assessment or probability of presence or absence of a genotype, phenotype, genetic variation, medical condition, and/or subject identification), sometimes includes an associated confidence parameter, and sometimes includes a measure of performance for a test used to generate the outcome and/or classification. A report sometimes includes a recommendation for a follow-up procedure (e.g., a procedure that confirms the outcome or classification). A report sometimes includes a visual representation of a chromosome or portion thereof (e.g., a chromosome ideogram or karyogram), and sometimes shows a visualization of a duplication and/or deletion region for a chromosome (e.g., a visualization of a whole chromosome for a chromosome deletion or duplication; a visualization of a whole chromosome with a deleted region or duplicated region shown; a visualization of a portion of chromosome duplicated or deleted; a visualization of a portion of a chromosome remaining in the event of a deletion of a portion of a chromosome) identified for a test sample.

A report can be displayed in a suitable format that facilitates determination of presence or absence of a genotype, phenotype, genetic variation, medical condition, and/or subject identification by a health professional or other qualified individual. Non-limiting examples of formats suitable for use for generating a report include digital data, a graph, a 2D graph, a 3D graph, and 4D graph, a picture (e.g., a jpg, bitmap (e.g., bmp), pdf, tiff, gif, raw, png, the like or suitable format), a pictograph, a chart, a table, a bar graph, a pie graph, a diagram, a flow chart, a scatter plot, a map, a histogram, a density chart, a function graph, a circuit diagram, a block diagram, a bubble map, a constellation diagram, a contour diagram, a cartogram, spider chart, Venn diagram, nomogram, and the like, or combination of the foregoing.

A report may be generated by a computer and/or by human data entry, and can be transmitted and communicated using a suitable electronic medium (e.g., via the internet, via computer, via facsimile, from one network location to another location at the same or different physical sites), or by another method of sending or receiving data (e.g., mail service, courier service and the like). Non-limiting examples of communication media for transmitting a report include auditory file, computer readable file (e.g., pdf file), paper file, laboratory file, medical record file, or any other medium described in the previous paragraph. A laboratory file or medical record file may be in tangible form or electronic form (e.g., computer readable form), in certain embodiments. After a report is generated and transmitted, a report can be received by obtaining, via a suitable communication medium, a written and/or graphical representation comprising an outcome and/or classification, which upon review allows a healthcare professional, law enforcement professional, or other qualified individual to make a determination as to one or more characteristics of a sample or source, or presence or absence of a genotype, phenotype, genetic variation, medical condition, and/or subject identification for a test sample.

An outcome and/or classification may be provided by and obtained from a laboratory (e.g., obtained from a laboratory file). A laboratory file can be generated by a laboratory that carries out one or more tests for determining one or more characteristics of a sample or source and/or presence or absence of a genotype, phenotype, genetic variation, medical condition, and/or subject identification for a test sample. Laboratory personnel (e.g., a laboratory manager) can analyze information associated with test samples (e.g., test profiles, reference profiles, test values, reference values, level of deviation, patient information) underlying an outcome and/or classification. For calls pertaining to presence or absence of a genotype, phenotype, genetic variation, medical condition, and/or subject identification that are close or questionable, laboratory personnel can re-run the same procedure using the same (e.g., aliquot of the same sample) or different test sample from a test subject. A laboratory may be in the same location or different location (e.g., in another country) as personnel assessing the presence or absence of a genotype, phenotype, genetic variation, medical condition, and/or subject identification from the laboratory file. For example, a laboratory file can be generated in one location and transmitted to another location in which the information for a test sample therein is assessed by a healthcare professional, law enforcement professional, or other qualified individual, and optionally, transmitted to the subject from which the test sample was obtained. A laboratory sometimes generates and/or transmits a laboratory report containing a classification of presence or absence of genomic instability, a genotype, phenotype, a genetic variation, medical condition, and/or subject identification for a test sample. A laboratory generating a laboratory test report sometimes is a certified laboratory, and sometimes is a laboratory certified under the Clinical Laboratory Improvement Amendments (CLIA).

### Machines, Software and Interfaces

Certain processes and methods described herein (e.g., selecting a subset of sequence reads, generating a sequence reads profile, processing sequence read data, processing sequence read quantifications, determining one or more characteristics of a sample based on sequence read data or a sequence read profile) often are too complex for performing in the mind and cannot be performed without a computer, microprocessor, software, module or other machine. Methods described herein may be computer-implemented methods, and one or more portions of a method sometimes are performed by one or more processors (e.g., microprocessors), computers, systems, apparatuses, or machines (e.g., microprocessor-controlled machine).

Computers, systems, apparatuses, machines and computer program products suitable for use often include, or are utilized in conjunction with, computer readable storage media. Non-limiting examples of computer readable storage media include memory, hard disk, CD-ROM, flash memory device and the like. Computer readable storage media generally are computer hardware, and often are non-transitory computer-readable storage media. Computer readable storage media are not computer readable transmission media, the latter of which are transmission signals per se.

Provided herein are computer readable storage media with an executable program stored thereon, where the program instructs a microprocessor to perform a method described herein. Provided also are computer readable storage media with an executable program module stored thereon, where the program module instructs a microprocessor to perform part of a method described herein. Also provided herein are systems, machines, apparatuses and computer program products that include computer readable storage media with an executable program stored thereon, where the program instructs a microprocessor to perform a method described herein. Provided also are systems, machines and apparatuses that include computer readable storage media with an executable program module stored thereon, where the program module instructs a microprocessor to perform part of a method described herein.

Also provided are computer program products. A computer program product often includes a computer usable medium that includes a computer readable program code embodied therein, the computer readable program code adapted for being executed to implement a method or part of a method described herein. Computer usable media and readable program code are not transmission media (i.e., transmission signals per se). Computer readable program code often is adapted for being executed by a processor, computer, system, apparatus, or machine.

In some embodiments, methods described herein (e.g., selecting a subset of sequence reads, generating a sequence reads profile, processing sequence read data, processing sequence read quantifications, determining one or more characteristics of a sample based on sequence read data or a sequence read profile) are performed by automated methods. In some embodiments, one or more steps of a method described herein are carried out by a microprocessor and/or computer, and/or carried out in conjunction with memory. In some embodiments, an automated method is embodied in software, modules, microprocessors, peripherals and/or a machine comprising the like, that perform methods described herein. As used herein, software refers to computer readable program instructions that, when executed by a microprocessor, perform computer operations, as described herein.

Machines, software and interfaces may be used to conduct methods described herein. Using machines, software and interfaces, a user may enter, request, query or determine options for using particular information, programs or processes (e.g., processing sequence read data, processing sequence read quantifications, and/or providing an outcome), which can involve implementing statistical analysis algorithms, statistical significance algorithms, statistical algorithms, iterative steps, validation algorithms, and graphical representations, for example. In some embodiments, a data set may be entered by a user as input information, a user may download one or more data sets by suitable hardware media (e.g., flash drive), and/or a user may send a data set from one system to another for subsequent processing and/or providing an outcome (e.g., send sequence read data from a sequencer to a computer system for sequence read processing; send processed sequence read data to a computer system for further processing and/or yielding an outcome and/or report).

A system typically comprises one or more machines. Each machine comprises one or more of memory, one or more microprocessors, and instructions. Where a system includes two or more machines, some or all of the machines may be located at the same location, some or all of the machines may be located at different locations, all of the machines may be located at one location and/or all of the machines may be located at different locations. Where a system includes two or more machines, some or all of the machines may be located at the same location as a user, some or all of the machines may be located at a location different than a user, all of the machines may be located at the same location as the user, and/or all of the machine may be located at one or more locations different than the user.

A system sometimes comprises a computing machine and a sequencing apparatus or machine, where the sequencing apparatus or machine is configured to receive physical nucleic acid and generate sequence reads, and the computing apparatus is configured to process the reads from the sequencing apparatus or machine. The computing machine sometimes is configured to determine an outcome from the sequence reads (e.g., a characteristic of a sample).

A user may, for example, place a query to software which then may acquire a data set via internet access, and in certain embodiments, a programmable microprocessor may be prompted to acquire a suitable data set based on given parameters. A programmable microprocessor also may prompt a user to select one or more data set options selected by the microprocessor based on given parameters. A programmable microprocessor may prompt a user to select one or more data set options selected by the microprocessor based on information found via the internet, other internal or external information, or the like. Options may be chosen for selecting one or more data feature selections, one or more statistical algorithms, one or more statistical analysis algorithms, one or more statistical significance algorithms, iterative steps, one or more validation algorithms, and one or more graphical representations of methods, machines, apparatuses, computer programs or a non-transitory computer-readable storage medium with an executable program stored thereon.

Systems addressed herein may comprise general components of computer systems, such as, for example, network servers, laptop systems, desktop systems, handheld systems, personal digital assistants, computing kiosks, and the like. A computer system may comprise one or more input means such as a keyboard, touch screen, mouse, voice recognition or other means to allow the user to enter data into the system. A system may further comprise one or more outputs, including, but not limited to, a display screen (e.g., CRT or LCD), speaker, FAX machine, printer (e.g., laser, ink jet, impact, black and white or color printer), or other output useful for providing visual, auditory and/or hardcopy output of information (e.g., outcome and/or report).

In a system, input and output components may be connected to a central processing unit which may comprise among other components, a microprocessor for executing program instructions and memory for storing program code and data. In some embodiments, processes may be implemented as a single user system located in a single geographical site. In certain embodiments, processes may be implemented as a multi-user system. In the case of a multi-user implementation, multiple central processing units may be connected by means of a network. The network may be local, encompassing a single department in one portion of a building, an entire building, span multiple buildings, span a region, span an entire country or be worldwide. The network may be private, being owned and controlled by a provider, or it may be implemented as an internet based service where the user accesses a web page to enter and retrieve information. Accordingly, in certain embodiments, a system includes one or more machines, which may be local or remote with respect to a user. More than one machine in one location or multiple locations may be accessed by a user, and data may be mapped and/or processed in series and/or in parallel. Thus, a suitable configuration and control may be utilized for mapping and/or processing data using multiple machines, such as in local network, remote network and/or “cloud” computing platforms.

A system can include a communications interface in some embodiments. A communications interface allows for transfer of software and data between a computer system and one or more external devices. Non-limiting examples of communications interfaces include a modem, a network interface (such as an Ethernet card), a communications port, a PCMCIA slot and card, and the like. Software and data transferred via a communications interface generally are in the form of signals, which can be electronic, electromagnetic, optical and/or other signals capable of being received by a communications interface. Signals often are provided to a communications interface via a channel. A channel often carries signals and can be implemented using wire or cable, fiber optics, a phone line, a cellular phone link, an RF link and/or other communications channels. Thus, in an example, a communications interface may be used to receive signal information that can be detected by a signal detection module.

Data may be input by a suitable device and/or method, including, but not limited to, manual input devices or direct data entry devices (DDEs). Non-limiting examples of manual devices include keyboards, concept keyboards, touch sensitive screens, light pens, mouse, tracker balls, joysticks, graphic tablets, scanners, digital cameras, video digitizers and voice recognition devices. Non-limiting examples of DDEs include bar code readers, magnetic strip codes, smart cards, magnetic ink character recognition, optical character recognition, optical mark recognition, and turnaround documents.

In some embodiments, output from a sequencing apparatus or machine may serve as data that can be input via an input device. In certain embodiments, sequence read information may serve as data that can be input via an input device. In certain embodiments, mapped sequence reads may serve as data that can be input via an input device. In certain embodiments, nucleic acid fragment size (e.g., length) may serve as data that can be input via an input device. In certain embodiments, output from a nucleic acid capture process (e.g., genomic region origin data) may serve as data that can be input via an input device. In certain embodiments, a combination of nucleic acid fragment size (e.g., length) and output from a nucleic acid capture process (e.g., genomic region origin data) may serve as data that can be input via an input device. In certain embodiments, simulated data is generated by an in silico process and the simulated data serves as data that can be input via an input device. The term “in silico” refers to research and experiments performed using a computer. In silico processes include, but are not limited to, mapping sequence reads and processing mapped sequence reads according to processes described herein.

A system may include software useful for performing a process or part of a process described herein, and software can include one or more modules for performing such processes (e.g., sequencing module, logic processing module, data display organization module). The term “software” refers to computer readable program instructions that, when executed by a computer, perform computer operations. Instructions executable by the one or more microprocessors sometimes are provided as executable code, that when executed, can cause one or more microprocessors to implement a method described herein. A module described herein can exist as software, and instructions (e.g., processes, routines, subroutines) embodied in the software can be implemented or performed by a microprocessor. For example, a module (e.g., a software module) can be a part of a program that performs a particular process or task. The term “module” refers to a self-contained functional unit that can be used in a larger machine or software system. A module can comprise a set of instructions for carrying out a function of the module. A module can transform data and/or information. Data and/or information can be in a suitable form. For example, data and/or information can be digital or analogue. In certain embodiments, data and/or information sometimes can be packets, bytes, characters, or bits. In some embodiments, data and/or information can be any gathered, assembled or usable data or information. Non-limiting examples of data and/or information include a suitable media, pictures, video, sound (e.g. frequencies, audible or non-audible), numbers, constants, a value, objects, time, functions, instructions, maps, references, sequences, reads, mapped reads, levels, ranges, thresholds, signals, displays, representations, or transformations thereof. A module can accept or receive data and/or information, transform the data and/or information into a second form, and provide or transfer the second form to a machine, peripheral, component or another module. A microprocessor can, in certain embodiments, carry out the instructions in a module. In some embodiments, one or more microprocessors are required to carry out instructions in a module or group of modules. A module can provide data and/or information to another module, machine or source and can receive data and/or information from another module, machine or source.

A computer program product sometimes is embodied on a tangible computer-readable medium, and sometimes is tangibly embodied on a non-transitory computer-readable medium. A module sometimes is stored on a computer readable medium (e.g., disk, drive) or in memory (e.g., random access memory). A module and microprocessor capable of implementing instructions from a module can be located in a machine or in a different machine. A module and/or microprocessor capable of implementing an instruction for a module can be located in the same location as a user (e.g., local network) or in a different location from a user (e.g., remote network, cloud system). In embodiments in which a method is carried out in conjunction with two or more modules, the modules can be located in the same machine, one or more modules can be located in different machine in the same physical location, and one or more modules may be located in different machines in different physical locations.

A machine, in some embodiments, comprises at least one microprocessor for carrying out the instructions in a module. Sequence read quantifications (e.g., allele counts) sometimes are accessed by a microprocessor that executes instructions configured to carry out a method described herein. Sequence read quantifications that are accessed by a microprocessor can be within memory of a system, and the sequence read counts can be accessed and placed into the memory of the system after they are obtained. In some embodiments, a machine includes a microprocessor (e.g., one or more microprocessors) which microprocessor can perform and/or implement one or more instructions (e.g., processes, routines and/or subroutines) from a module. In some embodiments, a machine includes multiple microprocessors, such as microprocessors coordinated and working in parallel. In some embodiments, a machine operates with one or more external microprocessors (e.g., an internal or external network, server, storage device and/or storage network (e.g., a cloud)). In some embodiments, a machine comprises a module (e.g., one or more modules). A machine comprising a module often is capable of receiving and transferring one or more of data and/or information to and from other modules.

In certain embodiments, a machine comprises peripherals and/or components. In certain embodiments, a machine can comprise one or more peripherals or components that can transfer data and/or information to and from other modules, peripherals and/or components. In certain embodiments, a machine interacts with a peripheral and/or component that provides data and/or information. In certain embodiments, peripherals and components assist a machine in carrying out a function or interact directly with a module. Non-limiting examples of peripherals and/or components include a suitable computer peripheral, I/O or storage method or device including but not limited to scanners, printers, displays (e.g., monitors, LED, LCT or CRTs), cameras, microphones, pads (e.g., IPADs, tablets), touch screens, smart phones, mobile phones, USB I/O devices, USB mass storage devices, keyboards, a computer mouse, digital pens, modems, hard drives, jump drives, flash drives, a microprocessor, a server, CDs, DVDs, graphic cards, specialized I/O devices (e.g., sequencers, photo cells, photo multiplier tubes, optical readers, sensors, etc.), one or more flow cells, fluid handling components, network interface controllers, ROM, RAM, wireless transfer methods and devices (Bluetooth, WiFi, and the like), the world wide web (www), the internet, a computer and/or another module.

Software often is provided on a program product containing program instructions recorded on a computer readable medium, including, but not limited to, magnetic media including floppy disks, hard disks, and magnetic tape; and optical media including CD-ROM discs, DVD discs, magneto-optical discs, flash memory devices (e.g., flash drives), RAM, floppy discs, the like, and other such media on which the program instructions can be recorded. In online implementation, a server and web site maintained by an organization can be configured to provide software downloads to remote users, or remote users may access a remote system maintained by an organization to remotely access software. Software may obtain or receive input information. Software may include a module that specifically obtains or receives data (e.g., a data receiving module that receives sequence read data and/or mapped read data) and may include a module that specifically processes the data (e.g., a processing module that processes received data (e.g., filters, normalizes, provides an outcome and/or report). The terms “obtaining” and “receiving” input information refers to receiving data (e.g., sequence reads, mapped reads) by computer communication means from a local, or remote site, human data entry, or any other method of receiving data. The input information may be generated in the same location at which it is received, or it may be generated in a different location and transmitted to the receiving location. In some embodiments, input information is modified before it is processed (e.g., placed into a format amenable to processing (e.g., tabulated)).

Software can include one or more algorithms in certain embodiments. An algorithm may be used for processing data and/or providing an outcome or report according to a finite sequence of instructions. An algorithm often is a list of defined instructions for completing a task. Starting from an initial state, the instructions may describe a computation that proceeds through a defined series of successive states, eventually terminating in a final ending state. The transition from one state to the next is not necessarily deterministic (e.g., some algorithms incorporate randomness). By way of example, and without limitation, an algorithm can be a search algorithm, sorting algorithm, merge algorithm, numerical algorithm, graph algorithm, string algorithm, modeling algorithm, computational genometric algorithm, combinatorial algorithm, machine learning algorithm, cryptography algorithm, data compression algorithm, parsing algorithm and the like. An algorithm can include one algorithm or two or more algorithms working in combination. An algorithm can be of any suitable complexity class and/or parameterized complexity. An algorithm can be used for calculation and/or data processing, and in some embodiments, can be used in a deterministic or probabilistic/predictive approach. An algorithm can be implemented in a computing environment by use of a suitable programming language, non-limiting examples of which are C, C++, Java, Perl, Python, Fortran, and the like. In some embodiments, an algorithm can be configured or modified to include margin of errors, statistical analysis, statistical significance, and/or comparison to other information or data sets (e.g., applicable when using a neural net or clustering algorithm).

In certain embodiments, several algorithms may be implemented for use in software. These algorithms can be trained with raw data in some embodiments. For each new raw data sample, the trained algorithms may produce a representative processed data set or outcome. A processed data set sometimes is of reduced complexity compared to the parent data set that was processed. Based on a processed set, the performance of a trained algorithm may be assessed based on sensitivity and specificity, in some embodiments. An algorithm with the highest sensitivity and/or specificity may be identified and utilized, in certain embodiments.

In certain embodiments, simulated (or simulation) data can aid data processing, for example, by training an algorithm or testing an algorithm. In some embodiments, simulated data includes hypothetical various samplings of different groupings of sequence reads. Simulated data may be based on what might be expected from a real population or may be skewed to test an algorithm and/or to assign a correct classification. Simulated data also is referred to herein as “virtual” data. Simulations can be performed by a computer program in certain embodiments. One possible step in using a simulated data set is to evaluate the confidence of identified results, e.g., how well a random sampling matches or best represents the original data. One approach is to calculate a probability value (p-value), which estimates the probability of a random sample having better score than the selected samples. In some embodiments, an empirical model may be assessed, in which it is assumed that at least one sample matches a reference sample (with or without resolved variations). In some embodiments, another distribution, such as a Poisson distribution for example, can be used to define the probability distribution.

A system may include one or more microprocessors in certain embodiments. A microprocessor can be connected to a communication bus. A computer system may include a main memory, often random access memory (RAM), and can also include a secondary memory. Memory in some embodiments comprises a non-transitory computer-readable storage medium. Secondary memory can include, for example, a hard disk drive and/or a removable storage drive, representing a floppy disk drive, a magnetic tape drive, an optical disk drive, memory card and the like. A removable storage drive often reads from and/or writes to a removable storage unit. Non-limiting examples of removable storage units include a floppy disk, magnetic tape, optical disk, and the like, which can be read by and written to by, for example, a removable storage drive. A removable storage unit can include a computer-usable storage medium having stored therein computer software and/or data.

A microprocessor may implement software in a system. In some embodiments, a microprocessor may be programmed to automatically perform a task described herein that a user could perform. Accordingly, a microprocessor, or algorithm conducted by such a microprocessor, can require little to no supervision or input from a user (e.g., software may be programmed to implement a function automatically). In some embodiments, the complexity of a process is so large that a single person or group of persons could not perform the process in a timeframe short enough for determining one or more characteristics of a sample.

In some embodiments, secondary memory may include other similar means for allowing computer programs or other instructions to be loaded into a computer system. For example, a system can include a removable storage unit and an interface device. Non-limiting examples of such systems include a program cartridge and cartridge interface (such as that found in video game devices), a removable memory chip (such as an EPROM, or PROM) and associated socket, and other removable storage units and interfaces that allow software and data to be transferred from the removable storage unit to a computer system.

Provided herein, in certain embodiments, are systems, machines and apparatuses comprising one or more microprocessors and memory, which memory comprises instructions executable by the one or more microprocessors and which instructions executable by the one or more microprocessors are configured to generate allele quantifications and a set of genotype likelihoods, and, based on the set of genotype likelihoods, generate a genotype.

Provided herein, in certain embodiments, are machines comprising one or more microprocessors and memory, which memory comprises instructions executable by the one or more microprocessors and which memory comprises sequence reads aligned to a reference genome, and which instructions executable by the one or more microprocessors are configured to generate allele quantifications and a set of genotype likelihoods, and, based on the set of genotype likelihoods, generate a genotype.

Provided herein, in certain embodiments, are non-transitory computer-readable storage media with an executable program stored thereon, where the program instructs a microprocessor to perform the following: (a) access sequence reads aligned to a reference genome, (b) generate allele quantifications and a set of genotype likelihoods, and (c) based on the set of genotype likelihoods, generate a genotype.

Provided herein, in certain embodiments, are systems, machines and apparatuses comprising one or more microprocessors and memory, which memory comprises instructions executable by the one or more microprocessors and which instructions executable by the one or more microprocessors are configured to generate allele quantifications and a haplotype pair likelihood set, and, based on the haplotype pair likelihood set, generate a genotype.

Provided herein, in certain embodiments, are machines comprising one or more microprocessors and memory, which memory comprises instructions executable by the one or more microprocessors and which memory comprises sequence reads aligned to a reference genome, and which instructions executable by the one or more microprocessors are configured to generate allele quantifications and a haplotype pair likelihood set, and, based on the haplotype pair likelihood set, generate a genotype.

Provided herein, in certain embodiments, are non-transitory computer-readable storage media with an executable program stored thereon, where the program instructs a microprocessor to perform the following: (a) access sequence reads aligned to a reference genome, (b) generate allele quantifications and a haplotype pair likelihood set, and (c) based on the haplotype pair likelihood set, generate a genotype.

Provided herein, in certain embodiments, are systems, machines and apparatuses comprising one or more microprocessors and memory, which memory comprises instructions executable by the one or more microprocessors and which instructions executable by the one or more microprocessors are configured to generate allele quantifications from a mixture of nucleic acid from an unknown subject and a known subject, estimate a fraction of nucleic acid from an unknown subject in a mixture, generate a set of genotype likelihoods, and, based on the set of genotype likelihoods, generate a genotype.

Provided herein, in certain embodiments, are machines comprising one or more microprocessors and memory, which memory comprises instructions executable by the one or more microprocessors and which memory comprises sequence reads aligned to a reference genome, and which instructions executable by the one or more microprocessors are configured to generate allele quantifications from a mixture of nucleic acid from an unknown subject and a known subject, estimate a fraction of nucleic acid from an unknown subject in the mixture, generate a set of genotype likelihoods, and, based on the set of genotype likelihoods, generate a genotype.

Provided herein, in certain embodiments, are non-transitory computer-readable storage media with an executable program stored thereon, where the program instructs a microprocessor to perform the following: (a) access sequence reads aligned to a reference genome, which reads are from a sample comprising a mixture of nucleic acid from an unknown subject and a known subject, (b) generate allele quantifications, (c) estimate a fraction of nucleic acid from an unknown subject in the mixture, (d) generate a set of genotype likelihoods, and (e) based on the set of genotype likelihoods, generate a genotype.

Provided herein, in certain embodiments, are systems, machines and apparatuses comprising one or more microprocessors and memory, which memory comprises instructions executable by the one or more microprocessors and which instructions executable by the one or more microprocessors are configured to generate allele quantifications for a haplotype group from a mixture of nucleic acid from an unknown subject and a known subject, estimate a fraction of nucleic acid from an unknown subject in a mixture, generate a haplotype pair likelihood set and, based on the haplotype pair likelihood set, generate a genotype.

Provided herein, in certain embodiments, are machines comprising one or more microprocessors and memory, which memory comprises instructions executable by the one or more microprocessors and which memory comprises sequence reads aligned to a reference genome, and which instructions executable by the one or more microprocessors are configured to generate allele quantifications for a haplotype group from a mixture of nucleic acid from an unknown subject and a known subject, estimate a fraction of nucleic acid from an unknown subject in a mixture, generate a haplotype pair likelihood set and, based on the haplotype pair likelihood set, generate a genotype.

Provided herein, in certain embodiments, are non-transitory computer-readable storage media with an executable program stored thereon, where the program instructs a microprocessor to perform the following: (a) access sequence reads aligned to a reference genome, which reads are from a sample comprising a mixture of nucleic acid from an unknown subject and a known subject, (b) generate allele quantifications for a haplotype group, (c) estimate a fraction of nucleic acid from an unknown subject in the mixture, (d) generate a haplotype pair likelihood set, and (e) based on the haplotype pair likelihood set, generate a genotype.

### Kits

Provided in certain embodiments are kits. The kits may include any components and compositions described herein (e.g., sequencing adapters and components/subcomponents thereof, oligonucleotides, oligonucleotide components/regions, nucleic acids, primers, enzymes) useful for performing any of the methods described herein, in any suitable combination. Kits may further include any reagents, buffers, or other components useful for carrying out any of the methods described herein.

Kits may include components for capturing nucleic acid (e.g., cell free nucleic acid, damaged or degraded nucleic acid, fragmented nucleic acid) from a sample (e.g., a forensic sample). Kits for capturing nucleic acid from a forensic sample may be configured such that a user provides the sample nucleic acid.

Components of a kit may be present in separate containers, or multiple components may be present in a single container. Suitable containers include a single tube (e.g., vial), one or more wells of a plate (e.g., a 96-well plate, a 384-well plate, and the like), and the like.

Kits may also comprise instructions for performing one or more methods described herein and/or a description of one or more components described herein. For example, a kit may include instructions for using sequencing adapters, or components thereof, to capture nucleic acid from a sample (e.g., a forensic sample) and/or to produce a nucleic acid library. Instructions and/or descriptions may be in printed form and may be included in a kit insert. In some embodiments, instructions and/or descriptions are provided as an electronic storage data file present on a suitable computer readable storage medium, e.g., portable flash drive, DVD, CD-ROM, diskette, and the like. A kit also may include a written description of an internet location that provides such instructions or descriptions.

### EXAMPLES

The examples set forth below illustrate certain implementations and do not limit the technology.

### Example 1: A Fast Procedure for Genotype Inference from Low Coverage and Fragmented DNA Sequence Data and Haplotype Panels

Databases of genotype information for millions of individuals are available for search and analysis. These databases include GEDMATCH and FAMILYTREEDNA. The genotype data in these databases often is contributed by users of direct to consumer (DTC) genetic testing companies such as, for example, 23ANDME or ANCESTRYDNA. The original concept for the more open databases was to provide a platform for genealogy enthusiasts to do DNA based relative finding across platforms. For example, such databases allow a user who may have ANCESTRYDNA genotype data to find a cousin who may have 23ANDME genotype data.

Recently, law enforcement have recognized the potential of genetic genealogy to assist with solving crime. It is often possible to perform a genotype analysis on a forensic sample like blood or semen. Typically, the same genotype array technology used by direct to consumer companies is used to genotype forensic samples. However, many forensic samples (e.g., hair, bone) do not contain enough good quality DNA for use on a genotype array.

This Example describes a procedure that produces accurate genotypes from very low coverage shotgun sequence data. Furthermore, this approach works even when the DNA sequence data is derived from highly fragmented and chemically damaged DNA molecules, as is often found in forensic samples. In certain instances, sequencing libraries are generated from highly fragmented and chemically damaged DNA molecules using scaffold adapters described herein.

The approach to infer genotypes at a defined set of target site positions is outlined below:

1. Generate DNA sequence reads using low coverage shotgun sequencing. Typically, about 2 fold coverage of the genome is used as input data. This fold coverage input level may decrease as imputation panels grow. A 2 fold coverage of the genome means that each position in the genome is observed, on average, 2 times. However, as each DNA sequence read is from a random position on the genome, some genomic positions are not observed in any DNA sequence read. Some genomic positions are observed once or only a few times.

2. Align the DNA sequence reads to a reference genome to determine which allelic observations are present in the data at known polymorphic positions. Known polymorphic positions are available from large human genome sequencing projects (e.g., the 1000 Genomes project).

3. Filter the allelic observations to remove sites that are likely in error because they are nearby a known, high-frequency insertion or deletion polymorphism. Mis-alignment between DNA sequence reads and a reference genome can lead to mis-calling of allelic observations. This problem is exacerbated when there is an insertion or deletion difference between the reference genome and the DNA sequence that is being aligned. To mitigate this problem, regions of known, high-frequency insertion or deletion polymorphisms are removed from the list of observations as they may be called incorrectly.

4. Use haplotype frequencies between each target site and each linked site observed from the input panel (1000 Genomes, for example) to determine the likelihood of the observed alleles under each possible target site genotype. The degree of information from each linked site is related to the linkage disequilibrium between it and the target site, and to how many allelic observations were made at the linked site. Additionally, allelic observations at the target site itself can be handled in this framework by considering the target site as perfectly linked to itself. The algorithmic details of this procedure are as follows:

a) Generate a list of genomic sites/markers for the assay. This list of genomic sites may be referred to as a target list. A target list typically derives from markers present on ANCESTRYDNA, 23ANDME, FAMILTYTREE DNA or other direct-to-consumer array platforms.

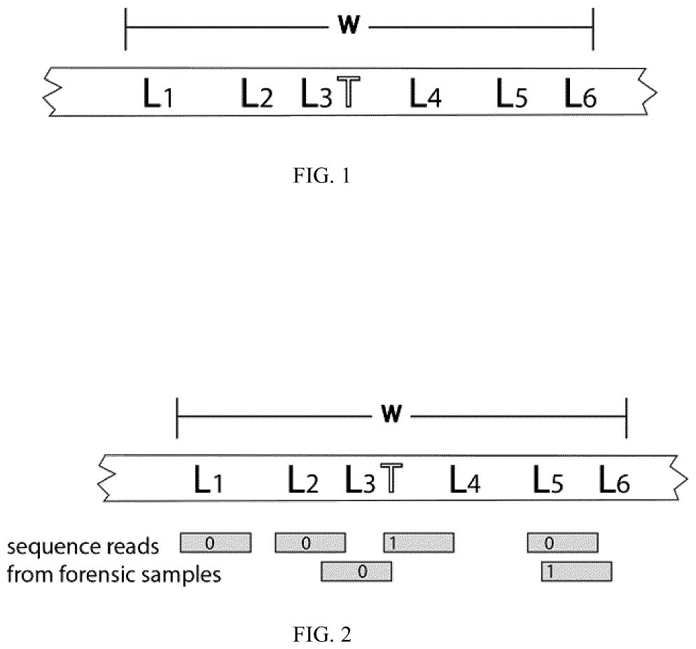

b) Generate a table of linked sites (L=L1, L2, . . . Ln) for each target site. These are sites nearby one or more target sites (see, e.g., FIG. 1). In this Example, the window for linked sites is 10 kb upstream and 10 kb downstream for each target site. The number of linked sites for each target site varies (e.g., tens to hundreds of linked sites per target site). The list of linked sites may be generated based on any large panel of human genome variation (e.g., the 1000 Genomes Project data as used in this Example).

The table of target sites and linked sites include haplotype frequencies of each linked site allele and the target site allele(s) to which it is linked. The format of this table in the current implementation is a tab-delimited file with the following columns:
1. Chr of target site
2. position of target site
3. ID of target site (usually an rsID)
4. Chr of linked site
5. Position of linked site
6. ID of linked site (usually an rsID)
7. Reference allele at linked site (expressed as a particular nucleotide base)
8. Alternative allele at linked site (expressed as a particular nucleotide base)
9. T0L0 frequency (haplotype frequency of target=ref, linked=ref)
10. T0L1 frequency (haplotype frequency of target=ref, linked=alt)
11. T1 L0 frequency (haplotype frequency of target=alt, linked=ref)
12. T1 L1 frequency (haplotype frequency of target=alt, linked=alt)

The above table essentially is a fixed table that is input to a genotype inference algorithm. That is, it generally need only be generated once.

In the above table, 0 refers to the reference allele and 1 refers to the alternative allele. Haplotype frequencies can be used to measure the amount of linkage disequilibrium and generally refer to counts of pairs of alleles (i.e., haplotypes) in a population. Haplotype frequencies may be referred to as haplotype counts. The haplotype frequencies in columns 9-12 are obtained from a database (e.g., any database containing haplotype frequency data). In this Example, the haplotype frequencies in columns 9-12 are obtained from the 1000 Genomes Project public database.

A target site is considered a site that is linked to itself. In this implementation, there are only T0L0 and T1 L1 haplotype counts, and columns 10 and 11 necessarily have value 0. In this way, each target site is perfectly linked to itself. As described below, this allows use of the allelic observations at target sites in the same mathematical framework as allelic observations at linked sites. In certain implementations, a blacklist is used to remove target and linked sites. A blacklist of regions may be constructed around known insertion or deletion polymorphisms. These sites sometimes generate incorrect alignments and lead to incorrect allele calls at target or linked sites.

c) Map sequence data to a reference genome to generate allelic observations at target sites and linked sites (see, e.g., FIG. 2). In this Example, the BWA-MEM aligner is used, which can allow multiple mismatches, depending on the length of the sequence being aligned. In certain implementations, sequences are filtered to exclude those whose alignment score is below a certain threshold. In certain implementations, the quality score of each base is used to exclude those that are more likely to be errors, and certain implementations incorporate a fixed background of base-calling error. The allelic observations at target sites and linked sites are added to the haplotype table generating the following new columns at each Linked site line.
13. Counts of L0 observations, i.e., reads carrying the reference allele
14. Counts of L1 observations, i.e., reads carrying the alternative allele

The counts in columns 13 and 14 are observed counts of alleles in a test sample (e.g., a forensic sample).

d) At this point, all of the required information is now in the table. Use the information (i.e., columns 13 and 14) from the allelic observations at the target site and each linked site along with the linkage information (i.e., columns 9-12) from the panel to compute the likelihood of each possible target site genotype. There are three possible unphased genotypes (phased genotype data is not needed for this genotyping method) for each bi-allelic target site:
T00=homozygous for the reference allele
T01=heterozygous
T11=homozygous for the alternative allele

The likelihood for each unphased genotype at each target site is computed using a Bayesian approach. The probability of the data under each target site genotype model is determined. Then, that probability is multiplied by the prior probability of each genotype using the target site genotype frequencies and the Hardy-Weinberg assumption of genotype frequencies given allele frequencies. The Hardy-Weinberg assumption is given allele frequencies of p and q=1−p for a bi-allelic site, the probability of each possible genotype is: homozygous for p=p*p; heterozygous for p, q=2*p*q; homozygous for q=q*q. These values are used as prior probabilities in the Bayesian formulas below (i.e., the term in parenthesis that is squared; the bit in the parenthesis is a way of measuring p for the reference allele). The entire term in parenthesis yields a particular allele frequency of the target site reference allele (in equation (1)) or target site alternative allele (in equation (2)). For example, in equation (1) below, T0Lᵢ0+T0Lᵢ1 is divided by all four haplotype frequencies (T0Lᵢ0, T0Lᵢ1, T1Lᵢ0 and T1Lᵢ1). In other words, the number of haplotypes that carry T0—the reference allele at the target site—is divided by all the haplotypes. That provides the frequency of the reference allele at the target site.

The likelihood L of genotype 00 at target site Tis calculated as follows:

$$L ( T 00 ) = {P ( D \vert T 00 )}^{A} \times {P ( T 00 )}^{B} = {{\prod }_{{L}_{i}} ( \begin{matrix}{L}_{i} 0 + {L}_{i} 1 \\ {L}_{i} 0\end{matrix} )}_{C} \times {{PL}_{i} {0}^{{L}_{i} 0}}_{D} \times {{( 1 - {PL}_{i} 0 )}^{{L}_{i} 1}}_{E} \times {{( \frac{T 0 {L}_{i} 0 + T 0 {L}_{i} 1}{T 0 {L}_{i} 0 + T 0 {L}_{i} 1 + T 1 {L}_{i} 0 + T 1 {L}_{i} 1} )}^{2}}_{F} \qquad ( 1 )$$

### Where:

Operation A is the probability of the observed data (D) given the genotype at target site T is 00.
Operation B is the probability that the genotype at the target site T is 00 (also referred to as the “prior” for genotype 00 at the target site).
The product of operations C, D, and E are equal to operation A. Operations C, D, and E are a way to calculate operation A given the observations at the linked sites and the haplotype panel data. More specifically, operations C, D, and E are the binomial sampling probabilities for the observations at each linked site L; given the haplotype information. The product term refers to each site evaluated independently and then all sites are multiplied together.
Operation F is the term used to calculate operation B. Given the haplotype counts in the table, this is a way to learn the frequency of homozygous reference at the target site. The term in parenthesis calculates the frequency of the 0 allele at site T. Taking the square of that gives the frequency of homozygotes in the population, under the Hardy-Weinberg assumption.
L(T00) is the likelihood of genotype 00 at target site T.
D refers to the data, i.e., the observed alleles in the last two columns (columns 13 and 14).
Lᵢ0 is the count of reference alleles observed at linked site Lᵢ.
Lᵢ1 is the count of alternative alleles observed at linked site Lᵢ.
PLᵢ0 is the probability of observing the reference allele at linked site Lᵢ, given allele TO. The probability is determined from haplotype frequencies. This probability can be adjusted to account for sequencing error (e.g., using a fixed background rate of sequencing error).
T0ᵢL0, T0Lᵢ1, T1Lᵢ0 and T1Lᵢ1 are the haplotype frequencies listed under columns 9-12, respectively.

The likelihoods for the two other genotypes, T01 and T11, are calculated similarly. T01 is calculated under the model that observed alleles are equally likely to derive from either chromosome.

The likelihood L of genotype 11 at target site Tis calculated as follows:

$$L ( T 11 ) = {P ( D \vert T 11 )}^{G} \times {P ( T 11 )}^{H} = {{\prod }_{{L}_{i}} ( \begin{matrix}{L}_{i} 0 + {L}_{i} 1 \\ {L}_{i} 0\end{matrix} )}_{I} \times {{PL}_{i} {0}^{{L}_{i} 0}}_{J} \times {{( 1 - {PL}_{i} 0 )}^{{L}_{i} 1}}_{K} \times {{( \frac{T 1 {L}_{i} 0 + T 1 {L}_{i} 1}{T 0 {L}_{i} 0 + T 0 {L}_{i} 1 + T 1 {L}_{i} 0 + T 1 {L}_{i} 1} )}^{2}}_{L} \qquad ( 2 )$$

### Where:

Operation G is the probability of the observed data (D) given the genotype at target site Tis 11.
Operation H is the probability that the genotype at the target site T is 11 (also referred to as the “prior” for genotype 11 at the target site).
The product of operations I, J, and K are equal to operation G. Operations I, J, and K are a way to calculate operation G given the observations at the linked sites and the haplotype panel data. More specifically, operations I, J, and K are the binomial sampling probabilities for the observations at each linked site L; given the haplotype information. The product term refers to each site evaluated independently and then all sites are multiplied together.
Operation L is the term used to calculate operation H. Given the haplotype counts in the table, this is a way to learn the frequency of homozygous alternative at the target site. The term in parenthesis calculates the frequency of the 1 allele at site T. Taking the square of that gives the frequency of homozygotes in the population, under the Hardy-Weinberg assumption.
L(T11) is the likelihood of genotype 11 at target site T.
D refers to the data, i.e., the observed alleles in the last two columns (columns 13 and 14).
Lᵢ0 is the count of reference alleles observed at linked site Lᵢ.
Lᵢ1 is the count of alternative alleles observed at linked site Lᵢ.
PLᵢ0 is the probability of observing the reference allele at linked site Lᵢ, given allele T1. The probability is determined from haplotype frequencies. This probability can be adjusted to account for sequencing error (e.g., using a fixed background rate of sequencing error).
T0ᵢL0, T0Lᵢ1, T1Lᵢ0 and T1Lᵢ1 are the haplotype frequencies listed under columns 9-12, respectively.

Finally, the likelihood L that the target site T is heterozygous (01) for the reference and alternative alleles is calculated according to a process derived from equation (1) and equation (2), and by assuming each observed allele has probability of 0.5 of coming from either parental genome copy. Thus:

$$L ( T 01 ) = P ( D \vert T 01 ) \times P ( T 01 ) = {\prod }_{} {L}_{i} ( \begin{matrix}{L}_{i} 0 + {L}_{i} 1 \\ {L}_{i} 0\end{matrix} ) \times {PL}_{i} {0}^{{L}_{i} 0} \times {( 1 - {PL}_{i} 0 )}^{{L}_{i} 0} \times ( 2 \times ( \frac{T 0 {L}_{i} 0 + T 0 {L}_{i} 1}{T 0 {L}_{i} 0 + T 0 {L}_{i} 1 + T 1 {L}_{i} 0 + T 1 {L}_{i} 1} ) \times ( \frac{T 1 {L}_{i} 0 + T 1 {L}_{i} 1}{T 0 {L}_{i} 0 + T 0 {L}_{i} 1 + T 1 {L}_{i} 0 + T 1 {L}_{i} 1} ) \qquad ( 3 )$$

where:

$${PL}_{i} 0 = ( 0.5 \times \frac{T 0 {L}_{i} 0}{T 0 {L}_{i} 0 + T 0 {L}_{i} 1} ) + ( 0.5 \times \frac{T 1 {L}_{i} 0}{T 1 {L}_{i} 0 + T 1 {L}_{i} 1} )$$

In equation (3), the PLᵢ0 terms are calculated by assuming each observation at the linked site has equal probability of coming from either of the two haplotypes since the target site is heterozygous (half the DNA must be maternally derived and half must be paternally derived). The term at the end of equation 3 (starting with (2× . . . )) is the Hardy-Weinberg term for probability of being a heterozygote given an allele frequency (i.e., 2*p*q).

e) Determine the genotype at each target site by choosing the most likely genotype as calculated above. The likelihood of the most likely genotype is compared to the likelihood of the second most likely genotype to generate a likelihood ratio. Genotype calls can be filtered on this ratio, calling only genotypes with an arbitrarily high likelihood ratio. When this likelihood ratio is below the cutoff, it is still possible to call one of the alleles to output a partial genotype.

5. Because each DNA sequence read is independent data from other sequence reads, genotype likelihoods calculated from data at each linked site are treated as independent observations. A composite likelihood is generated by multiplying all linked site likelihoods for each target site genotype. Optionally, one can filter for a level of linkage disequilibrium and observed coverage. Comparison of the composite likelihoods of each target site genotype (homozygous reference, heterozygous, and homozygous alternative) yields a genotype likelihood ratio. This number can be used to filter results at any desired confidence level.

6. Convert genotypes into a file format suitable for downstream analysis (e.g., upload to an open genetic genealogy service).

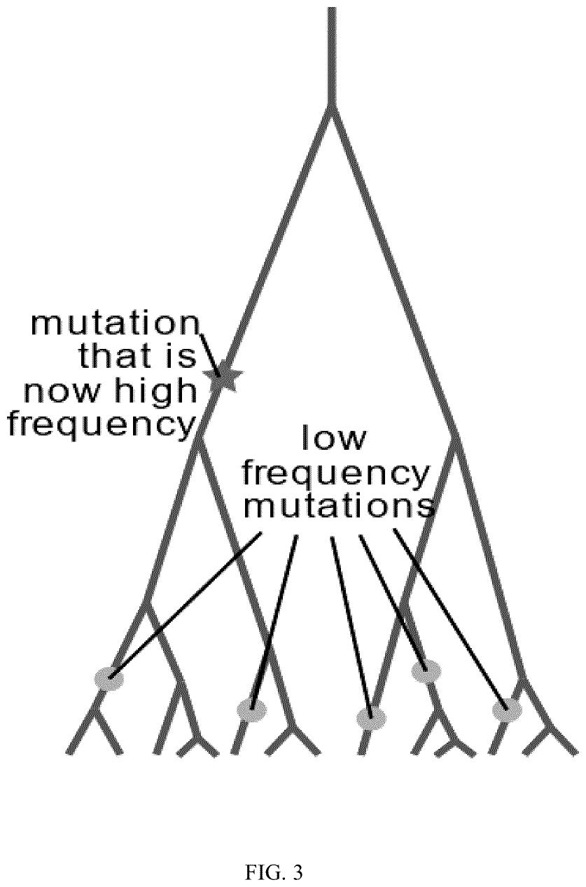

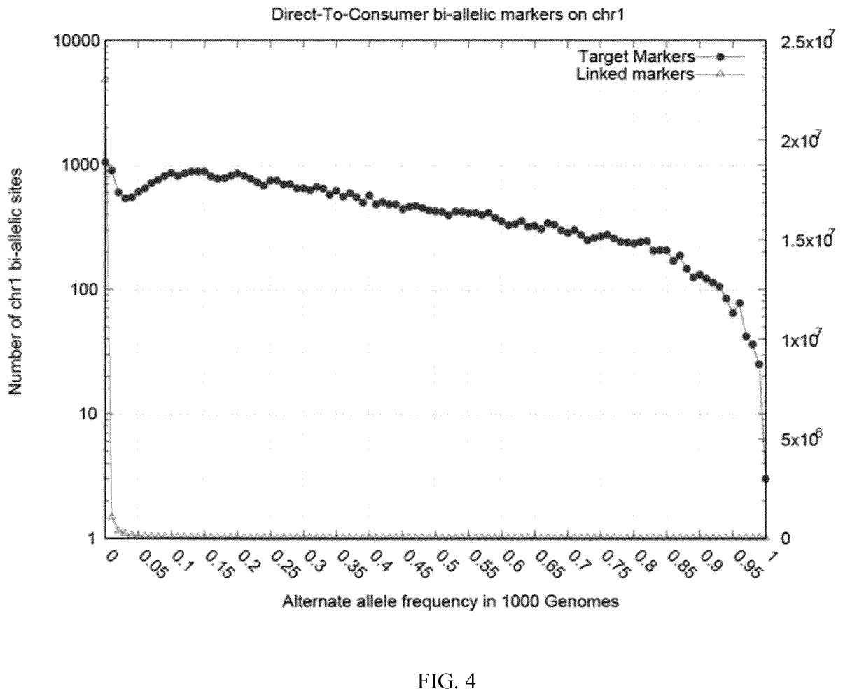

This framework of using low-coverage sequence data (about 2-fold genome coverage) at target sites and linked sites works well for certain conditions of interest. If the target site is a position of high minor-allele frequency, it is expected that the mutation generating the minor allele is old. Therefore, the mutation sits higher in the phylogenetic tree than a mutation of lower population frequency. Random genome data generates information at many sites of lower allele frequency due to the distribution of allele frequencies in humans, i.e., most genetic variation is rare. There is generally more information from rare alleles about nearby high-frequency alleles than the other way around. This is illustrated in FIG. 3, which shows haplotypes at any genomic region exist within the context of a phylogenetic tree, although the topology of the tree may not be known. Target sites (e.g., from direct to consumer arrays) typically have high allele frequencies and are thus older in time and deeper in the tree. Most genetic variation exists as lower frequency alleles and thus falls toward the bottom of the tree. As shown in FIG. 3, it is often possible to determine which upper branch of the tree a haplotype sits on given some information about lower branches. The opposite is not true. Thus, target site genotypes often can be ascertained from one or more low-frequency linked sites, but generally not the opposite. Since genotypes are determined at target sites that were selected for direct to consumer arrays, i.e., mostly high minor-allele-frequency sites, using data from large panels of more rare alleles can generate accurate genotypes.

### Results

Nucleic acid from a forensic test sample was converted to single-stranded DNA and a sequencing library was generated using scaffold adapters described herein. Briefly, nucleic acid extracted from a forensic test sample was incubated with a solution containing SSB at 95° C. for 5 minutes and shock cooled on ice for 2 minutes. Scaffold adapters compatible with Illumina-brand sequencers were added directly to the nucleic acid-SSB mixture, followed by the addition of a master mix containing T4 polynucleotide kinase, T4 DNA ligase, T4 DNA ligase buffer and PEG 8000. The mixture was incubated at 37° C. for 1 hour. The ligation product was purified using SPRI breads to remove excess adapters and short unligated product. The purified ligation product was amplified and multiplexing barcodes referred to as indexes were added in a PCR reaction. The PCR product was purified using 1.2×18% SPRI beads. The purified PCR product represented the completed sequencing library, which was sequenced on an Illumina sequencing platform.

A genotype calling strategy was implemented using the genotyping method described above. Specifically, the genotyping strategy described in this Example was used to determine genotypes at sites on a direct-to-consumer platform using low-coverage (i.e., about 2 fold) shotgun sequencing data. Comparison to the genotype array data as downloaded from the direct-to-consumer platform (from the same sample source) was done to determine the overall level of genotype concordance. Analysis was restricted to bi-allelic sites. Target sites less than 4 base-pairs from a known insertion or deletion variant were removed due to difficulties with alignment with low-coverage data around insertions or deletions. Typically, direct to consumer arrays genotype about 700,000 target sites. The approach described in this Example, using suitable cutoffs, calls about 92% of the target sites that are bi-allelic SNPs. Typically, all or most of the target site genotype calls generated by the approach described in this Example are needed for a positive identification of a subject from low coverage sequencing data generated for nucleic acid in a test sample (e.g., a forensic sample originating from the subject).

Genotyping method calls (i.e., results generated by the genotyping method described in this Example) were compared to results generated from an existing genotyping method (i.e., IMPUTE2), and the results are shown in Table 1 below. The comparison showed the genotyping approach described in this Example has a higher concordance with genotype array data from the same donor vs. the existing genotyping approach.

TABLE 1

| Genotype array | Imputation software | Genotyping method | IMPUTE2-based |
|---|---|---|---|
| genotype | genotype | calls | method calls |
| Homozygous | Homozygous (correct allele) | 349,113 | 340,748 |
| site | Homozygous (incorrect allele) | 1,577 | 2,604 |
| | Heterozygous | 16,923 | 15,513 |
| | 1 allele called (correct) | 51,809 | 100,397 |
| | 1 allele called (incorrect) | 1,571 | 1,111 |
| Heterozygous | Heterozygous | 155,188 | 153,888 |
| site | Homozygous | 7,343 | 3,542 |
| | 1 allele called (correctly) | 27,347 | 29,937 |
| | 1 allele called (incorrectly) | 0 | 47 |

### Example 2: An Alternative Approach for Genotype Calling from Low-Coverage Sequence Data

The method described in Example 1 uses information from sites that are in linkage disequilibrium with a target site to improve genotype calling. That method considers observed data at each linked site as an independent measure of alleles present at a target site. An alternative approach described in this Example uses haplotypes—sections of linked alleles—around a target site. Large panels of haplotypes are available from 1000 Genomes Project and other sources. In this approach, instead of considering the data at each linked site separately, haplotypes are considered in aggregate to generate target site genotype calls.

Below is an example workflow for this method.

1. Around each target site, haplotype sets are generated using observed haplotypes from an external reference panel such as 1000 Genomes. Each target site is described by a set of haplotypes. The sites used to describe haplotypes can be:
a) selected as commonly present in DNA recovered from certain types of samples (e.g., hair);
b) selected for high linkage disequilibrium with the target site;
c) selected for suitable mapping characteristics (e.g., avoiding repetitive regions, insertion-deletion polymorphisms, and other genome features that disrupt accurate mapping); and/or
d) spaced such that a single read does not give information about multiple sites thus avoiding over counting information from single reads.

2. DNA sequence reads from a sample (e.g., hair) are mapped to the reference human genome.

3. For each target site, it is assumed the sample is contributed from a single, diploid individual. Thus, the haplotype pair that is most probable is found given the observed bases at each site, for each haplotype around each target.

The probability of a pair of haplotypes is calculated using a Bayesian approach. The probability of each haplotype pair, given the data is the probability of the data, given each haplotype pair times the probability of the haplotype pair. The probability of the haplotype pair can be learned from the haplotype frequencies in the reference panel.

Thus, given a set of observed haplotypes:

$${H}_{i} = [ 0 \vert 1 , 0 \vert 1 , … ]$$

Where alleles are encoded by 0=reference allele and 1=alternative allele and Hᵢₛ is the s^{th} allele on the i^{th} haplotype,
The probability that the sample derives from any particular pair of haplotypes, i and j, given observed data, D, can be derived:

$$P ( {H}_{i} , {H}_{j} \vert D ) = \frac{P ( D \vert {H}_{i} , {H}_{j} ) \times P ( {H}_{i} , {H}_{j} )}{P ( D )} \qquad ( A )$$

P(Hᵢ, Hⱼ) is observed from the reference panel. That is, it is the probability of this particular haplotype pair in the panel, independent of any data.

P(D|Hᵢ, Hⱼ) is the probability of the observed data, given that it derives from this particular haplotype pair. This term can be calculated by considering what genotype should be observed at each site, s, across the region, given that these two haplotypes are present. The probability of the data, D, is calculated at each site and the product is taken across all sites, thusly:

$$P ( D \vert {H}_{i} , {H}_{j} ) = {\prod }_{s = 1}^{n} P ( {D}_{s} \vert {H}_{is} , {H}_{js} ) \qquad ( B )$$

Below is an additional example workflow for this method.

Given a set of observed haplotypes, as shown below:

TARGET 60523 T

| ## | TARGET 60523 | T | G | rs112920234 |
|---|---|---|---|---|
| 10 | 60523 | T | G | rs112920234 |
| 10 | 60753 | C | T | rs554788161 |
| 10 | 60803 | T | G | rs536478188 |
| 10 | 60969 | C | A | rs61838556 |
| 10 | 61020 | G | C | rs115033199 |
| 10 | 61331 | A | G | rs548639866 |
| 10 | 61386 | G | A | rs536439816 |
| 10 | 61614 | G | C | rs554243250 |
| 10 | 61694 | T | G | rs546443136 |
| 10 | 61836 | G | T | rs563066628 |
| 10 | 62010 | C | T | rs568474105 |
| 10 | 62068 | A | C | rs538488924 |
| 10 | 62450 | G | A | rs539912981 |
| 10 | 62554 | T | C | rs577354278 |

| ## | Haplotypes |
|---|---|
| 2394 | 00010000000000 |
| 2346 | 00000000000000 |
| 72 | 10000000001000 |
| 63 | 00000100000000 |
| 49 | 00100010100000 |
| 25 | 00011000000000 |
| 18 | 10000000000000 |
| 14 | 00000001000000 |
| 8 | 00000000000100 |
| 4 | 00010000000010 |
| 3 | 00010000000100 |
| 3 | 00000000000001 |
| 2 | 00000000010000 |
| 2 | 00010010100000 |
| 1 | 11000000001000 |
| 1 | 00001000000000 |
| 1 | 01000000000000 |

where the columns in the haplotype matrix are the sites, as listed in the order above the haplotypes and the genotype of a site at a haplotype is encoded as 0=reference and 1=alternative allele.

For each target site (genomic position for which a genotype is deduced), a haplotype description is generated that describes the linked genomic variants in a population. A haplotype description can be from 1000 Genomes or any large panel of known genome sequences. The sites that go into the haplotype description can be chosen such that they are in linkage disequilibrium with the target site and are generally recoverable from a particular type of test sample (e.g., hair DNA). The haplotypes can be described as a matrix where the rows are unique haplotypes and the columns are the genomic positions in the haplotypes. Each matrix element describes the allele of a particular site on a particular haplotype (see Table 2 below, where 0 refers to a reference allele and 1 refers to an alternative allele).

TABLE 2

| | Genomic | Genomic | Genomic | Genomic | Genomic |
|---|---|---|---|---|---|
| | position i | position ii | position iii | position iv | position v |
| Haplotype A | 0 | 0 | 0 | 0 | 0 |
| Haplotype B | 1 | 0 | 0 | 0 | 0 |
| Haplotype C | 1 | 1 | 0 | 0 | 0 |
| Haplotype D | 1 | 1 | 1 | 0 | 0 |
| Haplotype E | 1 | 1 | 1 | 1 | 0 |
| Haplotype F | 1 | 1 | 1 | 1 | 1 |
| Etc. | | | | | |

For a given sample (e.g., from a hair) reads are aligned to a reference genome and collect allelic observations at linked sites in a haplotype around a target region.

For a given sample, for each pair of haplotypes in the haplotype matrix, the probability of the observed sequence data given that they derive from that pair of haplotypes is calculated. This can be done for all pairs of haplotypes in the matrix in an all versus all comparison; the pairs where both haplotypes are the same are also included in the comparison. For example, in Table 2, a haplotype pair may be any two haplotypes from A-F (e.g., haplotype pair AB, BC, AC, . . . or AA, BB, CC, . . . ). Humans are diploid species that have two copies of each chromosome region, i.e., haplotype. Thus, the data is derived from two haplotypes. One component of this method is to determine what the two haplotypes were.

For any combination of haplotypes, it can be deduced what the target genotype is. The target site genotype can be determined by finding the haplotype pair that most probably explains the observed sample data. Once that haplotype pair is found, the target site genotype is determined by taking the target site genotype from each of the haplotypes in this haplotype pair. For example, in Table 2, if the target site is at genomic position iv, and the most likely haplotype pair given the data is haplotype pair AE, then the genotype at the target site is 0,1.

A useful variation of this procedure is instead of finding a most probable pair of haplotypes for explaining the observed data, the probability across all haplotype pairs can be aggregated. Each haplotype pair corresponds to one of three possible target genotypes (homozygous reference, heterozygous, and homozygous alternative). The probability of the data given a haplotype pair, once calculated as above, is added to the aggregate probability of the corresponding target site genotype. The target site genotype is then selected as the one with the highest aggregate probability. In this procedure, instead of finding the “best two” haplotypes for explaining the data, a comprehensive census is taken across all haplotype combinations.

### Example 3: A Method for Deconvoluting and Imputing Genotypes from a Mixture of Two Samples

In this Example, sequence data may be abundant, however it is derived from the nucleic acid of two subjects (a mixed sample). In most instances, one of the subjects can be genotyped completely from a different sample. This may be a victim and perpetrator mixture, for example. In such instances, DNA from the victim is readily available and it is straightforward to learn his/her genotype completely from a pure victim sample. The perpetrator has some DNA in the mixed sample. Typically, for an unknown perpetrator, additional DNA and/or a pure DNA sample cannot be obtained from the perpetrator. Accordingly, it is difficult to accurately genotype the perpetrator DNA from this mixture. However, there are certain genetic sites that can be genotyped well. These sites can be used to then impute the genotype of the perpetrator at other sites, e.g., using certain methods described in Example 1.

The details in this Example describe how to find the sites that can be accurately genotyped. First, the mixture proportion of the mixed sample is learned (e.g., how much victim DNA is in the sample and how much perpetrator DNA is in the sample). This step includes a probabilistic model for learning the mixture proportion, as described below. Then, this Example describes how to obtain the unknown genotype (e.g., perpetrator's genotype), given the mixture proportion. The description points out that when the known subject (e.g., victim) is homozygous (has only one kind of allele) at a particular genetic site, then it may be possible to determine the genotype the unknown subject (e.g., perpetrator) at that site. For example, if an allele is observed in the mixture that differs from what the known subject (e.g., victim) is homozygous for, then the unknown subject (e.g., perpetrator) is not homozygous for the same allele as the known subject (e.g., victim). The remaining steps in this Example describe how linked data may be used to impute genotypes (similar to the process described in Example 1). Typically, the linked sites that can be obtained from a mixed sample is a subset of what could be obtained from a clean (non-mixed) sample of the unknown subject (e.g., perpetrator).

This Example describes a method for deconvoluting and imputing genotypes at common SNP sites from shot-gun sequencing of 1) a DNA mixture of two samples, and 2) a pure sample of DNA from one of the samples in the mixture. In certain instances, this procedure can be performed without using the pure sample. Deconvoluted and imputed genotypes can then be used to search a database of genotyping arrays.

Direct-to-consumer genomic testing has led to the creation of several databases of dense genotyping data from the general public. For each person in the database, the genotype data typically includes 1 million to 2 million single-nucleotide polymorphisms (SNPs), where each SNP genotype is a pair of the alleles A, C, G, and T. Typically, these arrays assay bi-allelic variants, meaning that only two of the possible four bases A, C, G, and T are observed in the population. Further, these are typically common variants, meaning that the less common of the two alleles appears in >1% of the human population, making them more informative for searching purposes than rare variants. The large number of informative SNPs means that a database of genotypes can be searched effectively with only a subset of matching data; not every SNP must be accurately genotyped in order to achieve a high degree of certainty about identity, parent/child, sibling, and cousin relationships between a proband genotype and hits in the database.

Many forensic samples yield mixtures of two different individuals, particularly in the case of violent attack. These samples typically cannot be genotyped by the SNP genotype arrays used for consumer genetics, as the signal at each SNP site is an intensity measure of each of the two alleles. The method described in this Example uses whole-genome shotgun sequencing of a mixture sample to impute the genotype at common variant sites, using a technique similar to the method for imputing genotypes from low-coverage low-quality DNA samples described in Example 1. Areas of application for this method include, for example, forensics, laboratory quality control (QC), detection and identification of chimeric individuals for research, clinical, or consumer genomics, and the like.

The analysis described in this Example includes the following workflow:
1. Estimate the mixture fraction (i.e., the proportion of DNA in the mixture derived from each sample—known and unknown samples)
2. Genotype a subset of sites for which the contaminant (unknown sample) has a strong likelihood of each assigned genotype being correct.
3. Intersect the unknown sample's strong likelihood genotypes with a database of rare population variants that are informative for common SNP sites
4. Impute the genotype of common sites in the unknown sample using the high quality genotypes of the rare variants.

For the above workflow, input data generally includes 1) sequence reads from a mixture of two samples (known and unknown) and 2) sequence reads and/or genotype data for the known sample. Variations of input data/workflow may include, for example, workflows with or without data for the known sample, data from targeted sequencing of rare variants, genotyping microarrays for the unknown sample, and various sequencing depths of the mixture sample (e.g., starting with ˜150× whole genome sequencing (WGS) coverage of the mixture).

In a pure, unmixed sample, genotyping WGS data involves identifying sites of potential variation, where the DNA sequence reads differ from the reference genome in a consistent pattern. At a known SNP site, the sequence from an individual read uniformly matches the reference genome (homozygous reference, or 0/0), is split between the reference allele and the alternative allele with an expected variant allele fraction of 50% (heterozygous, or 0/1), or uniformly matches the alternative allele (homozygous non-reference, or 1/1).

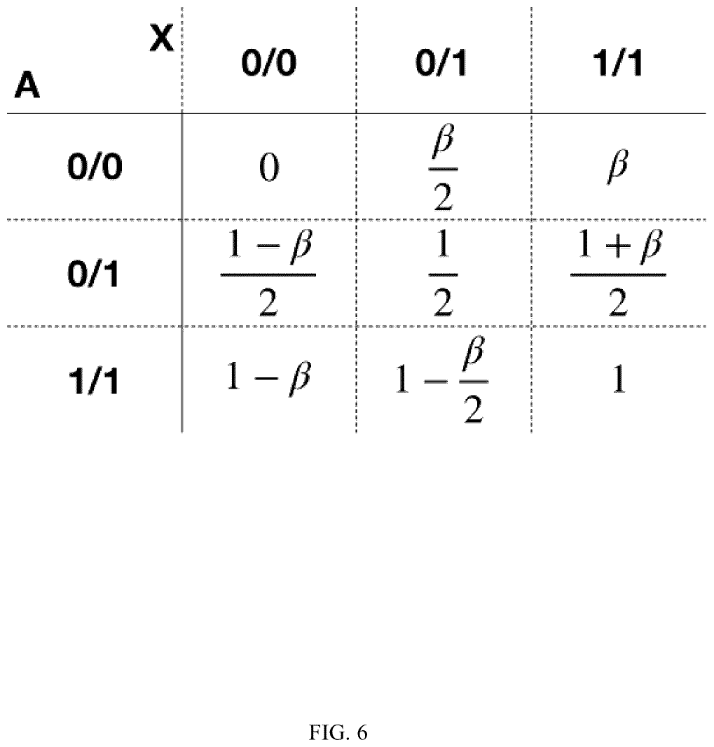

For a mixed sample, the unknown fraction, p, (i.e., the DNA fraction of the mixture from the unknown sample) influences the expected variant allele fraction (VAF) of the alternative allele, according to both the known sample's genotype and the unknown sample's genotype, as shown in FIG. 6 (A refers to the known sample, X refers to the unknown sample).

There are three modalities of the expected variant allele fraction (VAF) as β varies:
β less than 0.5: VAFs at SNP site resemble those of the known sample (A), though variability of the VAFs results in variant calling algorithms calling more heterozygosity due to the contamination of the unknown sample (X).
β close to 0.5: in the mixture, the expected VAFs matrix is close to symmetric, meaning that without the genotype of the known sample (A), an individual site's joint A/X genotype generally cannot be determined.
β greater than 0.5: the VAFs at SNP sites in the mixture mostly resembles those of a pure sample of the unknown sample (X). A traditional variant caller genotypes most sites as X's genotype, with increased heterozygous calls due to the contamination of the known sample (A).

An example workflow for the first modality (β less than 0.5) is described below.

### Step 1: Initial Estimation of β

SNPs with very low population allele frequencies (PAF), such as those below 5%, are very likely to have a genotype for X that is homozygous reference (0/0). Therefore, these SNPs with A=0/1 or A=1/1 can be used to estimate the value of p. For example, the value of p may be estimated according to a deviation or shift from the expected VAF value (e.g., a shift from a VAF of 0.5 for a heterozygous genotype (A=0/1); or a shift from a VAF of 1.0 for a homozygous genotype (A=1/1). A quantification of the deviation or shift represents the amount of DNA from X (unknown sample) in the mixed sample.

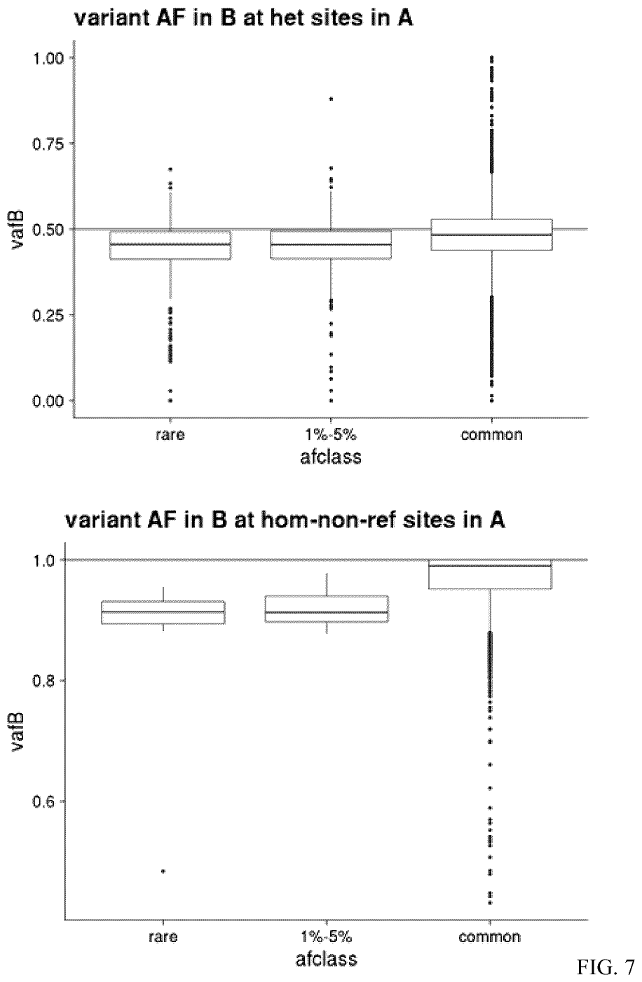

With the example data shown in FIG. 7, rare (PAF<1%) SNVs that are heterozygous in A provide an estimate of p=0.117 (top panel, FIG. 7), and rare SNVs at homozygous non-reference sites in A provide an estimate of p=0.108 (bottom panel, FIG. 7). This estimate of p may be used in a probability model to genotype X.

### Step 2: Genotype X

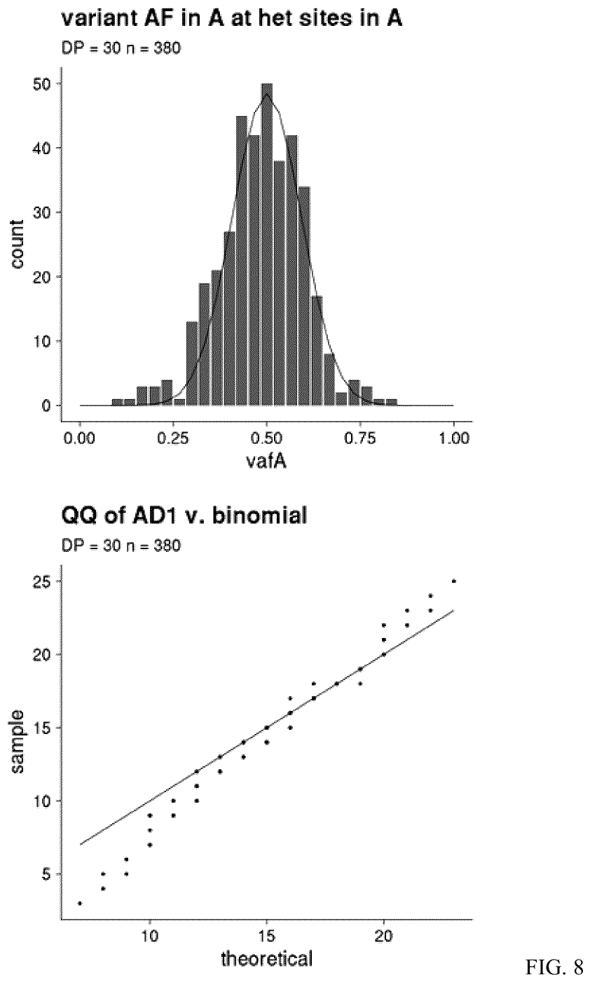

X may be genotyped (where possible) using a variety of methods. In general, a greater power to genotype X is present where A is homozygous. This is illustrated in the plots from the estimation of p (FIG. 7), where the VAF of SNPs with rare PAF shows clear separation from homozygous non-reference SNPs, but the VAF distributions overlap for A's heterozygous sites. Theoretically, a binomial distribution can be justified, and empirically this distribution fits somewhat as shown by a histogram and quantile-quantile plot (FIG. 8). For one sample's whole-genome sequence data, heterozygous sites were selected that have a sequencing read depth of exactly thirty reads. The VAF for each of these sites was calculated, and fits a binomial distribution

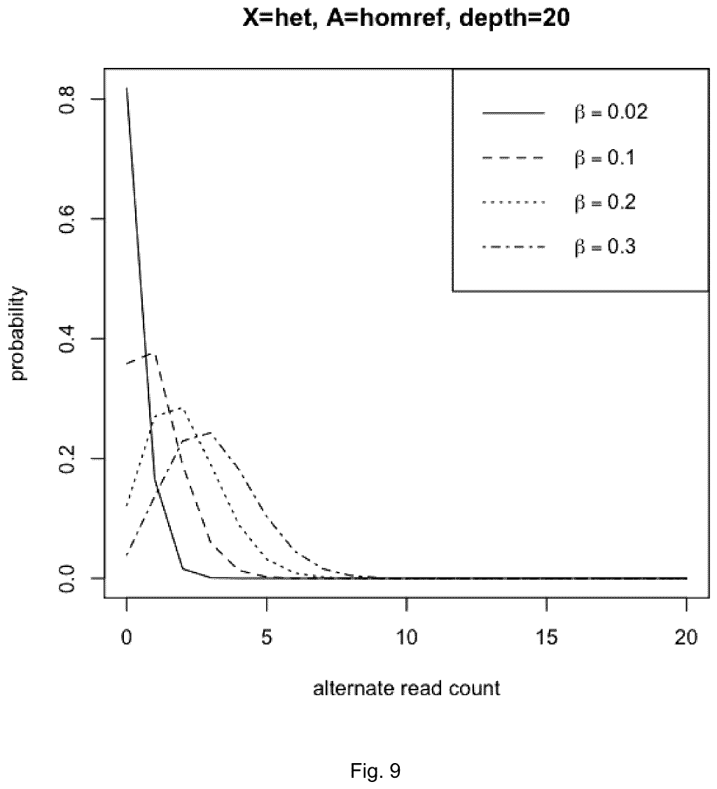

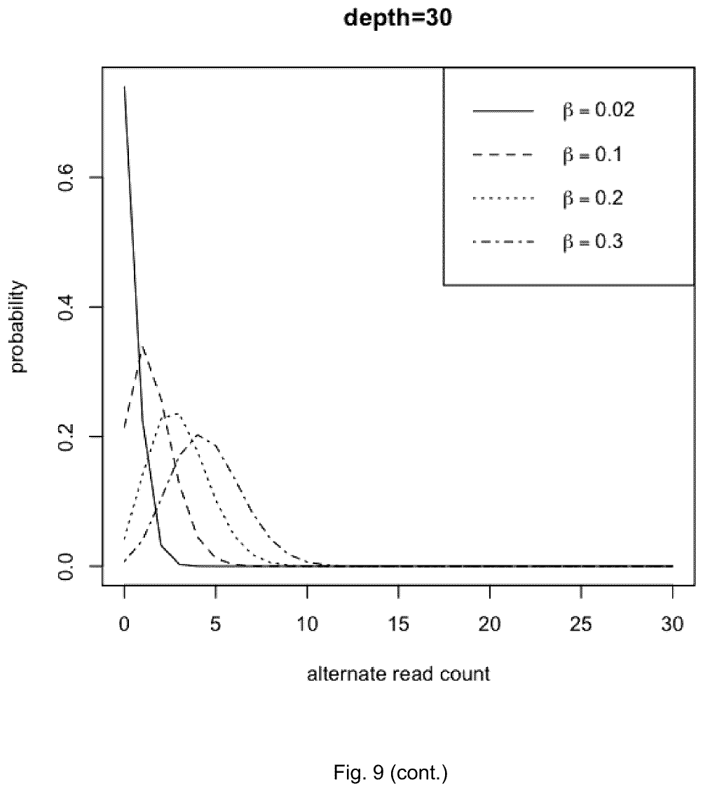

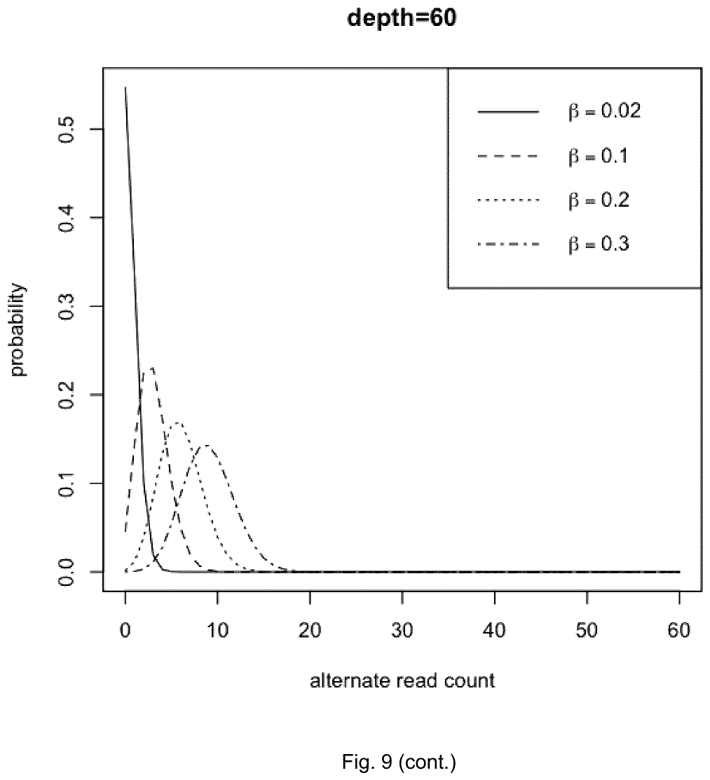

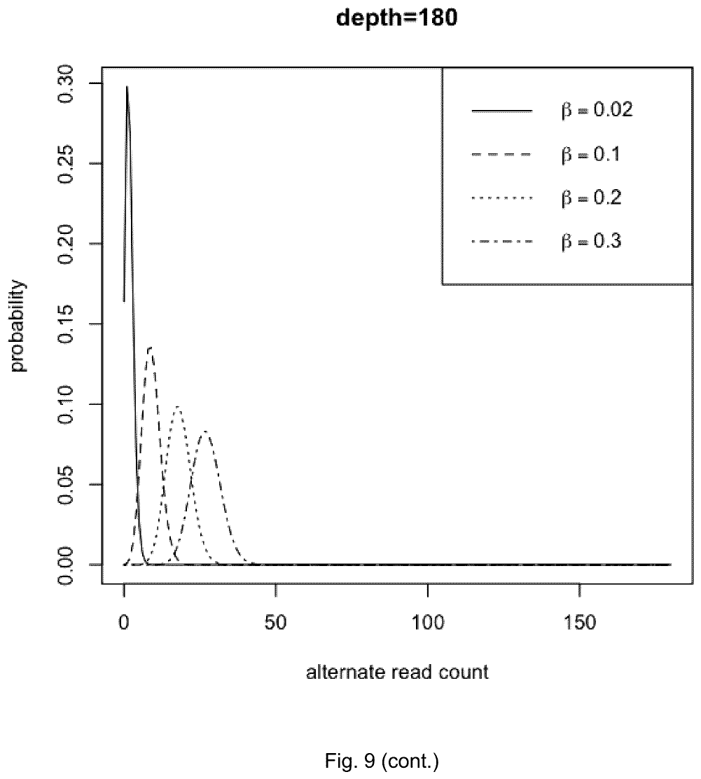

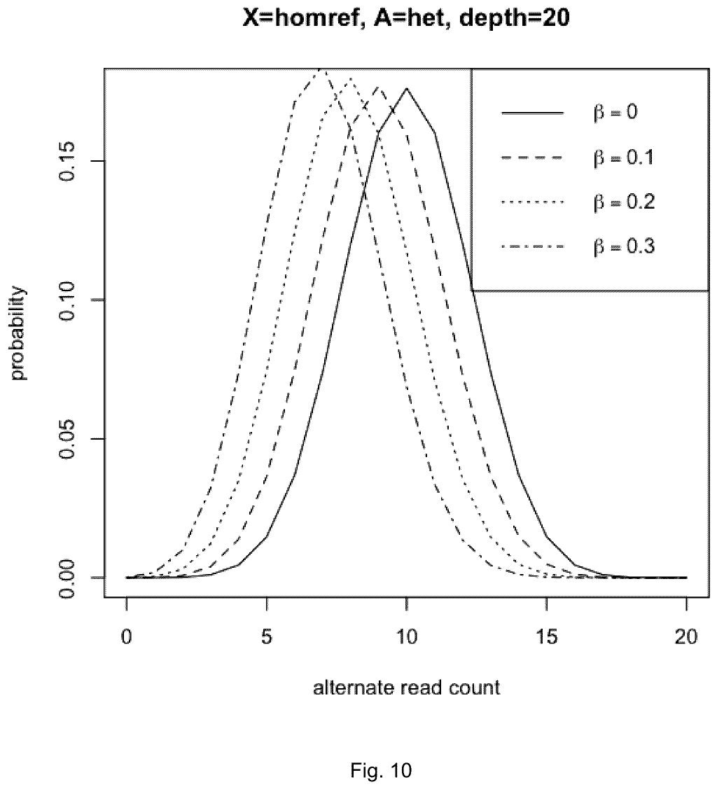

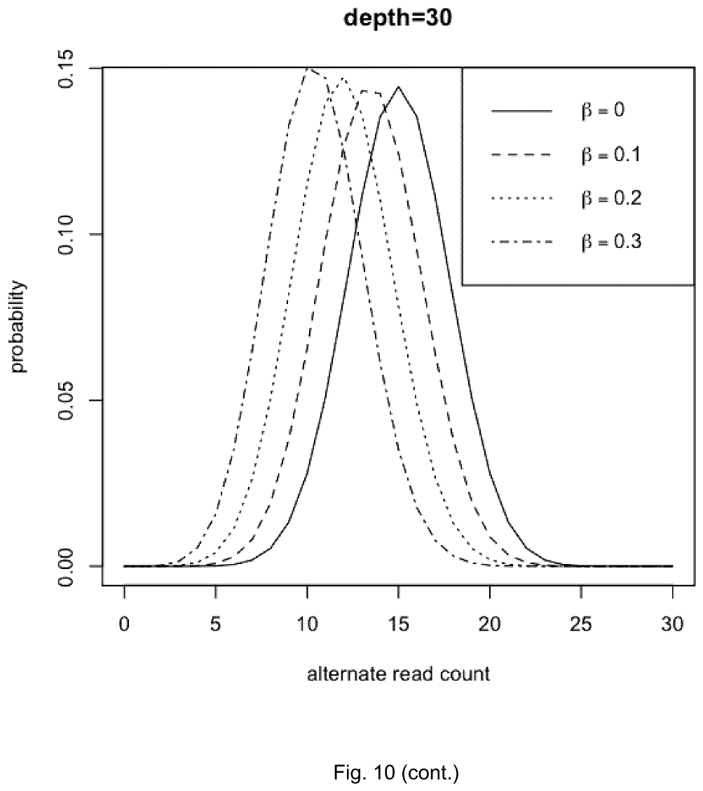

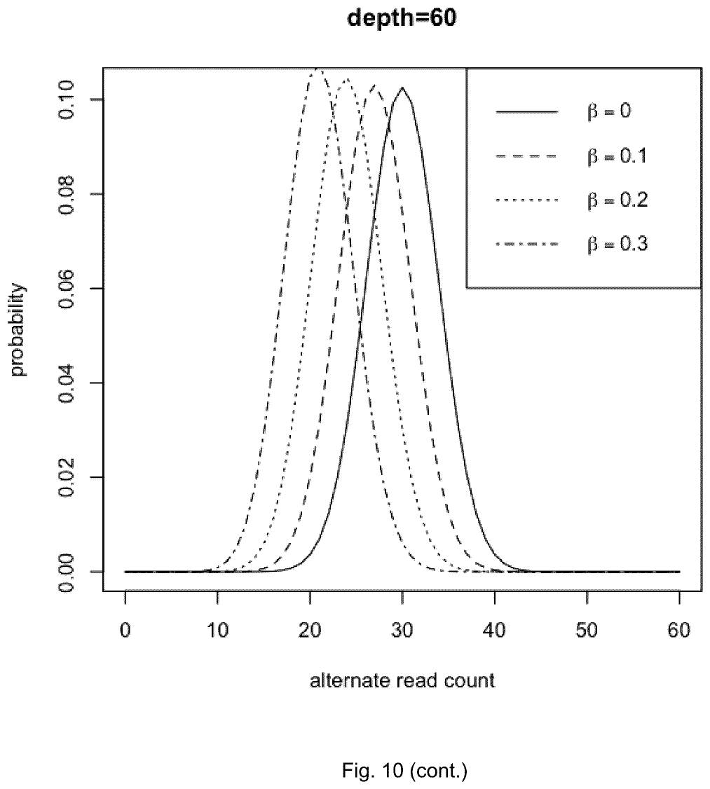

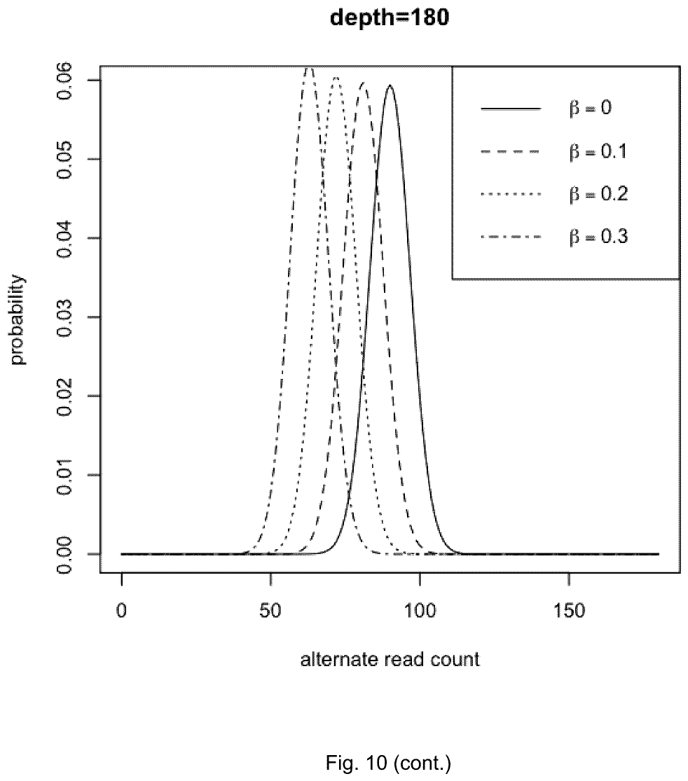

The variance of the binomial distribution is n(β)(1−R), where n is the number of reads observed, and is maximized for p=0.5. The power to detect the genotype of X in the mixture can be modeled by the separation of the binomial distributions along a row of FIG. 6. Considering the case of A=0/0, homozygous reference sites in A, FIG. 9 shows theoretical binomial distributions of read depths for various mixture percentages. The curve for p=0.02 is used as comparison for potential read error. As the curves separate from the p=0.02 curve, sites become “high likelihood” for the purposes of steps 2 and 3 above. Considering the case of A=0/1, heterozygous reference/alternative sites in A, FIG. 10 shows decreased separation at the same mixture percentages compared to FIG. 9.

Sites where A is homozygous generally have more power to detect X's genotype, but the likelihood of all genotypes can be calculated according to a standard probabilistic model (e.g., see the description of a genotype-mixture-inference-model provided in Example 4).

Variations of Step 2 of the workflow described in this Example may include one or more of the following:
include population allele frequency as part of the calling probability model
simultaneously solving with geographic population stratifications, to remove possible bias towards a population
show how this changes as β=0.5 and β>0.5
proportion of heterozygous to homozygous sites, to identify the number of individuals in a mixture
   Step 3: Intersect with Informative Rare Variants

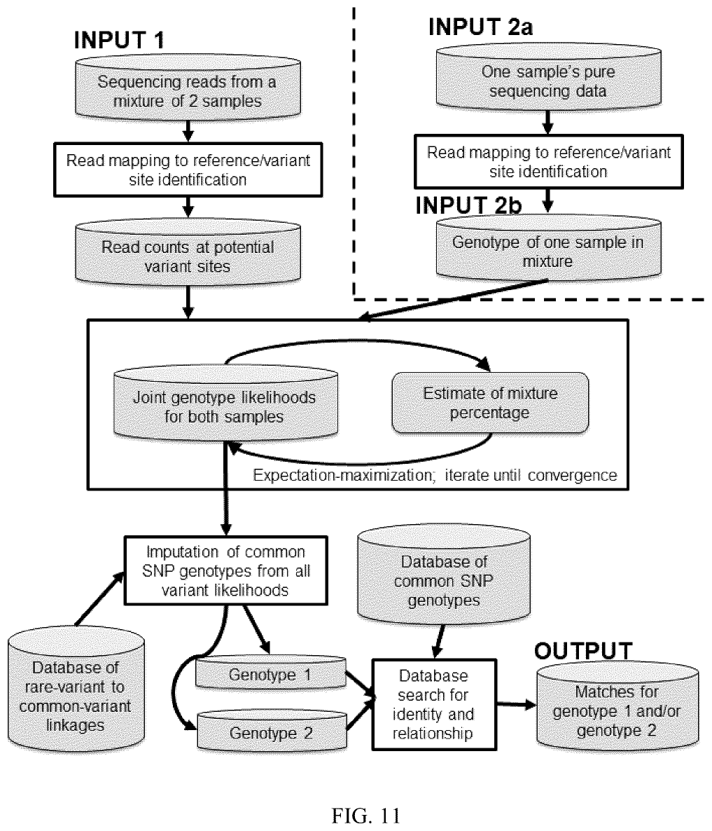

Since the distribution of population allele fraction includes primarily rare variants, most of the high quality genotypes in Step 2 are derived from rare variants. The list of high quality genotypes is intersected with a database that associates rare variants to genotypes of common sites (e.g., database of rare-variant to common-variant linkages (see FIG. 11); an intersection generally refers to a lookup in a database of linkages).

### Step 4: Impute Genotype of Common Sites

Genotypes of common sites in the unknown sample are imputed using high quality genotypes of the rare variants. Detailed explanations of this process are provided below (e.g., step 5d of the example workflow below, and in Example 4). Example workflow for mixture deconvolution

The purpose of the workflow summarized below is to infer genotypes at a set of target loci in the genome from a mixture sample, where only one component of the mixture is known. In this example θ instead of β is used for the mixture parameter. The outline of steps is:

1. Generate DNA sequence reads for the mixture sample using high coverage shotgun sequencing. More coverage may be needed from the mixture sample than would be required from a pure sample, such as 60 fold or 100 fold coverage, for example.

2. Align the mixture DNA sequence reads to a reference genome. For each site in database of known polymorphic positions (e.g., the 1000 Genomes Project), record the counts of reads supporting the reference allele and the alternative allele.

3. Using a pure sample of one of the components of the mixture, generate a genotype for that pure sample at each of the loci in the database of polymorphic positions (input 2A or 2B in FIG. 11).

4. Estimate the mixture percentage in the mixture sample. Repeat the following process from many different initial mixture percentage guesses, and choose the iteration that provides the best likelihood of the data:
a. Choose an initial guess for the mixture percentage, from a sampling of points between 0.0 and 1.0, and from initial guesses such as those based on the shift from expected counts as shown in FIG. 7.
b. At each locus where the known sample has a non-reference allele, count the reads in the mixture sample that support each allele. These counts are used to compute the numerical likelihood of the unknown sample's genotype, Lᵢ, at that locus i. This quantity is P(D|Lᵢ, θ), where D is the data, θ is the mixture percentage.
c. Using the likelihoods of the unknown sample genotype, Lᵢ, and the EM equations (provided in Example 4), calculate the new estimate of the mixture percentage, θ.
d. If this is the first time through this loop, or if the total likelihood has changed by more than a pre-set threshold (for example, 0.01%), go back to step b using the new mixture estimate.
e. Otherwise, the estimation process has completed. Save the likelihoods of the unknown sample, P(D|Lᵢ,θ) for use in imputation during Step 5.

5. Estimate a genotype for the unknown sample, using the genotype likelihoods from the previous step, and a database of known genotypes (e.g. 1000 Genomes Project). The steps below show one type method of imputation, as was used in Example 1. Other existing methods of genotype imputation or genotype refinement, such as Beagle or GLIMPSE, can also be used.
a. Generate a list of target polymorphic sites, for example the sites used on genotype arrays for direct-to-consumer companies such as ANCESTRYDNA, 23ANDME, and the like.
b. Using a database of polymorphic sites with haplotypes (e.g. 1000 Genomes Project), generate a table of linked polymorphic sites (L=L1, L2, . . . , Ln) for each target sites, with each linked site within a window of the target site. For each linked site, record the counts of the linked site haplotype with the target site haplotype, a two by two table.
c. Filter linked sites of any that may be in error due to a proximity to a known, high-frequency insertion or deletion polymorphism. Such sites are prone to cause errors in genotype likelihoods of the unknown sample.
d. Now the target site genotype may be imputed for the unknown sample, by finding which of T00, T01, and T11 maximizes the posterior probability of the genotype given the data, P(T|D). As with Example 1, the assumption is that all linked sites are independent in the observation. The genotype which maximizes the posterior probability is then also maximized by Π_{L}_{ᵢ}_{∈L}Σ_{L}_{ᵢ}P(T, Lᵢ)P(D|Lᵢ, θ). The first factor, P(T, Lᵢ) is estimated from the observed co-occurrence of the genotypes in the genotype database.

6. Convert the imputed genotypes of target sites into a file format suitable for downstream analysis, a text file representation of the target SNP sites that is formatted according to one of the direct-to-consumer file formats.

### Example 4: Genotype-Mixture-Inference-Model

This Example provides a detailed description of the process described in Step 2 of Example 3. In particular, this Example uses an expectation-maximization (EM) approach to learn the genotypes of the two contributors to a mixture. EM is a type of iterative algorithm that can find optimal parameters given a model that depends on these parameters and some data.

Here, the model is that the data come from two, diploid individuals who are mixed at some proportion. That proportion is also estimated by the algorithm. Then, it finds the model parameters (mixture proportion and genotypes) that best explain the observed data.

Most of the description of this method includes implementation details for how this particular problem can be cast as an EM problem.

### Mixture of Genotypes Setup

Inputs: a mixture sample that has been sequenced, along with the genotypes of one of the inputs to the sample. The sequencing data is represented as a set of biallelic diploid loci, indexed 1 . . . n, with the known sample's genotype, Y, being represented at each locus as Yᵢ∈{0,1,2}, the number of non-reference alleles. The sequencing data at locus i is a sequence of bases and their error probabilities, Xᵢ={(bᵢⱼ, eᵢⱼ)|j=1 . . . dᵢ}, where the total read depth is dᵢ at locus i, where Xᵢ refers to all the base calls and base call qualities at locus i, j is the read index, bᵢⱼ is the base call from read j, and, eᵢⱼ is the base call quality for that base call. Optionally, a population allele frequency of the alternative allele at locus i, P(Zᵢ), for both homozygous and heterozygous genotypes is included, which is the probability of observing genotype Zᵢ in the population.

Outputs: an estimate of the fraction of the unknown genotype, θ, is calculated as well as the posterior genotype probability for the unknown sample, P(Zᵢ|X, Y), where Zᵢ is unknown sample genotype, X is the mixture read data, and Y is the pure sample read data.

The unknown genotypes are imputed with the expectation-maximization (EM) algorithm. The latent variables Z have a distribution estimated in the E-step, using an initial estimate θ⁰, and θ are maximized during the M-step.

### Intuition on the Inference Problem

In a single sample's sequencing data, the expected read data for biallelic diploid variants is 100% reference, 50% reference, or 0% reference, depending on whether the sample is homozygous reference, heterozygous, or homozygous alternative, respectively. Base-calling or mapping errors result in deviations from these expected percentages, and for heterozygous sites, the variance in sampling results in a much higher variance in allele fraction than at homozygous sites.

In a single sample, the expectation is on the order of 3,000,000 sites are non-reference, approximately twice as many heterozygous sites as homozygous non-reference sites. A quality control step for single-sample sequencing to detect mixtures, or contamination, looks for samples with a heterozygous/homozygous ratio higher than 2.

In a mixture of two samples, sequencing data (whether whole genome or targeted), far more loci have VAFs at percentages other than 0%, 50%, or 100%. The ability to see this separation of loci is related to the product θD, the mixture percentage times the average depth of coverage (replacing θ with 1−θ when theta>0.5), as depth increases for constant θ.

### Assumptions

Each loci is independent, and the probability space factorizes similarly. In particular P(X, Z)=Πᵢ₌₁ⁿP(Xᵢ, Zᵢ) and P(Z)=Πᵢ₌₁ⁿP(Zᵢ), where X is the mixture read data, Z are all the genotypes of the unknown sample, i is a particular locus, n is the number of loci, Xᵢ is the mixture read data at locus i, and Zᵢ is the genotype of the unknown sample at locus i. Note that this ignores the linkage between sites, but is used for computational ease at this stage of the estimation of deconvolution. Later steps of the deconvolution exploit the dependence of Zᵢ. This can be viewed as a further haploblock latent variable, but the assumption is that the estimate of θ is fairly stable and not dependent on sharing haploblock information. However, as the value θD, this haploblock structure may allow pooling data from multiple loci to provide stronger inferences.

The errors at a given locus are independent of each other and of all other variables.

### Expectation Maximization

The likelihood of the data L is a function of θ:

$$\begin{matrix}L ( θ ; X , Y ) & = & log P ( X \vert θ , Y ) & ( 4 ) \\ & = & log {\sum }_{Z} P ( X , Z \vert θ , Y ) & ( 5 ) \\ & = & log {\sum }_{{Z}_{i}} {\prod }_{i = 1}^{n} P ( {X}_{i} , {Z}_{i} \vert θ , {Y}_{i} ) & ( 6 ) \\ & = & log {\prod }_{i = 1}^{n} {\sum }_{{Z}_{i}} P ( {X}_{i} , {Z}_{i} \vert θ , {Y}_{i} ) & ( 7 ) \\ & = & {\prod }_{i = 1}^{n} log {\sum }_{{Z}_{i}} P ( {X}_{i} , {Z}_{i} \vert θ , {Y}_{i} ) & ( 8 ) \\ & = & {\prod }_{i = 1}^{n} log {\sum }_{{Z}_{i}} P ( {X}_{i} , \vert {Z}_{i} , θ , {Y}_{i} ) P ( {Z}_{i} ) & ( 9 )\end{matrix}$$

where:
L is the likelihood function,
θ is the unknown mixture fraction,
X is the read data from the mixture sample,
Y is the genotype of the known sample,
P is the probability model function,
Z is the genotype of the unknown sample,
i is a locus index,
n is the number of loci,
Xᵢ is the mixture read data at locus i,
Zᵢ is the genotype of the unknown sample at locus i, and
Yᵢ is the genotype of the known sample at locus i.

For any probability distribution Qᵢ(Zᵢ), over each Zᵢ, over each Zᵢ, the unknown genotype at locus i,

$$\begin{matrix}L ( θ ; X , Y ) & = & {\prod }_{i = 1}^{n} log {\sum }_{{Z}_{i}} P ( {X}_{i} , \vert {Z}_{i} , θ , {Y}_{i} ) P ( {Z}_{i} ) & ( 10 ) \\ & = & {\prod }_{i = 1}^{n} log {\sum }_{{Z}_{i}} {Q}_{i} ( {Z}_{i} ) \frac{P ( {X}_{i} , \vert {Z}_{i} , θ , {Y}_{i} ) P ( {Z}_{i} )}{{Q}_{i} ( {Z}_{i} )} & ( 11 ) \\ & \geq & {\prod }_{i = 1}^{n} {\sum }_{{Z}_{i}} {Q}_{i} ( {Z}_{i} ) log \frac{P ( {X}_{i} , \vert {Z}_{i} , θ , {Y}_{i} ) P ( {Z}_{i} )}{{Q}_{i} ( {Z}_{i} )} & ( 12 )\end{matrix}$$

where:
L is the likelihood function,
θ is the unknown mixture fraction,
X is the read data from the mixture sample,
Y is the genotype of the known sample,
P is the probability model function,
i is a locus index,
n is the number of loci,
Xᵢ is the mixture read data at locus i,
Zᵢ is the genotype of the unknown sample at locus i,
Yᵢ is the genotype of the known sample at locus i, and
Qᵢ is the probability distribution at locus i.
   where Jensen's inequality is used, with f(X)=log x as the concave function, and Qᵢ(Zᵢ) as the probability distribution for the expectation of

$$\frac{P ( … )}{{Q}_{i} ( {Z}_{i} )} .$$

The prior inequality is only equal when the value

$$\frac{P ( … )}{{Q}_{i} ( {Z}_{i} )}$$

is a constant, which happens only if Qᵢ(Zᵢ) is proportional to P(Xᵢ|Yᵢ, Z_{I}, θ) over the values of Zᵢ. So given some guess θ^{t}, in the Expectation step the following is set:

$$\begin{matrix}{Q}_{i}^{t} ( {Z}_{i} ) & = & \frac{P ( {X}_{i} \vert {Y}_{i} , {Z}_{i} , {θ}^{t} ) P ( {Z}_{i} )}{{\sum }_{{Z}_{i}} P ( {X}_{i} \vert {Y}_{i} , {Z}_{i} , {θ}^{t} ) P ( {Z}_{i} )} & ( 13 ) \\ & = & P ( {Z}_{i} \vert {X}_{i} , {Y}_{i} , {θ}^{t} ) & ( 14 )\end{matrix}$$

where:
θ is the unknown mixture fraction,
P is the probability model function,
t is the iteration number,
i is a locus index,
Xᵢ is the mixture read data at locus i,
Zᵢ is the genotype of the unknown sample at locus i,
Yᵢ is the genotype of the known sample at locus i, and
Qᵢ^{t} is the expected probability distribution over the unknown genotype at locus i and iteration t, and then a new θ^{(t+1)} is chosen by maximizing the likelihood function in the Maximization Step:

$${θ}^{( t + 1 )} := {{argmax L}_{θ}}^{t} ( θ , X , Y ) \qquad ( 15 )$$

$$= {argmax}_{θ} {\sum }_{i = 1}^{n} {\sum }_{{Z}_{i}} {Q}_{i}^{t} ( {Z}_{i} ) log \frac{P ( {X}_{i} , \vert {Z}_{i} , θ , {Y}_{i} ) P ( {Z}_{i} )}{{Q}_{i}^{t} ( {Z}_{i} )} \qquad ( 16 )$$

where:
θ is the unknown mixture fraction,
P is the probability model function,
t is the iteration number,
i is a locus index,
n is the number of loci,
Xᵢ is the mixture read data at locus i,
Zᵢ is the genotype of the unknown sample at locus i,
Yᵢ is the genotype of the known sample at locus i, and
Qᵢ^{t} is the expected probability distribution over the unknown genotype at locus i and iteration t,
argmaxL_{θ}^{t} is the mixture fraction which maximizes the likelihood function at iteration t,
argmax_{θ} is the value of theta which maximizes the following expression,
   and iterative application of these increases the likelihood.

### Expectation Step

The distributions Qᵢ^{t}(Zᵢ) are calculated by normalizing P(Xᵢ, |Zᵢ, θ^{t}, Yᵢ)P(Zᵢ), to sum to one across the values of Zᵢ, at each locus i, where P is the conditional probability of the mixture read data, i is a locus index, t is the iteration number of the EM algorithm, θ is the mixture fraction, Xᵢ is the read data at locus i, Yᵢ is the known sample genotype at locus i, and Zᵢ is the genotype of the unknown sample at locus i. The population allele frequency spectrum, P(Zᵢ), can either be taken as uniform or estimated from variant databases.

For a read j at locus i, the read may either come from the reference allele (aᵢⱼ=0) or from the alternative allele (aᵢⱼ=1). The chances of these being related to both the mixture percentage and the two genotypes at that locus:

| Yᵢ | Zᵢ | P(aᵢⱼ\|Xᵢ, Yᵢ, θ) | $\frac{d}{d θ}$ |
|---|---|---|---|
| 0 | 0 | 0 | 0 |
| 0 | 1 | $\frac{θ}{2}$ | 0.5 |
| 0 | 2 | θ | 1 |
| 1 | 0 | $\frac{1}{2} - \frac{θ}{2}$ | −0.5 |
| 1 | 1 | $\frac{1}{2}$ | 0 |
| 1 | 2 | $\frac{1}{2} + \frac{θ}{2}$ | 0.5 |
| 2 | 0 | 1 − θ | −1 |
| 2 | 1 | $1 - \frac{θ}{2}$ | −0.5 |
| 2 | 2 | 1 | 0 |

where P is the conditional probability of the mixture read data, i is a locus index, θ is the unknown mixture fraction, Xᵢ is the mixture read data at locus i, Yᵢ is the genotype of the known sample at locus i, Zᵢ is the genotype of the unknown sample at locus i, aᵢⱼ is an allele for read j at locus i, and

$$\frac{d}{d θ}$$

is notation for derivative.

The per-site likelihood can then be broken up by the two options:

$$P ( {X}_{i} \vert {Y}_{i} , {Z}_{i} , θ ) = {\prod }_{j = 1}^{{d}_{i}} P ( {b}_{ij} , {e}_{ij} \vert {Y}_{i} , {Z}_{i} , θ ) \qquad ( 17 )$$

$$= {\prod }_{j = 1}^{{d}_{i}} {\sum }_{{a}_{ij} = 0}^{1} P ( {b}_{ij} , {e}_{ij ,} {a}_{ij} \vert {Y}_{i} , {Z}_{i} , θ ) \qquad ( 18 )$$

$$= {\prod }_{j = 1}^{{d}_{i}} {\sum }_{{a}_{ij} = 0}^{1} P ( {b}_{ij} , {e}_{ij ,} \vert {a}_{ij} , {Y}_{i} , {Z}_{i} , θ ) P ( {a}_{ij} \vert {Y}_{i} , {Z}_{i} , θ ) \qquad ( 19 )$$

$$= {\prod }_{j = 1}^{{d}_{i}} {\sum }_{{a}_{ij} = 0}^{1} P ( {b}_{ij} {e}_{ij} \vert {a}_{ij} ) P ( {a}_{ij} \vert {Y}_{i} , {Z}_{i} , θ ) \qquad ( 20 )$$

where P is the conditional probability of the data, i is a locus index, θ is the mixture fraction, Xᵢ is the mixture sample read data, Yᵢ is the known sample genotype, Zᵢ is the unknown sample genotype, aᵢⱼ is whether read j at locus i comes from the alternative allele, dᵢ is the read depth at locus i, j is the read index, bᵢⱼ is the base call from read j at locus i, eᵢⱼ is the base call quality score for read j at locus i, and the assumption is that the distribution of error rates is independent of any variables of interest (assumption 2), and therefore does not have an impact on genotyping or mixture estimation.

Two different read data likelihood models for P(bᵢⱼ, eᵢⱼ|aᵢⱼ) are presented here. The base call quality score model may provide more accuracy, but leads to a difficult to maximize likelihood function. The simpler quality-ignoring likelihood is a binomial likelihood function. Other models are also possible in the larger framework of mixture deconvolution.

### Base Quality Score Method

Read base qualities are typically reported as a value q which relates to the probability of an erroneous call to a specific other base

$$e = {10}^{\frac{- q}{10}} / 3.$$

For any read j at locus j, the base call bᵢⱼ is either the reference allele (0), variant allele (1), or in error (2 or 3).

$$P ( {b}_{ij} , {e}_{ij} \vert {a}_{ij} ) = \{ \begin{matrix}1 - 3 {e}_{ij} & {b}_{ij} = {a}_{ij} \\ {e}_{ij} & otherwise\end{matrix} \qquad ( 21 a )$$

reordering the reads so the index j starts with the rᵢ reads that match the reference allele, followed by the vᵢ reads that match the variant alternative allele, and finally followed by the reads that neither reference nor alternative, and using the notation f=P(aᵢⱼ|Yᵢ,Zᵢ,θ),

$$P ( {X}_{i} \vert {Y}_{i} , {Z}_{i} , θ ) = {\prod }_{j = 1}^{{d}_{i}} {\sum }_{{a}_{ij} = 0}^{1} P ( {b}_{ij} {e}_{ij} \vert {a}_{ij} ) P ( {a}_{ij} \vert {Y}_{i} , {Z}_{i} , θ ) \qquad ( 21 b )$$

$$= ( {\prod }_{j = 1}^{{r}_{i}} ( 1 - f ) ( 1 - 3 {e}_{ij} ) + {fe}_{ij} )$$

$$\cdot ( {\prod }_{j = 1 + {r}_{j}}^{{r}_{i} + {v}_{i}} f ( 1 - 3 {e}_{ij} ) + ( 1 - f ) {e}_{ij} )$$

$$\cdot ( {\prod }_{j = 1 + {r}_{j} + {v}_{j}}^{{d}_{i}} {e}_{ij} ) \qquad ( 21 c )$$

where P is the conditional probability of the data, i is a locus index, θ is the mixture fraction, Xᵢ is the mixture sample read data, Yᵢ is the known sample genotype, Zᵢ is the unknown sample genotype, aᵢⱼ is whether read j at locus i comes from the alternative allele, dᵢ is the read depth at locus i, j is the read index, bᵢⱼ is the base call from read j at locus i, eᵢⱼ is the base call quality score for read j at locus i, rᵢ represents reads that match the reference allele at locus i, vᵢ represents reads that match the variant alternative allele at locus i, rⱼ is a reference allele read, and v₁ is a variant allele read.

Though this may calculated in order to estimate Qᵢ^{t}(Zᵢ), where Qᵢ^{t} is the expected probability distribution for the unknown genotype at locus i, the M-step is challenging.

Simple Method with Quality Ignored

Ignoring base qualities, one can arrive at a much simpler emission probability:

$$P ( {b}_{ij} , {e}_{ij} \vert {a}_{ij} ) = \{ \begin{matrix}1 & {b}_{ij} = {a}_{ij} \\ 0 & otherwise\end{matrix} \qquad ( 22 a )$$

And again rearranging the read indexing (without loss of generality), and ignoring that are neither reference nor allele, and using f=P(aᵢⱼ|Yᵢ,Zᵢ,θ):

$$P ( {X}_{i} \vert {Y}_{i} , {Z}_{i} , θ ) = {\prod }_{j = 1}^{{d}_{i}} {\sum }_{{a}_{ij} = 0}^{1} P ( {b}_{ij} {e}_{ij} \vert {a}_{ij} ) P ( {a}_{ij} \vert {Y}_{i} , {Z}_{i} , θ ) \qquad ( 22 b )$$

$$∝ {( 1 - f )}^{{r}_{i}} {f}^{{v}_{i}}$$

where P is the conditional probability of the data, i is a locus index, θ is the mixture fraction, Xᵢ is the mixture sample read data, Yᵢ is the known sample genotype, Zᵢ is the unknown sample genotype, aᵢⱼ is whether read j at locus i comes from the alternative allele, dᵢ is the read depth at locus i, j is the read index, bᵢⱼ is the base call from read j at locus i, eᵢⱼ is the base call quality score for read j at locus i, rᵢ represents reads that match the reference allele at locus i, and vᵢ represents reads that match the variant alternative allele at locus i.

### Maximization Step

The previous E-step provided Qᵢ^{t}(Zᵢ), where Qᵢ^{t} is the expected probability distribution over the unknown genotype at locus i and iteration t, and Zᵢ is the unknown sample genotype, to use in the following maximization problem:

$${θ}^{( t + 1 )} \text{:=} {argmax}_{θ} {\sum }_{i = 1}^{n} {\sum }_{{Z}_{i}} {Q}_{i}^{t} ( {Z}_{i} ) log \frac{P ( {X}_{i} , \vert {Z}_{i} , θ , {Y}_{i} ) P ( {Z}_{i} )}{{Q}_{i}^{t} ( {Z}_{i} )} \qquad ( 23 )$$

$$= {argmax}_{θ} {\sum }_{i = 1}^{n} {\sum }_{{Z}_{i}} {Q}_{i}^{t} ( {Z}_{i} ) log P ( {X}_{i} , \vert {Z}_{i} , θ , {Y}_{i} ) - log \frac{P ( {Z}_{i} )}{{Q}_{i}^{t} ( {Z}_{i} )} \qquad ( 24 )$$

$$= {argmax}_{θ} {\sum }_{i = 1}^{n} {\sum }_{{Z}_{i}} {Q}_{i}^{t} ( {Z}_{i} ) log P ( {X}_{i} , \vert {Z}_{i} , θ , {Y}_{i} ) \qquad ( 25 )$$

where P is the conditional probability of the data, i is a locus index, t is the iteration number, n is the number of loci, θ is the mixture fraction, Xᵢ is the mixture sample read data, Yᵢ is the known sample genotype, Zᵢ is the unknown sample genotype, Qᵢ^{t} is the expected probability distribution over the unknown genotype at locus i and iteration t, argmax_{θ} is the value of theta which maximizes the following expression, and second part of the summand is dropped since it is constant with respect to θ. This is maximized when:

$$0 = \frac{\partial }{\partial θ} [ {\sum }_{i = 1}^{n} {\sum }_{{Z}_{i}} {Q}_{i}^{t} ( {Z}_{i} ) log P ( {X}_{i} , \vert {Z}_{i} , θ , {Y}_{i} ) ] \qquad ( 26 )$$

$$= {\sum }_{i = 1}^{n} {\sum }_{{Z}_{i}} {Q}_{i}^{t} ( {Z}_{i} ) \frac{\partial }{\partial θ} log P ( {X}_{i} , \vert {Z}_{i} , θ , {Y}_{i} ) \qquad ( 27 )$$

where P is the conditional probability of the data, i is a locus index, t is the iteration number, n is the number of loci, θ is the mixture fraction, Xᵢ is the mixture sample read data, Yᵢ is the known sample genotype, Zᵢ is the unknown sample genotype, Qᵢ^{t} is the expected probability distribution over the unknown genotype at locus i and iteration t, and ∂ is the partial derivative.

Using the base likelihood and f=P(aᵢⱼ|Yᵢ,Zᵢ,θ), where f is convenience notation, P is the conditional probability of the data, i is a locus index, θ is the mixture fraction, Yᵢ is the known sample genotype, Zᵢ is the unknown sample genotype, and aᵢⱼ is whether read j at locus i comes from the alternative allele, is dependent on both the genotypes and the mixture parameter.

$$\frac{\partial }{\partial θ} log P ( {X}_{i} , \vert {Y}_{i} , {Z}_{i} , θ ) = \frac{\partial }{\partial θ} [ log [ {( 1 - f )}^{{r}_{i}} {f}^{{v}_{i}} ] ] \qquad ( 28 )$$

$$= \frac{\partial }{\partial θ} [ {r}_{i} log ( 1 - f ) + {v}_{i} log f ] \qquad ( 29 )$$

$$= - {r}_{i} \frac{\partial f}{\partial θ} \frac{1}{1 - f} + {v}_{i} \frac{\partial f}{\partial θ} \frac{1}{f} \qquad ( 30 )$$

where P is the conditional probability of the data, i is a locus index, n is the number of loci, θ is the mixture fraction, Xᵢ is the mixture sample read data, Yᵢ is the known sample genotype, Zᵢ is the unknown sample genotype, ∂ is the partial derivative, rᵢ represents reads that match the reference allele at locus i, vᵢ represents reads that match the variant alternative allele at locus i, and f is convenience notation.

| Yᵢ | Zᵢ | f | $\frac{\partial f}{\partial θ}$ | $\frac{\partial }{\partial θ} log P ( {X}_{i} \vert {Y}_{i} , {Z}_{i} , θ )$ |
|---|---|---|---|---|
| 0 | 0 | 0 | 0 | 0 |
| 0 | 1 | $\frac{θ}{2}$ | 0.5 | ${r}_{i} \frac{1}{θ - 2} + {v}_{i} \frac{1}{θ}$ |
| 0 | 2 | θ | 1 | ${r}_{i} \frac{1}{θ - 1} + {v}_{i} \frac{1}{θ}$ |
| 1 | 0 | $\frac{1}{2} - \frac{θ}{2}$ | −0.5 | ${r}_{i} \frac{1}{θ + 1} + {v}_{i} \frac{1}{θ - 1}$ |
| 1 | 1 | $\frac{1}{2}$ | 0 | 0 |
| 1 | 2 | $\frac{1}{2} + \frac{θ}{2}$ | 0.5 | ${r}_{i} \frac{1}{θ - 1} + {v}_{i} \frac{1}{θ + 1}$ |
| 2 | 0 | 1 − θ | −1 | ${r}_{i} \frac{1}{θ} + {v}_{i} \frac{1}{θ - 1}$ |
| 2 | 1 | $1 - \frac{θ}{2}$ | −0.5 | ${r}_{i} \frac{1}{θ} + {v}_{i} \frac{1}{θ - 2}$ |
| 2 | 2 | 1 | 0 | 0 |

where P is the conditional probability of the data, i is a locus index, n is the number of loci, θ is the mixture fraction, Xᵢ is the mixture sample read data, Yᵢ is the known sample genotype, Zᵢ is the unknown sample genotype, ∂ is the partial derivative, rᵢ represents reads that match the reference allele at locus i, vᵢ represents reads that match the variant alternative allele at locus i, and f is convenience notation.

By grouping terms of the likelihood into convenience terms A, B, C, and D:

$$A = ( {\sum }_{\{ i \vert {Y}_{i} = 0 \}} {Q}_{i} ( 1 ) {r}_{i} ) + ( {\sum }_{\{ i \vert {Y}_{i} = 2 \}} {Q}_{i} ( 1 ) {v}_{i} ) \qquad ( 31 )$$

$$B = ( {\sum }_{\{ i \vert {Y}_{i} = 0 \}} {Q}_{i} ( 2 ) {r}_{i} ) + ( {\sum }_{\{ i \vert {Y}_{i} = 1 \}} {Q}_{i} ( 0 ) {v}_{i} + {Q}_{i} ( 2 ) {r}_{i} ) + ( {\sum }_{\{ i \vert {Y}_{i} = 2 \}} {Q}_{i} ( 0 ) {v}_{i} ) \qquad ( 32 )$$

$$C = ( {\sum }_{\{ i \vert {Y}_{i} = 0 \}} {Q}_{i} ( 1 ) {v}_{i} + {Q}_{i} ( 2 ) {v}_{i} ) + ( {\sum }_{\{ i \vert {Y}_{i} = 2 \}} {Q}_{i} ( 0 ) {r}_{i} + {Q}_{i} ( 1 ) {r}_{i} ) \qquad ( 33 )$$

$$D = {\sum }_{\{ i \vert {Y}_{i} = 1 \}} {Q}_{i} ( 0 ) {r}_{i} + {Q}_{i} ( 2 ) {v}_{i} \qquad ( 34 )$$

where Qᵢ is the probability distribution at locus i, i is a locus index, Yᵢ is the known sample genotype, rᵢ represents reads that match the reference allele at locus i, and vᵢ represents reads that match the variant alternative allele at locus i,

The maximum likelihood then satisfies:

$$0 = \frac{A}{0 - 2} + \frac{B}{0 - 1} + \frac{C}{0} + \frac{D}{0 + 1} \qquad ( 35 )$$

$$= ( A + B + C + D ) {θ}^{3} + ( - B - 2 C - 3 D ) {θ}^{2} + ( - A - 2 B - C + 2 D ) θ + 2 C \qquad ( 36 )$$

Another way to express this is using the equation

$$f = \frac{θ {Z}_{i} + ( 1 - θ ) {Y}_{i}}{2}$$

(where f is convenience notation, θ is the mixture fraction, Zᵢ is the unknown sample genotype, and Yᵢ is the known sample genotype) and expressing the derivative of the likelihood in terms of theta, Zᵢ, and Yᵢ without a table, but it does not lead to a simple polynomial to solve.

$$\frac{\partial }{\partial θ} log P ( {X}_{i} \vert {Y}_{i} , {Z}_{i} , θ ) = - {r}_{i} \frac{\partial }{\partial θ} \frac{1}{1 - f} + {v}_{i} \frac{\partial }{\partial θ} \frac{1}{f} \qquad ( 37 )$$

$$= - {r}_{i} \frac{{Z}_{i} - {Y}_{i}}{2} {( \frac{2 - θ {Z}_{i} - ( 1 - θ ) {Y}_{i}}{2} )}^{- 1} + {v}_{i} \frac{{Z}_{i} - {Y}_{i}}{2} {( \frac{θ {Z}_{i} + ( 1 - θ ) {Y}_{i}}{2} )}^{- 1} \qquad ( 38 )$$

$$= ( {Z}_{i} - {Y}_{i} ) [ {v}_{i} \frac{1}{θ {Z}_{i} + ( 1 - θ ) {Y}_{i}} - {r}_{i} \frac{1}{2 - θ {Z}_{i} - ( 1 - θ ) {Y}_{i}} ] \qquad ( 39 )$$

$$= ( {Z}_{i} - {Y}_{i} ) [ {v}_{i} \frac{1}{1 + ( {Z}_{i} - {Y}_{i} ) θ} - {r}_{i} \frac{1}{1 - ( {Z}_{i} - {Y}_{i} ) θ} ] \qquad ( 40 )$$

where P is the conditional probability of the data, i is a locus index, θ is the mixture fraction, Xᵢ is the mixture sample read data, Yᵢ is the known sample genotype, Zᵢ is the unknown sample genotype, ∂ is the partial derivative, rᵢ represents reads that match the reference allele at locus i, vᵢ represents reads that match the variant alternative allele at locus i, and f is convenience notation.

### Initial Conditions, Convergence, Restarts

The EM algorithm is only guaranteed to find a local minimum, and the likelihood space can have several local minimum in this sort of setting. For example, with this dataset:

| Yᵢ | Rᵢ | Vᵢ |
|---|---|---|
| 0 | 68 | 32 |
| 0 | 63 | 37 |
| 0 | 63 | 37 |
| 0 | 69 | 31 |
| 0 | 71 | 29 |

where:
Yᵢ is the known sample genotype,
Rᵢ is the number of reference reads at locus i, and
Vᵢ is the number of variant (alternative) reads at locus i.

There are two local minimum, corresponding to Zᵢ=1 and Zᵢ=2 (where Zᵢ is the unknown sample genotype)

| θ | logL |
|---|---|
| 0.3324563 | −458.4552 |
| 0.6640000 | −458.4784 |

where:
θ is the mixture fraction and L is the likelihood.
EM also converges very quickly in all observed cases:

| logL | θ | iteration |
|---|---|---|
| −946.6150 | 0.3320000 | 1 |
| −458.4555 | 0.3324434 | 2 |
| −458.4552 | 0.3324560 | 3 |
| −458.4552 | 0.3324563 | 4 |
| −458.4552 | 0.3324563 | 5 |

Initial starting points include θ∈{0.01,0.05,0.15, . . . ,0.95,0.99}.

### Example 5: An Alternative Method for Deconvolution Using Haplotype Likelihoods

To deconvolute mixtures, an imputation method similar to that described in Example 2 may be used, where pairs of haplotypes may be used to infer the genotype at a target site T. To do this, it is determined which pair of haplotypes has the highest likelihood, and the genotype of the target site is chosen from that haplotype pair. To assign a likelihood to a haplotype pair, it is observed that a haplotype pair assigns a genotype L; to each locus i: homozygous reference, heterozygous, or homozygous non-reference (alternative). In Example 4, the three likelihoods of the unknown subject's genotype are calculated at site i, as P(Xᵢ+Yᵢ, Zᵢ, θ), where Zᵢ is the unknown sample's genotype, Xᵢ is the known read data, Yᵢ is the known sample's genotype, and θ is the mixture percentage. To calculate the likelihood of a haplotype pair Hₚ, where p is an index across all possible pairs, and where G(Hₚ,i) is the genotype of the haplotype pair site i, equation 41 may be used:

$$P ( {H}_{p} ) = {\prod }_{i} P ( {X}_{i} \vert {Y}_{i} , {Z}_{i} = G ( {H}_{p} , i ) , θ ) \qquad ( 41 )$$

An example workflow is provided:

1) Collect a DNA sample that is a mixture of two different individuals. One of these individuals, referred to as the “known sample” or “known subject” has known genotypes at all loci in a database of haplotypes, such as the 1000 Genomes Project. The other individual, the “unknown sample” or “unknown subject” is genotyped with this method.

2) The mixture sample is sequenced, either by WGS or other method that covers most or all loci in the haplotype database, resulting in read count data X; at all loci i in the haplotype database.

3) The following process is started from a variety of beginning guesses for the mixture fraction of the DNA sample
a. Using the mixture fraction guess, estimate the probability distribution for the genotype of the unknown sample at all loci, as in Example 4.
b. Using the probability distribution of the unknown genotypes, estimate a new mixture fraction.
c. Evaluate the likelihood of the data. If the likelihood has changed more than a threshold amount, return to step 3a with the new mixture fraction percentage.

4) Take the mixture fraction percentage and genotype likelihoods, P(Xᵢ|Yᵢ,Zᵢ=Lᵢ,θ), from step 3 that resulted in the best overall likelihood.

5) Evaluate all haplotype pair likelihoods using the unknown sample genotype likelihoods P(Xᵢ|Yᵢ,Zᵢ=Lᵢ,θ).

6) Generate the probability that the unknown sample is homozygous reference, heterozygous, or homozygous alternative at a target site. To do this for any particular genotype, the probabilities of the haplotypes that are compatible with that genotype at the target site are summed up.

7) Generate a genotype for the unknown sample by establishing probability cutoffs and determining whether each target site is homozygous reference, heterozygous, homozygous alternative, or refrain from calling a genotype at a loci, depending on the strength of the probabilities.

8) Use the genotype to search a database of population genotypes for related individuals.

### Example 6: Deconvolution of a Mixture without a Known Genotype

In Example 4, a distribution is inferred over an unknown sample's genotype Zᵢ at each locus i, by using a known Yᵢ to calculate the likelihood. If Yᵢ is unknown, a distribution may be inferred over the Cartesian product

$${Y}_{i} \times {Z}_{i} = \{ ( 0 , 0 ) , ( 0 , 1 ) , ( 0 , 2 ) , ( 1 , 0 ) , ( 1 , 1 ) , ( 1 , 2 ) , ( 2 , 0 ) , ( 2 , 1 ) , ( 2 , 2 ) \} .$$

The method of Example 4 can then be used, by summing over Yᵢ×Zᵢ in all instances where the sum is only over Zᵢ, for example in equations (12) and (25).

An example workflow is provided:

1) Collect a DNA sample that is a mixture of two different individuals. One of these individuals, is the “sample of interest,” (SOI), and the other is the confounding sample (CS).

2) The mixture sample is sequenced, either by WGS or other method that covers most or all loci in the haplotype database, resulting in read count data Xᵢ at all loci i in the haplotype database.

3) The following process is started from a variety of beginning guesses for the mixture fraction of the DNA sample
a. Using the mixture fraction guess, and at each locus, estimate the joint probability of the genotypes of the SOI and the CS, as in Example 4 but with the EM modifications described in the first paragraph.
b. Using the probability distribution over the unknown genotypes, estimate a new mixture fraction.
c. Evaluate the likelihood of the data. If the likelihood has changed more than a threshold amount, return to step 3a with the new mixture fraction percentage.

4) Of the sets of mixture fraction and genotype probability distributions in step 3, choose the one that generated the maximum likelihood across all starting mixing fractions.

5) Use an imputation method (either the pairwise linkage or haplotype method), to generate genotypes for the SOI or CS at all target loci.

6) Use the genotype to search a database of population genotypes for individuals related to the SOI.

### Example 7: Deconvolution of a Mixture of More than Two Genotypes

The methods described in Examples 4 and 6 may be extended to infer genotypes of an arbitrary number of individuals in an arbitrary number of mixed samples.

An example workflow is provided:

1) Collect a set of DNA mixtures, such that between any two mixtures, there is at least one individual that is shared between the mixtures.

2) Each mixture sample is sequenced, either by WGS or other method that covers all loci in the haplotype database, resulting in read count data Xᵢₖ at all loci i in the haplotype database, across each mixture k.

3) The following process is started from a variety of beginning guesses for the mixture fraction guesses for each mixture:
a. Using the mixture fraction guess, and at each locus, estimate the joint probability of the genotypes of all individuals in the mixture, using the modifications described below.
b. Using the probability distribution over the unknown genotypes, estimate new mixture proportions for each mixture.
c. Evaluate the likelihood of the data across all samples. If the likelihood has changed more than a threshold amount, return to step 3a with the new mixture fraction percentage.

4) Of the sets of mixture proportions and genotype probability distributions in step 3, choose the one that generated the maximum likelihood across all starting mixing fractions.

5) Use an imputation method (either the pairwise linkage or haplotype method), to generate genotypes for every individual in any of the mixtures.

6) Use the genotype to search a database of population genotypes for individuals related to any of the individuals in a mixture of interest.

Let p be the number of individuals, and s the number of samples, with Aⱼₖ the fraction of individual j in sample k with the constraint that each column sums to 1 and each entry is non-negative. Let Xᵢₖ be the read count data for locus i in sample k. Finally, let Gᵢⱼ represent the genotype at locus i for individual j. Then the log likelihood function becomes:

$$\begin{matrix}L ( A ; X ) = log P ( X \vert A ) \\ = log {\sum }_{G} P ( X \vert A , G ) P ( G ) \\ = log {\prod }_{i = 1}^{n} {\prod }_{j = 1}^{p} {\prod }_{k = 1}^{s} {\sum }_{{G}_{ij}} P ( {X}_{ik} \vert {A}_{jk} , {G}_{ij} ) P ( {G}_{ij} ) \\ = {\prod }_{i = 1}^{n} {\prod }_{j = 1}^{p} {\prod }_{k = 1}^{s} log {\sum }_{{G}_{ij}} P ( {X}_{ik} \vert {A}_{jk} , {G}_{ij} ) P ( {G}_{ij} )\end{matrix}$$

In one example, P(Xᵢₖ|Aⱼₖ, Gᵢⱼ) is defined with a binomial distribution as in Example 4, with the binomial frequency parameter defined to be Σⱼ₌₁^{p}AⱼₖGᵢⱼ/(2p), that is the expected fraction of alternative alleles, using the weighted average of individuals' genotypes at locus i, according to the estimated fraction Aⱼₖ of individual j in sample k.

This function can be maximized for particular columns of A at a time, under the constraint that each column sums to 1 and that the entries of A are non-negative, using numerical methods. In particular, convex optimizers can be used to efficiently solve this since the log-likelihood function is concave for columns of A. This optimization step accomplishes the M step as in Example 4. And given a particular value of A, the likelihood for each Gᵢⱼ can be calculated using the binomial likelihood function, to calculate the E step, as in Example 4.

When there is prior knowledge that some samples contain nucleic acid from a subset of the individuals, this can be incorporated into the EM algorithm by setting entries of Aⱼₖ to 0, when it is known that individual j is not present in sample k, and also not including Aⱼₖ in the optimization problem during the M-step. For example, if A₂₁ is set to 0, A₁₁ must be 1, and since column 1 is fixed, the genotype matrix for row 1 is fixed. In this setting, the optimization problem becomes identical to Example 4.

As another example, if sample 1 is known to contain nucleic acid from individuals 1 and 2, and sample 2 contains nucleic acid from individuals 2 and 3, then the EM algorithm gains additional confidence in the genotyping of individual 2 in sample 1 from the genotyping data in sample 2, which in turn gains stronger likelihoods for all samples' genotypes through this information sharing.

### Example 8: Mixture Deconvolution Via One or More Genotyping Microarrays

DNA microarrays that are used for genotyping use a differential signal between the two alleles at each locus to genotype a sample. For example, two different probes on an array may each probe for one of two alleles at a biallelic locus, and the intensity of fluorescence of these two probes can be converted into a ratio of the majority allele fraction detected in the sample, referred to as theta herein. Often there is a measure of total signal at the site which is a conversion of the sum of the two alleles' signals, referred to as R herein.

The conversion of raw DNA microarray signal to theta and R values can be accomplished using a variety of techniques that may or may not include: chip-wide signal normalization procedures, GC correction procedures, chip design corrections for adjacent probe signals, and the like. These normalization techniques may apply at the batch level and/or to individual DNA chips. The conversion from normalized signal values to theta and/or R can involve many different mathematical conversions, from division of one allele by the sum of both alleles' signal, to arctanh conversion, sigmoid conversions, and the like.

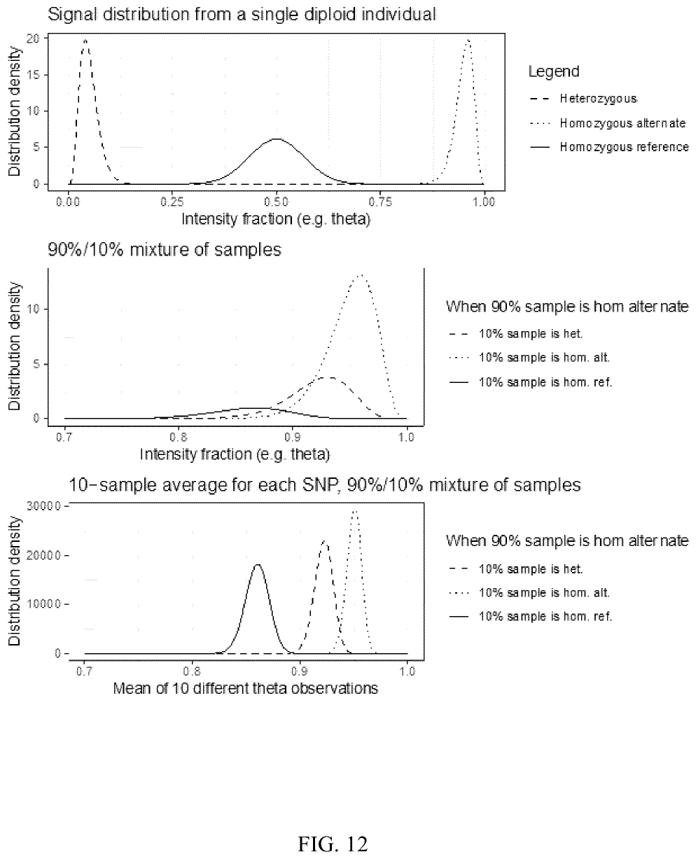

Given a theta and R for a locus on a single SNP chip, the distribution of signals from homozygous reference, heterozygous, and homozygous alternative genotypes are typically very distinct (FIG. 12, top panel). Though there may be variance in the exact theta value of a heterozygous locus, for example, the range of theta values for a heterozygous locus has insignificant overlap with either of the homozygous signal distributions. This facilitates highly accurate genotyping from a single array, often using only the theta value.

However, when the sample is a mixture of two different individuals, each locus is a combination of two different genotypes, and the signal distribution for an entire chip contains signals coming from nine different distributions of signals, for each possible pair of one of the three genotypes: individual A is one of (i) homozygous reference, (ii) heterozygous, (iii) homozygous alternative, as is individual B in the mixture. FIG. 12, middle panel, shows what three of these nine distributions for the theta value may look like for a 90%/10% mixture of two different individuals. Not shown are the distributions for R intensity values for these distributions, in addition to the raw signals that were used to generate the normalized theta and R values.

When multiple microarrays are run on the same sample, these multiple observations can be combined to result in a new value per locus such that the 9 distributions show better separation. The bottom panel of FIG. 12 shows one such combination of signal, the mean theta value of a locus across 10 microarrays on the same mixture. The variance of the mean value is much smaller than the variance of any individual observation, allowing clear separation of the distributions and straightforward deconvolution of the genotypes of the two individuals.

The following workflow is used to deconvolute a mixture sample of DNA:

1) Hybridize a DNA mixture of two different individuals to one or more genotyping microarrays.

2) Perform normalization on the microarray signals, using quantile normalization, background correction, and transformation to allele fraction (theta) and overall intensity (R) values for each locus on each microarray.

3) Determine summary statistics for a probability model of each locus across the microarrays. For example, Normal distribution probability model is used, determine the mean and variance for each locus. As another example, if a Beta distribution is used as a probability model for theta values, determine alpha and beta parameters for each locus using standard estimation methods.

4) Determine summary statistics for a probability distribution of per-microarray observations. For example, the theta and R values can be modeled as a two-dimensional Gaussian distribution, such that each microarray can be associated with a mean theta and a mean R value along with a 2×2 covariance matrix, and the parameters can be estimated by standard statistical techniques for each microarray. As another example, a separate Beta distribution can be used to model each microarray's theta values, and an independent negative binomial can be used to model the R values, and each microarray can have parameters estimated for each distribution with standard statistical methods.

5) Determine summary statistics for a probability model of the mixture percentage, using the locus and microarray summary statistics above. For example, a hierarchical Bayesian mixture model can be used to represent each locus's theta value on each microarray, and inference for the mixture parameters can use standard Bayesian methods.

6) Determine cutoffs on all the values associated with a locus, from raw and normalized data, to the summary statistics, to either convert a locus to a genotype pair, or to filter out a locus from determining a call. For example, if a Bayesian hierarchical model is used in step 5, the model allows inference of P(gᵢ|D), the posterior probability of genotype gᵢ (0, 1, or 2 alternate alleles) given the data D. Further, the odds ratio can be calculated for each value of gᵢ versus other possible values of gᵢ. A cutoff of, for example, >=10 on these odds ratios can result in a certain number of loci being genotyped. In another example a cutoff of >=5 can be established for heterozygous genotypes, and >=10 for homogenous genotypes, if it is desirable to include a greater number of heterozygous genotypes to meet a target heterozygosity ratio. (In step 8, some database search methods may require a certain fraction of calls to be heterozygous). Other cutoffs can be established for quality control. For example, a locus with more than 2 R values that are further than 3 standard deviations away from the mean can be excluded as outliers. As another example, a microarray with a standard deviation of R values that is outside established quality bounds can have its values excluded from the analysis.

7) Construct a genotype for individual A of the mixture and a second genotype for individual B of the mixture, using function from step 6. For each locus, the locus can be filtered out due to QC cutoffs, filtered out due to poor data support, or can establish a genotype pair for individual A and individual B. All the passing genotypes from individual A can be assembled into one file for database search. All the passing genotypes from individual B can be assembled into a second file for database search.

8) Use a genotype file from step 7 to search a database of genotypes to determine a match.

Variant of the Microarray Procedure with Additional Mixtures

If additional samples are available of the mixture that have different percentages, or a pure sample from one of the component individuals is available, then one or more microarrays can be hybridized to this additional mixture or pure sample. The genotypes and mixture percentages can be used to strengthen a Bayesian hierarchical model of the genotypes. An additional sample with a different mixture percentages can be particularly useful in the case when the initial mixture is close to 50%, when the distributions for genotype pairs such as (heterozygous, heterozygous) are indistinguishable from (homozygous reference, homozygous alternative).

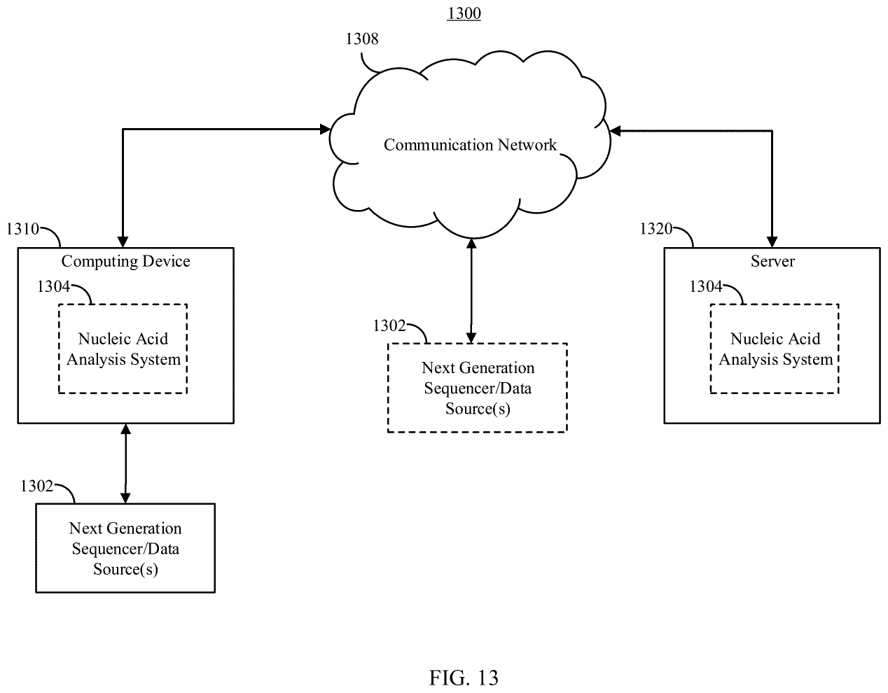

FIG. 13 shows an example 1300 of a system for analyzing nucleic acid in accordance with some embodiments of the disclosed subject matter. As shown in FIG. 13, a computing device 1310 can receive sequencing results indicating genetic information (e.g., DNA, RNA, etc.) that is present in a sample (e.g., a sample including genetic material from multiple subjects of the same species or closely related species, including at least one unknown subject) from a data source 1302 that generated and/or stores such data, and/or from an input device. In some embodiments, computing device 1310 can execute at least a portion of a nucleic acid analysis system 1304. In some embodiments, nucleic acid analysis system 1304 can infer a genotype information associated with one or more subjects that contributed genetic material to the sample, which can potentially be used for various applications. For example, nucleic acid analysis system 1304 can infer from a mixed sample that includes genetic material from a known subject (e.g., a crime victim) and genetic material from an unknown subject (e.g., a criminal), genotype information associated with the criminal can be used to try to identify the criminal (e.g., by searching a database of genetic information for the criminal and/or individuals related to the criminal). In a more particular example, nucleic acid analysis system 1304 can infer from multiple mixed samples that each includes genetic material from a particular known subject and genetic material from an unknown subject, genotype information associated with each unknown subject. As another example, nucleic acid analysis system 1304 can infer genotype information from a mixed sample of food (e.g., ground beef, bagged leafy green vegetables, etc.), which can be used to identify a number of sources of genetic material (e.g., number of genetically distinct cows in the ground beef, number of spinach plants in a bag of spinach), and/or identify a source of the different samples (e.g., animals in the ground beef, spinach plants in a bag of spinach). In such an example, results produced by nucleic acid analysis system 1304 can be used to identify the strains of a food borne illness and their potential source(s). As yet another example, nucleic acid analysis system 1304 can infer genotype information from a mixed sample of remains (e.g., from a mass grave), which can be used to identify a number of sources of genetic material and/or identify a source of different samples. As still another example, nucleic acid analysis system 1304 can infer genotype information from a sample that may have been contaminated (e.g., cross contaminated). In such an example, nucleic acid analysis system 1304 can determine a likelihood that multiple subjects contributed genetic material to a sample, and/or can be used to identify a source of contamination.

In some embodiments, system 1300 can use one or more techniques described above in connection with genotyping, aligning, classification, etc., and/or one or more techniques described above in connection with Examples 1 to 8 to infer genotype information from a sample (e.g., a mixed sample) assemble reads in results received from data source 1302 into sequences (e.g., sequences corresponding to oligos in the library).

Additionally or alternatively, in some embodiments, computing device 1310 can communicate information about genetic information (e.g., genetic sequence results generated by a next generation sequencing device, aligned reads associated with a particular library) from data source 1302 to a server 1320 over a communication network 1308 and/or server 1320 can receive genetic information from data source 1302 (e.g., directly and/or using communication network 1308), which can execute at least a portion of nucleic acid analysis system 1304. In such embodiments, server 1320 can return analysis results to computing device 1310 (and/or any other suitable computing device) indicative of genotype information associated with one or more subjects that contributed genetic material to the sample and/or results of a database search based on the genotype information.

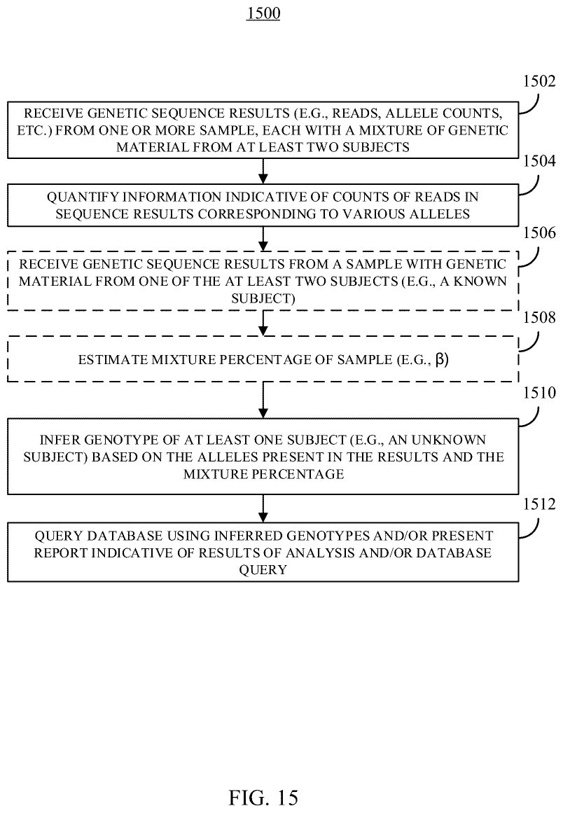

In some embodiments, computing device 1310 and/or server 1320 can be any suitable computing device or combination of devices, such as a desktop computer, a laptop computer, a smartphone, a tablet computer, a wearable computer, a server computer, a virtual machine being executed by a physical computing device, a specialty device (e.g., a next generation sequencing device), etc. As described below in connection with FIG. 15, in some embodiments, computing device 1310 and/or server 1320 can receive genetic sequencing results (e.g., corresponding to a sample or samples) from one or more data sources (e.g., data source 1302), and can infer a genotype of one or more subjects that contributed genetic material to the sample.

In some embodiments, data source 1302 can be any suitable source or sources of genetic data. For example, data source 1302 can be a next generation sequencing device or devices that generate a large number of reads from a sample. As another example, data source 1302 can be a data store configured to store genetic data, which may be aligned genetic data or unaligned reads.

In some embodiments, data source 1302 can be local to computing device 1310. For example, data source 1302 can be incorporated with computing device 1310. As another example, data source 1302 can be connected to computing device 1310 by one or more cables, a direct wireless link, etc. Additionally or alternatively, in some embodiments, data source 1302 can be located locally and/or remotely from computing device 1310, and provide data to computing device 1310 (and/or server 1320) via a communication network (e.g., communication network 1308).

In some embodiments, communication network 1308 can be any suitable communication network or combination of communication networks. For example, communication network 1308 can include a Wi-Fi network (which can include one or more wireless routers, one or more switches, etc.), a peer-to-peer network (e.g., a Bluetooth network), a cellular network (e.g., a 3G network, a 4G network, a 5G network, etc., complying with any suitable standard, such as CDMA, GSM, LTE, LTE Advanced, WiMAX, 5G NR, etc.), a wired network, etc. In some embodiments, communication network 1308 can be a local area network, a wide area network, a public network (e.g., the Internet), a private or semi-private network (e.g., a corporate or university intranet), any other suitable type of network, or any suitable combination of networks. Communications links shown in FIG. 13 can each be any suitable communications link or combination of communications links, such as wired links, fiber optic links, Wi-Fi links, Bluetooth links, cellular links, etc.

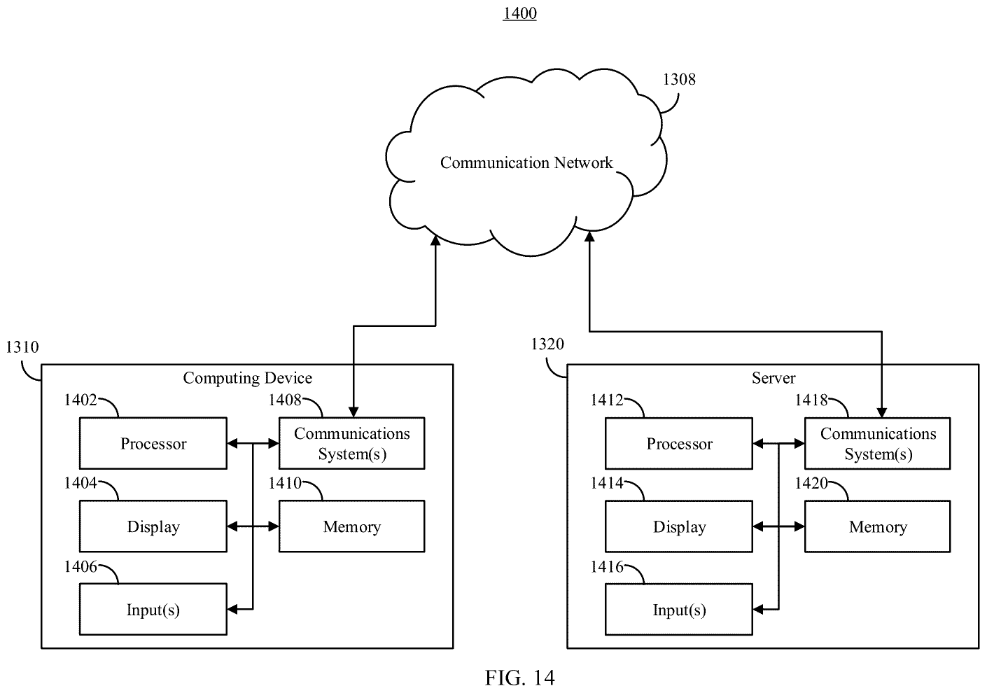

FIG. 14 shows an example of hardware that can be used to implement computing device 1310, and a server 1320, shown in FIG. 13 in accordance with some embodiments of the disclosed subject matter. As shown in FIG. 14, in some embodiments, computing device 1310 can include a processor 1402, a display 1404, one or more inputs 1406, one or more communication systems 1408, and/or memory 1410. In some embodiments, processor 1402 can be any suitable hardware processor or combination of processors, such as a central processing unit (CPU), a graphics processing unit (GPU), a microcontroller (MCU), an application specification integrated circuit (ASIC), a field programmable gate array (FPGA), etc. In some embodiments, display 1404 can include any suitable display devices, such as a computer monitor, a touchscreen, a television, etc. In some embodiments, inputs 1406 can include any suitable input devices and/or sensors that can be used to receive user input, such as a keyboard, a mouse, a touchscreen, a microphone, etc.

In some embodiments, communications systems 1408 can include any suitable hardware, firmware, and/or software for communicating information over communication network 1308 and/or any other suitable communication networks. For example, communications systems 1408 can include one or more transceivers, one or more communication chips and/or chip sets, etc. In a more particular example, communications systems 1408 can include hardware, firmware and/or software that can be used to establish a Wi-Fi connection, a Bluetooth connection, a cellular connection, an Ethernet connection, etc.

In some embodiments, memory 1410 can include any suitable storage device or devices that can be used to store instructions, values, etc., that can be used, for example, by processor 1402 to present content using display 1404, to communicate with server 1320 via communications system(s) 1408, etc. Memory 1410 can include any suitable volatile memory, non-volatile memory, storage, or any suitable combination thereof. For example, memory 1410 can include RAM, ROM, EEPROM, one or more flash drives, one or more hard disks, one or more solid state drives, one or more optical drives, etc. In some embodiments, memory 1410 can have encoded thereon a computer program for controlling operation of computing device 1310. In such embodiments, processor 1402 can execute at least a portion of the computer program to present content (e.g., user interfaces, graphics, tables, reports, etc.), receive genetic sequence results, information, and/or content from data source 1302, receive information (e.g., content, genetic information, etc.) from server 1320, transmit information to server 1320, etc.

In some embodiments, server 1320 can include a processor 1412, a display 1414, one or more inputs 1416, one or more communications systems 1418, and/or memory 1420. In some embodiments, processor 1412 can be any suitable hardware processor or combination of processors, such as a CPU, a GPU, an MCU, an ASIC, an FPGA, etc. In some embodiments, display 1414 can include any suitable display devices, such as a computer monitor, a touchscreen, a television, etc. In some embodiments, inputs 1416 can include any suitable input devices and/or sensors that can be used to receive user input, such as a keyboard, a mouse, a touchscreen, a microphone, etc.

In some embodiments, communications systems 1418 can include any suitable hardware, firmware, and/or software for communicating information over communication network 1308 and/or any other suitable communication networks. For example, communications systems 1418 can include one or more transceivers, one or more communication chips and/or chip sets, etc. In a more particular example, communications systems 1418 can include hardware, firmware and/or software that can be used to establish a Wi-Fi connection, a Bluetooth connection, a cellular connection, an Ethernet connection, etc.

In some embodiments, memory 1420 can include any suitable storage device or devices that can be used to store instructions, values, etc., that can be used, for example, by processor 1412 to present content using display 1414, to communicate with one or more computing devices 110, etc. Memory 1420 can include any suitable volatile memory, non-volatile memory, storage, or any suitable combination thereof. For example, memory 1420 can include RAM, ROM, EEPROM, one or more flash drives, one or more hard disks, one or more solid state drives, one or more optical drives, etc. In some embodiments, memory 1420 can have encoded thereon a server program for controlling operation of server 1320. In such embodiments, processor 1412 can execute at least a portion of the server program to transmit information and/or content (e.g., a user interface, graphs, tables, reports, etc.) to one or more computing devices 1310, receive genetic sequencing results, information, and/or content from one or more computing devices 1310, receive instructions from one or more devices (e.g., a personal computer, a laptop computer, a tablet computer, a smartphone, etc.), etc.

FIG. 15 shows an example of a process 1500 for analyzing nucleic acid in accordance with some embodiments of the disclosed subject matter. At 1502, process 1500 can receive genetic sequence results associated with one or more samples from any suitable source. In some embodiments, the one or more samples can include a mixture of genetic material from at least one unknown subject, and may include genetic material from a known subject. For example, the sample can include a mixture of genetic material from a known subject and one or more unknown subjects. As another example, the sample can include a mixture of genetic material from multiple unknown subjects.

In some embodiments, the results received by process 1500 at 1502 can include any suitable data formatted in any suitable format. For example, the results can include unaligned reads. As another example, the results can include aligned reads. In some embodiments, the genetic sequencing results can be results that were generated with any suitable sequencing depth. For example, the results can be generated at a relatively low sequence depth (e.g. 2× genome coverage) up to a relatively high sequence depth (e.g. >1000× genome coverage). For example, if a single sample is being analyzed with multiple unknown subjects, the results can be generated at a relatively high sequence depth. As another example, if multiple samples are being analyzed with each including one known subject, the results for each sample can be generated at a relatively low sequence depth.

In some embodiments, the results can represent genetic information from across an entire genome. For example, as described above in connection with Examples 1-3 and 5, the results can represent genetic information that includes data at known polymorphic positions, such as a source of positions (e.g., the 1000 Genomes project for human genome data). Additionally or alternatively, in some embodiments, the results can represent genetic information from selected portions of a genome. For example, the results can represent genetic information associated with particular genes. In such an example, the results associated with particular genes can be generated using any suitable technique or combination of techniques, such as target enrichment techniques.

In some embodiments, at 1502, process 1500 can include generating the genetic sequencing results (e.g., using data source 1302). Alternatively, in some embodiments, process 1500 can receive genetic sequencing results that have already been generated.

At 1504, process 1500 can quantify information indicative of counts of reads in sequence results corresponding to various alleles. In some embodiments, process 1500 can use any suitable technique or combination of techniques to quantify the results, such as techniques described herein (e.g., described above in connection with Examples 1 to 8).

At 1506, process 1500 can receive genetic sequence results from a sample with genetic material from a known subject. In some embodiments, the results received at 1506 can include any suitable data formatted in any suitable format (e.g., as described above in connection with 1502). In some embodiments, 1506 can be omitted. For example, if the sample includes genetic material from only unknown subjects, process 1500 can omit 1506.

At 1508, process 1500 can estimate a mixture percentage of the sample between at least two subjects using any suitable technique or combination of techniques. For example, process 1500 can use techniques described above in connection with Examples 3 to 8 to estimate a mixture percentage (e.g., sometimes referred to herein as β or θ), such as expectation maximization (EM) techniques. As another example, Bayesian hierarchical modeling or a neural network can be used to estimate the mixture percentage.

In some embodiments, when more than two samples are involved, expectation maximization, Bayesian hierarchical modeling, and/or neural networks can be used to estimate the mixture percentage of each component of the sample. In some embodiments, when the number of mixture components is unknown, a Dirichlet process can be used to estimate the number of samples in the mixture. The Dirichlet process can both cluster loci based on their variant allele fractions, and estimate the mixture percentage of each component. Additionally, the Dirichlet process can produce a posterior probability for the genotype of each sample in the mixture at each locus.

At 1510, process 1500 can infer a genotype(s) of at least one subject based on alleles present in the results and based on the mixture percentage estimated at 1508. In some embodiments, process 1500 can use any suitable technique or combination of techniques to infer the genotype of the at least one subject. For example, process 1500 can infer one or more genotypes for the unknown subject (and/or multiple unknown subjects) using one or more techniques described above in connection with Examples 3 to 8.

In some embodiments, the genotypes can be used to predict the number of subjects (e.g., subjects of the same species and/or closely related species) that contributed genetic material to the sample using any suitable technique or combination of techniques. For example, a Dirichlet Process can be used to estimate the number of genetically distinct cows in a sample of ground beef.

In some embodiments, at 1510, process 1500 can infer genotypes for multiple samples, which may or may not include genetic materials from the same unknown subject.

At 1512, process 1500 can submit at least a subset of the genotypes associated with an unknown subject to a database of genetic information (e.g., to identify the subject and/or individuals related to the individual) and/or can present a report indicative of results of the analysis and/or results of the query. For example, process 1500 can generate (e.g., by nucleic acid analysis system 1304) and/or present a report (e.g., using a display associated with computing device 1310) that includes information indicative of genotypes of one or more unknown subjects, the number of subjects that contributed genetic material to the sample, a mixture percentage, database query results, etc.

### Certain Implementations

Following are non-limiting examples of certain implementations of technology described herein.

A1. A method for generating a genotype for a target genomic locus for an unknown subject, comprising:
a) for a first test sample comprising a mixture of nucleic acid from the unknown subject and a known subject, obtaining sequence reads aligned to a reference genome;
b) from the sequence reads, quantifying a linked reference allele and quantifying a linked alternative allele, thereby generating allele quantifications for a linked genomic locus;
c) estimating the fraction of nucleic acid from the unknown subject in the mixture;
d) for the unknown subject, generating a genotype likelihood for the linked genomic locus given the fraction estimated in (c) and according to the allele quantifications in (b);
e) for the unknown subject, generating a set of genotype likelihoods for a target reference allele and a target alternative allele at the target genomic locus based on the linked genomic locus genotype likelihood generated in (d); and
f) for the unknown subject, generating a genotype at the target genomic locus based on the set of genotype likelihoods generated in (e).

A2. The method of embodiment A1, further comprising obtaining a genotype for the linked genomic locus for the known subject.

A3. The method of embodiment A2, wherein obtaining a genotype for the linked genomic locus for the known subject comprises obtaining sequence reads aligned to a reference genome for a second test sample comprising nucleic acid from the known subject and no nucleic acid from the unknown subject.

A4. The method of any one of embodiments A1 to A3, wherein estimating the fraction of nucleic acid from the unknown subject in the mixture in (c) is based on a deviation from an expected variant allele frequency for the known subject.

A5. The method of any one of embodiments A1 to A4, wherein estimating the fraction of nucleic acid from the unknown subject in the mixture in (c) is based on a probabilistic model.

A6. The method of any one of embodiments A1 to A5, wherein estimating the fraction of nucleic acid from the unknown subject in the mixture in (c) is based on incremental estimates.

A7. The method of any one of embodiments A1 to A6, wherein estimating the fraction of nucleic acid from the unknown subject in the mixture in (c) is based on incremental estimates between 0% to 100%.

A8. The method of embodiment A6 or A7, wherein the incremental estimates are 5% incremental estimates.

A9. The method of embodiment A6 or A7, wherein the incremental estimates are 1% incremental estimates.

A10. The method of any one of embodiments A1 to A9, wherein the fraction of nucleic acid from the unknown subject in the mixture is estimated in (c) according to an expectation-maximization (EM) algorithm.

A11. The method of any one of embodiments A1 to A10, wherein the genotype likelihood for the linked genomic locus is generated in (d) according to an expectation-maximization (EM) algorithm.

A12. The method of embodiment A10 or A11, wherein the fraction of nucleic acid from the unknown subject in the mixture is estimated in (c) according to an expectation-maximization (EM) algorithm and the genotype likelihood for the linked genomic locus is generated in (d) according to an expectation-maximization (EM) algorithm.

A13. The method of any one of embodiments A1 to A12, wherein a plurality of likelihoods for a plurality of linked genomic loci are generated in (d).

A14. The method of embodiment A13, further comprising generating a total likelihood based on the plurality of likelihoods.

A15. The method of any one of embodiments A1 to A14, comprising repeating parts (c) and (d), wherein each repeat of parts (c) and (d) comprises iteratively generating a different estimate of the fraction of nucleic acid from the unknown subject in the mixture according to a new genotype likelihood or a total likelihood.

A16. The method of embodiment A15, comprising repeating parts (c) and (d) until the total likelihood value changes by a percentage that is less than a threshold value.

A17. The method of embodiment A16, wherein the threshold value is 0.01%.

A18. The method of any one of embodiments A1 to A17, wherein the genotype likelihood generated in (d) is for a locus where the known subject has an alternative allele.

A19. The method of embodiment A18, wherein the known subject is homozygous for a linked alternative allele at the locus.

A20. The method of embodiment A18, wherein the known subject is heterozygous for a linked alternative allele and a linked reference allele at the locus.

A21. The method of any one of embodiments A1 to A20, wherein generating a set of genotype likelihoods for a target reference allele and a target alternative allele at the target genomic locus in (e) is based on 1) the linked genomic locus genotype likelihood generated in (d), and 2) a probability of a genotype at the target genomic locus based on prior probabilities of the target reference allele and the target alternative allele.

A22. The method of embodiment A21, wherein the probability of a genotype at the target genomic locus is based, in part, on haplotype frequencies for (i) the target reference allele and the linked reference allele, (ii) the target reference allele and the linked alternative allele, (iii) the target alternative allele and the linked reference allele, and (iv) the target alternative allele and the linked alternative allele.

A23. The method of any one of embodiments A1 to A22, wherein the set of genotype likelihoods in (e) comprises one or more likelihoods for genotypes chosen from homozygous for the target reference allele, heterozygous for the target reference allele and the target alternative allele, and homozygous for the target alternative allele.

A24. The method of any one of embodiments A1 to A23, wherein (b) further comprises quantifying a target reference allele and quantifying a target alternative allele, thereby generating allele quantifications for the target genomic locus.

A25. The method of any one of embodiments A1 to A24, wherein (d) further comprises for the known subject, generating a genotype likelihood for the linked genomic locus.

A26. The method of any one of embodiments A1 to A25, wherein (e) further comprises for the known subject, generating a set of genotype likelihoods for a target reference allele and a target alternative allele at the target genomic locus.

A27. The method of embodiment A26, wherein (f) further comprises for the known subject, generating a genotype at the target genomic locus based on the set of genotype likelihoods generated for the known subject.

A28. The method of any one of embodiments A23 to A27, comprising generating a genotype likelihood for a target genomic locus, wherein the likelihood (P(D/T,θ)) of the data (D) at a mixture percentage (θ) for a genotype (T) for the unknown subject is generated according to a process represented by equation (1′):

$$P ( D \vert T , θ ) = {\prod }_{i} {\sum }_{{L}_{i} ∈ 0 , 1 , 2} P ( {D}_{i} \vert {L}_{i} , {Y}_{i} , θ ) P ( {L}_{i} \vert T ) \qquad ( {1}^{′} )$$

wherein
D is sequence read data from the mixture,
θ is the mixture percentage,
i is a linked locus to T,
Dᵢ is sequence read data from the mixture at locus i,
Lᵢ is a genotype of the unknown subject at locus i, and
Yᵢ is a genotype of the known subject at locus i.

A29. The method of embodiment A28, wherein P(Dᵢ|Lᵢ, Yᵢ, θ) is determined according to equation (2′):

$$P ( {D}_{i} \vert {L}_{i} , {Y}_{i} , θ ) ∼ Binomial ( \vert {D}_{i} \vert , \frac{{L}_{i} θ + {Y}_{i} ( 1 - θ )}{2} ) \qquad ( {2}^{′} )$$

wherein
Binomial is a standard binomial distribution.

A30. The method of embodiment A28 or A29, wherein P(Lᵢ|T) is determined according to equation (3′):

$$P ( {L}_{i} \vert T ) = \frac{haplofreq ( {L}_{i} , T )}{haplofreq ( T )} \qquad ( {3}^{′} )$$

wherein
haplofreq(Lᵢ, T) is a population frequency of co-occurrence of genotypes Lᵢ and T in the same haplotype, from a population database, and
haplofreq(T) is a population frequency of genotype T, from a population database.

A31. The method of embodiment A30, wherein haplofreq(Lᵢ, T) is determined according to Lᵢ0T0, Lᵢ1T0, Lᵢ0T1 and Lᵢ1T1, which are haplotype frequencies for (i) a linked reference allele and a target reference allele, (ii) a linked alternative allele and a target reference allele, (iii) a linked reference allele and a target alternative allele, and (iv) a linked alternative allele and a target alternative allele.

A32. The method of embodiment A28 or A29, wherein P(Lᵢ|T) is determined according to one or more haplotype frequencies chosen from Lᵢ0T0, Lᵢ1T0, Lᵢ0T1 and Lᵢ1T1, where Lᵢ0T0 is a haplotype frequency for a linked reference allele and a target reference allele, Lᵢ1T0 is a haplotype frequency for a linked alternative allele and a target reference allele, Lᵢ0T1 is a haplotype frequency for a linked reference allele and a target alternative allele, and Lᵢ1T1 is a haplotype frequency for a linked alternative allele and a target alternative allele.

A33. The method of embodiment A32, wherein P(Lᵢ|T) comprises four possibilities: P(Lᵢ=0|T=0), P(Lᵢ=1|T=0), P(Lᵢ=0|T=1), and P(Lᵢ=1|T=1), where P(Lᵢ=0|T=0)=Lᵢ0T0/(Lᵢ0T0+Lᵢ1T0), P(Lᵢ=1|T=0)=Lᵢ1T0/(Lᵢ0T0+Lᵢ1T0), P(Lᵢ=0|T=1)=Lᵢ0T1/(Lᵢ0T1+Lᵢ1T1), and P(Lᵢ=1|T=1)=Lᵢ1T1/(Lᵢ0T1+Lᵢ1T1).

A34. The method of any one of embodiments A28 to A33, wherein only the target genomic locus T is used as the linked genomic locus or the plurality of linked genomic loci.

A35. The method of any one of embodiments A1 to A34, wherein the genotype generated in (f) is an unphased genotype.

A36. The method of any one of embodiments A1 to A35, wherein the genotype generated in (f) is for a single nucleotide polymorphism (SNP).

A37. The method of any one of embodiments A1 to A36, wherein the genotype generated in (f) is for a bi-allelic single nucleotide polymorphism (SNP).

A38. The method of any one of embodiments A1 to A37, wherein (b) comprises quantifying a plurality of linked reference alleles and quantifying a plurality of linked alternative alleles, thereby generating a plurality of allele quantifications for a plurality of linked genomic loci.

A39. The method of embodiment A38, wherein the plurality of linked genomic loci comprises loci within about 10 kilobases upstream and about 10 kilobases downstream of the target genomic locus.

A40. The method of embodiment A38 or A39, wherein the plurality of linked genomic loci comprises about 10 linked genomic loci to about 1000 linked genomic loci.

A41. The method of any one of embodiments A38 to A40, wherein a plurality of genotype likelihoods for the linked genomic loci is generated according to the plurality of allele quantifications for the plurality of linked genomic loci.

A42. The method of embodiment A41, wherein a plurality of genotype likelihood sets for the target genomic locus is generated according to the plurality of genotype likelihoods for the linked genomic loci.

A43. The method of embodiment A42, wherein a composite genotype likelihood is generated for each genotype from the plurality of genotype likelihood sets.

A44. The method of embodiment A42 or A43, wherein the genotype at the target genomic locus is generated based on the plurality of genotype likelihood sets and/or the composite genotype likelihoods.

A45. The method of any one of embodiments A1 to A44, comprising generating a plurality of genotypes at a plurality of target genomic loci for the unknown subject.

A46. The method of any one of embodiments A1 to A45, comprising generating a plurality of genotypes at a plurality of target genomic loci for the known subject.

A47. The method of embodiment A45 or A46, wherein the plurality of target genomic loci comprises about 100,000 loci or more.

A48. The method of embodiment A45 or A46, wherein the plurality of target genomic loci comprises about 600,000 loci or more.

A49. The method of any one of embodiments A45 to A48, wherein each genotype in the plurality of genotypes is generated independently from the other genotypes in the plurality of genotypes.

A50. The method of any one of embodiments A1 to A49, wherein generating the genotype at the target genomic locus does not comprise generating a haplotype for two or more target genomic loci.

A51. The method of any one of embodiments A1 to A50, wherein generating the genotype at the target genomic locus does not comprise generating a haplotype for a target genomic locus and one or more linked genomic loci.

A52. The method of any one of embodiments A45 to A51, further comprising identifying the unknown subject based on the plurality of genotypes generated for the unknown subject.

A53. The method of any one of embodiments A1 to A52, wherein the method comprises prior to (b) filtering sequence reads.

A54. The method of embodiment A53, wherein the sequence reads are filtered by removing sequence reads that align to a reference genome locus that is within 1 to 10 bases of an insertion polymorphism or a deletion polymorphism.

A55. The method of embodiment A54, wherein the sequence reads are filtered by removing sequence reads that align to a reference genome locus that is within 4 bases of an insertion polymorphism or a deletion polymorphism.

A56. The method of any one of embodiments A45 to A55, wherein the method comprises filtering the target genomic loci.

A57. The method of embodiment A56, wherein the target genomic loci are filtered by removing genomic loci that are within 1 to 10 bases of an insertion polymorphism or a deletion polymorphism.

A58. The method of embodiment A57, wherein the target genomic loci are filtered by removing genomic loci that are within 4 bases of an insertion polymorphism or a deletion polymorphism.

A59. The method of any one of embodiments A1 to A58, further comprising prior to (a) sequencing the nucleic acid in the first test sample by a sequencing process, thereby generating sequence reads.

A60. The method of embodiment A59, wherein the sequencing process is a whole genome sequencing process.

A61. The method of embodiment A59, wherein the sequencing process is a genome-wide sequencing process.

A62. The method of any one of embodiments A59 to A61, wherein the sequencing process is a non-targeted sequencing process.

A63. The method of any one of embodiments A59 to A62, wherein the sequencing process is a massively parallel sequencing process.

A64. The method of any one of embodiments A59 to A63, wherein the sequencing process is performed at about 50-fold coverage.

A65. The method of any one of embodiments A59 to A63, wherein the sequencing process is performed at about 100-fold coverage.

A66. The method of any one of embodiments A59 to A65, further comprising aligning the sequence reads to a reference genome, thereby generating aligned sequence reads.

A67. The method of any one of embodiments A59 to A66, further comprising prior to sequencing the nucleic acid in the first test sample, producing a sequencing library.

A68. The method of embodiment A67, wherein producing a sequencing library comprises generating single-stranded nucleic acid (ssNA) from the nucleic acid in the first test sample.

A69. The method of embodiment A68, wherein producing a sequencing library comprises combining the ssNA with a plurality of scaffold adapter species, or components thereof.

A70. The method of embodiment A69, wherein the scaffold adapter components comprise (i) an oligonucleotide and (ii) a scaffold polynucleotide comprising an ssNA hybridization region and an oligonucleotide hybridization region.

A71. The method of embodiment A70, wherein the ssNA and the plurality of scaffold adapter species, or components thereof, are combined under conditions in which the scaffold polynucleotide is hybridized to (i) an ssNA terminal region and (ii) the oligonucleotide, thereby forming hybridization products in which an end of the oligonucleotide is adjacent to an end of the ssNA terminal region.

A72. The method of embodiment A71, further comprising covalently linking the adjacent ends of the oligonucleotide and the ssNA terminal region, thereby generating covalently linked hybridization products.

A73. The method of any one of embodiments A70 to A72, wherein the ssNA hybridization region in the scaffold polynucleotide of each scaffold adapter species comprises a unique sequence.

A74. The method of any one of embodiments A70 to A73, wherein the ssNA hybridization region in the scaffold polynucleotide of each scaffold adapter species comprises a random sequence.

A75. The method of any one of embodiments A69 to A74, wherein one or both native ends of the ssNA are present when the ssNA is combined with the plurality of scaffold adapter species, or components thereof.

A76. The method of any one of embodiments A1 to A75, wherein the first test sample is a forensic sample.

A77. The method of any one of embodiments A1 to A75, wherein the first test sample is a non-forensic sample.

A78. The method embodiment A76 or A77, wherein the first test sample comprises hair.

A79. The method of embodiment A76 or A77, wherein the first test sample comprises blood.

A80. The method of embodiment A76 or A77, wherein the first test sample comprises semen.

A81. The method of any one of embodiments A1 to A80, wherein the first test sample is from two human subjects.

A82. The method of any one of embodiments A1 to A81, wherein the nucleic acid in the first test sample comprises cell free nucleic acid.

A83. The method of any one of embodiments A1 to A82, wherein the nucleic acid in the first test sample comprises degraded or damaged nucleic acid.

A84. The method of any one of embodiments A1 to A83, wherein the nucleic acid in the first test sample comprises fragmented nucleic acid.

A85. The method of any one of embodiments A1 to A84, wherein the nucleic acid in the first test sample comprises single-stranded nucleic acid, double-stranded nucleic acid, or single-stranded nucleic acid and double-stranded nucleic acid.

A86. The method of any one of embodiments A1 to A85, wherein the sequence reads are generated from single-stranded nucleic acid fragments, double-stranded nucleic acid fragments, or single-stranded nucleic acid fragments and double-stranded nucleic acid fragments, from the first test sample.

A87. The method of any one of embodiments A1 to A86, wherein the one or more or all of (a), (b), (c), (d), (e), and (f) are performed by a computer.

B1. A method for generating a genotype for a target genomic locus for an unknown subject, comprising:
a) for a first test sample comprising a mixture of nucleic acid from the unknown subject and a known subject, obtaining sequence reads aligned to a reference genome;
b) for a haplotype group comprising a target genomic locus and a plurality of linked genomic loci, quantifying a linked reference allele and quantifying a linked alternative allele for each linked genomic locus in the group according to the sequence reads generated in (a), thereby generating allele quantifications for each linked genomic locus in the haplotype group;
c) estimating the fraction of nucleic acid from the unknown subject in the mixture;
d) for the unknown subject, generating genotype likelihoods for the plurality of linked genomic loci in the haplotype group given the fraction estimated in (c) and according to the allele quantifications in (b);
e) for the unknown subject, generating a haplotype pair likelihood set for the haplotype group according to the genotype likelihoods generated in (d); and
f) for the unknown subject, generating a genotype at the target genomic locus based on the haplotype pair likelihood set generated in (e).

B2. The method of embodiment B1, further comprising obtaining genotypes for the plurality of linked genomic loci in the haplotype group for the known subject.

B3. The method of embodiment B2, wherein obtaining genotypes for the plurality of linked genomic loci in the haplotype group for the known subject comprises obtaining sequence reads aligned to a reference genome for a second test sample comprising nucleic acid from the known subject and no nucleic acid from the unknown subject.

B4. The method of any one of embodiments B1 to B3, wherein estimating the fraction of nucleic acid from the unknown subject in the mixture in (c) is based on a deviation from an expected variant allele frequency for the known subject.

B5. The method of any one of embodiments B1 to B4, wherein estimating the fraction of nucleic acid from the unknown subject in the mixture in (c) is based on a probabilistic model.

B6. The method of any one of embodiments B1 to B5, wherein estimating the fraction of nucleic acid from the unknown subject in the mixture in (c) is based on incremental estimates.

B7. The method of any one of embodiments B1 to B6, wherein estimating the fraction of nucleic acid from the unknown subject in the mixture in (c) is based on incremental estimates between 0% to 100%.

B8. The method of embodiment B6 or B7, wherein the incremental estimates are 5% incremental estimates.

B9. The method of embodiment B6 or B7, wherein the incremental estimates are 1% incremental estimates.

B110. The method of any one of embodiments B1 to B9, wherein the fraction of nucleic acid from the unknown subject in the mixture is estimated in (c) according to an expectation-maximization (EM) algorithm.

B111. The method of any one of embodiments B1 to B110, wherein the genotype likelihoods for the linked genomic loci are generated in (d) according to an expectation-maximization (EM) algorithm.

B112. The method of embodiment B110 or B111, wherein the fraction of nucleic acid from the unknown subject in the mixture is estimated in (c) according to an expectation-maximization (EM) algorithm and the genotype likelihoods for the linked genomic loci are generated in (d) according to an expectation-maximization (EM) algorithm.

B113. The method of any one of embodiments B1 to B113, comprising repeating parts (c) and (d), wherein each repeat of parts (c) and (d) comprises iteratively generating a different estimate of the fraction of nucleic acid from the unknown subject in the mixture according to new genotype likelihoods.

B14. The method of any one of embodiments B1 to B113, wherein (d) further comprises generating a probability distribution of the genotype likelihoods for the linked genomic loci.

B15. The method of embodiment B14, comprising repeating parts (c) and (d), wherein each repeat of parts (c) and (d) comprises iteratively generating a different estimate of the fraction of nucleic acid from the unknown subject in the mixture according to a new probability distribution of the genotype likelihoods.

B16. The method of embodiment B14 or B15, comprising repeating parts (c) and (d) until the likelihood value changes by a percentage that is less than a threshold value.

B17. The method of embodiment B16, wherein the threshold value is 0.01%.

B118. The method of any one of embodiments B1 to B117, wherein the genotype likelihoods generated in (d) are for one or more loci where the known subject has an alternative allele.

B19. The method of embodiment B118, wherein the known subject is homozygous for a linked alternative allele at one or more loci in the haplotype group.

B20. The method of embodiment B118, wherein the known subject is heterozygous for a linked alternative allele and a linked reference allele at one or more loci in the haplotype group.

B21. The method of any one of embodiments B1 to B20, wherein generating a haplotype pair likelihood set for the haplotype group in (e) is based on 1) genotype likelihoods generated in (d), and 2) the fraction of nucleic acid from the unknown subject in the mixture estimated in (c).

B22. The method of any one of embodiments B2 to B21, a haplotype pair likelihood set for the haplotype group is generated (e) from a plurality of haplotype pair likelihoods each generated according to (i) sequence read data generated in (a), (ii) the known subject's genotype at a given locus, (iii) the unknown subject's genotype at a given locus, (iv) the fraction of nucleic acid from the unknown subject in the mixture estimated in (c), and (v) a genotype of the haplotype pair at a given locus.

B23. The method of any one of embodiments B2 to B22, a haplotype pair likelihood set for the haplotype group is generated (e) from a plurality of haplotype pair likelihoods each generated according to equation 41:

$$P ( {H}_{p} ) = {\prod }_{i} P ( {X}_{i} \vert {Y}_{i} , {Z}_{i} = G ( {H}_{p} , i ) , θ ) \qquad ( 41 )$$

wherein:
P(Hₚ) is the likelihood of a given haplotype pair,
Xᵢ is sequence read data generated in (a),
Yᵢ is the known subject's genotype,
Zᵢ is the unknown subject's genotype,
i is a locus,
θ is the fraction of nucleic acid from the unknown subject in the mixture estimated in (c), and
G(Hₚ, i) is the genotype of the haplotype pair at locus i.

B24. The method of any one of embodiments B1 to B23, wherein (f) comprises generating a probability that the unknown subject is homozygous reference, heterozygous, or homozygous alternative at the target genomic locus.

B25. The method of embodiment B24, wherein generating the probability comprises summing probabilities of haplotypes that are compatible with one or more genotypes at the target genomic locus.

B26. The method of any one of embodiments B1 to B25, wherein (f) comprises generating one or more probability cutoffs and determining whether the genotype at the target genomic locus is homozygous reference, heterozygous, or homozygous alternative.

B27. The method of any one of embodiments B1 to B26, wherein (e) further comprises identifying the most probable haplotype pair from the haplotype pair likelihood set.

B28. The method of embodiment B27, wherein the genotype at the target genomic locus is generated in (f) according to the most probable haplotype pair.

B29. The method of any one of embodiments B1 to B26, wherein (e) further comprises aggregating the haplotype pair likelihoods across all haplotype pairs for the haplotype group, thereby generating aggregate likelihoods.

B30. The method embodiment B29, wherein the genotype at the target genomic locus is generated in (f) according to the highest aggregate likelihood.

B31. The method of any one of embodiments B1 to B30, wherein the genotype generated in (f) is an unphased genotype.

B32. The method of any one of embodiments B1 to B31, wherein the genotype generated in (f) is for a single nucleotide polymorphism (SNP).

B33. The method of any one of embodiments B1 to B32, wherein the genotype generated in (f) is for a bi-allelic single nucleotide polymorphism (SNP).

B34. The method of any one of embodiments B1 to B33, wherein (b) comprises, for a plurality of haplotype groups each comprising a target genomic locus and a plurality of linked genomic loci, quantifying a linked reference allele and quantifying a linked alternative allele for each linked genomic locus in each group according to the sequence reads generated in (a), thereby generating allele quantifications for each linked genomic locus for each group in the plurality of haplotype groups.

B35. The method of embodiment B34, wherein a plurality of haplotype pair likelihood sets are generated in (e) according to the allele quantifications for each linked genomic locus for each group in the plurality of haplotype groups.

B36. The method of embodiment B35, wherein a plurality of genotypes at a plurality of target genomic loci are generated in (f) based on the plurality of haplotype pair likelihood sets.

B37. The method of embodiment B36, wherein each genotype in the plurality of genotypes is generated independently from the other genotypes in the plurality of genotypes.

B38. The method of embodiment B36 or B37, further comprising identifying the unknown subject based on the plurality of genotypes generated for the unknown subject.

B39. The method of any one of embodiments B36 to B38, wherein the method comprises filtering the target genomic loci.

B40. The method of embodiment B39, wherein the target genomic loci are filtered by removing genomic loci that are within 1 to 10 bases of an insertion polymorphism or a deletion polymorphism.

B41. The method of embodiment B40, wherein the target genomic loci are filtered by removing genomic loci that are within 4 bases of an insertion polymorphism or a deletion polymorphism.

B42. The method of any one of embodiments B1 to B41, wherein generating the genotype at the target genomic locus does not comprise generating a haplotype group comprising two or more target genomic loci.

B43. The method of any one of embodiments B1 to B42, wherein the plurality of linked genomic loci in the haplotype group comprises loci within about 10 kilobases upstream and about 10 kilobases downstream of the target genomic locus.

B44. The method of any one of embodiments B1 to B43, wherein the plurality of linked genomic loci in the haplotype group comprises about 10 linked genomic loci to about 1000 linked genomic loci.

B45. The method of any one of embodiments B1 to B43, wherein the plurality of linked genomic loci in the haplotype group comprises about 5 linked genomic loci to about 50 linked genomic loci.

B46. The method of any one of embodiments B1 to B45, wherein each locus in the plurality of linked genomic loci in the haplotype group is at least about 70 bases away from other loci in the haplotype group.

B47. The method of any one of embodiments B1 to B46, wherein the method comprises prior to (b) filtering sequence reads.

B48. The method of embodiment B47, wherein the sequence reads are filtered by removing sequence reads that align to a reference genome locus that is within 1 to 10 bases of an insertion polymorphism or a deletion polymorphism.

B49. The method of embodiment B48, wherein the sequence reads are filtered by removing sequence reads that align to a reference genome locus that is within 4 bases of an insertion polymorphism or a deletion polymorphism.

B50. The method of any one of embodiments B1 to B49, further comprising prior to (a) sequencing the nucleic acid in the first test sample by a sequencing process, thereby generating sequence reads.

B51. The method of embodiment B50, wherein the sequencing process is a whole genome sequencing process.

B52. The method of embodiment B50, wherein the sequencing process is a genome-wide sequencing process.

B53. The method of any one of embodiments B50 to B52, wherein the sequencing process is a non-targeted sequencing process.

B54. The method of any one of embodiments B50 to B53, wherein the sequencing process is a massively parallel sequencing process.

B55. The method of any one of embodiments B50 to B54, wherein the sequencing process is performed at about 50-fold coverage.

B56. The method of any one of embodiments B50 to B54, wherein the sequencing process is performed at about 100-fold coverage.

B57. The method of any one of embodiments B50 to B56, further comprising aligning the sequence reads to a reference genome, thereby generating aligned sequence reads.

B58. The method of any one of embodiments B50 to B57, further comprising prior to sequencing the nucleic acid in the first test sample, producing a sequencing library.

B59. The method of embodiment B58, wherein producing a sequencing library comprises generating single-stranded nucleic acid (ssNA) from the nucleic acid in the first test sample.

B60. The method of embodiment B59, wherein producing a sequencing library comprises combining the ssNA with a plurality of scaffold adapter species, or components thereof.

B61. The method of embodiment B60, wherein the scaffold adapter components comprise (i) an oligonucleotide and (ii) a scaffold polynucleotide comprising an ssNA hybridization region and an oligonucleotide hybridization region.

B62. The method of embodiment B61, wherein the ssNA and the plurality of scaffold adapter species, or components thereof, are combined under conditions in which the scaffold polynucleotide is hybridized to (i) an ssNA terminal region and (ii) the oligonucleotide, thereby forming hybridization products in which an end of the oligonucleotide is adjacent to an end of the ssNA terminal region.

B63. The method of embodiment B62, further comprising covalently linking the adjacent ends of the oligonucleotide and the ssNA terminal region, thereby generating covalently linked hybridization products.

B64. The method of any one of embodiments B61 to B63, wherein the ssNA hybridization region in the scaffold polynucleotide of each scaffold adapter species comprises a unique sequence.

B65. The method of any one of embodiments B61 to B64, wherein the ssNA hybridization region in the scaffold polynucleotide of each scaffold adapter species comprises a random sequence.

B66. The method of any one of embodiments B60 to B65, wherein one or both native ends of the ssNA are present when the ssNA is combined with the plurality of scaffold adapter species, or components thereof.

B67. The method of any one of embodiments B1 to B66, wherein the first test sample is a forensic sample.

B68. The method of any one of embodiments B1 to B66, wherein the first test sample is a non-forensic sample.

B69. The method embodiment B67 or B68, wherein the first test sample comprises hair.

B70. The method of embodiment B67 or B68, wherein the first test sample comprises blood.

B71. The method of embodiment B67 or B68, wherein the first test sample comprises semen.

B72. The method of any one of embodiments B1 to B71, wherein the first test sample is from two human subjects.

B73. The method of any one of embodiments B1 to B72, wherein the nucleic acid in the first test sample comprises cell free nucleic acid.

B74. The method of any one of embodiments B1 to B73, wherein the nucleic acid in the first test sample comprises degraded or damaged nucleic acid.

B75. The method of any one of embodiments B1 to B74, wherein the nucleic acid in the first test sample comprises fragmented nucleic acid.

B76. The method of any one of embodiments B1 to B75, wherein the nucleic acid in the first test sample comprises single-stranded nucleic acid, double-stranded nucleic acid, or single-stranded nucleic acid and double-stranded nucleic acid.

B77. The method of any one of embodiments B1 to B76, wherein the sequence reads are generated from single-stranded nucleic acid fragments, double-stranded nucleic acid fragments, or single-stranded nucleic acid fragments and double-stranded nucleic acid fragments, from the first test sample.

B78. The method of any one of embodiments B1 to B77, wherein the one or more or all of (a), (b), (c), (d), (e), and (f) are performed by a computer.

C1. A method for generating a genotype for a target genomic locus for an unknown subject, comprising:
a) for a test sample comprising a mixture of nucleic acid from two or more unknown subjects, obtaining sequence reads aligned to a reference genome;
b) from the sequence reads, quantifying a linked reference allele and quantifying a linked alternative allele, thereby generating allele quantifications for a linked genomic locus;
c) estimating the fraction of nucleic acid from a first unknown subject in the mixture;
d) for the first unknown subject and a second unknown subject, generating a combined genotype likelihood for the linked genomic locus given the fraction estimated in (c) and according to the allele quantifications in (b);
e) for the first unknown subject, generating a set of genotype likelihoods for a target reference allele and a target alternative allele at the target genomic locus based on the combined genotype likelihood for the linked genomic locus generated in (d); and
f) for the first unknown subject, generating a genotype at the target genomic locus based on the set of genotype likelihoods generated in (e).

C2. The method of embodiment C1, comprising any one or more of the features of embodiments A2 to A87.

D1. A method for generating a genotype for a target genomic locus for an unknown subject, comprising:
a) for a first test sample comprising a mixture of nucleic acid from two or more unknown subjects, obtaining sequence reads aligned to a reference genome;
b) for a haplotype group comprising a target genomic locus and a plurality of linked genomic loci, quantifying a linked reference allele and quantifying a linked alternative allele for each linked genomic locus in the group according to the sequence reads generated in (a), thereby generating allele quantifications for each linked genomic locus in the haplotype group;
c) estimating the fraction of nucleic acid from a first unknown subject in the mixture;
d) for the first unknown subject and a second unknown subject, generating combined genotype likelihoods for the plurality of linked genomic loci in the haplotype group given the fraction estimated in (c) and according to the allele quantifications in (b);
e) generating a haplotype pair likelihood set for the haplotype group according to the combined genotype likelihoods generated in (d); and
f) for the first unknown subject, generating a genotype at the target genomic locus based on the haplotype pair likelihood set generated in (e).

D2. The method of embodiment D1, comprising any one or more of the features of embodiments B2 to B78.

E1. A method for generating a genotype for a target genomic locus for an unknown subject, comprising:
a) for a plurality of test samples each comprising a mixture of nucleic acid from two or more unknown subjects, obtaining sequence reads aligned to a reference genome;
b) from the sequence reads, and for each test sample, quantifying a linked reference allele and quantifying a linked alternative allele, thereby generating allele quantifications for a linked genomic locus;
c) for each test sample, estimating the fraction of nucleic acid from a first unknown subject in the mixture;
d) for each test sample, and for the first unknown subject and at least one further unknown subject, generating a combined genotype likelihood for the linked genomic locus given the fraction estimated in (c) and according to the allele quantifications in (b);
e) for the first unknown subject, generating a set of genotype likelihoods for a target reference allele and a target alternative allele at the target genomic locus based on the combined genotype likelihood for the linked genomic locus generated in (d); and
f) for the first unknown subject, generating a genotype at the target genomic locus based on the set of genotype likelihoods generated in (e).

E2. The method of embodiment E1, wherein the combined genotype likelihood is generated in (d) according to the following:

$$\begin{matrix}L ( A ; X ) = log P ( X \vert A ) \\ = log {\sum }_{G} P ( X \vert A , G ) P ( G ) \\ = log {\prod }_{i = 1}^{n} {\prod }_{j = 1}^{p} {\prod }_{k = 1}^{s} {\sum }_{{G}_{ij}} P ( {X}_{ik} \vert {A}_{jk} , {G}_{ij} ) P ( {G}_{ij} ) \\ = {\prod }_{i = 1}^{n} {\prod }_{j = 1}^{p} {\prod }_{k = 1}^{s} log {\sum }_{{G}_{ij}} P ( {X}_{ik} \vert {A}_{jk} , {G}_{ij} ) P ( {G}_{ij} )\end{matrix}$$

wherein p is the number of subjects, s the number of test samples, Aⱼₖ is an estimated fraction of subject j in test sample k, Xᵢₖ is sequence read data for locus i in sample k, and Gᵢⱼ is a genotype at locus i for individual j.

E3. The method of embodiment E2, wherein P(Xᵢₖ|Aⱼₖ, Gᵢⱼ) is a binomial distribution, with a binomial frequency parameter Σⱼ₌₁^{p}AⱼₖGᵢⱼ/(2p).

E4. The method of embodiment E3, wherein Σⱼ₌₁^{p}AⱼₖGᵢⱼ/(2p) is an expected fraction of alternative alleles, using a weighted average of subjects' genotypes at locus i, according to the estimated fraction Aⱼₖ of subject j in test sample k.

E5. The method of any one of embodiments E1 to E4, comprising any one or more of the features of embodiments A2 to A87.

F1. A method for generating a genotype for a target genomic locus for an unknown subject, comprising:
a) for a plurality of test samples each comprising a mixture of nucleic acid from two or more unknown subjects, obtaining sequence reads aligned to a reference genome;
b) for each test sample, and for a haplotype group comprising a target genomic locus and a plurality of linked genomic loci, quantifying a linked reference allele and quantifying a linked alternative allele for each linked genomic locus in the group according to the sequence reads generated in (a), thereby generating allele quantifications for each linked genomic locus in the haplotype group;
c) for each test sample, estimating the fraction of nucleic acid from a first unknown subject in the mixture;
d) for each test sample, and for the first unknown subject and at least one further unknown subject, generating combined genotype likelihoods for the plurality of linked genomic loci in the haplotype group given the fraction estimated in (c) and according to the allele quantifications in (b);
e) generating a haplotype pair likelihood set for the haplotype group according to the combined genotype likelihoods generated in (d); and
f) for the first unknown subject, generating a genotype at the target genomic locus based on the haplotype pair likelihood set generated in (e).

F2. The method of embodiment F1, wherein each combined genotype likelihood is generated in (d) according to the following:

$$\begin{matrix}L ( A ; X ) = log P ( X \vert A ) \\ = log {\sum }_{G} P ( X \vert A , G ) P ( G ) \\ = log {\prod }_{i = 1}^{n} {\prod }_{j = 1}^{p} {\prod }_{k = 1}^{s} {\sum }_{{G}_{ij}} P ( {X}_{ik} \vert {A}_{jk} , {G}_{ij} ) P ( {G}_{ij} ) \\ = {\prod }_{i = 1}^{n} {\prod }_{j = 1}^{p} {\prod }_{k = 1}^{s} log {\sum }_{{G}_{ij}} P ( {X}_{ik} \vert {A}_{jk} , {G}_{ij} ) P ( {G}_{ij} )\end{matrix}$$

wherein p is the number of subjects, s the number of test samples, Aⱼₖ is an estimated fraction of subject j in test sample k, Xᵢₖ is sequence read data for locus i in sample k, and Gᵢⱼ is a genotype at locus i for individual j.

F3. The method of embodiment F2, wherein P(Xᵢₖ|Aⱼₖ, Gᵢⱼ) is a binomial distribution, with a binomial frequency parameter Eⱼ₌₁^{p}AⱼₖGᵢⱼ/(2p).

F4. The method of embodiment F3, wherein Σⱼ₌₁^{p}AⱼₖGᵢⱼ/(2p) is an expected fraction of alternative alleles, using a weighted average of subjects' genotypes at locus i, according to the estimated fraction Aⱼₖ of subject j in test sample k.

F5. The method of any one of embodiments F1 to F4, comprising any one or more of the features of embodiments B2 to B78.

G1. A method for generating a plurality of genotypes for a plurality of target genomic loci for an unknown subject, comprising:
a) obtaining, for a first test sample comprising a mixture of nucleic acid from a first subject and a second subject, sequence reads aligned to a reference genome;
b) from the sequence reads for the first test sample, quantifying a plurality of linked reference alleles, quantifying a plurality of linked alternative alleles, quantifying a plurality of target reference alleles, and quantifying a plurality of target alternative alleles, thereby generating a plurality of allele quantifications for a plurality of linked genomic loci and a plurality of allele quantifications for the plurality of target genomic loci, wherein i) the plurality of linked genomic loci comprises loci within about 10 kilobases upstream and about 10 kilobases downstream of a target genomic locus, and ii) a plurality of target genomic loci comprises at least 100,000 loci;
c) estimating a fraction of nucleic acid from the first subject in the mixture;
d) for the first subject, generating a plurality of genotype likelihoods for the plurality of linked genomic loci and the plurality of target genomic loci given the fraction estimated in (c) and according to the plurality of allele quantifications in (b);
e) for the first subject, generating a plurality of genotype likelihoods for a target reference allele and a target alternative allele at each of the plurality of target genomic locus based on the plurality of linked genomic locus genotype likelihoods and the plurality of target genomic locus genotype likelihoods generated in (d); and
f) for the first subject, generating a plurality of genotypes each corresponding to a respective target gnomic locus of the plurality of target genomic loci based on each set of genotype likelihoods generated in (e).

G2. The method of embodiment G1, further comprising obtaining, for a second test sample comprising nucleic acid from the second subject and comprising no nucleic acid from the first subject, sequence reads aligned to the reference genome.

G3. The method of embodiment G1, further comprising obtaining, for a second test sample, genotypes for the plurality of linked genomic loci and the plurality of target genomic loci for the second subject.

G4. The method of any one of embodiments G1 to G3, wherein estimating the fraction of nucleic acid from the first subject comprises using an expectation maximization (EM) algorithm to iteratively estimate the fraction of nucleic acid from the first subject.

G5. The method of any one of embodiments G1 to G3, wherein estimating the fraction of nucleic acid from the first subject comprises using a Dirichlet process to estimate the fraction of nucleic acid from the first subject.

G6. The method of any one of embodiments G1 to G5, wherein a genotype likelihood of the set of genotype likelihoods is for a locus where the second subject has an alternative allele.

G7. The method of embodiment G6, wherein the second subject is homozygous for a linked alternative allele at the locus.

G8. The method of embodiment G6, wherein the second subject is heterozygous for a linked alternative allele and a linked reference allele at the locus.

G9. The method of any one of embodiments G1 to G8, wherein a genotype of the plurality of genotypes is a single nucleotide polymorphism (SNP).

G10. The method of any one of embodiments G1 to G8, wherein a genotype of the plurality of genotypes is an unphased genotype.

G11. The method of any one of embodiments G1 to G10, further comprising sequencing nucleic acid in the first test sample using a sequencing process, thereby generating the sequence reads.

G12. The method of embodiment G1, wherein generating the plurality of genotype likelihoods comprises computing a probability

$$P ( D \vert T , θ ) = {\prod }_{i} {\sum }_{{L}_{i} ∈ 0 , 1 , 2} P ( {D}_{i} \vert {L}_{i} , {Y}_{i} , θ ) P ( {L}_{i} \vert T )$$

where
D is sequence read data from the mixture,
θ is the fraction of nucleic acid from the first subject,
i is a linked locus to T,
Dᵢ is sequence read data from the mixture at locus i,
Lᵢ is a genotype of the unknown subject at locus i, and
Yᵢ is a genotype of the second subject at locus i.

G13. The method of embodiment G1, further comprising generating a haplotype pair likelihood for a haplotype groupd based on at least one genotype likelihood of the plurality of genotype likelihoods based on a probability

$$P ( {H}_{p} ) = {\prod }_{i} P ( {X}_{i} \vert {Y}_{i} , {Z}_{i} = G ( {H}_{p} , i ) , θ ) \qquad ( 41 )$$

where:
P(Hₚ) is the likelihood of a given haplotype pair,
Xᵢ is sequence read data generated in (a),
Yᵢ is the second subject's genotype,
Zᵢ is the first subject's genotype,
i is a locus,
θ is the fraction of nucleic acid from the unknown subject in the mixture estimated in (c), and
G(Hₚ, i) is the genotype of the haplotype pair at locus i.

G14. The method of embodiment G1, further comprising generating a probability that the first subject is homozygous reference, heterozygous, or homozygous alternative at a target genomic locus of the plurality of genomic loci.

G15. The method of embodiment G14, wherein generating a probability that the first subject is homozygous reference, heterozygous, or homozygous alternative comprises summing probabilities of haplotypes that are compatible with one or more genotypes at the target genomic locus.

G16. The method of embodiment G1, further comprising:
generating a combined likelihood for a linked genomic locus of the plurality of linked genomic loci based on the estimated fraction of nucleic acid from the first subject in the mixture.

G17. The method of embodiment G16, wherein generating the combinated likelihood comprises calculating a likelihood

$$L ( A ; X ) = {\prod }_{i = 1}^{n} {\prod }_{j = 1}^{p} {\prod }_{k = 1}^{s} log {\sum }_{{G}_{ij}} P ( {X}_{ik} \vert {A}_{jk} , {G}_{ij} ) P ( {G}_{ij} )$$

where p is a number of subjects, s a number of test samples, Aⱼₖ is an estimated fraction of subject j in test sample k, Xᵢₖ is sequence read data for locus i in sample k, and Gᵢⱼ is a genotype at locus i for individual j.

G18. The method of any one of embodiments G1 to G17, wherein the first subject is an unknown subject and the second subject is a known subject.

G19. The method of any one of embodiments G1 to G17, wherein the first subject is an unknown subject and the second subject is an unknown subject.

G20. The method of any one of embodiments G1 to G19, wherein the first subject and the second subject are members of the same species.

G21. The method of any one of embodiments G1 to G20, further comprising:
querying a database using the plurality of genotypes; and
identifying the first subject based on results of the database query.

G22. A method for generating a plurality of genotypes for a plurality of target genomic loci for an unknown subject, comprising:
receiving aligned sequence reads generated from a sample comprising a mixture of genetic material from a plurality of subjects;
generating information indicative of counts of reads in the aligned sequence reads corresponding to various alleles;
estimating a fraction of genetic material in the sample from a first subject of the plurality of subjects;
generating a plurality of genotype likelihoods for the first subject based on the fraction of genetic material in the sample from a first subject and the information indicative of counts of reads; and
generating a plurality of genotypes for the first subject based on the plurality of genotype likelihoods.

G23. The method of embodiment G22, further comprising obtaining, for a second test sample comprising nucleic acid from a second subject and comprising no nucleic acid from the first subject, sequence reads aligned to a reference genome.

G24. The method of embodiment G22, further comprising obtaining, for a second test sample, genotypes for a plurality of linked genomic loci and a plurality of target genomic loci for a second subject.

G25. The method of any one of embodiments G22 to G24, wherein estimating the fraction of genetic material in the sample from the first subject comprises using an expectation maximization (EM) algorithm to iteratively estimate the fraction of genetic material in the sample from the first subject.

G26. The method of any one of embodiments G22 to G24, wherein estimating the fraction of the fraction of genetic material in the sample from the first subject comprises using a Dirichlet process to estimate the fraction of genetic material in the sample from the first subject.

G27. The method of any one of embodiments G22 to G26, wherein a genotype likelihood of the plurality of genotype likelihoods is for a locus where the known subject has an alternative allele.

G28. The method of embodiment G27, wherein a second subject is homozygous for a linked alternative allele at the locus.

G29. The method of embodiment G27, wherein a second subject is heterozygousfor a linked alternative allele and a linked reference allele at the locus.

G30. The method of any one of embodiments G22 to G29, wherein a genotype of the plurality of genotypes is a single nucleotide polymorphism (SNP).

G31. The method of any one of embodiments G22 to G29, wherein a genotype of the plurality of genotypes is an unphased genotype.

G32. The method of any one of embodiments G22 to G31, further comprising sequencing nucleic acid in the sample using a sequencing process, thereby generating the sequence reads.

G33. The method of embodiment G22, wherein generating the plurality of genotype likelihoods comprises computing a probability

$$P ( D \vert T , θ ) = {\prod }_{i} {\sum }_{{L}_{i} ∈ 0 , 1 , 2} P ( {D}_{i} \vert {L}_{i} , {Y}_{i} , θ ) P ( {L}_{i} \vert T )$$

where
D is sequence read data from the mixture,
θ is the the fraction of genetic material in the sample from the first subject,
i is a linked locus to T,
Dᵢ is sequence read data from the mixture at locus i,
Lᵢ is a genotype of the first subject at locus i, and
Yᵢ is a genotype of the second subject at locus i.

G34. The method of embodiment G22, further comprising generating a haplotype pair likelihood for a haplotype groupd based on at least one genotype likelihood of the plurality of genotype likelihoods based on a probability

$$P ( {H}_{p} ) = {\prod }_{i} P ( {X}_{i} \vert {Y}_{i} , {Z}_{i} = G ( {H}_{p} , i ) , θ )$$

where:
P(Hₚ) is the likelihood of a given haplotype pair,
Xᵢ is sequence read data generated in (a),
Yᵢ is the second subject's genotype,
Zᵢ is the first subject's genotype,
i is a locus,
θ is the the fraction of genetic material in the sample from the first subject in the mixture estimated in (c), and
G(Hₚ, i) is the genotype of the haplotype pair at locus i.

G35. The method of embodiment G22, further comprising generating a probability that the first subject is homozygous reference, heterozygous, or homozygous alternative at a target genomic locus of the plurality of genomic loci.

G36. The method of embodiment G35, wherein generating a probability that the first subject is homozygous reference, heterozygous, or homozygous alternative comprises summing probabilities of haplotypes that are compatible with one or more genotypes at the target genomic locus.

G37. The method of embodiment G22, further comprising:
generating a combined likelihood for a linked genomic locus of the plurality of linked genomic loci based on the estimated the fraction of genetic material in the sample from the first subject in the mixture.

G38. The method of embodiment G37, wherein generating the combinated likelihood comprises calculating a likelihood

$$L ( A ; X ) = {\prod }_{i = 1}^{n} {\prod }_{j = 1}^{p} {\prod }_{k = 1}^{s} log {\sum }_{{G}_{ij}} P ( {X}_{ik} \vert {A}_{jk} , {G}_{ij} ) P ( {G}_{ij} )$$

where p is a number of subjects, s a number of test samples, Aⱼₖ is an estimated fraction of subject j in test sample k, Xᵢₖ is sequence read data for locus i in sample k, and G; is a genotype at locus i for individual j.

G39. The method of any one of embodiments G22 to G38, wherein the first subject is an unknown subject and a second subject is a known subject.

G40. The method of any one of embodiments G22 to G38, wherein the first subject is an unknown subject and a second subject is an unknown subject.

G41. The method of any one of embodiments G22 to G40, wherein the first subject and a second subject are members of the same species.

G42. The method of any one of embodiments 22 to 41, further comprising:
querying a database using the plurality of genotypes; and
identifying the first subject based on results of the database query.

G43. A system comprising: a processor; and a memory communicatively coupled to the at least one processor, wherein the processor and memory are configured to: perform a method of any of embodiments G1 to G42.

G44. A non-transitory computer-readable medium storing computer-executable code, comprising code for causing a computer to cause a processor to: perform a method of any of embodiments G1 to G42.

The entirety of each patent, patent application, publication and document referenced herein is incorporated by reference. Citation of patents, patent applications, publications and documents is not an admission that any of the foregoing is pertinent prior art, nor does it constitute any admission as to the contents or date of these publications or documents. Their citation is not an indication of a search for relevant disclosures. All statements regarding the date(s) or contents of the documents is based on available information and is not an admission as to their accuracy or correctness.

The technology has been described with reference to specific implementations. The terms and expressions that have been utilized herein to describe the technology are descriptive and not necessarily limiting. Certain modifications made to the disclosed implementations can be considered within the scope of the technology. Certain aspects of the disclosed implementations suitably may be practiced in the presence or absence of certain elements not specifically disclosed herein.

Each of the terms “comprising,” “consisting essentially of,” and “consisting of” may be replaced with either of the other two terms. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%; e.g., a weight of “about 100 grams” can include a weight between 90 grams and 110 grams). Use of the term “about” at the beginning of a listing of values modifies each of the values (e.g., “about 1, 2 and 3” refers to “about 1, about 2 and about 3”). When a listing of values is described the listing includes all intermediate values and all fractional values thereof (e.g., the listing of values “80%, 85% or 90%” includes the intermediate value 86% and the fractional value 86.4%).

Certain implementations of the technology are set forth in the claim(s) that follow(s).

1. A method for generating a plurality of genotypes for a plurality of target genomic loci for an unknown subject, comprising:
a) obtaining, for a first test sample comprising a mixture of nucleic acid from a first subject and a second subject, sequence reads aligned to a reference genome;
b) from the sequence reads for the first test sample, quantifying a plurality of linked reference alleles, quantifying a plurality of linked alternative alleles, quantifying a plurality of target reference alleles, and quantifying a plurality of target alternative alleles, thereby generating a plurality of allele quantifications for a plurality of linked genomic loci and a plurality of allele quantifications for the plurality of target genomic loci, wherein i) the plurality of linked genomic loci comprises loci within about 10 kilobases upstream and about 10 kilobases downstream of a target genomic locus, and ii) a plurality of target genomic loci comprises at least 100,000 loci;
c) estimating a fraction of nucleic acid from the first subject in the mixture;
d) for the first subject, generating a plurality of genotype likelihoods for the plurality of linked genomic loci and the plurality of target genomic loci given the fraction estimated in (c) and according to the plurality of allele quantifications in (b);
e) for the first subject, generating a plurality of genotype likelihoods for a target reference allele and a target alternative allele at each of the plurality of target genomic locus based on the plurality of linked genomic locus genotype likelihoods and the plurality of target genomic locus genotype likelihoods generated in (d); and
f) for the first subject, generating a plurality of genotypes each corresponding to a respective target gnomic locus of the plurality of target genomic loci based on each set of genotype likelihoods generated in (e).

2. The method of claim 1, further comprising obtaining, for a second test sample comprising nucleic acid from the second subject and comprising no nucleic acid from the first subject, sequence reads aligned to the reference genome.

3. The method of claim 1, further comprising obtaining, for a second test sample, genotypes for the plurality of linked genomic loci and the plurality of target genomic loci for the second subject.

4. The method of claim 1, wherein estimating the fraction of nucleic acid from the first subject comprises using an expectation maximization (EM) algorithm to iteratively estimate the fraction of nucleic acid from the first subject.

5. The method of claim 1, wherein estimating the fraction of nucleic acid from the first subject comprises using a Dirichlet process to estimate the fraction of nucleic acid from the first subject.

6. The method of claim 1, wherein a genotype likelihood of the set of genotype likelihoods is for a locus where the second subject has an alternative allele.

7. The method of claim 6, wherein the second subject is homozygous for a linked alternative allele at the locus.

8. The method of claim 6, wherein the second subject is heterozygous for a linked alternative allele and a linked reference allele at the locus.

9. The method of claim 1, wherein a genotype of the plurality of genotypes is a single nucleotide polymorphism (SNP).

10. The method of claim 1, wherein a genotype of the plurality of genotypes is an unphased genotype.

11. The method of claim 1, further comprising sequencing nucleic acid in the first test sample using a sequencing process, thereby generating the sequence reads.

12. (canceled)

13. (canceled)

14. The method of claim 1, further comprising generating a probability that the first subject is homozygous reference, heterozygous, or homozygous alternative at a target genomic locus of the plurality of genomic loci.

15. The method of claim 14, wherein generating a probability that the first subject is homozygous reference, heterozygous, or homozygous alternative comprises summing probabilities of haplotypes that are compatible with one or more genotypes at the target genomic locus.

16. The method of claim 1, further comprising:
generating a combined likelihood for a linked genomic locus of the plurality of linked genomic loci based on the estimated fraction of nucleic acid from the first subject in the mixture.

17. (canceled)

18. The method of claim 1, wherein the first subject is an unknown subject and the second subject is a known subject.

19. The method of claim 1, wherein the first subject is an unknown subject and the second subject is an unknown subject.

20. The method of claim 1, wherein the first subject and the second subject are members of the same species.

21. The method of claim 1, further comprising:
querying a database using the plurality of genotypes; and
identifying the first subject based on results of the database query.

22-42. (canceled)

43. A system comprising:
a processor configured to:
a) obtain, for a first test sample comprising a mixture of nucleic acid from a first subject and a second subject, sequence reads aligned to a reference genome;
b) from the sequence reads for the first test sample, quantify a plurality of linked reference alleles, quantifying a plurality of linked alternative alleles, quantifying a plurality of target reference alleles, and quantifying a plurality of target alternative alleles, thereby generating a plurality of allele quantifications for a plurality of linked genomic loci and a plurality of allele quantifications for the plurality of target genomic loci, wherein i) the plurality of linked genomic loci comprises loci within about 10 kilobases upstream and about 10 kilobases downstream of a target genomic locus, and ii) a plurality of target genomic loci comprises at least 100,000 loci;
c) estimate a fraction of nucleic acid from the first subject in the mixture;
d) for the first subject, generate a plurality of genotype likelihoods for the plurality of linked genomic loci and the plurality of target genomic loci given the fraction estimated in (c) and according to the plurality of allele quantifications in (b);
e) for the first subject, generate a plurality of genotype likelihoods for a target reference allele and a target alternative allele at each of the plurality of target genomic locus based on the plurality of linked genomic locus genotype likelihoods and the plurality of target genomic locus genotype likelihoods generated in (d); and
f) for the first subject, generate a plurality of genotypes each corresponding to a respective target gnomic locus of the plurality of target genomic loci based on each set of genotype likelihoods generated in (e).

44. (canceled)
